(19) United States
(12) Patent Application Publication
(10) Pub. No.: US 2016/0115467 A1
(43) Pub. Date: Apr. 28, 2016

(54) CHIMERIC FVII-XTEN MOLECULES AND USES THEREOF

(71) Applicant: BIOGEN MA INC., Cambridge, MA (US)

(72) Inventor: Joe SALAS, Wayland, MA (US)

(73) Assignee: Biogen MA Inc., Cambridge, MA (US)

(21) Appl. No.: 14/894,101

(22) PCT Filed: May 30, 2014
(86) PCT No.: PCT/US14/40370

§ 371 Date: Nov. 25, 2015

Related U.S. Application Data
(60) Provisional application No. 61/829,878, filed on May 31, 2013.
(60) Provisional application No. 61/883,707, filed on Sep. 27, 2013.
(60) Provisional application No. 61/901,954, filed on Nov. 8, 2013.
(60) Provisional application No. 61/988,105, filed on May 2, 2014.

(51) Int. Cl. C12N 9/64; C07K 14/435; C07K 16/28

(52) U.S. Cl. CPC C12N 9/6437; C07K 16/2848; C07K 14/435; C07K 2317/76; C07K 2317/565; C07K 2317/622; C07K 2317/626; C07K 2317/54; C07K 2317/55; C07K 2317/24; C07K 2319/74; C12Y 304/21021

(57) ABSTRACT

The present invention provides chimeric FVII molecules comprising FVII, an XTEN polypeptide, and an antibody C and antigen-binding molecules thereof which specifically bind the α and/or β subunits of the non-active form of the GPIIb/IIIIa receptor. The antibodies and antigen-binding molecules can be genetically fused and/or conjugated to heterologous moieties, e.g., half-life extending moiety. The invention also includes methods of producing and using the chimeric molecules.

### BACKGROUND OF THE INVENTION

Clotting factors have been administered to patients to improve hemostasis for some time. The advent of recombinant DNA technology has significantly improved treatment for patients with clotting disorders, allowing for the development of safe and consistent protein therapeutics. For example, recombinant activated Factor VII (“FVII”) has become widely used for the treatment of major bleeding, such as that which occurs in patients having hemophilia A or B, deficiency of coagulation Factor XI, FVII, defective platelet function, thrombocytopenia, or von Willebrand's disease.

Although such recombinant molecules are effective, there is a need for improved versions which localize the therapeutic to sites of coagulation, have improved pharmacokinetic properties, have improved manufacturability, have reduced thrombogenicity, or have enhanced activity, or more than one of these characteristics.

Treatment of hemophilia by replacement therapy is targeting restoration of clotting activity. There are plasma-derived and recombinant clotting factor products available to treat bleeding episodes on-demand or to prevent bleeding episodes from occurring by treating prophylactically. Based on the half-life of these products, treatment regimens require frequent intravenous administration. Such frequent administration is painful and inconvenient. Strategies to extend the half-life of clotting factors include pegylation (Rostin J, et al., Bioconj. Chem. 2000; 11:387-96), glycopegylation (Stennicke H R, et al., Thromb. Haemost. 2008; 100:920-8), formulation with pegylated liposomes (Spira J, et al., Blood 2006; 108:3668-3673, Pan J, et al., Blood 2009; 114:2802-2811) and conjugation with albumin (Schulte S., Thromb. Res. 2008; 122 Suppl 4:S14-9).

Recombinant activated FVII (rFVIIa; NOVOSEVEN®) is used to treat bleeding episodes in (i) hemophilia patients with neutralizing antibodies against FVIII or FIX (inhibitors), (ii) patients with FVII deficiency, or (iii) patients with hemophilia A or B with inhibitors undergoing surgical procedures. However, NOVOSEVEN® displays poor efficacy. Repeated doses of FVIIa at high concentration are often required to control a bleed, due to its low affinity for activated platelets, short half-life, and poor enzymatic activity in the absence of tissue factor. Accordingly, there is an unmet medical need for better treatment and prevention options for hemophilia patients with FVIII and FIX inhibitors and/or with FVII deficiency.

### BRIEF SUMMARY OF THE INVENTION

The present invention discloses a chimeric FVII molecule comprising FVII, an XTEN polypeptide, and an anti-GPIIb/IIIa antibody or antigen-binding molecule thereof, wherein the anti-GPIIb/IIIa antibody or antigen-binding molecule thereof exhibits one or more of the following characteristics:

(a) the anti-GPIIb/IIIa antibody or antigen-binding molecule thereof specifically binds to the same GPIIb/IIIa epitope as an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;

(b) the anti-GPIIb/IIIa antibody or antigen-binding molecule thereof competitively inhibits GPIIb/IIIa binding to an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4; or

(c) the anti-GPIIb/IIIa antibody or antigen-binding molecule thereof comprises at least one, at least two, at least three, at least four, at least five, or at least six complementarity determining regions (CDR) or variants thereof selected from the CDRs of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4.

For example, the anti-GPIIb/IIIa antibody or antigen-binding molecule thereof can comprise six CDRs or variants thereof of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4.

In one aspect, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 25, 31, 37, 43 or 111;

(ii) a variable heavy chain CDR-2 (VH-CDR2) sequence at least about 60%, 70%, 80%, 90%, 95%, or 100% identical to any one of SEQ ID NOS:26, 32, 38, 44, or 112;

(iii) a variable heavy chain CDR-3 (VH-CDR3) sequence at least about 60%, 70%, 80%, 90%, 95%, or 100% identical to any one of SEQ ID NOS: 27, 33, 39, 45, or 113;

(iv) a variable light chain CDR-1 (VL-CDR1) sequence at least about 60%, 70%, 80%, 90%, 95%, or 100% identical to any one of SEQ ID NOS: 28, 34, 40, 117, or 114;

(v) a variable light chain CDR-2 (VL-CDR2) sequence at least about 60%, 70%, 80%, 90%, 95%, or 100% identical to any one of SEQ ID NOS: 29, 35, 41, 118, or 115; and,

(vi) a variable light chain CDR-3 (VL-CDR3) sequence at least about 60, 70, 80, 90, or 95% identical to any one of SEQ ID NOS: 30, 36, 42, 119, or 116.

In another aspect, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises:

(i) a VH-CDR1 comprising the consensus sequence X₁YAMS wherein X₁ represents amino acid residues Thr (T), Ser (S), or Ala (A);

(ii) a VH-CDR2 comprising the consensus sequence SIX₂X₃GX₄X₅TYX₆X₇DSVKX₈ wherein X₂ represents amino acid residues Ser (S) or Asn (N), X₃ represents amino acid residues Ser (S) or Gly (G), X₄ represents amino acid residues Ser (S) or Gly (G), X₅ represents amino acid residues Ser (S), Asn (N), or Thr (T), X₆ represents amino acid residues Tyr (Y) or Phe (F), X₇ represents amino acid residues Leu (L) or Pro (P), and X₈ represents amino acids Gly (G) or Arg (R);

(iii) a VH-CDR3 comprising the consensus sequence GGDYGYAX₉DY, wherein X₉ represents amino acid residues Leu (L) or Met (M);

(iv) a VL-CDR1 comprising the sequence RASSSVNYMY (SEQ ID NO: 28);

(v) a VL-CDR2 comprising the sequence YTSNLAP (SEQ ID NO: 29); and,

(vi) a VL-CDR3 comprising the sequence QQFSSSPWT (SEQ ID NO: 30).

In one embodiment, the anti-GPIIb/IIIa antibody or antigen-binding molecule thereof useful for the chimeric molecule comprises:

(i) a VH-CDR1 sequence selected from the group consisting of SEQ ID NOS: 25, 31, 37, 43, and 111;

(ii) a VH-CDR2 sequence selected from the group consisting of SEQ ID NOS: 26, 32, 38, 44, and 112;

(iii) a VH-CDR3 sequence selected from the group consisting of SEQ ID NOS: 27, 33, 39, 45, and 113;

(iv) a VL-CDR1 sequence selected from the group consisting of SEQ ID NOS: 28, 34, 40, 117, and 114;

(v) a VL-CDR2 sequence selected from the group consisting of SEQ ID NOS: 29, 35, 41, 118, and 115; and,

(vi) a VL-CDR3 sequence selected from the group consisting of SEQ ID NOS: 30, 36, 42, 119, and 116.

In another embodiment, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH comprising an amino acid sequence at least about 80%, 85%, 90%, 95%, or 100% identical to any one of SEQ ID NOS: 1, 3, 5, 7, or 97 and a VL comprising an amino acid sequence at least about 80%, 85%, 90%, 95%, or 100% identical to any one of SEQ ID NOS: 2, 4, 6, 99, or 98. In other embodiments, the anti-GPIIb/IIIa antibody or antigen-binding molecule thereof comprises a VH comprising the amino acid sequence of SEQ ID NO: 1 and a VL comprising the amino acid sequence of SEQ ID NO: 2 (34D10 antibody). In still other embodiments, the anti-GPIIb/IIIa antibody or antigen-binding molecule thereof comprises a VH comprising the amino acid sequence of SEQ ID NO: 3 and a VL comprising the amino acid sequence of SEQ ID NO: 4 (2A2 antibody). In some embodiments, the anti-GPIIb/IIIa antibody or antigen-binding molecule thereof comprises a VH comprising the amino acid sequence of SEQ ID NO: 5 and a VL comprising the amino acid sequence of SEQ ID NO: 6 (36A8 antibody). In certain embodiments, the anti-GPIIb/IIIa antibody or antigen-binding molecule thereof comprises a VH comprising the amino acid sequence of SEQ ID NO: 7 and a VL comprising the amino acid sequence of SEQ ID NO: 99 (4B11 antibody). In other embodiments, the anti-GPIIb/IIIa antibody or antigen-binding molecule thereof comprises a VH comprising the amino acid sequence of SEQ ID NO: 97 and a VL comprising the amino acid sequence of SEQ ID NO: 98 (35D1 antibody). The anti-GPIIb/IIIa antibody or antigen-binding molecule thereof can bind to an epitope located in the extracellular domain of the alpha subunit of GPIIb/IIIa or the extracellular domain of the GPIIb/IIIa complex. In certain embodiments, the anti-GPIIb/IIIa antibody or antigen-binding molecule thereof does not compete with fibrinogen for binding to GPIIb/IIIa.

In some aspects, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 46, 52, 120, or 126;

(ii) a variable heavy chain CDR-2 (VH-CDR2) sequence at least about 60%, 70%, 80%, 90%, 95%, or 100% identical to any one of SEQ ID NOS: 47, 53, 121, or 127;

(iii) a variable heavy chain CDR-3 (VH-CDR3) sequence at least about 60%, 70%, 80%, 90%, 95%, or 100% identical to any one of SEQ ID NOS: 48, 54, 122, or 128;

(iv) a variable light chain CDR-1 (VL-CDR1) sequence at least about 60%, 70%, 80%, 90%, 95%, or 100% identical to any one of SEQ ID NOS: 49, 55, 123, or 129;

(v) a variable light chain CDR-2 (VL-CDR2) sequence at least about 60%, 70%, 80%, 90%, 95%, or 100% identical to any one of SEQ ID NOS: 50, 56, 124, or 130; and,

(vi) a variable light chain CDR-3 (VL-CDR3) sequence at least about 60%, 70%, 80%, 90%, 95%, or 100% identical to any one of SEQ ID NO: 51, 57, 125, or 131.

In other aspects, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises: a VH comprising an amino acid sequence at least about 80%, 85%, 90%, 95%, or 100% identical to any one of SEQ ID NOS: 8, 10, 100, or 102, and a VL comprising an amino acid sequence at least about 80%, 85%, 90%, 95%, or 100% identical to any one of SEQ ID NOS: 9, 11, 101, or 103. In one embodiment, the anti-GPIIb/IIIa antibody or antigen-binding molecule thereof comprises a VH comprising the amino acid sequence of SEQ ID NO: 8 and a VL comprising the amino acid sequence of SEQ ID NO: 9 (1H6 antibody). In another embodiment, the anti-GPIIb/IIIa antibody or antigen-binding molecule thereof comprises: a VH comprises the amino acid sequence of SEQ ID NO: 10 and a VL comprising the amino acid sequence of SEQ ID NO: 11 (38A8 antibody). In other embodiments, the anti-GPIIb/IIIa antibody or antigen-binding molecule thereof comprises a VH comprising the amino acid sequence of SEQ ID NO: 100 and a VL comprising the amino acid sequence of SEQ ID NO: 101 (38G8 antibody). In some embodiments, the anti-GPIIb/IIIa antibody or antigen-binding molecule thereof comprises a VH comprising the amino acid sequence of SEQ ID NO: 102 and a VL comprising the amino acid sequence of SEQ ID NO: 103 (21F10 antibody). In certain embodiments, the anti-GPIIb/IIIa antibody or antigen-binding molecule thereof binds to an epitope located in the extracellular domain of the alpha subunit of GPIIb/IIIa. In other embodiments, the anti-GPIIb/IIIa antibody or antigen-binding molecule thereof competes with fibrinogen for binding to GPIIb/IIIa.

In certain aspects, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises:

(i) a variable heavy chain CDR-1 (VH-CDR1) sequence at least about 60%, 70%, 80%, 90%, 95%, or 100% identical to SEQ ID NO: 58;

(ii) a variable heavy chain CDR-2 (VH-CDR2) sequence at least about 60%, 70%, 80%, 90%, 95%, or 100% identical to SEQ ID NO: 59;

(iii) a variable heavy chain CDR-3 (VH-CDR3) sequence at least about 60%, 70%, 80%, 90%, 95%, or 100% identical to SEQ ID NO: 60;

(iv) a variable light chain CDR-1 (VL-CDR1) sequence at least about 60%, 70%, 80%, 90%, 95%, or 100% identical to SEQ ID NO: 61;

(v) a variable light chain CDR-2 (VL-CDR2) sequence at least about 60%, 70%, 80%, 90%, 95%, or 100% identical to SEQ ID NO: 62; and,

(vi) a variable light chain CDR-3 (VL-CDR3) sequence at least about 60%, 70%, 80%, 90%, 95%, or 100% identical to SEQ ID NO: 63.

In one embodiment, the anti-GPIIb/IIIa antibody or antigen-binding molecule thereof comprises a VH comprising an amino acid sequence at least about 80%, 85%, 90%, 95%, or 100% identical to SEQ ID NO: 12 and a VL comprising an amino acid sequence at least about 80%, 85%, 90%, 95%, or 100% identical to SEQ ID NO: 13 (18F7 antibody). In another embodiment, the anti-GPIIb/IIIa antibody or antigen-binding molecule thereof binds to an epitope located in the extracellular domain of the alpha subunit of GPIIb/IIIa. In other embodiments, the anti-GPIIb/IIIa antibody or antigen-binding molecule thereof competes with fibrinogen for binding to GPIIb/IIIa.

Also provided is a chimeric molecule comprising FVII, an XTEN polypeptide, and an anti-GPIIb/IIIa antibody or antigen-binding molecule thereof, wherein the anti-GPIIb/IIIa antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 64, 70, or 135;

(ii) a variable heavy chain CDR-2 (VH-CDR2) sequence at least about 60%, 70%, 80%, 90%, 95%, or 100% identical to any one of SEQ ID NOS: 65, 71, or 136;

(iii) a variable heavy chain CDR-3 (VH-CDR3) sequence at least about 60%, 70%, 80%, 90%, 95%, or 100% identical to any one of SEQ ID NOS: 66, 72, or 137;

(iv) a variable light chain CDR-1 (VL-CDR1) sequence at least about 60%, 70%, 80%, 90%, 95%, or 100% identical to any one of SEQ ID NOS: 67, 132, or 138;

(v) a variable light chain CDR-2 (VL-CDR2) sequence at least about 60%, 70%, 80%, 90%, 95%, or 100% identical to any one of SEQ ID NOS: 68, 133, or 139; and,

(vi) a variable light chain CDR-3 (VL-CDR3) sequence at least about 60%, 70%, 80%, 90%, 95%, or 100% identical to any one of SEQ ID NOS: 69, 134, or 140.

In other aspects, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises:

(i) a VH-CDR1 comprising the sequence SYWIE (SEQ ID NO: 64);

(ii) a VH-CDR2 comprising the consensus sequence EILPGX14GX15TKYNX16KFKG (SEQ ID NO:______) wherein X14 represents amino acid residues Ser (S) or Thr (T), X15 represents amino acid residues Ile (I) or Tyr (Y), and X16 represents amino acid residues Asp (D) or Glu (E);

(iii) a VH-CDR3 comprising the sequence LISYYYAMDY (SEQ ID NO: 66);

(iv) a VL-CDR1 comprising the sequence RASQDISNYLN (SEQ ID NO: 67);

(v) a VL-CDR2 comprising the sequence YTSRLHS (SEQ ID NO: 68); and,

(vi) a VL-CDR3 comprising the sequence QQGNTLPPT (SEQ ID NO: 69).

In one embodiment, the anti-GPIIb/IIIa antibody or antigen-binding molecule thereof comprises a VH comprising an amino acid sequence at least about 80%, 85%, 90%, 95%, or 100% identical to any one of SEQ ID NOS: 14, 16, or 105 and a VL comprising an amino acid sequence at least about 80%, 85%, 90%, 95%, or 100% identical to any one of SEQ ID NOS: 15, 104, or 106. In another embodiment, the anti-GPIIb/IIIa antibody or antigen-binding molecule thereof comprises a VH comprising the amino acid sequence of SEQ ID NO: 14 and a VL comprising the amino acid sequence of SEQ ID NO: 15 (12B2 antibody). In other embodiments, the anti-GPIIb/IIIa antibody or antigen-binding molecule thereof comprises a VH comprising the amino acid sequence of SEQ ID NO: 16 and a VL comprising the amino acid sequence of SEQ ID NO: 104 (38F6 antibody). In some embodiments, the anti-GPIIb/IIIa antibody or antigen-binding molecule thereof comprises a VH comprising the amino acid sequence of SEQ ID NO: 105 and a VL comprising the amino acid sequence of SEQ ID NO: 106 (13C1 antibody). In still other embodiments, the anti-GPIIb/IIIa antibody or antigen-binding molecule thereof binds to an epitope located in the extracellular domain of the beta subunit of GPIIb/IIIa. In yet other embodiments, the GPIIb/IIIa antibody or antigen-binding molecule thereof does not compete with fibrinogen for binding to GPIIb/IIIa.

In other aspects, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 73, 76, 79, 85, or 147;

(ii) a variable heavy chain CDR-2 (VH-CDR2) sequence at least about 60%, 70%, 80%, 90%, 95%, or 100% identical to any one of SEQ ID NOS: 74, 77, 80, 86, or 148;

(iii) a variable heavy chain CDR-3 (VH-CDR3) sequence at least about 60%, 70%, 80%, 90%, 95%, or 100% identical to any one of SEQ ID NOS: 75, 78, 81, 87, or 149;

(iv) a variable light chain CDR-1 (VL-CDR1) sequence at least about 60%, 70%, 80%, 90%, 95%, or 100% identical to any one of SEQ ID NOS: 141, 144, 82, 88, or 150;

(v) a variable light chain CDR-2 (VL-CDR2) sequence at least about 60%, 70%, 80%, 90%, 95%, or 100% identical to any one of SEQ ID NOS: 142, 145, 83, 89, or 151; and,

(vi) a variable light chain CDR-3 (VL-CDR3) sequence at least about 60%, 70%, 80%, 90%, 95%, or 100% identical to any one of SEQ ID NO: 143, 146, 84, 90, or 152.

In one embodiment, the anti-GPIIb/IIIa antibody or antigen-binding molecule thereof comprises a VH comprising an amino acid sequence at least 80%, 85%, 90%, 95%, or 100% identical to any one of SEQ ID NOS: 17, 18, 19, 21, or 109 and a VL comprising an amino acid sequence at least 80%, 85%, 90%, 95%, or 100% identical to any one of SEQ ID NOS: 107, 108, 20, 22, or 110. In another embodiment, the anti-GPIIb/IIIa antibody or antigen-binding molecule thereof comprises a VH comprising the amino acid sequence of SEQ ID NO: 17 and a VL comprising the amino acid sequence of SEQ ID NO: 107 (5C4 antibody). In other embodiments, the anti-GPIIb/IIIa antibody or antigen-binding molecule thereof comprises a VH comprising the amino acid sequence of SEQ ID NO: 18 and a VL comprising the amino acid sequence of SEQ ID NO: 108 (23C10 antibody). In still other embodiments, the anti-GPIIb/IIIa antibody or antigen-binding molecule thereof comprises a VH comprising the amino acid sequence of SEQ ID NO: 109 and a VL comprising the amino acid sequence of SEQ ID NO: 110 (37C7 antibody). In yet other embodiments, the anti-GPIIb/IIIa antibody or antigen-binding molecule thereof comprises a VH comprising the amino acid sequence of SEQ ID NO: 19 and a VL comprising the amino acid sequence of SEQ ID NO: 20 (28C2 antibody). In some embodiments, the anti-GPIIb/IIIa antibody or antigen-binding molecule thereof comprises a VH comprising the amino acid sequence of SEQ ID NO: 21 and a VL comprising the amino acid sequence of SEQ ID NO: 22 (9D6 antibody). In certain embodiments, the anti-GPIIb/IIIa antibody or antigen-binding molecule thereof binds to an epitope located in the extracellular domain of the beta subunit of GPIIb/IIIa. In other embodiments, the anti-GPIIb/IIIa antibody or antigen-binding molecule thereof competes with fibrinogen for binding to GPIIb/IIIa.

In some aspects, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises:

(i) a variable heavy chain CDR-1 (VH-CDR1) sequence at least about 60%, 70%, 80%, 90%, 95%, or 100% identical to SEQ ID NO: 91;

(ii) a variable heavy chain CDR-2 (VH-CDR2) sequence at least about 60%, 70%, 80%, 90%, 95%, or 100% identical to SEQ ID NO: 92;

(iii) a variable heavy chain CDR-3 (VH-CDR3) sequence at least about 60%, 70%, 80%, 90%, 95%, or 100% identical to SEQ ID NO: 93;

(iv) a variable light chain CDR-1 (VL-CDR1) sequence at least about 60%, 70%, 80%, 90%, 95%, or 100% identical to SEQ ID NO: 94;

(v) a variable light chain CDR-2 (VL-CDR2) sequence at least about 60%, 70%, 80%, 90%, 95%, or 100% identical to SEQ ID NO: 95; and,

(vi) a variable light chain CDR-3 (VL-CDR3) sequence at least about 60%, 70%, 80%, 90%, 95%, or 100% identical to SEQ ID NO: 96.

In one embodiment, the anti-GPIIb/IIIa antibody or antigen-binding molecule thereof comprises a VH comprising an amino acid sequence at least about 80%, 85%, 90%, 95%, or 100% identical to SEQ ID NO: 23 and a VL comprising an amino acid sequence at least about 80%, 85%, 90%, 95%, or 100% identical to SEQ ID NO: 24 (28F4 antibody). In another embodiment, the anti-GPIIb/IIIa antibody or antigen-binding molecule thereof binds to an epitope located in the extracellular domain of the beta subunit of GPIIb/IIIa.

In other embodiments, the anti-GPIIb/IIIa antibody or antigen-binding molecule thereof comprises: (a) a single chain Fv (“scFv”); (b) a diabody; (c) a minibody; (d) a polypeptide chain of an antibody; (e) F(ab′)2; or (f) F(ab).

In some aspects of the invention, the chimeric molecule further comprises an optional linker between FVII and the XTEN polypeptide, between FVII and the anti-GPIIb/IIIa antibody or antigen-binding molecule thereof, or between the XTEN polypeptide and the anti-GPIIb/IIIa antibody or antigen-binding molecule thereof.

In one embodiment, a chimeric molecule comprises a formula selected from the group consisting of: (i) FVII-(L1)-X-(L2)-Tm; (ii) FVII-(L1)-Tm-(L2)-X; (iii) Tm-(L1)-X-(L2)-FVII; (iv) Tm-(L1)-FVII-(L2)-X; (v) X-(L1)-Tm-(L2)-FVII; and (vi) X-(L1)-FVII-(L2)-Tm; wherein FVII comprises activated FVII (“FVIIa”); X is the XTEN polypeptide; Tm is the anti-GPIIb/IIIa antibody or antigen-binding molecule thereof; L1 is a first optional linker, and L2 is a second optional linker.

In another embodiment, a chimeric molecule comprises a first polypeptide chain and a second polypeptide chain, which are associated with each other,

(a) wherein the first polypeptide chain comprises a light chain of FVII and the XTEN polypeptide and the second polypeptide chain comprises a heavy chain of FVII and the anti-GPIIb/IIIa antibody or antigen-binding molecule thereof;

(b) wherein the first polypeptide chain comprises a light chain of FVII and the anti-GPIIb/IIIa antibody or antigen-binding molecule thereof and the second polypeptide chain comprises a heavy chain of FVII and the XTEN polypeptide;

(c) wherein the first polypeptide chain comprises a light chain of FVII, the XTEN polypeptide, and the anti-GPIIb/IIIa antibody or antigen-binding molecule thereof, in any order, and the second chain comprises a heavy chain of FVII; or

(d) wherein the first polypeptide chain comprises a light chain of FVII and the second chain comprises a heavy chain of FVII, the XTEN polypeptide, and the anti-GPIIb/IIIa antibody or antigen-binding molecule thereof, in any order.

In other embodiments, a chimeric molecule comprises a first polypeptide chain and a second polypeptide chain, which are associated with each other,

(a) wherein the first polypeptide chain comprises the formula of FVII_{L}-X or X-FVII_{L} and the second polypeptide chain comprises the formula of FVII_{H}-Tm or Tm-FVII_{H},

(b) wherein the first polypeptide chain comprise the formula of FVII_{L}-Tm or Tm-FVII_{L} and the second polypeptide chain comprises the formula of FVII_{H}-X or X-FVII_{H};

(c) wherein the first polypeptide chain comprise the formula of FVII_{L} and the second polypeptide chain comprises the formula of FVII_{H}-X-Tm or Tm-X-FVII_{H};

(d) wherein the first polypeptide chain comprise the formula of FVII_{L} and the second polypeptide chain comprises the formula of FVII_{H}-Tm-X or X-Tm-FVII_{H};

(e) wherein the first polypeptide chain comprise the formula of FVII_{L}-Tm-X or X-Tm-FVII_{L} or and the second polypeptide chain comprises the formula of FVII_{H}; or

(f) wherein the first polypeptide chain comprise the formula of FVII_{L}-X-Tm or Tm-X-FVII_{L} and the second polypeptide chain comprises the formula of FVII_{H},

wherein FVII_{H} is a heavy chain of FVII; Tm is an anti-GPIIb/IIIa antibody or antigen-binding molecule thereof; FVII_{L} is a light chain of FVII; and X is an XTEN polypeptide.

In some embodiments, a chimeric molecule comprises a formula selected from the group consisting of:

(a) X-FVII_{L}:FVII_{H}-Tm;

(b) Tm-FVII_{L}:FVII_{H}-X;

(c) FVII_{L}:FVII_{H}-X-Tm or Tm-X-FVII_{H}:FVII_{L};

(d) FVII_{L}:FVII_{H}-Tm-X or X-Tm-FVII_{H}:FVII_{L};

(e) FVII_{L}-X-Tm:FVII_{H} or FVII_{H}:Tm-X-FVII_{L}; and

FVII_{L}-Tm-X:FVII_{H} or FVII_{H}:Tm-X-FVII_{L}, wherein FVII_{H} is a heavy chain of FVII; Tm is an anti-GPIIb/IIIa antibody or antigen-binding molecule thereof; FVII_{L} is a light chain of FVII; X is an XTEN polypeptide; and (:) is an association between two polypeptide chains.

The association between the first polypeptide chain and the second polypeptide chain can be a covalent bond, e.g., a disulfide bond, or a non-covalent bond.

In certain aspects of the invention, a chimeric molecule comprises a single polypeptide chain, which comprises, from N terminus to C terminus, (a) a light chain of FVII, the XTEN polypeptide, a protease cleavage site, a heavy chain of FVII, and the anti-GPIIb/IIIa antibody or antigen-binding molecule thereof; or (b) a light chain of FVII, the anti-GPIIb/IIIa antibody or antigen-binding molecule thereof, a protease cleavage site, a heavy chain of FVII, and the XTEN polypeptide. The protease cleavage site can be an intracellular processing site, which can be processed by a proprotein convertase.

In some aspects, a chimeric molecule comprises a first polypeptide chain and a second polypeptide chain, which are associated with each other,

(a) wherein the first polypeptide chain comprises a light chain of FVII and an XTEN polypeptide and the second chain polypeptide chain comprises a heavy chain of FVII and a targeting moiety, which binds to a platelet;

(b) wherein the first polypeptide chain comprises a light chain of FVII and a targeting moiety, which binds to a platelet, and the second polypeptide chain comprises a heavy chain of FVII and an XTEN polypeptide;

(c) wherein the first polypeptide chain comprises a light chain of FVII and the second polypeptide chain comprises a heavy chain of FVII, an XTEN polypeptide, and a targeting moiety, which binds to a platelet; or

(d) wherein the first polypeptide chain comprises a light chain of FVII and the second polypeptide chain comprises a heavy chain of FVII, a targeting moiety, which binds to a platelet, or an XTEN polypeptide.

In other aspects, a chimeric protein comprises a first polypeptide chain and a second polypeptide chain, which are associated with each other,

(a) wherein the first polypeptide chain comprises a formula of FVII_{L}-Tm or Tm-FVII_{L} and the second polypeptide chain comprises FVII_{H}-X or X-FVII_{H};

(b) wherein the first polypeptide chain comprises a formula of FVII_{L}-X or X-FVII_{L} and the second polypeptide chain comprises a formula of FVII_{H}-Tm or Tm-FVII_{H};

(c) wherein the first polypeptide chain comprises the formula of FVII_{L} and the second polypeptide chain comprises a formula of FVII_{H}-X-Tm or Tm-X-FVII_{H}; or

(d) wherein the first polypeptide chain comprises the formula of FVII_{L} and the second polypeptide chain comprises a formula of FVII_{H}-Tm-X or X-Tm-FVII_{H},

wherein FVII_{H} is a heavy chain of FVII; Tm is a targeting moiety, which binds to a platelet; FVII_{L} is a light chain of FVII; and X is an XTEN polypeptide.

Also provided is a chimeric molecule comprising a formula selected from the group consisting of:

(a) X-FVII_{L}:FVII_{H}-Tm or Tm-FVII_{H}: FVII_{L}-X;

(b) Tm-FVII_{L}:FVII_{H}-X or X-FVII_{H}: FVII_{L}-Tm;

(c) FVII_{L}:FVII_{H}-X-Tm or Tm-X-FVII_{H}:FVII_{L}; and

(d) FVII_{L}:FVII_{H}-Tm-X or X-Tm-FVII_{H}:FVII_{L}; wherein FVII_{H} is a heavy chain of FVII; Tm is a targeting moiety, which binds to a platelet; FVII_{L} is a light chain of FVII; X is an XTEN polypeptide; and (:) is an association between two polypeptide chains. The association between the first polypeptide chain and the second polypeptide chain can be a covalent bond, e.g., a disulfide bond, or a non-covalent bond.

In certain aspects, a chimeric molecule comprises a single polypeptide chain, which comprises, from N terminus to C terminus,

(a) a light chain of FVII, an XTEN polypeptide, a protease cleavage site, a heavy chain of FVII, and a targeting moiety which binds to a platelet;

(b) a light chain of FVII, a targeting moiety which binds to a platelet, a protease cleavage site, a heavy chain of FVII, and an XTEN polypeptide;

(c) a light chain of FVII, a protease cleavage site, a heavy chain of FVII, an XTEN polypeptide, and a targeting moiety which binds to a platelet; or

(d) a light chain of FVII, a protease cleavage site, a heavy chain of FVII, a targeting moiety which binds to a platelet, and an XTEN polypeptide. The protease cleavage site can be an intracellular processing site processed by a proprotein convertase.

The targeting moiety useful for the chimeric molecules can be selected from the group consisting of: an antibody or antigen-binding molecule thereof, a receptor binding portion of a receptor, and a peptide, which binds to a platelet. For example, the targeting moiety can selectively bind to a resting platelet or an activated platelet. In some embodiments, the targeting moiety selectively binds to a target selected from the group consisting of: GPIba, GPVI, GPIX, a nonactive form of glycoprotein IIb/IIIa (“GPIIb/IIIa”), an active form of GPIIb/IIIa, P selectin, GMP-33, LAMP-1, LAMP-2, CD40L, LOX-1, and any combinations thereof.

In some embodiments, the half-life of FVII in chimeric molecules is increased compared to FVIIa consisting of the heavy chain and the light chain. In other embodiments, the clotting activity of FVII in chimeric molecules is equal to or greater than FVIIa consisting of the heavy chain and the light chain. The clotting activity can be measured by a ROTEM assay, an aPTT assay, or any known assays.

In certain embodiments, the XTEN polypeptide comprises an AE motif, an AG motif, an AD motif, an AM motif, an AQ motif, an AF motif, a BC motif, a BD motif, or any combinations thereof. For example, the XTEN polypeptide for the chimeric molecules can comprise about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, about 2004 amino acids, or any combinations thereof. In a particular embodiment, the XTEN polypeptide is selected from the group consisting of: AE42, AE72, AE864, AE576, AE288, AE144, AG864, AG576, AG288, AG144, and any combinations thereof.

In some embodiments, the chimeric molecule further comprises a linker, wherein the linker connects any components of the chimeric molecule, e.g., the light chain of FVII with the XTEN polypeptide, the heavy chain of FVII with the targeting moiety, or both or the light chain of FVII with the targeting moiety, the light chain of FVII with the XTEN polypeptide, or both. In other embodiments, the linker comprises a peptide having the formula [(Gly)ₓ-Ser_{y}]_{z}, where x is from 1 to 4, y is 0 or 1, and z is from 1 to 50.

In certain embodiments, a chimeric molecule further comprises a heterologous moiety fused to a heavy chain of FVII, a light chain of FVII, an XTEN polypeptide, a targeting moiety, or any combinations thereof. The heterologous moiety can be a polypeptide moiety or a non-polypeptide moiety and further extends the half-life of FVII.

In some embodiment, the heterologous moiety extends the half-life of the chimeric molecule when administered to a subject compared to a FVII not comprising the heterologous moiety. In a particular embodiment, the heterologous moiety can be selected from the group consisting of albumin, albumin binding polypeptide or fatty acid, Fc, transferrin, PAS, the C-terminal peptide (CTP) of the β subunit of human chorionic gonadotropin, polyethylene glycol (PEG), hydroxyethyl starch (HES), albumin-binding small molecules, vWF, an additional XTEN polypeptide, and any combinations thereof.

Also provided is a pharmaceutical composition comprising the chimeric molecules, a polynucleotide or a set of polynucleotides encoding the chimeric molecules, a vector comprising the polynucleotide or the set of polynucleotides, a set of vectors comprising the set of polynucleotides, a host cell comprising the vector or the set of vectors, or methods of making the chimeric molecules comprising transfecting a host cell with the vector or the set of vectors and culturing the cell in a medium under suitable conditions for expressing the chimeric molecule.

Further provided is a method of reducing a frequency or degree of a bleeding episode or preventing an occurrence of a bleeding episode in a subject in need thereof comprising administering the chimeric molecule, the polynucleotide, the set of polynucleotides, the vector, the set of vectors, or the host cell. In some embodiments, the subject has developed or has the capacity to develop an inhibitor against FVIII, FIX, or both, e.g., a neutralizing antibody against FVIII, FIX, or both.

### BRIEF DESCRIPTION OF THE DRAWINGS/FIGURES

FIG. 1 is schematic diagrams of chimeric molecules comprising FVII, an XTEN polypeptide (X), and an scFv derived from an anti-GPIIb/IIIa antibody, e.g., 34D10. FVII can be activated FVII. The XTEN polypeptide, FVII, and the scFv can be linked by one or more optional linkers. ScFv in the chimeric molecules can contain V_{H} and V_{L} in any order, i.e., V_{H}-V_{L} or V_{L}-V_{H}.

FIGS. 2A-2D show diagrams of chimeric molecules comprising two polypeptide chains. FIG. 2A shows a schematic diagram of a chimeric molecule (e.g., FVII-211) comprising two polypeptide chains associated with each other, the first chain comprising a light chain of FVII and an XTEN polypeptide, which are optionally fused by a linker, and the second chain comprising a heavy chain of FVII and an scFv derived from an anti-GPIIb/IIIa antibody, which are fused by another linker. FIG. 2B is a schematic diagram of a chimeric molecule comprising two polypeptide chains associated with each other, the first chain comprising a light chain of FVII and an scFv derived from an anti-GPIIb/IIIa antibody and the second chain comprising a heavy chain of FVII and an XTEN polypeptide, wherein the light chain of FVII is fused to the scFv by a first optional linker and the heavy chain of FVII is fused to the XTEN polypeptide by a second optional linker. FIG. 2C. is a schematic diagram of a chimeric molecule (e.g., FVII-200) comprising two polypeptide chains, the first chain comprising a light chain of FVII and the second chain comprising a heavy chain of FVII, an XTEN polypeptide, and an scFv derived from an anti-GPIIb/IIIa antibody, wherein the heavy chain is fused to the XTEN polypeptide by a first optional linker and the XTEN polypeptide is fused to the scFv by a second optional linker. FIG. 2D is a schematic diagram of a chimeric molecule comprising two polypeptide chains associated with each other, the first chain comprising a light chain of FVII and the second chain comprising a heavy chain of FVII, an scFv derived from an anti-GPIIb/IIIa antibody, and an XTEN polypeptide, wherein the heavy chain is fused to the scFv by a first optional linker and the scFv is fused to the XTEN polypeptide by a second optional linker. ScFv in the chimeric molecules can contain V_{H} and V_{L} in any order, i.e., V_{H}-V_{L} or V_{L}-V_{H}.

FIGS. 3A and 3B show identity matrices corresponding to heavy chain variable domain (VH) sequences (FIG. 3A) and light chain variable domain (VL) sequences (FIG. 3B) of antibodies against GPIIb/IIIa in which the shaded cells indicate which antibodies share identical VL or VH sequences.

FIG. 4 shows a ClustalX multiple sequence alignment of the VH sequences of the 1H6, 38A8, 12B2, 38F6, 2A2, 36A8, 4B11, 34D10, 28F4, 23C10, 28C2, 5C4, 9D6, and 18F7 antibodies, indicating the location of the complementarity determining regions (CDR). The location of each CDR (CDR1, CDR2, and CDR3) according to the EU index is indicated. The location of identical, conserved and partially conserved amino acid residues is indicated below the alignment.

FIG. 5 shows a ClustalX multiple sequence alignment of the VL sequences of the 28C2, 9D6, 1H6, 38A8, 12B2, 18F7, 28F4, 34D10, 36A8, and 2A2 antibodies, indicating the location of CDR1, CDR2, and CDR3 according to the EU index. The location of identical, conserved and partially conserved amino acid residues is indicated below the alignment.

FIG. 6 shows percentage identity matrices corresponding to the sequences included in the ClustalX multiple sequence alignments shown in FIG. 5 (top matrix) and FIG. 4 (bottom matrix).

FIG. 7 shows ClustalX multiple sequence alignments corresponding to the VH sequences in FIG. 4 clustered according to their specificity for the α or β subunit of GPIIb/IIIa.

FIG. 8 shows ClustalX multiple sequence alignments corresponding to the VL sequences in FIG. 5 clustered according to their specificity for the α or β subunit of GPIIb/IIIa.

FIG. 9 shows ClustalX multiple sequence alignments corresponding to the VH sequences in FIG. 4 clustered according to their ability to compete with fibrinogen for binding to GPIIb/IIIa.

FIG. 10 shows ClustalX multiple sequence alignments corresponding to the VL sequences in FIG. 5 clustered according to their ability to compete with fibrinogen for binding to GPIIb/IIIa.

FIG. 11A shows FVIIa activity of rFVIIa and rFVII-XTEN measured by soluble tissue factor dependent prothrombin time (sTF-PT) assay (FIG. 11A). rFVIIa is recombinantly-produced activated FVII, and rFVIIa-XTEN is activated FVII, in which the heavy chain of FVII is fused to an XTEN polypeptide. X axis is time in hours, and y axis is FVIIa activity recovery from plasma in dosed HemA mice (%). FIG. 11B shows the clotting time (CT) of rFVIIa and rFVIIa-XTEN measured by ROTEM assays. X axis shows concentration in nM of indicated protein spiked in the citrated human hemophilia A blood, and y axis shows the clotting time recorded by ROTEM; clotting was initiated by Calcium.

FIG. 12A shows platelet-binding in human whole blood. Proteins were spiked in diluted (1:50) whole blood, stained with FITC-anti-FVII and APC-CD42b, and the FVII median fluorescent value (FVII MPV) on platelets was measured by flow-cytometry analysis. Three constructs (FVII-200 (circle), FVII-189 (triangle), and FVII-211 (x), which are described above) were tested for platelet binding. X axis shows the concentration of the proteins spiked in human blood, and y axis shows FVII median fluorescent value (FVII MFV) representing the relative amount of proteins that bind to platelets. FIG. 12B shows the clotting activity of rFVIIa (circle) and FVII-200 (triangle) measured by ROTEM assay. X axis shows concentration in nM and y axis is clotting time.

FIG. 13A shows the platelet-binding of FVII-200 (circle), FVII-189 (triangle), and FVII-211 (x) in blood from αIIb transgenic mice. X axis shows concentration in nM, and y axis shows FVII median fluorescent value (FVII MFV) on the platelets. FIG. 13B shows FVII recovery of rFVII-200 (circle), FVII-211 (x), and FVII-179 (triangle) on platelets from human αIIb transgenic mice as a function of time following protein administration. Mice were dosed at 5 nmol/kg of the indicated protein and the FVII median fluorescent valume on platelets was generated by flow cytometry analysis. The % FVII recovery refers to the % of remaining FVII MFV on platelets related to the value at 5 min after dosing.

FIGS. 14A to 14F show schematic diagrams of chimeric molecules comprising FVII, an XTEN polypeptide, and a platelet targeting moiety. FIG. 14A shows a chimeric protein comprising a light chain of FVII covalently associated with a heavy chain of FVII, which is further fused to an scFv derived from the PDG13 antibody (i.e., a platelet targeting antibody). FIG. 14B shows a chimeric protein comprising a light chain of FVII covalently associated with a heavy chain of FVII, which is further fused to an XTEN polypeptide. FIGS. 14C and 14D show a light chain covalently associated with a heavy chain of FVII, which is further linked to an scFv derived from the PDG13 antibody and an XTEN polypeptide (FIG. 14C) and to an XTEN polypeptide and an scFv derived from the PDG13 antibody (FIG. 14D). FIG. 14E shows a chimeric molecule comprising a first polypeptide chain and a second polypeptide chain, wherein the first polypeptide chain comprises a FVII light chain fused to an scFv derived from the PDG13 antibody and the second polypeptide chain comprises a FVII heavy chain fused to an XTEN polypeptide. FIG. 14F shows a chimeric molecule comprising two polypeptide chain, the first chain comprising a FVII light chain fused to an XTEN polypeptide and the second chain comprising a FVII heavy chain fused to an scFv derived from the PDG13 antibody.

FIGS. 15A to 15D show ROTEM assay comparing clotting time of rFVIIa with the chimeric FVII molecules. FIG. 15A shows clotting time of rFVIIa, FVII-165, and FVII-178 in human hemophilia blood simulated with anti-FVIII antibodies. Citrated blood from normal human donors was treated with anti-FVIII antibodies and indicated proteins. Clot was initiated by Calcium and the clotting time was recorded by ROTEM machine. BL, baseline from naive blood; BL+Ab, baseline level with anti-FVIII antibodies treatment. FIG. 15B shows the clotting time of rFVIIa and FVII-179 in citrated human hemophilia A blood by ROTEM; the clotting was initiated by Calcium. FIGS. 15C and 15D show the clotting time of rFVIIa, FVII-175, 177, 178 in citrated human hemophilia A blood by ROTEM, and the clotting was initiated by tissue factor and Calcium.

FIG. 16 shows the platelet-bound FVIIa clearance in platelet/NSG mice. Both FVII-211 and FVII-179 constructs are described above. NSG mice were dosed at 25 nmol/kg of either FVII-211 or FVII-179 at 30 min after human platelet transfusion. The median fluorescent value of human platelet-bound FVII at each time point normalized to that volume at 5 min after the protein administration. X axis shows time (hour) after dosing with FVII-211 or FVII-179, and y axis shows FVII recovery on transfused human platelets (%).

FIG. 17A shows the sequence of XTEN AE288. FIG. 17B shows the stability of GFP-XTEN in in vitro monkey plasma (upper row), in vivo monkey samples (middle), and in vitro rat kidney homogenate (lower row) that was detected by an anti-GFP antibody. The schematic diagrams of rFVIIa and rFVIIa-XTEN are shown at the bottom.

FIG. 18 shows FVIIa plasma activity of rFVIIa and rFVII-XTEN. rFVIIa is recombinantly-produced activated FVII, and rFVIIa-XTEN is activated FVII, in which the heavy chain of FVII is fused to an XTEN polypeptide. X axis is time in hours, and y axis is dose normalized FVIIa plasma activity (%).

FIG. 19 shows FVIIa plasma activity of rFVIIa and FVII-200. FVII-200 contains activated FVII (i.e., a heavy chain and a light chain), the heavy chain of the activated FVII being fused to an XTEN sequence (e.g., AE288), which is further fused to an scFv from an anti-GPIIb/IIIa antibody. X axis is time in hours, and y axis is dose normalized FVIIa plasma activity (%).

FIGS. 20A-C show the activity of platelet targeted rFVIIa-XTEN variants determined by sTF-PT method. FIG. 20A shows a schematic diagram of Configuration A, which comprises FVII light chain fused to XTENs (i.e., 72 amino acids, 144 amino acids, or 288 amino acids) and FVII heavy chain fused to 34D10 scFv by a linker, a table of three constructs, i.e., FVII-227, FVII-228, and FVII-211, and sTF-PT assay of the constructs and rFVIIa and FVII-189. The construct of FVII-189 is described above. FIG. 20B shows a schematic diagram of Configuration B, which comprises FVII light chain and FVII heavy chain fused to 34D10 scFv by XTENs (i.e., 72 amino acids, 144 amino acids, or 288 amino acids), a table of three constructs, i.e., FVII-231, FVII-232, and FVII-200, and sTF-PT assay of the constructs and rFVIIa. FIG. 20C shows a schematic diagram of Configuration C, which comprises FVII light chain fused to XTENs (i.e., 42 amino acids, 72 amino acids, or 72 amino acids) and FVII heavy chain fused to 34D10 scFv by XTENs (i.e., 72 amino acids, 42 amino acids, or 72 amino acids), a table of three constructs, i.e., FVII-242, FVII-243, and FVII-238, and sTF-PT assay of the constructs and rFVIIa.

FIGS. 21A-D shows activity of platelet targeted rFVIIa-XTEN variants determined by ROTEM method. FIG. 21A shows the schematic diagram and XTEN linkage and length of FVII-227, FVII-228, and FVII-211 as well as the FVII activity of FVII-227, FVII-228, and FVII-211. FIG. 21B shows the schematic diagram and XTEN linkage and length of FVII-231, FVII-232, and FVII-200 and the FVII activity of FVII-231, FVII-232, and FVII-200. FIG. 21C shows the schematic diagram and XTEN linkage and length of FVII-242, FVII-243, and FVII-238 and the FVII activity of FVII-242, FVII-243, and FVII-238. The FVII activity is shown by fold difference of FVII activity compared to rFVIIa. FIG. 21D shows the schematic diagram of FVII-200 and comparison of the FVII activity among FVII-189 (FVII light chain: FVII heavy chain-targeting moiety), FVII-165 (FVII light chain: FVII heavy chain-XTEN), and FVII-200 (FVII light chain: FVII heavy chain-XTEN-targeting moiety). The FVII activity is shown by fold difference of FVII activity compared to rFVIIa. ScFv in the constructs can contain V_{H} and V_{L} in any order, i.e., V_{H}-V_{L} or V_{L}-V_{H}.

FIG. 22 shows that a single XTEN with amino acids of 288 is sufficient for PK improvement. Various FVIIa-XTEN constructs, i.e., FVIIaXTEN864, FVIIaXTEN288, and rFVIIa) were administered to HemA mice. The FVII activity in plasma from dosed animals was measured by sTF-PT assay. The x axis shows time in hours, and the y axis shows normalized activity recovery (%).

FIG. 23 shows that reducing XTEN length to 144 or 72 increased the clearance rate. The FVII activities of three constructs, i.e, FVII-200/Hc-XTEN288, FVII-232/Hc-XTEN144, and FVII-231/Hc-XTEN72 from the plasma of dosed animals were measured by sTF-PT assay. The x axis shows time in hours, and the y axis shows normalized activity recovery (%).

FIG. 24 shows comparative data for two XTENs of 72 each vs a single XTEN 288. The FVII activity of FVII-238/Hc-XTEN72/Lc-XTEN72 was compared with that of FVII-200/Hc-XTEN288 and rFVIIa. The y axis shows the normalized activity recovery (%).

FIG. 25 shows the comparative data for two XTENs of 72 each vs a single XTEN 288. The proteins were administrated in αIIb transgenic mice via tail vein injection. Whole blood was collected and stained with fluorescent labeled the antibodies to visualized platelet and FVII by flow cytometry. The platelet-bound protein concentration was measured by quantifying the median fluorescent intensity (MFI) on platelets and expressed as the percentage of recovery in relation to the MFI at 5 min post dosing. The recovery (%) of FVII-238/Hc-XTEN72/Lc-XTEN72 was compared with that of FVII-200/Hc-XTEN288 and FVII-189 (without any XTEN).

FIG. 26 shows comparative data on XTEN length on heavy and light chain: platelet PK in αIIb transgenic mice, and the recovery was calculated similarly as FIG. 25. The recovery (%) of FVII-238/Hc-XTEN72/Lc-XTEN72 was compared with that of FVII-243/Hc-XTEN42/Lc-XTEN72, and FVII-242/Hc-XTEN72/Lc-XTEN42.

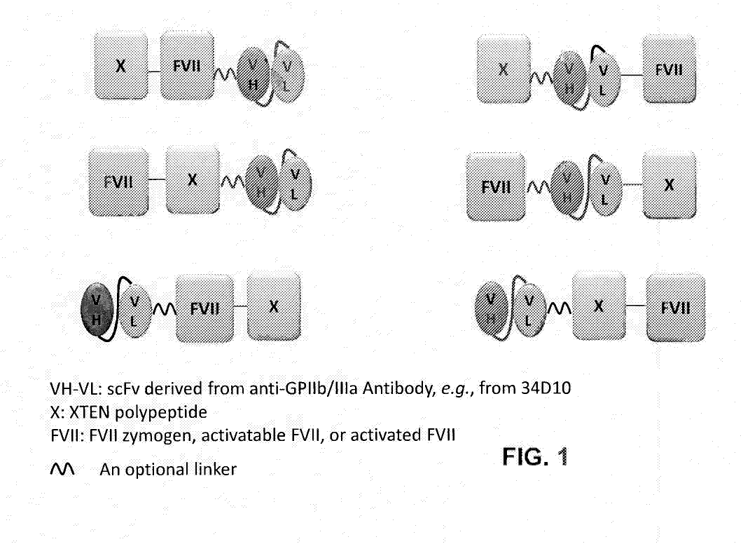

### DETAILED DESCRIPTION

The present invention relates to chimeric molecules comprising FVII, an XTEN polypeptide, and a targeting moiety that binds to a platelet (e.g., an anti-GPIIb/IIIa antibody or antigen-binding molecule thereof disclosed in section II.A.1). The present invention is based, at least in part, on the development of novel ways to enhance the efficacy, pharmacokinetic properties, and/or manufacturability of clotting factors. The chimeric molecule is developed in a way to have improved procoagulant activities at the site of coagulation as well as improved pharmacokinetic properties. For use in bypass therapy, exogenous clotting factors are only efficacious when given in the activated form. However, such activated clotting factors are rapidly inactivated by endogenous pathways (e.g., antithrombin III, TFPI), leading to clearance of the active form and a short effective half-life. Giving higher doses does not solve this problem as it can result in thrombogenic effects. Thus, in one embodiment, the invention pertains to an activity-enhanced chimeric FVII molecule constructs which comprise FVII fused to a targeting moiety that brings the clotting factor at the site of injury. These molecules also contain PK enhancing moiety, i.e., an XTEN polypeptide, which can improve various pharmacokinetic properties, e.g., half-life.

Exemplary constructs of the invention are illustrated in the accompanying Figures and sequence listing. In one embodiment, the invention pertains to a polypeptide having the structure as set forth in the Figures. In another embodiment, the invention pertains to a polypeptide having the sequence set forth in the accompanying sequence listing or the nucleic acid molecule encoding such polypeptides. In one embodiment, the invention pertains to a mature form of a polypeptide having the sequence set forth in the accompanying sequence listing. It will be understood that these constructs and nucleic acid molecules encoding them can be used to improve hemostasis in a subject.

In order to provide a clear understanding of the specification and claims, the following definitions are provided below.

### DEFINITIONS

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described herein with the language “comprising,” otherwise analogous embodiments described in terms of “consisting of” and/or “consisting essentially of” are also provided.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art relevant to the range or element at issue.

The amount of broadening from the strict numerical boundary depends upon many factors. For example, some of the factors to be considered can include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” can mean±5%, or ±10%, depending on the relevant technology. Also, the disclosure of ranges is intended as a continuous range including every value between the minimum and maximum values recited.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments of the disclosure, which can be by reference to the specification as a whole. Accordingly, the terms defined immediately below are more fully defined by reference to the specification in its entirety. Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “amino acid chain,” or any other term used to refer to a chain or chains of two or more amino acids, are included within the definition of “polypeptide,” and the term “polypeptide” can be used instead of, or interchangeably with any of these terms.

As used herein the term “protein” is intended to encompass a molecule comprised of one or more polypeptides, which can in some instances be associated by bonds other than amide bonds.

Polypeptides can be either monomers or multimers. For example, in one embodiment, an antibody, an antigen-binding molecule thereof, or a chimeric molecule of the invention can be a dimeric polypeptide. A dimeric antibody, an antigen-binding molecule thereof can comprise two polypeptide chains or can consist of one polypeptide chain (e.g., in the case of an scFc molecule). In one embodiment, the dimers can be a homodimer, comprising two identical monomeric subunits or polypeptides (e.g., two identical Fc moieties or two identical biologically active moieties). In another embodiment, the dimers are heterodimers, comprising two non-identical monomeric subunits or polypeptides (e.g., comprising two different clotting factors or portions thereof or one clotting factor only). See, e.g., U.S. Pat. No. 7,404,956, incorporated herein by reference.

The terms “polypeptide” and “protein” are also intended to refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

A polypeptide which is “isolated” is a polypeptide which is in a form not found in nature. Isolated polypeptides include those which have been purified to a degree that they are no longer in a form in which they are found in nature. In some embodiments, a polypeptide which is isolated is substantially pure.

“Derivatives” of GPIIb/IIIa antibodies, antigen-binding molecules thereof, or chimeric molecules of the invention are polypeptides or proteins which have been altered so as to exhibit additional features not found on the native polypeptide or protein. Also included as “derivatives” are those peptides that contain one or more naturally occurring amino acid derivatives of the twenty standard amino acids. A polypeptide or amino acid sequence “derived from” a designated polypeptide or protein refers to the origin of the polypeptide. In one embodiment, the polypeptide or amino acid sequence which is derived from a particular sequence has an amino acid sequence that is essentially identical to that sequence or a portion thereof, wherein the portion consists of at least about 10 to about 20 amino acids, at least about 20 to about 30 amino acids, or at least about 30 to about 50 amino acids, or which is otherwise identifiable to one of ordinary skill in the art as having its origin in the sequence.

Polypeptides that are “variants” of another polypeptide can have one or more mutations relative to the starting polypeptide, e.g., one or more amino acid residues which have been substituted with another amino acid residue or which has one or more amino acid residue insertions or deletions. In one embodiment, the polypeptide comprises an amino acid sequence which is not naturally occurring. Such variants necessarily have less than 100% sequence identity or similarity with the starting polypeptide. In another embodiment, the variant will have an amino acid sequence from about 75% to less than 100% amino acid sequence identity or similarity with the amino acid sequence of the starting polypeptide, for example, from about 80% to less than 100%, from about 85% to less than 100%, from about 90% to less than 100% (e.g., 91%, 92%, 93%, 94%, 95%, 96%, 97%, 98%, 99%) and from about 95% to less than 100%, e.g., over the length of the variant molecule. In one embodiment, there is one amino acid difference between a starting polypeptide sequence and the sequence derived therefrom.

The term “fragment” when referring to GPIIb/IIIa antibodies, antigen-binding molecules thereof, chimeric molecules of the invention, or clotting factors refers to any polypeptides or proteins which retain at least some of the properties of the reference polypeptide or protein. Fragments of polypeptides include proteolytic fragments, as well as deletion fragments. For example, a fragment of an anti-GPIIb/IIIa antibody can specifically binds to the same epitope as the anti-GPIIb/IIIa antibody. Another example is a fragment of FVII, which has a clotting activity of FVII, e.g., FVII clotting activity comparable to rFVIIa.

The term “sequence” as used to refer to a protein sequence, a peptide sequence, a polypeptide sequence, or an amino acid sequence means a linear representation of the amino acid constituents in the polypeptide in an amino-terminal to carboxyl-terminal direction in which residues that neighbor each other in the representation are contiguous in the primary structure of the polypeptide.

The term “amino acid” includes alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V).

Non-traditional amino acids are also within the scope of the invention and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, and C). These amino acids can participate in hydrophobic interactions and electrostatic interactions. As used herein, the term “charged amino acid” includes amino acids that can have non-zero net charge on their side chains (e.g. R, K, H, E, and D). These amino acids can participate in hydrophobic interactions and electrostatic interactions.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions”, can be made, e.g. insertion of about three to about five or even up to about ten, fifteen, or twenty amino acid residues. The inserted residue(s) can be naturally occurring or non-naturally occurring as disclosed above. An “amino acid deletion” refers to the removal of at least one amino acid residue from a predetermined amino acid sequence.

A “conservative amino acid substitution” is one in which the amino acid residue is replaced with an amino acid residue having a similar side chain. Families of amino acid residues having similar side chains have been defined in the art, including basic side chains (e.g., Lys, Arg, and His), acidic side chains (e.g., Asp and Glu), uncharged polar side chains (e.g., Gly, Asn, Gln, Ser, Thr, Tyr, and Cys), nonpolar side chains (e.g., Ala, Val, Leu, Ile, Pro, Phe, Met, and Trp), beta-branched side chains (e.g., Thr, Val, and Ile) and aromatic side chains (e.g., Tyr, Phe, Trp, and His). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

Non-conservative substitutions include those in which (i) a residue having an electropositive side chain (e.g., Arg, His, or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, He, Phe, or Val), (iii) a cysteine or proline is substituted for, or by, any other residue, or (iv) a residue having a bulky hydrophobic or aromatic side chain (e.g., Val, He, Phe, or Trp) is substituted for, or by, one having a smaller side chain (e.g., Ala or Ser) or no side chain (e.g., Gly).

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences.

One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org (ClustalX is a version of the ClustalW2 program ported to the Windows environment). Another suitable program is MUSCLE, available from www.drive5.com/muscle. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

In one embodiment, the antibodies and antigen-binding molecules thereof, as well as the chimeric molecules of the invention can comprise an amino acid sequence derived from a human protein sequence. However, the antibodies and antigen-binding molecules thereof, as well as the chimeric molecules of the invention can comprise one or more amino acids from another mammalian species. In a particular embodiment, the antibodies and antigen-binding molecules thereof, as well as the chimeric molecules of the invention are not immunogenic.

As used herein, the terms “linked,” “fused”, or “fusion” refer to linkage via a peptide bonds (e.g., genetic fusion), chemical conjugation, or other means known in the art. For example, one way in which molecules or moieties can be linked employs peptide linkers which link the molecules or moieties via peptide bonds. The terms “genetically fused,” “genetically linked,” or “genetic fusion” are used interchangeably and refer to the co-linear, covalent linkage or attachment of two or more proteins, polypeptides, or fragments thereof via their individual peptide backbones, through genetic expression of a single polynucleotide molecule encoding those proteins, polypeptides, or fragments. Such genetic fusion results in the expression of a single contiguous genetic sequence.

Preferred genetic fusions are in frame, i.e., two or more open reading frames (ORFs) are fused to form a continuous longer ORF, in a manner that maintains the correct reading frame of the original ORFs. Thus, the resulting recombinant fusion protein is a single polypeptide containing two or more protein segments that correspond to polypeptides encoded by the original ORFs (which segments are not normally so joined in nature). In this case, the single polypeptide is cleaved during processing to yield dimeric molecules comprising two polypeptide chains.

As used herein the term “associated with” refers to a covalent or non-covalent bond formed between a first amino acid chain and a second amino acid chain. In one embodiment, the term “associated with” means a covalent, non-peptide bond or a non-covalent bond. In another embodiment, the term “associated with” refers to a covalent, non-peptide bond or a non-covalent bond that is not chemically crosslinked. In another embodiment, it means a covalent bond except a peptide bond. In some embodiments this association is indicated by a colon, i.e., (:). For example, when representing the structure of FVII, “FVII_{H}:FVII_{L}” refers to a dimer comprising a heavy chain of FVII_{H} disulfide bonded to a light chain of FVII_{L} in a N-terminus to C-terminus orientation.

Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, an hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity.

As used herein, the terms “chemically crosslinked” and “conjugated” are used interchangeably and refer to chemically linking by covalent bonds between acid side chains of amino acids, either directly or via a linker, e.g., a peptide linker. Chemical crosslinking does not include intramolecular or intermolecular disulfide bonds between Fc moieties of a dimeric Fc region, or non-engineered disulfide bonds between an amino acid of the activated clotting factor and an amino acid of the enhancer moiety. Chemical crosslinking generally takes place by addition of a cross-linking agent, e.g., a heterobifunctional crosslinking agent. Examples of chemical crosslinking includes one or more photo-reactive bonds by chemically connecting photo-Ile, photo-Met, and photo-Leu (see, Suchanek et al., (2005) Nature Methods, 2: 261-267).

The term “antibody” means an immunoglobulin molecule that recognizes and specifically binds to a target, such as a protein (e.g., the GPIIb/IIIa receptor, a subunit thereof, or the receptor complex), polypeptide, peptide, carbohydrate, polynucleotide, lipid, or combinations of the foregoing through at least one antigen recognition site within the variable region of the immunoglobulin molecule.

A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region. The heavy chain constant region is comprised of three domains, CH1, CH2, and CH3. Each light chain is comprised of a “light chain variable region” or “light chain variable domain” (abbreviated herein as VL) and a light chain constant region. The light chain constant region is comprised of one domain, C1. The VH and VL regions can be further subdivided into regions of hypervariability, termed Complementarity Determining Regions (CDR), interspersed with regions that are more conserved, termed framework regions (FW).

Each VH and VL region is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. As used herein, the term “antibody” encompasses intact polyclonal antibodies, intact monoclonal antibodies, antibody fragments (such as Fab, Fab′, F(ab′)2, and Fv fragments), single chain Fv (scFv), minibodies, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, radioisotopes, etc.

There are at least two techniques for determining the location of CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See e.g., Lefranc, M. P. et al., Dev. Comp. Immunol. 27: 55-77 (2003). The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDR sequences described herein correspond to the classical Kabat numbering locations, namely Kabat VH-CDR1 is at positions 31-35, VH-CDR2 is a positions 50-65, and VH-CDR3 is at positions 95-102. VL-CDR1, VL-CDR2, and VL-CDR3 also correspond to classical Kabat numbering locations, namely positions 14-24, 50-56 and 89-97, respectively.

The term “consensus sequence,” as used herein with respect to a CDR in the light chain (VL) or heavy chain (VH) variable regions, refers to a composite or genericized amino acid sequence defined based on information as to which amino acid residues are present at a given position based in multiple sequence alignments. Thus, in a “consensus sequence” for a VL or VH chain CDR1, CDR2, or CDR3, certain amino acid positions are occupied by one of multiple possible amino acid residues at that position. For example, if an arginine (R) or a serine (S) occur at a particular position X, then that particular position within the consensus sequence can be either arginine or serine (R or S). Such occurrence would be represented, for example, as _{N}-Z₁Z₂XₙZₜ₋₁Z_{t-C}, where Z_{1>t} are invariant amino acids in the multiple sequence alignment, X represent a position occupied by variant amino acids (e.g., R or S), and the subindex n is an ordinal. As used herein, referring to a polypeptide sequence as consisting of or comprising a consensus sequence means that the polypeptide sequence consists of or comprises one of the of multiple possible amino acid sequences represented by the consensus sequence.

The term “antigen-binding fragment” refers to a portion of an intact antibody and refers to the antigenic determining variable regions of an intact antibody. It is known in the art that the antigen-binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced.

The term “Fab′” refers to an antibody fragment that is essentially equivalent to that obtained by reduction of the disulfide bridge or bridges joining the two heavy chain pieces in the F(ab′)2 fragment. Such fragments can be enzymatically or chemically produced by fragmentation of an intact antibody, recombinantly produced from a gene encoding the partial antibody sequence, or it can be wholly or partially synthetically produced.

The term “F(ab′)2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced.

The term “Fv” refers to an antibody fragment that consists of one NH and one N domain held together by noncovalent interactions.

The term “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, or Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides. The term “humanized antibody” refers to an antibody derived from a non-human (e.g., murine) immunoglobulin, which has been engineered to contain minimal non-human (e.g., murine) sequences. The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

In one embodiment, an anti-GPIIb/IIIa antibody of the invention comprises an antibody variant. The term “antibody variant” or “modified antibody” includes an antibody which does not occur in nature and which has an amino acid sequence or amino acid side chain chemistry which differs from that of a naturally-derived antibody by at least one amino acid or amino acid modification as described herein. As used herein, the term “antibody variant” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen; heavy chain molecules joined to scFv molecules; single-chain antibodies; diabodies; triabodies; and antibodies with altered effector function and the like.

As used herein the term “scFv” or “scFv molecule” includes binding molecules which consist of one light chain variable domain (VL) or a portion thereof, and one heavy chain variable domain (VH) or a portion thereof, wherein each variable domain (or a portion thereof) is derived from the same or different antibodies. Single chain Fv molecules preferably comprise an scFv linker interposed between the VH domain and the VL domain. In one embodiment, scFv comprises (N-terminus) VH-optional scFv linker-VL (C-terminus). In another embodiment, scFv comprises (N-terminus) VL-optional scFv linker-VH (C-terminus). Exemplary scFv molecules are known in the art and are described, for example, in U.S. Pat. No. 5,892,019; Ho et al., Gene 77:51 (1989); Bird et al., Science 242:423 (1988); Pantoliano et al., Biochemistry 30:10117 (1991); Milenic et al., Cancer Research 51:6363 (1991); Takkinen et al., Protein Engineering 4:837 (1991).

The term “scFv linker” as used herein refers to a moiety interposed between the VL and VH domains of the scFv. The scFv linkers preferably maintain the scFv molecule in an antigen-binding conformation. In one embodiment, a scFv linker comprises or consists of an scFv linker peptide. In certain embodiments, an scFv linker peptide comprises or consists of a gly-ser peptide linker. In other embodiments, an scFv linker comprises a disulfide bond.

As used herein, the term “antigen-binding molecule” refers to a molecule comprising an anti-GPIIb/IIIa antibody fragment, variant, or derivative thereof, comprising at least one CDR from one or more of the anti-GPIIb/IIIa antibodies disclosed herein. In some embodiments, the antigen-binding molecule is a protein. In other embodiments, the antigen-binding molecule is a protein scaffold (e.g., a fibronectin type III domain) or non-protein scaffold comprising at least one CDR from one of the anti-GPIIb/IIIa antibodies disclosed herein. In some embodiments, the antigen-binding molecule is an anti-GPIIb/IIIa antibody identified according to the methods disclosed herein, comprising at least one CDR identical to one of the CDR sequences disclosed herein. The term “antigen-binding molecule” also encompasses any molecule comprising a VH and/or VL region from one or more of the anti-GPIIb/IIIa antibodies disclosed herein.

The term “polynucleotide” or “nucleotide” is intended to encompass a singular nucleic acid as well as plural nucleic acids and refers to an isolated nucleic acid molecule or construct, e.g., messenger RNA (mRNA) or plasmid DNA (pDNA). In certain embodiments, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. Examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid can include regulatory elements such as promoters, enhancers, ribosome binding sites, or transcription termination signals.

As used herein, a “coding region” or “coding sequence” is a portion of polynucleotide which consists of codons translatable into amino acids. Although a “stop codon” (tag, tga, or taa) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′terminus, encoding the carboxyl terminus of the resulting polypeptide.

Two or more coding regions of the present invention can be present in a single polynucleotide construct, e.g., on a single vector, or in separate polynucleotide constructs, e.g., on separate (different) vectors. It follows, then, that a single vector can contain just a single coding region, or comprise two or more coding regions, e.g., a single vector can separately encode a binding domain-A and a binding domain-B as described below. In addition, a vector, polynucleotide, or nucleic acid of the invention can encode heterologous coding regions, either fused or unfused to a nucleic acid encoding a binding domain of the invention. Heterologous coding regions include without limitation specialized elements or motifs, such as a secretory signal peptide or a heterologous functional domain.

The term “vector” or “expression vector” is used herein to mean vectors used in accordance with the present invention as a vehicle for introducing into and expressing a desired polynucleotide in a cell. As known to those skilled in the art, such vectors can easily be selected from the group consisting of plasmids, phages, viruses, and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems can be employed to produce the antibody, antigen-binding molecule thereof, or a chimeric molecule of the invention. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Additionally, cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. In one embodiment, an inducible expression system can be employed. Additional elements can also be needed for optimal synthesis of mRNA. These elements can include signal sequences, splice signals, as well as transcriptional promoters, enhancers, and termination signals. In one embodiment, a secretion signal, e.g., any one of several well characterized bacterial leader peptides (e.g., pelB, phoA, or ompA), can be fused in-frame to the N terminus of a polypeptide of the invention to obtain optimal secretion of the polypeptide. (Lei et al. (1988), Nature, 331:543; Better et al. (1988) Science, 240:1041; Mullinax et al., (1990). PNAS, 87:8095).

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

A “recombinant” polypeptide or protein refers to a polypeptide or protein produced via recombinant DNA technology. Recombinantly produced polypeptides and proteins expressed in host cells are considered isolated for the purpose of the invention, as are native or recombinant polypeptides which have been separated, fractionated, or partially or substantially purified by any suitable technique.

The term “host cell” refers to a cell that has been transformed with a vector constructed using recombinant DNA techniques and encoding at least one heterologous gene. In descriptions of processes for isolation of proteins from recombinant hosts, the terms “cell” and “cell culture” are used interchangeably to denote the source of protein unless it is clearly specified otherwise. In other words, recovery of protein from the “cells” can mean either from spun down whole cells, or from the cell culture containing both the medium and the suspended cells. The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, CHO cell line, BHK cell line, HEK cell line, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), PerC6 cells), HAK (hamster kidney line), SP2/O (mouse myeloma), P3x63-Ag3.653 (mouse myeloma), BFA-1clBPT (bovine endothelial cells), and RAJI (human lymphocyte). Host cell lines are typically available from commercial services, the American Tissue Culture Collection or from published literature.

### II. Chimeric FVII Molecules

### II.A. Chimeric Molecule Comprising Anti-GPIIb/IIIa Antibodies and XTEN

The present invention provides a chimeric molecule comprising FVII, an XTEN polypeptide, and an anti-GPIIb/IIIa antibody and antigen-binding molecules thereof that specifically bind to GPIIb/IIIa receptors located on the surface of platelets. The chimeric molecule is constructed to extend the circulating half-life of FVII and to improve binding affinity to activated platelets, thereby reducing the frequency of dosing. Therefore, the chimeric molecule of the present invention is a long-lasting and more potent form of a FVII variant combining half-life extension with activity improvement. In order to improve circulating half-life, rFVIIa is fused to an XTEN polypeptide, a hydrophilic and unstructured polypeptide that increases the hydrodynamic radius of the payload protein. In addition, the coagulation activity is enhanced by targeting rFVIIa to platelets with an anti-GPIIb/IIIa antibody or antigen-binding molecule thereof that binds to the platelet receptor αIIβ3 with high affinity.

A chimeric molecule can comprise FVII, an XTEN polypeptide, or an anti-GPIIb/IIIa antibody or antigen-binding molecule thereof as described in section II.A.1., in any order. In one embodiment, a chimeric molecule comprises, from N terminus to C terminus, FVII, an XTEN polypeptide, and an anti-GPIIb/IIIa antibody or an antigen-binding molecule thereof as described in section II.A.1. In another embodiment, a chimeric molecule comprises, from N terminus to C terminus, FVII, an anti-GPIIb/IIIa antibody or antigen-binding molecule thereof as described in section II.A.1., and an XTEN polypeptide. In other embodiments, a chimeric molecule comprises, from N terminus to C terminus, an XTEN polypeptide, FVII, and an anti-GPIIb/IIIa antibody or antigen-binding molecule thereof as described herein in section II.A.1. In still other embodiments, a chimeric molecule comprises, from N terminus to C terminus, an anti-GPIIb/IIIa antibody or antigen-binding molecule thereof as described in section II.A.1., FVII, and an XTEN polypeptide. In yet other embodiments, a chimeric molecule comprises, from N terminus to C terminus, an XTEN polypeptide, an anti-GPIIb/IIIa antibody or antigen-binding molecule thereof as described in section II.A.1., and FVII. In some embodiments, a chimeric molecule comprises, from N terminus to C terminus, an anti-GPIIb/IIIa antibody or antigen-binding molecule thereof as described in section II.A.1., an XTEN polypeptide, and FVII.

In certain embodiments, a chimeric molecule comprises a formula selected from the group consisting of (a) FVII-(L1)-X-(L2)-Tm; (b) FVII-(L1)-Tm-(L2)-X; (c) Tm-(L1)-X-(L2)-FVII; (d) Tm-(L1)-FVII-(L2)-X; (e) X-(L1)-Tm-(L2)-FVII; and (f) X-(L1)-FVII-(L2)-Tm; wherein FVII comprises FVIIa; X is an XTEN polypeptide; Tm is an anti-GPIIb/IIIa antibody or antigen-binding molecule thereof as described in section II.A.1.; L1 is a first optional linker, and L2 is a second optional linker. In some embodiments, a chimeric molecule is a single polypeptide chain or two polypeptide chains comprising a first polypeptide chain and a second polypeptide chain.

In one embodiment, a chimeric molecule comprises a first polypeptide chain and a second polypeptide chain, which are associated with each other, wherein the first polypeptide chain comprises a light chain of FVII and an XTEN polypeptide and the second polypeptide chain comprises a heavy chain of FVII and an anti-GPIIb/IIIa antibody or antigen-binding molecule thereof as described in section II.A.1. In another embodiment, a chimeric molecule comprises a first polypeptide chain and a second polypeptide chain, which are associated with each other, wherein the first polypeptide chain comprises a light chain of FVII and an anti-GPIIb/IIIa antibody or antigen-binding molecule thereof as described in section II.A.1. and the second polypeptide chain comprises a heavy chain of FVII and an XTEN polypeptide. In other embodiments, a chimeric molecule comprises a first polypeptide chain and a second polypeptide chain, which are associated with each other, wherein the first polypeptide chain comprises a light chain of FVII, an XTEN polypeptide, and an anti-GPIIb/IIIa antibody or antigen-binding molecule thereof as described in section II.A.1., in any order, and the second chain comprises a heavy chain of FVII. In still other embodiments, a chimeric molecule comprises a first polypeptide chain and a second polypeptide chain, which are associated with each other, wherein the first polypeptide chain comprises a light chain of FVII and the second chain comprises a heavy chain of FVII, an XTEN polypeptide, and an anti-GPIIb/IIIa antibody or antigen-binding molecule thereof as described in section II.A.1., in any order.

In certain embodiments, a chimeric molecule comprises a first polypeptide chain and a second polypeptide chain, which are associated with each other, wherein the first polypeptide chain comprises a formula of FVII_{L}-X or X-FVII_{L} and the second polypeptide chain comprises a formula of FVII_{H}-Tm or Tm-FVII_{H}. In some embodiments, a chimeric molecule comprises a first polypeptide chain and a second polypeptide chain, which are associated with each other, wherein the first polypeptide chain comprise a formula of FVII_{L}-Tm or Tm-FVII_{L} and the second polypeptide chain comprises a formula of FVII_{H}-X or X-FVII_{H}. In other embodiments, a chimeric molecule comprises a first polypeptide chain and a second polypeptide chain, which are associated with each other, wherein the first polypeptide chain comprise the formula of FVII_{L} and the second polypeptide chain comprises a formula of FVII_{H}-X-Tm or Tm-X-FVII_{H}. In still other embodiments, a chimeric molecule comprises a first polypeptide chain and a second polypeptide chain, which are associated with each other, wherein the first polypeptide chain comprise the formula of FVII_{L} and the second polypeptide chain comprises a formula of FVII_{H}-Tm-X or X-Tm-FVII_{H}. In yet other embodiments, a chimeric molecule comprises a first polypeptide chain and a second polypeptide chain, which are associated with each other, wherein the first polypeptide chain comprise a formula of FVII_{L}-Tm-X or X-Tm-FVII_{L} or and the second polypeptide chain comprises the formula of FVII_{H}. In some other embodiments, a chimeric molecule comprises a first polypeptide chain and a second polypeptide chain, which are associated with each other, wherein the first polypeptide chain comprise a formula of FVII_{L}-X-Tm or Tm-X-FVII_{L} and the second polypeptide chain comprises the formula of FVII_{H}. Each component of the chimeric molecules is noted as follows: FVII_{H} is a heavy chain of FVII; Tm is an anti-GPIIb/IIIa antibody or antigen-binding molecule thereof as described in section II.A.1.; FVII_{L} is a light chain of FVII; and X is an XTEN polypeptide.

In some embodiments, a chimeric molecule comprises a formula of X-FVII_{L}:FVII_{H}-Tm, X-FVII_{L}:Tm-FVII_{H}, FVII_{L}-X:FVII_{H}-Tm, FVII_{L}-X:Tm-FVII_{H}, Tm-FVII_{H}:X-FVII_{L}, Tm-FVII_{H}:FVII_{L}-X, FVII_{H}-Tm:FVII_{L}-X, or FVII_{H}-Tm:X-FVII_{L}, wherein FVII_{H} is a heavy chain of FVII; Tm is an anti-GPIIb/IIIa antibody or antigen-binding molecule thereof as described in section II.A.1.; FVII_{L} is a light chain of FVII; X is an XTEN polypeptide; and (:) is an association between two polypeptide chains. In yet other embodiments, a chimeric molecule comprises a formula of FVII_{L}:FVII_{H}-X-Tm; Tm-X-FVII_{H}:FVII_{L}; FVII_{L}:FVII_{H}-Tm-X; or X-Tm-FVII_{H}:FVII_{L}; wherein FVII_{H} is a heavy chain of FVII; Tm is an anti-GPIIb/IIIa antibody or antigen-binding molecule thereof described in section II.A.1.; FVII_{L} is a light chain of FVII; X is an XTEN polypeptide; and (:) is an association between two polypeptide chains.

In some embodiments, the first polypeptide chain and the second polypeptide chain are associated, e.g., via a covalent bond or a non-covalent bond. In other embodiments, the association between the first polypeptide chain and the second polypeptide chain is a covalent bond between the heavy chain and the light chain of the clotting factor. In a specific embodiment, the association between the first polypeptide chain and the second polypeptide chain is a disulfide bond.

The chimeric molecule of the present invention can be produced by a single polynucleotide chain encoding a single polypeptide chain or two or more polynucleotide chains encoding two or more polypeptide chains. In one embodiment, a single polypeptide chain encoded by a single polynucleotide chain can be processed into two or more polypeptide chains. In another embodiment, a chimeric molecule comprises a single polypeptide chain, which comprises, from N terminus to C terminus, a light chain of FVII, an XTEN polypeptide, a protease cleavage site, a heavy chain of FVII, and an anti-GPIIb/IIIa antibody or antigen-binding molecule thereof as described in section II.A.1. In another embodiment, a chimeric molecule comprises a single polypeptide chain, which comprises, from N terminus to C terminus, a light chain of FVII, an anti-GPIIb/IIIa antibody or antigen-binding molecule thereof as described in section II.A.1., a protease cleavage site, a heavy chain of FVII, and an XTEN polypeptide. In other embodiments, a chimeric molecule comprises a single polypeptide chain, which comprises, from N terminus to C terminus, a light chain of FVII, an optional protease cleavage site, a heavy chain of FVII, an XTEN polypeptide, and an anti-GPIIb/IIIa antibody or antigen-binding molecule thereof as described in section II.A.1. In still other embodiments, a chimeric molecule comprises a single polypeptide chain, which comprises, from N terminus to C terminus, a light chain of FVII, an optional protease cleavage site, a heavy chain of FVII, an anti-GPIIb/IIIa antibody or antigen-binding molecule thereof as described in section II.A.1., and an XTEN polypeptide. In yet other embodiments, a chimeric molecule comprises a single polypeptide chain, which comprises, from N terminus to C terminus, a light chain of FVII, an XTEN polypeptide, an anti-GPIIb/IIIa antibody or antigen-binding molecule thereof as described in section II.A.1., an optional protease cleavage site, and a heavy chain of FVII. In some embodiments, a chimeric molecule comprises a single polypeptide chain, which comprises, from N terminus to C terminus, a light chain of FVII, an anti-GPIIb/IIIa antibody or antigen-binding molecule thereof as described in section II.A.1., an XTEN polypeptide, an optional protease cleavage site, and a heavy chain of FVII. In other embodiments, the protease cleavage site is an intracellular processing site. The intracellular processing sites can be processed by any protease enzyme, e.g., a proprotein convertase, e.g., PC5, PACE, PC7, and any combinations thereof.

In some embodiments, the chimeric molecule comprises an amino acid sequence at least about 70%, 80%, 90%, 95%, 96%, 97%, 98%, or 99% identical to the amino acid sequence encoded by SEQ ID NO: 192 or SEQ ID NO: 193. In a particular embodiment, the chimeric molecule comprises the amino acid sequence encoded by SEQ ID NO: 192 or SEQ ID NO: 193.

### II.A.1 Anti-GPIIb/IIIa Antibody or Antigen-Binding Molecule Thereof

The terms “GPIIb/IIIa antibody,” “anti-GPIIb/IIIa antibody,” “anti-GPIIb/IIIa,” “antibody that binds to GPIIb/IIIa” and any grammatical variations thereof refer to an antibody that is capable of specifically binding to the GPIIb/IIIa receptor with sufficient affinity such that the antibody is useful as a part of a therapeutic agent or diagnostic reagent in targeting GPIIb/IIIa. The extent of binding of an anti-GPIIb/IIIa antibody disclosed herein to an unrelated, non-GPIIb/IIIa protein is less than about 10% of the binding of the antibody to GPIIb/IIIa as measured, e.g., by a radioimmunoassay (RIA), BIACORE™ (using recombinant GPIIb/IIIa as the analyte and antibody as the ligand, or vice versa), or other binding assays known in the art. In certain embodiments, an antibody that binds to GPIIb/IIIa has a dissociation constant (K_{D}) of ≦1 μM, ≦100 nM, ≦50 nM, ≦10 nM, ≦1 nM, ≦0.1 nM, ≦10 pM, ≦1 pM, or ≦0.1 pM.

As used herein, the terms “GPIIb/IIIa” and “GPIIb/IIIa receptor” refer to glycoprotein IIb/IIIa (also known as integrin αIIbβ3), an integrin complex found on platelets. Integrins are composed of two chains, an α subunit and a β subunit, which are held together by noncovalent bonds in a calcium dependent manner. GPIIb constitutes the α subunit, which comprises divalent cation binding domains, whereas GPIIIa is a pro typical β subunit (β3). On each circulating platelet, there are 35,000 to 100,000 GPIIb/IIIa complexes; most are distributed on the platelet surface, with a smaller pool in an internal reserve. The GPIIb/IIIa complex does not interact with its plasma ligands until platelets have been activated by exogenous agonists such as ADP or thrombin. When this occurs, an inside-out signal is generated that results in a conformational change in the extracellular portion of the complex that renders the molecule capable of binding fibrinogen and other ligands. See Uniprot entries P05106 (ITB3_HUMAN; GPIIIa: CD61; integrin beta-3; integrin (33) and P08514 (ITA2B_HUMAN; GPIIb; CD41; integrin alpha-2b; integrin al) as published in Universal Protein Resource (Uniprot) database release 2013_05 (May 1, 2013), which are incorporated by reference in their entireties.

A chimeric molecule of the invention can comprises FVII, an XTEN polypeptide and an anti-GPIIb/IIIa antibody or antigen-binding molecules thereof specifically binding to a GPIIb/IIIa epitope, which comprises or overlaps with the GPIIb/IIIa binding epitope of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4 (see TABLE 3). In one embodiment, a chimeric molecule comprises FVII, an XTEN polypeptide, and an anti-GPIIb/IIIa antibody or antigen-binding molecules thereof specifically binding to a GPIIb/IIIa epitope, which is the same GPIIb/IIIa binding epitope of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4 (see TABLE 3). As used herein, the term “epitope” designates a specific amino acid sequence, modified amino acid sequence, or protein secondary or tertiary structure which is specifically recognized by an antibody. The terms “specifically recognizing,” “specifically recognizes,” and any grammatical variants mean that the antibody or antigen-binding molecule thereof is capable of specifically interacting with and/or binding to at least two, at least three, or at least four amino acids of an epitope, e.g., a GPIIb/IIIa epitope. Such binding can be exemplified by the specificity of a “lock-and-key-principle.” Thus, specific motifs in the amino acid sequence of the antigen-binding domain the GPIIb/IIIa antibody or antigen-binding molecule thereof and the epitope bind to each other as a result of their primary, secondary or tertiary structure as well as the result of secondary modifications of the structure.

In another embodiment, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof competitively inhibits GPIIb/IIIa binding to an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4 (see TABLE 1). In other embodiments, the anti-GPIIb/IIIa antibody or antigen-binding molecule thereof which specifically binds to a GPIIb/IIIa epitope comprises at least one, at least two, at least three, at least four, or at least five complementarity determining regions (CDR) or variants thereof of an antibody selected from the group consisting of one or more of the 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4 antibodies disclosed in TABLE 1. In still other embodiments, the antibody or antigen-binding molecule thereof which specifically binds to a GPIIb/IIIa epitope comprises six CDRs or variants thereof of an antibody selected from the group consisting of one or more of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4 disclosed herein. In some embodiments, CDRs are independently selected from CDRs or variants thereof derived from the VH and/or VL region of one, two, three, four, or six antibodies selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4.

In certain embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to VH-CDR1 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;

(ii) a variable heavy chain CDR-2 (VH-CDR2) sequence at least about 60%, 70%, 80%, 90%, 95%, or 100% identical to VH-CDR2 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;

(iii) a variable heavy chain CDR-3 (VH-CDR3) sequence at least about 60%, 70%, 80%, 90%, 95%, or 100% identical to VH-CDR3 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;

(iv) a variable light chain CDR-1 (VL-CDR1) sequence at least about 60%, 70%, 80%, 90%, 95%, or 100% identical to VL-CDR1 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;

(v) a variable light chain CDR-2 (VL-CDR2) sequence at least about 60%, 70%, 80%, 90%, 95%, or 100% identical to VL-CDR2 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4, and/or

(vi) a variable light chain CDR-3 (VL-CDR3) sequence at least about 60, 70, 80, 90, or 95% identical to VL-CDR3 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4.

In certain embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to VH-CDR1 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;

(ii) a variable heavy chain CDR-2 (VH-CDR2) sequence at least about 60%, 70%, 80%, 90%, 95%, or 100% identical to VH-CDR2 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4; and

(iii) a variable heavy chain CDR-3 (VH-CDR3) sequence at least about 60%, 70%, 80%, 90%, 95%, or 100% identical to VH-CDR3 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4.

In other embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable light chain CDR-1 (VL-CDR1) sequence at least about 60%, 70%, 80%, 90%, 95%, or 100% identical to VL-CDR1 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;

(ii) a variable light chain CDR-2 (VL-CDR2) sequence at least about 60%, 70%, 80%, 90%, 95%, or 100% identical to VL-CDR2 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4, and

(iii) a variable light chain CDR-3 (VL-CDR3) sequence at least about 60, 70, 80, 90, or 95% identical to VL-CDR3 of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to VH-CDR1 of an antibody selected from the group consisting of 34D10, 2A2, 35D1, 36A8, 4B11, 1H6, 38G8, 21F10, 38A8, and 18F7;

(ii) a variable heavy chain CDR-2 (VH-CDR2) sequence at least about 60%, 70%, 80%, 90%, 95%, or 100% identical to VH-CDR2 of an antibody selected from the group consisting of 34D10, 2A2, 35D1, 36A8, 4B11, 1H6, 38G8, 21F10, 38A8, and 18F7;

(iii) a variable heavy chain CDR-3 (VH-CDR3) sequence at least about 60%, 70%, 80%, 90%, 95%, or 100% identical to VH-CDR3 of an antibody selected from the group consisting of 34D10, 2A2, 35D1, 36A8, 4B11, 1H6, 38G8, 21F10, 38A8, and 18F7;

(iv) a variable light chain CDR-1 (VL-CDR1) sequence at least about 60%, 70%, 80%, 90%, 95%, or 100% identical to VL-CDR1 of an antibody selected from the group consisting of 34D10, 2A2, 35D1, 36A8, 4B11, 1H6, 38G8, 21F10, 38A8, and 18F7;

(v) a variable light chain CDR-2 (VL-CDR2) sequence at least about 60%, 70%, 80%, 90%, 95%, or 100% identical to VL-CDR2 of an antibody selected from the group consisting of 34D10, 2A2, 35D1, 36A8, 4B11, 1H6, 38G8, 21F10, 38A8, and 18F7, and/or

(vi) a variable light chain CDR-3 (VL-CDR3) sequence at least about 60, 70, 80, 90, or 95% identical to VL-CDR3 of an antibody selected from the group consisting of 34D10, 2A2, 35D1, 36A8, 4B11, 1H6, 38G8, 21F10, 38A8, and 18F7.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to VH-CDR1 of an antibody selected from the group consisting of 12B2, 38F6, 13C1, 5C4, 23C10, 37C7, 28C2, 9D6, and 28F4;

(ii) a variable heavy chain CDR-2 (VH-CDR2) sequence at least about 60%, 70%, 80%, 90%, 95%, or 100% identical to VH-CDR2 of an antibody selected from the group consisting of 12B2, 38F6, 13C1, 5C4, 23C10, 37C7, 28C2, 9D6, and 28F4;

(iii) a variable heavy chain CDR-3 (VH-CDR3) sequence at least about 60%, 70%, 80%, 90%, 95%, or 100% identical to VH-CDR3 of an antibody selected from the group consisting of 12B2, 38F6, 13C1, 5C4, 23C10, 37C7, 28C2, 9D6, and 28F4;

(iv) a variable light chain CDR-1 (VL-CDR1) sequence at least about 60%, 70%, 80%, 90%, 95%, or 100% identical to VL-CDR1 of an antibody selected from the group consisting of 12B2, 38F6, 13C1, 5C4, 23C10, 37C7, 28C2, 9D6, and 28F4;

(v) a variable light chain CDR-2 (VL-CDR2) sequence at least about 60%, 70%, 80%, 90%, 95%, or 100% identical to VL-CDR2 of an antibody selected from the group consisting of 12B2, 38F6, 13C1, 5C4, 23C10, 37C7, 28C2, 9D6, and 28F4, and/or

(vi) a variable light chain CDR-3 (VL-CDR3) sequence at least about 60, 70, 80, 90, or 95% identical to VL-CDR3 of an antibody selected from the group consisting of 12B2, 38F6, 13C1, 5C4, 23C10, 37C7, 28C2, 9D6, and 28F4.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 25, 31, 37, 43, or 111;

(ii) a variable heavy chain CDR-2 (VH-CDR2) sequence at least about 60%, 70%, 80%, 90%, 95%, or 100% identical to any one of SEQ ID NOS:26, 32, 38, 44, or 112;

(iii) a variable heavy chain CDR-3 (VH-CDR3) sequence at least about 60%, 70%, 80%, 90%, 95%, or 100% identical to any one of SEQ ID NOS: 27, 33, 39, 45, or 113;

(iv) a variable light chain CDR-1 (VL-CDR1) sequence at least about 60%, 70%, 80%, 90%, 95%, or 100% identical to any one of SEQ ID NOS: 28, 34, 40, 117, or 114;

(v) a variable light chain CDR-2 (VL-CDR2) sequence at least about 60%, 70%, 80%, 90%, 95%, or 100% identical to any one of SEQ ID NOS: 29, 35, 41, 118, or 115; and,

(vi) a variable light chain CDR-3 (VL-CDR3) sequence at least about 60, 70, 80, 90, or 95% identical to any one of SEQ ID NOS: 30, 36, 42, 119, or 116.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 comprising the consensus sequence X₁YAMS wherein X₁ represents any amino acid residue, e.g., an amino acid residue with uncharged polar side chain or nonpolar side chain, e.g., Thr (T), Ser (S), or Ala (A);

(ii) a VH-CDR2 comprising the consensus sequence SIX₂X₃GX₄X₅T YX₆X₇DSVKX₈ wherein X₂ represents any amino acid residue, e.g., an amino acid residue with uncharged polar side chain, e.g., Ser (S) or Asn (N), X₃ represents any amino acid residue, e.g., an amino acid residue with uncharged polar side chain, e.g., Ser (S) or Gly (G), X₄ represents any amino acid residue, e.g., an amino acid residue with uncharged polar side chain, e.g., Ser (S) or Gly (G), X₅ represents any amino acid residue, e.g., an amino acid residue with uncharged polar side chain, e.g., Ser (S), Asn (N), or Thr (T), X₆ represents any amino acid residue, e.g., an amino acid residue with aromatic side chain, e.g., Tyr (Y) or Phe (F), X₇ represents any amino acid residue, e.g., an amino acid residue with nonpolar side chains, e.g., Leu (L) or Pro (P), and X₅ represents any amino acid residue, e.g., an amino acid residue with basic side chains or uncharged polar side chains, e.g., Gly (G) or Arg (R);

(iii) a VH-CDR3 comprising the consensus sequence GGDYGYAX₉DY, wherein X₉ represents any amino acid residue, e.g., an amino acid residue with nonpolar side chains, e.g., Leu (L) or Met (M);

(iv) a VL-CDR1 comprising the sequence RASSSVNYMY (SEQ ID NO: 28);

(v) a VL-CDR2 comprising the sequence YTSNLAP (SEQ ID NO: 29); and,

(vi) a VL-CDR3 comprising the sequence QQFSSSPWT (SEQ ID NO: 30).

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 sequence selected from the group consisting of SEQ ID NOS: 25, 31, 37, 43, and 111;

(ii) a VH-CDR2 sequence selected from the group consisting of SEQ ID NOS: 26, 32, 38, 44, and 112;

(iii) a VH-CDR3 sequence selected from the group consisting of SEQ ID NOS: 27, 33, 39, 45, and 113;

(iv) a VL-CDR1 sequence selected from the group consisting of SEQ ID NOS: 28, 34, 40, 117, and 114;

(v) a VL-CDR2 sequence selected from the group consisting of SEQ ID NOS: 29, 35, 41, 118, and 115; and,

(vi) a VL-CDR3 sequence selected from the group consisting of SEQ ID NOS: 30, 36, 42, 119, and 116.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises a VH region comprising an amino acid sequence at least about 80%, 85%, 90%, 95%, or 100% identical to any one of SEQ ID NOS: 1, 3, 5, 7, or 97 and a VL region comprising an amino acid sequence at least about 80%, 85%, 90%, 95%, or 100% identical to any one of SEQ ID NOS: 2, 4, 6, 99, or 98. In some embodiments, the antibody or antigen-binding molecule thereof comprises a VH region comprising the amino acid sequence of SEQ ID NO: 1 and a VL region comprising the amino acid sequence of SEQ ID NO: 2. In other embodiments, the antibody or antigen-binding molecule thereof comprises a VH region comprising the amino acid sequence of SEQ ID NO: 3 and a VL region comprising the amino acid sequence of SEQ ID NO: 4. In some embodiments, the antibody or antigen-binding molecule thereof comprises a VH region comprising the amino acid sequence of SEQ ID NO: 5 and a VL region comprising the amino acid sequence of SEQ ID NO: 6. In some embodiments, the antibody or antigen-binding molecule thereof comprises a VH region comprising the amino acid sequence of SEQ ID NO: 7 and a VL region comprising the amino acid sequence of SEQ ID NO: 99. In some embodiments, the antibody or antigen-binding molecule thereof comprises a VH region comprising the amino acid sequence of SEQ ID NO: 97 and a VL region comprising the amino acid sequence of SEQ ID NO: 98. In some embodiment, the anti-GPIIb/IIIa antibody or antigen-binding molecule thereof binds to a GPIIb/IIIa epitope located in the extracellular domain of the alpha subunit of GPIIb/IIIa or to a binding site formed by the extracellular domains of the GPIIb/IIIa complex. In some embodiments, the GPIIb/IIIa antibody or antigen-binding molecule thereof does not compete with fibrinogen for binding to GPIIb/IIIa.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 46, 52, 120, or 126;

(ii) a variable heavy chain CDR-2 (VH-CDR2) sequence at least about 60%, 70%, 80%, 90%, 95%, or 100% identical to any one of SEQ ID NOS: 47, 53, 121, or 127;

(iii) a variable heavy chain CDR-3 (VH-CDR3) sequence at least about 60%, 70%, 80%, 90%, 95%, or 100% identical to any one of SEQ ID NOS: 48, 54, 122, or 128;

(iv) a variable light chain CDR-1 (VL-CDR1) sequence at least about 60%, 70%, 80%, 90%, 95%, or 100% identical to any one of SEQ ID NOS: 49, 55, 123, or 129;

(v) a variable light chain CDR-2 (VL-CDR2) sequence at least about 60%, 70%, 80%, 90%, 95%, or 100% identical to any one of SEQ ID NOS: 50, 56, 124, or 130; and,

(vi) a variable light chain CDR-3 (VL-CDR3) sequence at least about 60%, 70%, 80%, 90%, 95%, or 100% identical to any one of SEQ ID NO: 51, 57, 125, or 131.

In some embodiments. a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 comprising the sequence NYLIE (SEQ ID NO: 46);

(ii) a VH-CDR2 comprising the sequence VINPGSGGTNYNEKFKG (SEQ ID NO: 47);

(iii) a VH-CDR3 comprising the sequence GRYEWYFDV (SEQ ID NO: 48);

(iv) a VL-CDR1 comprising the consensus sequence RASQDIX₁₀NYLN wherein X₁₀ represents any amino acid residue, e.g., an amino acid residue with uncharged polar side chain e.g., Ser (S) or Thr (T);

(v) a VL-CDR2 comprising the sequence YTSRLHS (SEQ ID NO:50); and,

(vi) a VL-CDR3 comprising the sequence QQGYTLPYT (SEQ ID NO:51).

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 sequence selected from the group consisting of SEQ ID NOS: 46, 52, 120, and 126;

(ii) a VH-CDR2 sequence selected from the group consisting of SEQ ID NOS: 47, 53, 121, and 127;

(iii) a VH-CDR3 sequence selected from the group consisting of SEQ ID NOS: 48, 54, 122, and 128;

(iv) a VL-CDR1 sequence selected from the group consisting of SEQ ID NOS: 49, 55, 123, and 129;

(v) a VL-CDR2 sequence selected from the group consisting of SEQ ID NOS: 50, 56, 124, and 130; and,

(vi) a VL-CDR3 sequence selected from the group consisting of SEQ ID NOS: 51, 57, 125, and 131.

In some embodiments, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH region comprising an amino acid sequence at least about 80%, 85%, 90%, 95%, or 100% identical to any one of SEQ ID NOS: 8, 10, 100, or 102 and a VL region comprising an amino acid sequence at least about 80%, 85%, 90%, 95%, or 100% identical to any one of SEQ ID NOS: 9, 11, 101, or 103. In some embodiments, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH region comprising the amino acid sequence of SEQ ID NO: 8 and a VL region comprising the amino acid sequence of SEQ ID NO: 9. In some embodiments, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH region comprising the amino acid sequence of SEQ ID NO: 10 and a VL region comprising the amino acid sequence of SEQ ID NO: 11. In some embodiments, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH region comprising the amino acid sequence of SEQ ID NO: 100 and a VL region comprising the amino acid sequence of SEQ ID NO: 101. In some embodiments, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH region comprising the amino acid sequence of SEQ ID NO: 102 and a VL region comprising the amino acid sequence of SEQ ID NO: 103. In some embodiments, the anti-GPIIb/IIIa antibody or antigen-binding molecule thereof binds to a GPIIb/IIIa epitope located in the extracellular domain of the alpha subunit of GPIIb/IIIa or to a binding site formed by the extracellular domains of the GPIIb/IIIa complex. In some embodiments, the anti-GPIIb/IIIa antibody or antigen-binding molecule thereof competes with fibrinogen for binding to GPIIb/IIIa.

In some embodiments a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to SEQ ID NO: 58;

(ii) a variable heavy chain CDR-2 (VH-CDR2) sequence at least about 60%, 70%, 80%, 90%, 95%, or 100% identical to SEQ ID NO: 59;

(iii) a variable heavy chain CDR-3 (VH-CDR3) sequence at least about 60%, 70%, 80%, 90%, 95%, or 100% identical to SEQ ID NO: 60;

(iv) a variable light chain CDR-1 (VL-CDR1) sequence at least about 60%, 70%, 80%, 90%, 95%, or 100% identical to SEQ ID NO: 61;

(v) a variable light chain CDR-2 (VL-CDR2) sequence at least about 60%, 70%, 80%, 90%, 95%, or 100% identical to SEQ ID NO: 62; and,

(vi) a variable light chain CDR-3 (VL-CDR3) sequence at least about 60%, 70%, 80%, 90%, 95%, or 100% identical to SEQ ID NO: 63.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 sequence comprising SEQ ID NO: 58;

(ii) a VH-CDR2 sequence comprising SEQ ID NO: 59;

(iii) a VH-CDR3 sequence comprising SEQ ID NO: 60;

(iv) a VL-CDR1 sequence comprising SEQ ID NO: 61;

(v) a VL-CDR2 sequence comprising SEQ ID NO: 62; and,

(vi) a VL-CDR3 sequence comprising SEQ ID NO: 63.

In some embodiments, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH region comprising an amino acid sequence at least about 80%, 85%, 90%, 95%, or 100% identical to SEQ ID NO: 12, and a VL region comprising an amino acid sequence at least about 80%, 85%, 90%, 95%, or 100% identical to SEQ ID NO: 13. In other embodiments, a chimeric molecule comprises FVII, an XTEN polypeptide, and an anti-GPIIb/IIIa antibody or antigen-binding molecule thereof, wherein the anti-GPIIb/IIIa antibody or antigen-binding molecule thereof comprises a VH region comprising the amino acid sequence of SEQ ID NO: 12, and a VL region comprising the amino acid sequence of SEQ ID NO: 13. In some embodiments, the epitope of the anti-GPIIb/IIIa antibody or antigen-binding molecule thereof is located in the extracellular domain of the alpha subunit of GPIIb/IIIa. In some embodiments, the anti-GPIIb/IIIa antibody or antigen-binding molecule thereof competes with fibrinogen for binding to GPIIb/IIIa.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 64, 70, or 135;

(ii) a variable heavy chain CDR-2 (VH-CDR2) sequence at least about 60%, 70%, 80%, 90%, 95%, or 100% identical to any one of SEQ ID NOS: 65, 71, or 136;

(iii) a variable heavy chain CDR-3 (VH-CDR3) sequence at least about 60%, 70%, 80%, 90%, 95%, or 100% identical to any one of SEQ ID NOS: 66, 72, or 137;

(iv) a variable light chain CDR-1 (VL-CDR1) sequence at least about 60%, 70%, 80%, 90%, 95%, or 100% identical to any one of SEQ ID NOS: 67, 132, or 138;

(v) a variable light chain CDR-2 (VL-CDR2) sequence at least about 60%, 70%, 80%, 90%, 95%, or 100% identical to any one of SEQ ID NOS: 68, 133, or 139; and,

(vi) a variable light chain CDR-3 (VL-CDR3) sequence at least about 60%, 70%, 80%, 90%, 95%, or 100% identical to any one of SEQ ID NOS: 69, 134, or 140.

In other embodiments, a chimeric molecule comprises FVII, an XTEN polypeptide, and an anti-GPIIb/IIIa antibody or antigen-binding molecule thereof, wherein the antibody or antigen-binding molecule thereof comprises: (i) a VH-CDR1 comprising the sequence SYWIE (SEQ ID NO: 64); (ii) a VH-CDR2 comprising the consensus sequence EILPGX₁₄GX₁₅TKYNX₁₆KFKG (SEQ ID NO: ______), wherein X₁₄ represents any amino acids, e.g., an amino acid residue with uncharged polar side chain, e.g., Ser (S) or Thr (T), X₁₅ represents any amino acids, e.g., an amino acid residue with uncharged polar side chains or beta-branched side chains, e.g., Ile (I) or Tyr (Y), and X₁₆ represents any amino acid, e.g., an amino acid residue with acidic side chains, e.g., Asp (D) or Glu (E); (iii) a VH-CDR3 comprising the sequence LISYYYAMDY (SEQ ID NO: 66); (iv) a VL-CDR1 comprising the sequence RASQDISNYLN (SEQ ID NO: 67); (v) a VL-CDR2 comprising the sequence YTSRLHS (SEQ ID NO: 68); and, (vi) a VL-CDR3 comprising the sequence QQGNTLPPT (SEQ ID NO: 69).

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 sequence selected from the group consisting of SEQ ID NOS: 64, 70, and 135;

(ii) a VH-CDR2 sequence selected from the group consisting of SEQ ID NOS: 65, 71, and 136;

(iii) a VH-CDR3 sequence selected from the group consisting of SEQ ID NOS: 66, 72, and 137;

(iv) a VL-CDR1 sequence selected from the group consisting of SEQ ID NOS: 67, 132, and 138;

(v) a VL-CDR2 sequence selected from the group consisting of SEQ ID NOS: 68, 133, and 139; and,

(vi) a VL-CDR3 sequence selected from the group consisting of SEQ ID NOS: 69, 134, and 140.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises: a VH region comprising an amino acid sequence at least about 80%, 85%, 90%, 95%, or 100% identical to any one of SEQ ID NOS: 14, 16, or 105 and a VL region comprising an amino acid sequence at least about 80%, 85%, 90%, 95%, or 100% identical to any one of SEQ ID NOS: 15, 104, or 106. In some embodiments, a chimeric molecule comprises FVII, an XTEN polypeptide, and an anti-GPIIb/IIIa antibody or antigen-binding molecule thereof, wherein the antibody or antigen-binding molecule thereof comprises: a VH region comprising the amino acid sequence of SEQ ID NO: 14 and a VL region comprising the amino acid sequence of SEQ ID NO: 15. In some embodiments, a chimeric molecule comprises FVII, an XTEN polypeptide, and an anti-GPIIb/IIIa antibody or antigen-binding molecule thereof, wherein the antibody or antigen-binding molecule thereof comprises: a VH region comprising the amino acid sequence of SEQ ID NO: 16 and a VL region comprising the amino acid sequence of SEQ ID NO: 104. In some embodiments, a chimeric molecule comprises FVII, an XTEN polypeptide, and an anti-GPIIb/IIIa antibody or antigen-binding molecule thereof, wherein the antibody or antigen-binding molecule thereof comprises: a VH region comprising the amino acid sequence of SEQ ID NO: 105 and a VL region comprising the amino acid sequence of SEQ ID NO: 106. In some embodiments, the anti-GPIIb/IIIa antibody or antigen-binding molecule thereof binds to a GPIIb/IIIa epitope located in the extracellular domain of the beta subunit of GPIIb/IIIa. In some embodiments, the anti-GPIIb/IIIa antibody or antigen-binding molecule thereof does not compete with fibrinogen for binding to GPIIb/IIIa.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to any one of SEQ ID NOS: 73, 76, 79, 85, or 147;

(ii) a variable heavy chain CDR-2 (VH-CDR2) sequence at least about 60%, 70%, 80%, 90%, 95%, or 100% identical to any one of SEQ ID NOS: 74, 77, 80, 86, or 148;

(iii) a variable heavy chain CDR-3 (VH-CDR3) sequence at least about 60%, 70%, 80%, 90%, 95%, or 100% identical to any one of SEQ ID NOS: 75, 78, 81, 87, or 149;

(iv) a variable light chain CDR-1 (VL-CDR1) sequence at least about 60%, 70%, 80%, 90%, 95%, or 100% identical to any one of SEQ ID NOS: 141, 144, 82, 88, or 150;

(v) a variable light chain CDR-2 (VL-CDR2) sequence at least about 60%, 70%, 80%, 90%, 95%, or 100% identical to any one of SEQ ID NOS: 142, 145, 83, 89, or 151; and,

(vi) a variable light chain CDR-3 (VL-CDR3) sequence at least about 60%, 70%, 80%, 90%, 95%, or 100% identical to any one of SEQ ID NO: 143, 146, 84, 90, or 152.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 comprising the consensus sequence TSGX₁₁GVG, wherein X₁₁ represents any amino acid residue, e.g., an amino acid residue with nonpolar side chains, e.g., Met (M) or Leu (L);

(ii) a VH-CDR2 comprising the consensus sequence HIWWDDDKRYNPX₁₂LKS, wherein X₁₂ represents any amino acid residue, e.g., an amino acid residue with nonpolar side chains or beta-branched side chains, e.g., Ala (A) or Thr (T);

(iii) a VH-CDR3 comprising the consensus sequence SHYX₁₃GTFYFDX₁₄, wherein X₁₃ represents any amino acid residue, e.g., an amino acid residue with uncharged polar side chain, e.g., Tyr (Y) or Asn (N), and X₁₄ represents any amino acid residue, e.g., an amino acid residue with aromatic side chain, e.g., Tyr (Y) or Phe (F); (iv) a VL-CDR1 comprising the sequence RASKSISKYLA (SEQ ID NO: 82);

(v) a VL-CDR2 comprising the sequence SGSTLQS (SEQ ID NO: 83); and,

(vi) a VL-CDR3 comprising the sequence QQHIEYPWT (SEQ ID NO: 84).

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 sequence selected from the group consisting of SEQ ID NOS: 73, 76, 79, 85, and 147;

(ii) a VH-CDR2 sequence selected from the group consisting of SEQ ID NOS: 74, 77, 80, 86, and 148;

(iii) a VH-CDR3 sequence selected from the group consisting of SEQ ID NOS: 75, 78, 81, 87, and 149;

(iv) a VL-CDR1 sequence selected from the group consisting of SEQ ID NOS: 141, 144, 82, 88, and 150;

(v) a VL-CDR2 sequence selected from the group consisting of SEQ ID NOS: 142, 145, 83, 89, and 151; and,

(vi) a VL-CDR3 sequence selected from the group consisting of SEQ ID NOS: 143, 146, 84, 90, and 152.

In some embodiments, a chimeric molecule comprises FVII, an XTEN polypeptide, and an anti-GPIIb/IIIa antibody or antigen-binding molecule thereof, wherein the antibody or antigen-binding molecule comprises: a VH region comprising an amino acid sequence at least 80%, 85%, 90%, 95%, or 100% identical to any one of SEQ ID NOS: 17, 18, 19, 21, or 109 and a VL region comprising an amino acid sequence at least 80%, 85%, 90%, 95%, or 100% identical to any one of SEQ ID NOS: 107, 108, 20, 22, or 110.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises: a VH region comprising the amino acid sequence of SEQ ID NO: 17 and a VL region comprising the amino acid sequence of SEQ ID NO: 107. In other embodiments, a chimeric molecule comprises FVII, an XTEN polypeptide, and an anti-GPIIb/IIIa antibody or antigen-binding molecule thereof, wherein the antibody or antigen-binding molecule thereof comprises: a VH region comprising the amino acid sequence of SEQ ID NO: 18 and a VL region comprising the amino acid sequence of SEQ ID NO: 108. In some embodiments, a chimeric molecule comprises FVII, an XTEN polypeptide, and an anti-GPIIb/IIIa antibody or antigen-binding molecule thereof, wherein the antibody or antigen-binding molecule comprises: a VH region comprising the amino acid sequence of SEQ ID NO: 109 and a VL region comprising the amino acid sequence of SEQ ID NO: 110. In other embodiments, a chimeric molecule comprises FVII, an XTEN polypeptide, and an anti-GPIIb/IIIa antibody or antigen-binding molecule thereof, wherein the antibody or antigen-binding molecule comprises: a VH region comprising the amino acid sequence of SEQ ID NO: 19 and a VL region comprising the amino acid sequence of SEQ ID NO: 20. In other embodiments, a chimeric molecule comprises FVII, an XTEN polypeptide, and an anti-GPIIb/IIIa antibody or antigen-binding molecule thereof, wherein the antibody or antigen-binding molecule comprises a VH region comprising the amino acid sequence of SEQ ID NO: 21 and a VL region comprising the amino acid sequence of SEQ ID NO: 22. In some embodiments, the anti-GPIIb/IIIa antibody or antigen-binding molecule thereof binds to a GPIIb/IIIa epitope located in the extracellular domain of the beta subunit of GPIIb/IIIa. In other embodiments, the anti-GPIIb/IIIa antibody or antigen-binding molecule thereof competes with fibrinogen for binding to GPIIb/IIIa.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a variable heavy chain CDR-1 (VH-CDR1) sequence at least about 60%, 70%, 80%, 90%, 95%, or 100% identical to SEQ ID NO: 91;

(ii) a variable heavy chain CDR-2 (VH-CDR2) sequence at least about 60%, 70%, 80%, 90%, 95%, or 100% identical to SEQ ID NO: 92;

(iii) a variable heavy chain CDR-3 (VH-CDR3) sequence at least about 60%, 70%, 80%, 90%, 95%, or 100% identical to SEQ ID NO: 93;

(iv) a variable light chain CDR-1 (VL-CDR1) sequence at least about 60%, 70%, 80%, 90%, 95%, or 100% identical to SEQ ID NO: 94;

(v) a variable light chain CDR-2 (VL-CDR2) sequence at least about 60%, 70%, 80%, 90%, 95%, or 100% identical to SEQ ID NO: 95; and,

(vi) a variable light chain CDR-3 (VL-CDR3) sequence at least about 60%, 70%, 80%, 90%, 95%, or 100% identical to SEQ ID NO: 96.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises:

(i) a VH-CDR1 sequence comprising SEQ ID NO: 91;

(ii) a VH-CDR2 sequence comprising SEQ ID NO: 92;

(iii) a VH-CDR3 sequence comprising SEQ ID NO: 93;

(iv) a VL-CDR1 sequence comprising SEQ ID NO: 94;

(v) a VL-CDR2 sequence comprising SEQ ID NOS: 95; and,

(vi) a VL-CDR3 sequence comprising SEQ ID NOS: 96.

In some embodiments, a chimeric molecule comprises FVII, an XTEN polypeptide, and an anti-GPIIb/IIIa antibody or antigen-binding molecule thereof, wherein the antibody or antigen-binding molecule thereof comprises: a VH region comprising an amino acid sequence at least about 80%, 85%, 90%, 95%, or 100% identical to SEQ ID NO: 23 and a VL region comprising an amino acid sequence at least about 80%, 85%, 90%, 95%, or 100% identical to SEQ ID NO: 24. In some embodiments, the anti-GPIIb/IIIa antibody or antigen-binding molecule thereof binds to a GPIIb/IIIa epitope located in the extracellular domain of the molecule thereof competes with fibrinogen for binding to GPIIb/IIIa.

In some embodiments, the anti-GPIIb/IIIa antibody or antigen-binding molecule thereof comprises or consists of (a) a single chain Fv (“scFv”); (b) a diabody; (c) a minibody; (d) a polypeptide chain of an antibody; (e) F(ab′)₂; or (f) F(ab). In certain embodiments, suitable anti-GPIIb/IIIa antibody or antigen-binding molecule thereof include, for example, any member of a specific binding pair, antibodies, monoclonal antibodies, or derivatives or analogs derived from the anti-GPIIb/IIIa antibody disclosed herein, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((scFv) fragments), diabodies, tribodies or tetrabodies, which typically are covalently linked or otherwise stabilized (i.e., leucine zipper or helix stabilized) scFv fragments.

### II.B. Chimeric Molecules Comprising Targeting Moiety and XTEN

The present invention also provides a chimeric molecule comprising a combination of a light chain of FVII, a heavy chain of FVII, an XTEN polypeptide, and a targeting moiety, which binds to a platelet. In order to improve the clotting activity and pharmacokinetic properties, the chimeric molecule can contain a half-life extending moiety, i.e., an XTEN polypeptide, and a platelet targeting moiety.

In one embodiment, a chimeric molecule comprises a first polypeptide chain and a second polypeptide chain, which are associated with each other, wherein the first polypeptide chain comprises a light chain of FVII and an XTEN polypeptide and the second chain polypeptide chain comprises a heavy chain of FVII and a targeting moiety, which binds to a platelet. In another embodiment, a chimeric molecule comprises a first polypeptide chain and a second polypeptide chain, which are associated with each other, wherein the first polypeptide chain comprises a light chain of FVII and a targeting moiety, which binds to a platelet, and the second polypeptide chain comprises a heavy chain of FVII and an XTEN polypeptide. In other embodiments, a chimeric molecule comprises a first polypeptide chain and a second polypeptide chain, which are associated with each other, wherein the first polypeptide chain comprises a light chain of FVII and the second polypeptide chain comprises a heavy chain of FVII, an XTEN polypeptide, and a targeting moiety, which binds to a platelet. In some embodiments, a chimeric molecule comprises a first polypeptide chain and a second polypeptide chain, which are associated with each other, wherein the first polypeptide chain comprises a light chain of FVII and the second polypeptide chain comprises a heavy chain of FVII, a targeting moiety, which binds to a platelet, or an XTEN polypeptide.

In certain embodiments, a chimeric molecule comprises a first polypeptide chain and a second polypeptide chain, which are associated with each other, wherein the first polypeptide chain comprises a formula of FVII_{L}-Tm or Tm-FVII_{L} and the second polypeptide chain comprises FVII_{H}-X or X-FVII_{H}. In some embodiments, a chimeric molecule comprises a first polypeptide chain and a second polypeptide chain, which are associated with each other, wherein the first polypeptide chain comprises a formula of FVII_{L}-X or X-FVII_{L} and the second polypeptide chain comprises a formula of FVII_{H}-Tm or Tm-FVII_{H}. In other embodiments, a chimeric molecule comprises a first polypeptide chain and a second polypeptide chain, which are associated with each other, wherein the first polypeptide chain comprises the formula of FVII_{L} and the second polypeptide chain comprises a formula of FVII_{H}-X-Tm or Tm-X-FVII_{H}. In yet other embodiments, a chimeric molecule comprises a first polypeptide chain and a second polypeptide chain, which are associated with each other, wherein the first polypeptide chain comprises the formula of FVII_{L} and the second polypeptide chain comprises a formula of FVII_{H}-Tm-X or X-Tm-FVII_{H}. Each component of the chimeric molecule is noted as follows: FVII_{H} is a heavy chain of FVII; Tm is a targeting moiety, which binds to a platelet; FVII_{L} is a light chain of FVII; and X is an XTEN polypeptide.

In other embodiments, a chimeric molecule comprises a formula of X-FVII_{L}:FVII_{H}-Tm, X-FVII_{L}:Tm-FVII_{H}, FVII_{L}-X:FVII_{H}-Tm, FVII_{L}-X:Tm-FVII_{H}, Tm-FVII_{H}:X-FVII_{L}, Tm-FVII_{H}:FVII_{L}-X, FVII_{H}-Tm:FVII_{L}-X, or FVII_{H}-Tm:X-FVII_{L}, wherein FVII_{H} is a heavy chain of FVII; Tm is a targeting moiety, which binds to a platelet; FVII_{L} is a light chain of FVII; X is an XTEN polypeptide; and (:) is an association between two polypeptide chains. In yet other embodiments, a chimeric molecule comprises a formula of FVII_{L}:FVII_{H}-X-Tm; Tm-X-FVII_{H}:FVII_{L}; FVII_{L}:FVII_{H}-Tm-X; or X-Tm-FVII_{H}:FVII_{L}; wherein FVII_{H} is a heavy chain of FVII; Tm is a targeting moiety, which binds to a platelet; FVII_{L} is a light chain of FVII; X is an XTEN polypeptide; and (:) is an association between two polypeptide chains.

In certain embodiments, the first polypeptide chain and the second polypeptide chain of the chimeric molecule are associated with each other. The association between the first polypeptide chain and the second polypeptide chain can be a covalent bond or a non-covalent bond. In some embodiments, the association between the first polypeptide chain and the second polypeptide chain is a covalent bond between the heavy chain and the light chain of FVII. In other embodiments, the association between the first polypeptide chain and the second polypeptide chain is a covalent bond, i.e., a disulfide bond.

In some embodiments, a chimeric molecule comprises a single polypeptide chain, which comprises, from N terminus to C terminus,

(a) a light chain of FVII, an XTEN polypeptide, a protease cleavage site, a heavy chain of FVII, and a targeting moiety which binds to a platelet;

(b) a light chain of FVII, a targeting moiety which binds to a platelet, a protease cleavage site, a heavy chain of FVII, and an XTEN polypeptide;

(c) a light chain of FVII, an optional protease cleavage site, a heavy chain of FVII, an XTEN polypeptide, and a targeting moiety which binds to a platelet; or

(d) a light chain of FVII, an optional protease cleavage site, a heavy chain of FVII, a targeting moiety which binds to a platelet, and an XTEN polypeptide. In one embodiment, the protease cleavage site comprises an intracellular processing site. In another embodiment, an intracellular processing site is processed by a proprotein convertase, e.g., PC5, PACE, PC7, and any combinations thereof.

In some embodiments, the chimeric molecule comprises an amino acid sequence at least about 70%, 80%, 90%, 95%, 96%, 97%, 98%, 99%, or 100% identical to the amino acid sequence encoded by SEQ ID NO: 191 or SEQ ID NO: 192.

In some embodiments, the chimeric molecule comprises an amino acid sequence at least about 70%, 80%, 90%, 95%, 96%, 97%, 98%, 99%, or 100% identical to the amino acid sequence encoded by SEQ ID NO: 188 or SEQ ID NO: 190.

In one embodiment, a chimeric molecule of the invention comprises a FVII light chain fused to an XTEN polypeptide having 72 amino acids (e.g., AE72, AE72_2, or AE72_3) and a FVII heavy chain fused to an scFv of an anti-GPIIb/IIIa antibody (e.g., 34D10). In another embodiment, a chimeric molecule of the invention comprises a FVII light chain fused to an XTEN polypeptide having 144 amino acids (e.g., AE144, AE144_2, AE144_3, or AG144) and a FVII heavy chain fused to an scFv of an anti-GPIIb/IIIa antibody (e.g., 34D10). In other embodiments, a chimeric molecule comprises a FVII light chain and a FVII heavy chain, wherein the FVII heavy chain is fused to an scFv of an anti-GPIIb/IIIa antibody (e.g., 34D10) by an XTEN polypeptide having 72 amino acids (e.g., AE72, AE72_2, or AE72_3). In some embodiments, a chimeric molecule comprises a FVII light chain and a FVII heavy chain, wherein the FVII heavy chain is fused to an scFv of anti-GPIIb/IIIa antibody (e.g., 34D10) by an XTEN polypeptide having 144 amino acids (e.g., AE144, AE144_2, AE144_3, or AG144). In certain embodiments, a chimeric molecule comprises a FVII light chain fused to an XTEN polypeptide having 42 amino acids (e.g., AE42, AE42_2, or AE42_3) and a FVII heavy chain fused to an scFv of an anti-GPIIb/IIIa antibody by an XTEN polypeptide having 72 amino acids (e.g., AE72, AE72_2, or AE72_3). In other embodiments, a chimeric molecule comprises a FVII light chain fused to an XTEN polypeptide having 72 amino acids (e.g., AE72, AE72_2, or AE72_3) and a FVII heavy chain fused to an scFv of an anti-GPIIb/IIIa antibody by an XTEN polypeptide having 42 amino acids (e.g., AE42, AE42_2, or AE42_3). In still other embodiments, a chimeric molecule comprises a FVII light chain fused to an XTEN polypeptide having 72 amino acids (e.g., AE72, AE72_2, or AE72_3) and a FVII heavy chain fused to an scFv of an anti-GPIIb/IIIa antibody by an XTEN polypeptide having 72 amino acids (e.g., AE72, AE72_2, or AE72_3).

In certain embodiments, the targeting moiety, which binds to a platelet, is selected from the group consisting of: an antibody or antigen-binding molecule thereof, a receptor binding portion of a receptor, and a peptide. In some embodiments, the targeting moiety selectively binds to a resting platelet or an activated platelet. In other embodiments, the targeting moiety selectively binds to a target selected from the group consisting of: GPIba, GPVI, GPIX, a nonactive form of glycoprotein IIb/IIIa (“GPIIb/IIIa”), an active form of GPIIb/IIIa, P selectin, GMP-33, LAMP-1, LAMP-2, CD40L, LOX-1, and any combinations thereof. In a specific embodiment, the targeting moiety is an antibody or antigen-binding molecule thereof, which binds to a GPIIb/IIIa epitope (“anti-GPIIb/IIIa antibody or antigen-binding molecule thereof”).

As used herein, the phrases “which binds to a platelet,” “binding to a platelet,” and variants thereof generally refer to the specific binding of (i) a GPIIb/IIIa antibody or antigen-binding molecule thereof or (ii) a chimeric molecule of the present disclosure to an antigenic site on the surface of the platelet, e.g., an epitope on the extracellular domains of the α and/or β subunits of the GPIIb/IIIa receptor. It would be known to a person skilled in the art that GPIIb/IIIa is present in two pools, a plasma membrane pool present in the platelet's resting state and an internal pool of GPIIb/IIIa which is expressed upon platelet activation. See, for example, Quinn et al., J. Pharmacol. Exp. Ther. 297:496-500 (2001). Accordingly, in some specific embodiments, and particularly for diagnostic uses where the platelet's plasma membrane can be permeabilized, the binding of a GPIIb/IIIa antibody or antigen-binding molecule thereof to platelets, or the binding of a chimeric molecule of the present disclosure to platelets can refer to binding to the plasma membrane pool and/or to the internal pool of GPIIb/IIIa.

In some embodiments, the targeting moiety in the chimeric molecule is selected from the group consisting of: an antibody or antigen-binding molecule thereof, a receptor binding portion of a receptor, and a peptide. In some embodiments, the targeting moiety selectively binds to a resting platelet or an activated platelet. In other embodiments, the targeting moiety selectively binds to a target selected from the group consisting of: GP1ba (Uniprot: E7ES66; E7ES66_HUMAN), GPVI (Uniprot: Q9HCN6; GPVI_HUMAN), GPIX (Uniprot: P14770; GPIX_HUMAN), a nonactive form of glycoprotein IIb/IIIa (“GPIIb/IIIa”), an active form of GPIIb/IIIa, P-selectin (Uniprot: Q14242; SELPL_HUMAN), GMP-33 (see, e.g., Damas et al., Thromb. Haemost. 86:887-93 (2001)), LAMP-1 (Uniprot: P11279; LAMP1_HUMAN), LAMP-2 (Uniprot: P13473; LAMP2_HUMAN), CD40L (Uniprot: P29965; CD40L_HUMAN), LOX-1 (Uniprot: P78380; OLR1_HUMAN), and any combinations thereof. The above referenced Uniprot identifiers correspond the entries published in the Universal Protein Resource (Uniprot) database release 2013_05 (May 1, 2013), and are incorporated by reference in their entireties. In certain embodiments, the targeting moiety comprises a GPIIb/IIIa antibody or antigen-binding molecule thereof. In specific embodiments, the GPIIb/IIIa antibody or antigen-binding molecule thereof is a GPIIb/IIIa antibody or antigen-binding molecule thereof disclosed in section II.A.1.

### II.C. XTEN Polypeptides

As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a chimeric molecule partner, XTENs can serve as a carrier, conferring certain desirable pharmacokinetic, physicochemical and pharmaceutical properties when linked to a clotting factor, a heavy chain of a clotting factor, a light chain or a clotting factor, a targeting moiety, or any other sequences or molecules on the chimeric molecule. Such desirable properties include but are not limited to enhanced pharmacokinetic parameters and solubility characteristics. As used herein, “XTEN” specifically excludes antibodies or antibody fragments such as single-chain antibodies or Fc fragments of a light chain or a heavy chain.

The chimeric molecules of the invention can include a single XTEN polypeptide or two or more XTEN polypeptides. In one embodiment, a chimeric molecule comprises FVII, a first XTEN polypeptide, a second XTEN polypeptide, and an anti-GPIIb/IIIa antibody or antigen-binding molecule thereof. The chimeric molecule thus can comprise a formula of FVII-(L1)-X1-(L2)-Tm-(L3)-X2, X2-(L1)-Tm-(L2)-X1-(L3)-FVII, FVII-(L1)-X1-(L2)-X2-(L3)-Tm, or Tm-(L3)-X2-(L2)-X1-(L1)-FVII, wherein FVII comprises FVIIa, X1 is a first XTEN polypeptide, X2 is a second XTEN polypeptide, Tm is an anti-GPIIb/IIIa antibody or antigen-binding molecule thereof as described in section II.A.1., L1 is a first optional linker, L2 is a second optional linker, and L3 is a third optional linker. In another embodiment, a chimeric molecule comprises two polypeptide chains associated with each other, the first polypeptide chain comprising a light chain of FVII and a first XTEN polypeptide the second polypeptide chain comprising a heavy chain of FVII, a second XTEN polypeptide, and a targeting moiety, which binds to a platelet, in any order. In other embodiments, a chimeric molecule comprises two polypeptide chains associated with each other, the first polypeptide chain comprising a light chain of FVII and the first XTEN polypeptide a second polypeptide chain comprising, from N-terminus to C-terminus, a heavy chain of FVII, a second XTEN polypeptide, and a targeting moiety, which binds to a platelet or a heavy chain of FVII, a targeting moiety, which binds to a platelet, and a second XTEN polypeptide.

In some embodiments, the XTEN sequence of the invention is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues.

The XTEN sequence of the invention can comprise one or more sequence motif of 9 to 14 amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids selected from the group consisting of glycine (G), alanine (A), serine (S), threonine (T), glutamate (E) and proline (P). See US 2010-0239554 A1,

In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of non-overlapping sequences selected from a single motif family selected from TABLE 1, resulting in a family sequence. As used herein, “family” means that the XTEN has motifs selected only from a single motif category from TABLE 1; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to FVII. In some embodiments of XTEN families, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology described above. In other embodiments, the XTEN comprises multiple units of motif sequences from two or more of the motif families of TABLE 1. These sequences can be selected to achieve desired physical/chemical characteristics, including such properties as net charge, hydrophilicity, lack of secondary structure, or lack of repetitiveness that are conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues. Additional, non-limiting, examples of XTENs linked to FVII are disclosed in U.S. Patent Publication No. 2012/0263701, which is incorporated herein by reference in its entirety.

TABLE 1
XTEN Sequence Motifs of 12 Amino Acids and
Motif Families

| Motif Family* | MOTIF SEQUENCE | SEQ ID NO: |
|---|---|---|
| AD | GESPGGSSGSES | 194 |
| AD | GSEGSSGPGESS | 195 |
| AD | GSSESGSSEGGP | 196 |
| AD | GSGGEPSESGSS | 197 |
| AE, AM | GSPAGSPTSTEE | 198 |
| AE, AM, AQ | GSEPATSGSETP | 199 |
| AE, AM, AQ | GTSESATPESGP | 200 |
| AE, AM, AQ | GTSTEPSEGSAP | 201 |
| AF, AM | GSTSESPSGTAP | 202 |
| AF, AM | GTSTPESGSASP | 203 |
| AF, AM | GTSPSGESSTAP | 204 |
| AF, AM | GSTSSTAESPGP | 205 |
| AG, AM | GTPGSGTASSSP | 206 |
| AG, AM | GSSTPSGATGSP | 207 |
| AG, AM | GSSPSASTGTGP | 208 |
| AG, AM | GASPGTSSTGSP | 209 |
| AQ | GEPAGSPTSTSE | 210 |
| AQ | GTGEPSSTPASE | 211 |
| AQ | GSGPSTESAPTE | 212 |
| AQ | GSETPSGPSETA | 213 |
| AQ | GPSETSTSEPGA | 214 |
| AQ | GSPSEPTEGTSA | 215 |
| BC | GSGASEPTSTEP | 216 |
| BC | GSEPATSGTEPS | 217 |
| BC | GTSEPSTSEPGA | 218 |
| BC | GTSTEPSEPGSA | 219 |
| BD | GSTAGSETSTEA | 220 |
| BD | GSETATSGSETA | 221 |
| BD | GTSESATSESGA | 222 |
| BD | GTSTEASEGSAS | 223 |

Denotes individual motif sequences that, when used together in various permutations, results in a “family sequence”

XTEN can have varying lengths. In one embodiment, the length of the XTEN polypeptide(s) is chosen based on the property or function to be achieved in the fusion protein. Depending on the intended property or function, XTEN can be short or intermediate length sequence or longer sequence that can serve as carriers. In certain embodiments, the XTEN include short segments of about 6 to about 99 amino acid residues, intermediate lengths of about 100 to about 399 amino acid residues, and longer lengths of about 400 to about 1000 and up to about 3000 amino acid residues. Thus, the XTEN linked to FVII (e.g., heavy chain or light chain) or a targeting moiety can have lengths of about 6, about 12, about 36, about 40, about 42, about 72, about 96, about 144, about 288, about 400, about 500, about 576, about 600, about 700, about 800, about 864, about 900, about 1000, about 1500, about 2000, about 2500, or up to about 3000 amino acid residues in length. In other embodiments, the XTEN sequences is about 6 to about 50, about 50 to about 100, about 100 to 150, about 150 to 250, about 250 to 400, about 400 to about 500, about 500 to about 900, about 900 to 1500, about 1500 to 2000, or about 2000 to about 3000 amino acid residues in length. The precise length of an XTEN polypeptide that can be linked to FVII (e.g., light chain or heavy chain) or a targeting moiety (Tm) can vary without adversely affecting the activity of FVII. In one embodiment, one or more of the XTEN used herein has about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, or about 2004 amino acids in length and can be selected from one or more of the XTEN family sequences; i.e., AD, AE, AF, AG, AM, AQ, BC, BD, or any combinations thereof.

In some embodiments, the XTEN polypeptide used in the invention is at least 60%, 70%, 80%, 85%, 90%, 91%, 92%, 93%, 94%, 95%, 96%, 97%, 98%, 99%, or 100% identical to a sequence selected from the group consisting of AE42, AG42, AE42_2, AE42_3, AE48, AM48, AE72, AE72_2, AE72_3, AG72, AE108, AG108, AE144, AF144, AE144_2, AE144_3, AG144, AE180, AG180, AE216, AG216, AE252, AG252, AE288, AG288, AE295, AE324, AG324, AE360, AG360, AE396, AG396, AE432, AG432, AE468, AG468, AE504, AG504, AF504, AE540, AG540, AF540, AD576, AE576, AF576, AG576, AE612, AG612, AE624, AE648, AG648, AG684, AE720, AG720, AE756, AG756, AE792, AG792, AE828, AG828, AD836, AE864, AF864, AG864, AE872, AE884, AM875, AE912, AM923, AM1318, BC864, BD864, AE948, AE1044, AE1140, AE1236, AE1332, AE1428, AE1524, AE1620, AE1716, AE1812, AE1908, AE2004A, AG948, AG1044, AG1140, AG1236, AG1332, AG1428, AG1524, AG1620, AG1716, AG1812, AG1908, AG2004, and any combinations thereof. See US 2010-0239554 A1.

In one embodiment, the XTEN sequence is at least 60%, 70%, 80%, 90%, 95%, 96%, 97%, 98%, 99% or 100% identical to an amino acid sequence selected from the group consisting of AE42, AE864, AE576, AE288, AE144, AG864, AG576, AG288, AG144, and any combinations thereof. In another embodiment, the XTEN sequence is selected from the group consisting of AE42, AE864, AE576, AE288, AE144, AG864, AG576, AG288, AG144, and any combinations thereof. In a specific embodiment, the XTEN sequence is AE288. The amino acid sequences for certain XTEN sequences of the invention are shown in TABLE 2.

TABLE 2
XTEN Sequences

| XTEN | Amino Acid Sequence |
|---|---|
| AE42 SEQ ID NO: 224 | GAPGSPAGSPTSTEEGTSESATPESGPGSEPATSGSETPASS |
| AE42_2 SEQ ID NO: | TGGGSPAGSPTSTEEGTSESATPESGPGSEPATSGSETPASS |
| 225 | |
| AE42_3 SEQ ID NO: | GTSESATPESGPGSEPATSGSETPGTSESATPESGPGSEPAT |
| 226 | |
| AE72 SEQ ID NO: 227 | GAP TSESATPESG PGSEPATSGS ETPGTSESAT |
| | PESGPGSEPA |
| | TSGSETPGTS ESATPESGPG TSTEPSEGSA PGASS |
| AE72_2 SEQ ID NO: | GTSESATPESGPGSEPATSGSETPGTSESATPESGPGSEPAT |
| 228 | SGSETPGTSESATPESGPGTSTEPSEGSAP |
| AE72_3 SEQ ID NO: | SPAGSPTSTEEGTSESATPESGPGSEPATSGSETPGTSESAT |
| 229 | PESGPGTSTEPSEGSAPGTSTEPSEGSAPG |
| AE144 | GSEPATSGSETPGTSESATPESGPGSEPATSGSETPGSPAGS |
| SEQ ID NO: 230 | PTSTEEGTSTEPSEG |
| | SAPGSEPATSGSETPGSEPATSGSETPGSEPATSGSETPGTS |
| | TEPSEGSAPGTSESA |
| | PESGPGSEPATSGSETPGTSTEPSEGSAP |
| AE144_2 | GTSESATPESGPGSEPATSGSETPGTSESATPESGPGSEPAT |
| SEQ ID NO: 231 | SGSETPGTSESATPESGPGTSTEPSEGSAPGSPAGSPTSTEE |
| | GTSESATPESGPGSEPATSGSETPGTSESATPESGPGSPAGS |
| | PTSTEEGSPAGSPTSTEE |
| AE144_3 | GSPAGSPTSTEEGTSESATPESGPGTSTEPSEGSAPGSPAGS |
| SEQ ID NO: 232 | PTSTEEGTSTEPSEGSAPGTSTEPSEGSAPGTSESATPESGP |
| | GSEPATSGSETPGSEPATSGSETPGSPAGSPTSTEEGTSESA |
| | TPESGPGTSTEPSEGSAP |
| AG144 | GTPGSGTASSSPGSSTPSGATGSPGSSPSASTGTGPGSSPSA |
| SEQ ID NO: 233 | STGTGPGASPGTSST |
| | GSPGASPGTSSTGSPGSSTPSGATGSPGSSPSASTGTGPGAS |
| | PGTSSTGSPGSSPSA |
| | STGTGPGTPGSGTASSSPGSSTPSGATGSP |
| AE288 | GTSESATPESGPGSEPATSGSETPGTSESATPESGPGSEPAT |
| SEQ ID NO: 234 | SGSETPGTSESATPESG |
| | PGTSTEPSEGSAPGSPAGSPTSTEEGTSESATPESGPGSEPA |
| | TSGSETPGTSESATPES |
| | GPGSPAGSPTSTEEGSPAGSPTSTEEGTSTEPSEGSAPGTSE |
| | SATPESGPGTSESATPE |
| | SGPGTSESATPESGPGSEPATSGSETPGSEPATSGSETPGSP |
| | AGSPTSTEEGTSTEPSE |
| | GSAPGT STEPSEGSAPGSEPATSGSETPGTSESATPESGPGT |
| | STEPSEGSAP |
| AG288 | PGASPGTSSTGSPGASPGTSSTGSPGTPGSGTASSSPGSSTPSGATGSPGTP |
| SEQ ID NO: 235 | GSGTASS |
| | SPGSSTPSGATGSPGTPGSGTASSSPGSSTPSGATGSPGSSTPSGATGSPGS |
| | SPSASTG |
| | TGPGSSPSASTGTGPGASPGTSSTGSPGTPGSGTASSSPGSSTPSGATGSPG |
| | SSPSAST |
| | GTGPGSSPSASTGTGPGASPGTSSTGSPGASPGTSSTGSPGSSTPSGATGSP |
| | GSSPSAS |
| | TGTGPGASPGTSSTGSPGSSPSASTGTGPGTPGSGTASSSPGSSTPSGATGS |
| AE576 | GSPAGSPTSTEEGTSESATPESGPGTSTEPSEGSAPGSPAGSPTSTEEGTST |
| SEQ ID NO: 236 | EPSEGSA |
| | PGTSTEPSEGSAPGTSESATPESGPGSEPATSGSETPGSEPATSGSETPGSP |
| | AGSPTST |
| | EEGTSESATPESGPGTSTEPSEGSAPGTSTEPSEGSAPGSPAGSPTSTEEGT |
| | STEPSEG |
| | SAPGTSTEPSEGSAPGTSESATPESGPGTSTEPSEGSAPGTSESATPESGPG |
| | SEPATSG |
| | SETPGTSTEPSEGSAPGTSTEPSEGSAPGTSESATPESGPGTSESATPESGP |
| | GSPAGSP |
| | TSTEEGTSESATPESGPGSEPATSGSETPGTSESATPESGPGTSTEPSEGSA |
| | PGTSTEP |
| | SEGSAPGTSTEPSEGSAPGTSTEPSEGSAPGTSTEPSEGSAPGTSTEPSEGS |
| | APGSPAG |
| | SPTSTEEGTSTEPSEGSAPGTSESATPESGPGSEPATSGSETPGTSESATPE |
| | SGPGSEP |
| | ATSGSETPGTSESATPESGPGTSTEPSEGSAPGTSESATPESGPGSPAGSPT |
| | STEEGSP |
| | AGSPTSTEEGSPAGSPTSTEEGTSESATPESGPGTSTEPSEGSAP |
| AG576 | PGTPGSGTASSSPGSSTPSGATGSPGSSPSASTGTGPGSSPSASTGTGPGSS |
| SEQ ID NO: 237 | TPSGATG |
| | SPGSSTPSGATGSPGASPGTSSTGSPGASPGTSSTGSPGASPGTSSTGSPGT |
| | PGSGTAS |
| | SSPGASPGTSSTGSPGASPGTSSTGSPGASPGTSSTGSPGSSPSASTGTGPG |
| | TPGSGTA |
| | SSSPGASPGTSSTGSPGASPGTSSTGSPGASPGTSSTGSPGSSTPSGATGSP |
| | GSSTPSG |
| | ATGSPGASPGTSSTGSPGTPGSGTASSSPGSSTPSGATGSPGSSTPSGATGS |
| | PGSSTPS |
| | GATGSPGSSPSASTGTGPGASPGTSSTGSPGASPGTSSTGSPGTPGSGTASS |
| | SPGASPG |
| | TSSTGSPGASPGTSSTGSPGASPGTSSTGSPGASPGTSSTGSPGTPGSGTAS |
| | SSPGSST |
| | PSGATGSPGTPGSGTASSSPGSSTPSGATGSPGTPGSGTASSSPGSSTPSGA |
| | TGSPGSS |
| | TPSGATGSPGSSPSASTGTGPGSSPSASTGTGPGASPGTSSTGSPGTPGSGT |
| | ASSSPGS |
| | STPSGATGSPGSSPSASTGTGPGSSPSASTGTGPGASPGTSSTGS |
| AE864 | GSPAGSPTSTEEGTSESATPESGPGTSTEPSEGSAPGSPAGSPTSTEEGTST |
| SEQ ID NO: 238 | EPSEGSA |
| | PGTSTEPSEGSAPGTSESATPESGPGSEPATSGSETPGSEPATSGSETPGSP |
| | AGSPTST |
| | EEGTSESATPESGPGTSTEPSEGSAPGTSTEPSEGSAPGSPAGSPTSTEEGT |
| | STEPSEG |
| | SAPGTSTEPSEGSAPGTSESATPESGPGTSTEPSEGSAPGTSESATPESGPG |
| | SEPATSG |
| | SETPGTSTEPSEGSAPGTSTEPSEGSAPGTSESATPESGPGTSESATPESGP |
| | GSPAGSP |
| | TSTEEGTSESATPESGPGSEPATSGSETPGTSESATPESGPGTSTEPSEGSA |
| | PGTSTEP |
| | SEGSAPGTSTEPSEGSAPGTSTEPSEGSAPGTSTEPSEGSAPGTSTEPSEGS |
| | APGSPAG |
| | SPTSTEEGTSTEPSEGSAPGTSESATPESGPGSEPATSGSETPGTSESATPE |
| | SGPGSEP |
| | ATSGSETPGTSESATPESGPGTSTEPSEGSAPGTSESATPESGPGSPAGSPT |
| | STEEGSP |
| | AGSPTSTEEGSPAGSPTSTEEGTSESATPESGPGTSTEPSEGSAPGTSESAT |
| | PESGPGS |
| | EPATSGSETPGTSESATPESGPGSEPATSGSETPGTSESATPESGPGTSTEP |
| | SEGSAPG |
| | SPAGSPTSTEEGTSESATPESGPGSEPATSGSETPGTSESATPESGPGSPAG |
| | SPTSTEE |
| | GSPAGSPTSTEEGTSTEPSEGSAPGTSESATPESGPGTSESATPESGPGTSE |
| | SATPESG |
| | PGSEPATSGSETPGSEPATSGSETPGSPAGSPTSTEEGTSTEPSEGSAPGTS |
| | TEPSEGS |
| | APGSEPATSGSETPGTSESATPESGPGTSTEPSEGSAP |
| AG864 | GASPGTSSTGSPGSSPSASTGTGPGSSPSASTGTGPGTPGSGTASSSPGSST |
| SEQ ID NO: 239 | PSGATGS |
| | PGSSPSASTGTGPGASPGTSSTGSPGTPGSGTASSSPGSSTPSGATGSPGTP |
| | GSGTASS |
| | SPGASPGTSSTGSPGASPGTSSTGSPGTPGSGTASSSPGSSTPSGATGSPGA |
| | SPGTSST |
| | GSPGTPGSGTASSSPGSSTPSGATGSPGSSPSASTGTGPGSSPSASTGTGPG |
| | SSTPSGA |
| | TGSPGSSTPSGATGSPGASPGTSSTGSPGASPGTSSTGSPGASPGTSSTGSP |
| | GTPGSGT |
| | ASSSPGASPGTSSTGSPGASPGTSSTGSPGASPGTSSTGSPGSSPSASTGTG |
| | PGTPGSG |
| | TASSSPGASPGTSSTGSPGASPGTSSTGSPGASPGTSSTGSPGSSTPSGATG |
| | SPGSSTP |
| | SGATGSPGASPGTSSTGSPGTPGSGTASSSPGSSTPSGATGSPGSSTPSGAT |
| | GSPGSST |
| | PSGATGSPGSSPSASTGTGPGASPGTSSTGSPGASPGTSSTGSPGTPGSGTA |
| | SSSPGAS |
| | PGTSSTGSPGASPGTSSTGSPGASPGTSSTGSPGASPGTSSTGSPGTPGSGT |
| | ASSSPGS |
| | STPSGATGSPGTPGSGTASSSPGSSTPSGATGSPGTPGSGTASSSPGSSTPS |
| | GATGSPG |
| | SSTPSGATGSPGSSPSASTGTGPGSSPSASTGTGPGASPGTSSTGSPGTPGS |
| | GTASSSP |
| | GSSTPSGATGSPGSSPSASTGTGPGSSPSASTGTGPGASPGTSSTGSPGASP |
| | GTSSTGS |
| | PGSSTPSGATGSPGSSPSASTGTGPGASPGTSSTGSPGSSPSASTGTGPGTP |
| | GSGTASS |
| | SPGSSTPSGATGSPGSSTPSGATGSPGASPGTSSTGSP |

In some embodiments wherein the XTEN has less than 100% of its amino acids consisting of 4, 5, or 6 types of amino acid selected from glycine (G), alanine (A), serine (S), threonine (T), glutamate (E) and proline (P), or less than 100% of the sequence consisting of the sequence motifs from Table 1 or the XTEN sequences of Table 2, the other amino acid residues of the XTEN are selected from any of the other 14 natural L-amino acids, but are preferentially selected from hydrophilic amino acids such that the XTEN sequence contains at least about 90%, 91%, 92%, 93%, 94%, 95%, 96%, 97%, 98%, or at least about 99% hydrophilic amino acids. An individual amino acid or a short sequence of amino acids other than glycine (G), alanine (A), serine (S), threonine (T), glutamate (E) and proline (P) may be incorporated into the XTEN to achieve a needed property, such as to permit incorporation of a restriction site by the encoding nucleotides, or to facilitate linking to a payload component, or incorporation of a cleavage sequence. The XTEN amino acids that are not glycine (G), alanine (A), serine (S), threonine (T), glutamate (E) and proline (P) are either interspersed throughout the XTEN sequence, are located within or between the sequence motifs, or are concentrated in one or more short stretches of the XTEN sequence such as at or near the N- or C-terminus. As hydrophobic amino acids impart structure to a polypeptide, the invention provides that the content of hydrophobic amino acids in the XTEN utilized in the conjugation constructs will typically be less than 5%, or less than 2%, or less than 1% hydrophobic amino acid content. Hydrophobic residues that are less favored in construction of XTEN include tryptophan, phenylalanine, tyrosine, leucine, isoleucine, valine, and methionine. Additionally, one can design the XTEN sequences to contain less than 5% or less than 4% or less than 3% or less than 2% or less than 1% or none of the following amino acids: methionine (to avoid oxidation), asparagine and glutamine (to avoid desamidation). In other embodiments, the amino acid content of methionine and tryptophan in the XTEN component used in the conjugation constructs is typically less than 5%, or less than 2%, and most preferably less than 1%. In other embodiments, the XTEN will have a sequence that has less than 10% amino acid residues with a positive charge, or less than about 7%, or less that about 5%, or less than about 2% amino acid residues with a positive charge, the sum of methionine and tryptophan residues will be less than 2%, and the sum of asparagine and glutamine residues will be less than 5% of the total XTEN sequence.

In further embodiments, the XTEN polypeptide used in the invention affects the physical or chemical property, e.g., pharmacokinetics, of the chimeric molecule of the present invention. The XTEN sequence used in the present invention can exhibit one or more of the following advantageous properties: conformational flexibility, enhanced aqueous solubility, high degree of protease resistance, low immunogenicity, low binding to mammalian receptors, or increased hydrodynamic (or Stokes) radii. In a specific embodiment, the XTEN polypeptide linked to FVII or a targeting moiety (e.g., an anti-GPIIb/IIIa antibody or antigen-binding molecule thereof) in this invention increases pharmacokinetic properties such as longer terminal half-life or increased area under the curve (AUC), so that the chimeric molecule described herein stays in vivo for an increased period of time compared to wild type clotting factor. In further embodiments, the XTEN polypeptide used in this invention increases pharmacokinetic properties such as longer terminal half-life or increased area under the curve (AUC), so that the clotting factor stays in vivo for an increased period of time compared to wild type FVIIa.

A variety of methods and assays can be employed to determine the physical/chemical properties of proteins comprising the XTEN polypeptide. Such methods include, but are not limited to analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Amau et al., Prot Expr and Purif 48, 1-13 (2006).

Additional examples of XTEN polypeptides that can be used according to the present invention and are disclosed in U.S. Pat. Nos. 7,855,279 and 7,846,445, US Patent Publication Nos. 2009/0092582 A1, 2010/0239554 A1, 2010/0323956 A1, 2011/0046060 A1, 2011/0046061 A1, 2011/0077199 A1, 2011/0172146 A1, 2013/0017997 A1, or 2012/0263701 A1, International Patent Publication Nos. WO 2010091122 A1, WO 2010144502 A2, WO 2010144508 A1, WO 2011028228 A1, WO 2011028229 A1, or WO 2011028344 A2; or International Application No. PCT/US2011/48517, filed Aug. 19, 2011.

### II.D. Chimeric Molecules Further Comprising Heterologous Moieties

The present disclosure also provides “chimeric molecules,” which is further fused and/or conjugated and/or otherwise associated with at least one heterologous moiety. Thus, a chimeric molecule disclosed herein (for example, a chimeric molecule comprising FVII, an XTEN polypeptide and at least one of the GPIIb/IIIa antibodies or antigen-binding molecules thereof as disclosed in section II.A. or a chimeric molecule comprising FVII_{H}, FVII_{L}, an XTEN polypeptide and a targeting moiety, which binds to a platelet, as disclosed in section II.B.) encompasses any molecule further comprising at least one heterologous moiety (e.g., a half-life extending moiety). In some embodiments, a chimeric molecule is a chimeric protein, i.e., a chimeric molecule in which all its components (heterologous moieties and/or linkers) are polypeptides. Other chimeric molecules can comprise non-polypeptide heterologous moieties (e.g., PEG, lipids, carbohydrates, nucleic acids, small molecule therapeutic agents, radionuclides, fluorescent probes, etc.) and/or non-polypeptide linkers.

As used herein the term “moiety” refers to a component part or constituent of a chimeric molecule of the present invention. As used herein, the term “heterologous moiety” refers to a moiety genetically fused, conjugated, and/or otherwise associated to any component of the chimeric molecules. In certain embodiments, a chimeric molecule can comprise, one, two, three, four, five, or more than five heterologous moieties.

The heterologous moiety or moieties of the chimeric molecules disclosed herein can comprise, consist of, or consist essentially of prophylactic and/or therapeutic agents (e.g., clotting factors), molecules capable of improving a pharmacokinetic (PK) property (e.g., plasma half-life extending moieties), detectable moieties (e.g., fluorescent molecules or radionuclides), etc.

In some embodiments, a heterologous moiety can modify a physicochemical property of a chimeric molecule lacking such heterologous moiety, for example, it can increase the hydrodynamic radius of a chimeric molecule. In other embodiments, the incorporation of a heterologous moiety into a chimeric molecule can improve one or more pharmacokinetic properties without significantly affecting its biological activity or function (e.g., procoagulant activity in chimeric molecules comprising FVII).

In some embodiments, the heterologous moiety is a polypeptide comprising, consisting essentially of, or consisting of at least about 10, 100, 200, 300, 400, 500, 600, 700, 800, 900, 1000, 1100, 1200, 1300, 1400, 1500, 1600, 1700, 1800, 1900, 2000, 2500, 3000, or 4000 amino acids. In other embodiments, the heterologous moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In other embodiments, a heterologous moiety increases stability of the chimeric molecule of the invention or a fragment thereof. As used herein, the term “stability” refers to an art-recognized measure of the maintenance of one or more physical properties of the chimeric molecule in response to an environmental condition (e.g., an elevated or lowered temperature). In certain embodiments, the physical property can be the maintenance of the covalent structure of the chimeric molecule (e.g., the absence of proteolytic cleavage, unwanted oxidation or deamidation). In other embodiments, the physical property can also be the presence of the chimeric molecule in a properly folded state (e.g., the absence of soluble or insoluble aggregates or precipitates). In one embodiment, the stability of the chimeric molecule is measured by assaying a biophysical property of the chimeric molecule, for example thermal stability, pH unfolding profile, stable removal of glycosylation, solubility, biochemical function (e.g., ability to bind to a protein, receptor or ligand), etc., and/or combinations thereof. In another embodiment, biochemical function is demonstrated by the binding affinity of the interaction. In one embodiment, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In some embodiments, the chimeric molecule comprises at last one heterologous moiety that is a “half-life extending moiety.” As used herein, the term “half-life extending moiety” refers to a heterologous moiety which increases the in vivo half-life of a protein, for example, a chimeric molecule. The term “half-life” refers to a biological half-life of a particular protein or polypeptide (e.g., a clotting factor or a chimeric molecule disclosed herein) in vivo. Half-life can be represented by the time required for half the quantity administered to a subject to be cleared from the circulation and/or other tissues in the animal. When a clearance curve of a given polypeptide or chimeric molecule of the invention is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, procoagulant compounds of the invention are monophasic, and thus do not have an alpha phase, but just the single beta phase. In certain embodiments, the term half-life as used herein refers to the half-life of the procoagulant compound in the β-phase. The typical β phase half-life of a human antibody in humans is 21 days. In vivo half-life of a chimeric molecule can be determined by any method known to those of skill in the art. In certain embodiments, the half-life extending moiety can comprise an attachment site for a non-polypeptide moiety (e.g., PEG).

Half-life extending moieties, as discussed below in detail, can comprise, for example, (i) an additional XTEN polypeptide, (ii) albumin, (iii) albumin binding polypeptide or fatty acid, (iv) Fc, (v) transferrin, (vi) PAS, (vii) the C-terminal peptide (CTP) of the β subunit of human chorionic gonadotropin, (viii) polyethylene glycol (PEG), (ix) hydroxyethyl starch (HES), (x) albumin-binding small molecules, (xi) vWF, (xii) a clearance receptor or fragment thereof which blocks binding of the chimeric molecule to a clearance receptor, or (xiii) any combinations thereof. In some embodiments, the half-life extending moiety comprises an Fc region. In other embodiments, the half-life extending moiety comprises two Fc regions fused by a linker. Exemplary heterologous moieties also include, e.g., FcRn binding moieties (e.g., complete Fc regions or portions thereof which bind to FcRn), single chain Fc regions (scFc regions, e.g., as described in U.S. Publ. No. 2008-0260738, and Intl. Publ. Nos. WO 2008-012543 and WO 2008-1439545), or processable scFc regions. In some embodiments, a heterologous moiety can include an attachment site for a non-polypeptide moiety such as polyethylene glycol (PEG), hydroxyethyl starch (HES), polysialic acid, or any derivatives, variants, or combinations of these moieties.

In certain embodiments, a chimeric molecule of the invention comprises at least one half-like extending moiety which increases the circulation half-life of the chimeric molecule with respect to the circulation half-life of the corresponding chimeric molecule lacking such heterologous moiety. Circulation half-life of a chimeric molecule can be determined by any method known to those of skill in the art, e.g., activity assays (chromogenic assay or one stage clotting aPTT assay), ELISA, etc.

In some embodiments, the presence of one or more half-life extending moiety results in the half-life of the chimeric molecule to be increased compared to the half-life of the corresponding chimeric molecule lacking such one or more half-life extending moieties. The half-life of the chimeric molecule comprising a half-life extending moiety is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the circulation half-life of the corresponding chimeric molecule lacking such half-life extending moiety.

In one embodiment, the half-life of the chimeric molecule comprising a half-life extending moiety is about 1.5-fold to about 20-fold, about 1.5 fold to about 15 fold, or about 1.5 fold to about 10 fold longer than the in vivo half-life of the corresponding chimeric molecule lacking such half-life extending moiety. In another embodiment, the half-life of chimeric molecule comprising a half-life extending moiety is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to the in vivo half-life of the corresponding chimeric molecule lacking such half-life extending moiety.

### II.D.1. Fc Region

In certain embodiments, the chimeric molecule comprises at least a heterologous moiety comprising a Fc region. “Fc” or “Fc region” as used herein means a functional neonatal Fc receptor (FcRn) binding partner comprising an Fc domain, variant, or fragment thereof, unless otherwise specified. An FcRn binding partner is any molecule that can be specifically bound by the FcRn receptor with consequent active transport by the FcRn receptor of the FcRn binding partner. Thus, the term Fc includes any variants of IgG Fc that are functional. The region of the Fc portion of IgG that binds to the FcRn receptor has been described based on X-ray crystallography (Burmeister et al., Nature 372:379 (1994), incorporated herein by reference in its entirety). The major contact area of the Fc with the FcRn is near the junction of the CH2 and CH3 domains. Fc-FcRn contacts are all within a single Ig heavy chain. FcRn binding partners include, but are not limited to, whole IgG, the Fc fragment of IgG, and other fragments of IgG that include the complete binding region of FcRn. An Fc can comprise the CH2 and CH3 domains of an immunoglobulin with or without the hinge region of the immunoglobulin. Also included are Fc fragments, variants, or derivatives which maintain the desirable properties of an Fc region in a chimeric molecule, e.g., an increase in half-life, e.g., in vivo half-life. Myriad mutants, fragments, variants, and derivatives are described, e.g., in PCT Publication Nos. WO2011/069164, WO2012/006623, WO2012/006635, or WO 2012/006633, all of which are incorporated herein by reference in their entireties.

In some embodiments, the chimeric molecule comprises FVII, a targeting moiety (e.g., a GPIIb/IIIa antibody or antigen-binding molecule thereof), an XTEN polypeptide, and an Fc region.

II.D.2. scFc (Single Chain Fc) Region

In one embodiment, the chimeric molecule comprises a heterologous moiety comprising one genetically fused Fc region or a portion thereof within a single polypeptide chain (i.e., a single-chain Fc (scFc) region). The unprocessed polypeptides comprise at least two immunoglobulin constant regions or portions thereof (e.g., Fc moieties or domains (e.g., 2, 3, 4, 5, 6, or more Fc moieties or domains)) within the same linear polypeptide chain that are capable of folding (e.g., intramolecularly or intermolecularly folding) to form one functional scFc region which is linked by an Fc peptide linker. For example, in one embodiment, a polypeptide of the invention is capable of binding, via its scFc region, to at least one Fc receptor (e.g., an FcRn, an FcγR receptor (e.g., FcγRIII), or a complement protein (e.g., C1q)) in order to improve half-life or trigger an immune effector function (e.g., antibody-dependent cytotoxicity (ADCC), phagocytosis, or complement-dependent cytotoxicity (CDCC) and/or to improve manufacturability).

In some embodiments, the chimeric molecule comprises a clotting factor (e.g., FVII), a targeting moiety (e.g., a GPIIb/IIIa antibody or antigen-binding molecule thereof), an XTEN polypeptide, and an scFc region.

### II.D.3. Albumins

In certain embodiments, the chimeric molecule comprises a heterologous moiety comprising albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. US2008/0194481, US2008/0004206, US2008/0161243, US2008/0261877, or US2008/0153751 or PCT Appl. Publ. Nos. WO2008/033413, WO2009/058322, or WO2007/021494, which are incorporated herein by reference in their entireties.

In some embodiments, the chimeric molecule comprises FVII, a targeting moiety (e.g., a GPIIb/IIIa antibody or antigen-binding molecule thereof), an XTEN polypeptide, and an albumin.

### II.D.4. Albumin Binding Polypeptides and Lipids

In certain embodiments, a heterologous moiety can comprise an albumin binding moiety, which comprises an albumin binding peptide, a bacterial albumin binding domain, an albumin-binding antibody fragment, or any combinations thereof. For example, the albumin binding protein can be a bacterial albumin binding protein, an antibody or an antibody fragment including domain antibodies (see, e.g., U.S. Pat. No. 6,696,245). An albumin binding protein, for example, can be a bacterial albumin binding domain, such as the one of streptococcal protein G (Konig and Skerra (1998) J. Immunol. Methods 218, 73-83). Other examples of albumin binding peptides that can be used as conjugation partner are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, H is, Ile, Leu, or Lys; Xaa₃ is Ala, Asp, Phe, Trp, or Tyr; and Xaa₄ is Asp, Gly, Leu, Phe, Ser, or Thr as described in U.S. Pub. No. US2003/0069395 or Dennis et al. (2002) J. Biol. Chem. 277, 35035-35043.

Domain 3 from streptococcal protein G, as disclosed by Kraulis et al., FEBS Lett. 378:190-194 (1996) and Linhult et al., Protein Sci. 11:206-213 (2002) is an example of a bacterial albumin-binding domain. Examples of albumin-binding peptides include a series of peptides having the core sequence DICLPRWGCLW (SEQ ID NO:______). See, e,g., Dennis et al., J. Biol. Chem. 2002, 277: 35035-35043 (2002). Examples of albumin-binding antibody fragments are disclosed in Muller and Kontermann, Curr. Opin. Mol. Ther. 9:319-326 (2007); Roovers et al., Cancer Immunol. Immunother. 56:303-317 (2007), and Holt et al., Prot. Eng. Design Sci., 21:283-288 (2008), which are incorporated herein by reference in their entireties. An example of such albumin binding moiety is 2-(3-maleimidopropanamido)-6-(4-(4-iodophenyl)butanamido) hexanoate (“Albu” tag) as disclosed by Trussel et al., Bioconjugate Chem. 20:2286-2292 (2009). Fatty acids, in particular long chain fatty acids (LCFA) and long chain fatty acid-like albumin-binding compounds can be used to extend the in vivo half-life of chimeric molecules of the invention. An example of a LCFA-like albumin-binding compound is 16-(1-(3-(9-(((2,5-dioxopyrrolidin-1-yloxy)carbonyloxy)-methyi)-7-sulfo-9H-fluoren-2-ylamino)-3-oxopropyl)-2,5-dioxopyrrolidin-3-ylthio) hexadecanoic acid (see, e.g., WO 2010/140148).

In some embodiments, the chimeric molecule comprises FVII, a targeting moiety (e.g., a GPIIb/IIIa antibody or antigen-binding molecule thereof), an XTEN polypeptide, and an albumin binding polypeptide or lipid.

### II.D.5. CTP

In certain embodiments, a chimeric molecule disclosed herein comprises at least one heterologous moiety comprising one β subunit of the C-terminal peptide (CTP) of human chorionic gonadotropin or fragment, variant, or derivative thereof. The insertion of one or more CTP peptides into a recombinant protein is known to increase the in vivo half-life of that protein. See, e.g., U.S. Pat. No. 5,712,122, incorporated by reference herein in its entirety.

Exemplary CTP peptides include DPRFQDSSSSKAPPPSLPSPSRLPGPSDTPIL (SEQ ID NO:______) or SSSSCKAPPPSLPSPSRLPGPSDTPILPQ (SEQ ID NO:______). See, e.g., U.S. Patent Appl. Publ. No. US 2009/0087411, incorporated by reference. In some embodiments, the chimeric molecule comprises two heterologous moieties that are CTP sequences. In some embodiments, three of the heterologous moieties are CTP sequences. In some embodiments, four of the heterologous moieties are CTP sequences. In some embodiments, five of the heterologous moieties are CTP sequences. In some embodiments, six or more of the heterologous moieties are CTP sequences.

In some embodiments, the chimeric molecule comprises FVII, a targeting moiety (e.g., a GPIIb/IIIa antibody or antigen-binding molecule thereof), an XTEN polypeptide, and a CTP.

### II.D.6. PAS

In other embodiments, at least one heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric molecule. Yet, the skilled person is aware that an amino acid polymer also can form random coil conformation when residues other than alanine, serine, and proline are added as a minor constituent in the PAS sequence.

The term “minor constituent” as used herein means that amino acids other than alanine, serine, and proline can be added in the PAS sequence to a certain degree, e.g., up to about 12%, i.e., about 12 of 100 amino acids of the PAS sequence, up to about 10%, i.e., about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids.

The amino acids different from alanine, serine and proline can be selected from the group consisting of Arg, Asn, Asp, Cys, Gln, Glu, Gly, His, Ile, Leu, Lys, Met, Phe, Thr, Trp, Tyr, and Val.

Under physiological conditions, the PAS sequence stretch forms a random coil conformation and thereby can mediate an increased in vivo and/or in vitro stability to the chimeric molecule. Since the random coil domain does not adopt a stable structure or function by itself, the biological activity mediated by the activated clotting factor in the chimeric molecule is essentially preserved. In other embodiments, the PAS sequences that form random coil domain are biologically inert, especially with respect to proteolysis in blood plasma, immunogenicity, isoelectric point/electrostatic behavior, binding to cell surface receptors or internalization, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA (SEQ ID NO: 155), AAPASPAPAAPSAPAPAAPS (SEQ ID NO: 156), APSSPSPSAPSSPSPASPSS (SEQ ID NO: 157), APSSPSPSAPSSPSPASPS (SEQ ID NO: 158), SSPSAPSPSSPASPSPSSPA (SEQ ID NO: 159), AASPAAPSAPPAAASPAAPSAPPA (SEQ ID NO: 160) and ASAAAPAAASAAASAPSAAA (SEQ ID NO: 161) or any combinations thereof. Additional examples of PAS sequences are known from, e.g., US Pat. Publ. No. 2010/0292130 and PCT Appl. Publ. No. WO2008/155134 A1.

In some embodiments, the chimeric molecule comprises FVII, a targeting moiety (e.g., a GPIIb/IIIa antibody or antigen-binding molecule thereof), an XTEN polypeptide, and a PAS.

### II.D.7. HAP

In certain embodiments, at least one heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200. See, e.g., Schlapschy M et al., Protein Eng. Design Selection, 20: 273-284 (2007).

In some embodiments, the chimeric molecule comprises FVII, a targeting moiety (e.g., a GPIIb/IIIa antibody or antigen-binding molecule thereof), an XTEN polypeptide, and a HAP.

### II.D.8. Transferrin

In certain embodiments, at least one heterologous moiety is transferrin or a peptide or fragment, variant, or derivative thereof. Any transferrin can be used to make the chimeric molecules of the invention. As an example, wild-type human TF (TF) is a 679 amino acid protein, of approximately 75 KDa (not accounting for glycosylation), with two main domains, N (about 330 amino acids) and C (about 340 amino acids), which appear to originate from a gene duplication. N domain comprises two subdomains, N1 domain and N2 domain, and C domain comprises two subdomains, C1 domain and C2 domain. See GenBank accession numbers NM001063, XM002793, M12530, XM039845, XM 039847 and S95936 (www.ncbi.nlm.nih.gov), all of which are herein incorporated by reference in their entirety. In one embodiment, the transferrin heterologous moiety includes a transferrin splice variant. In one example, a transferrin splice variant can be a splice variant of human transferrin, e.g., Genbank Accession AAA61140. In another embodiment, the transferrin portion of the chimeric molecule includes one or more domains of the transferrin sequence, e.g., N domain, C domain, N1 domain, N2 domain, C1 domain, C2 domain or any combinations thereof.

Transferrin transports iron through transferrin receptor (TfR)-mediated endocytosis. After the iron is released into an endosomal compartment and Tf-TfR complex is recycled to cell surface, the Tf is released back extracellular space for next cycle of iron transporting. Tf possesses a long half-life that is in excess of 14-17 days (Li et al., Trends Pharmacol. Sci. 23:206-209 (2002)). Transferrin fusion proteins have been studied for half-life extension, targeted deliver for cancer therapies, oral delivery and sustained activation of proinsulin (Brandsma et al., Biotechnol. Adv., 29: 230-238 (2011); Bai et al., Proc. Natl. Acad. Sci. USA 102:7292-7296 (2005); Kim et al., J. Pharmacol. Exp. Ther., 334:682-692 (2010); Wang et al., J. Controlled Release 155:386-392 (2011)).

In some embodiments, the chimeric molecule comprises FVII, a targeting moiety (e.g., a GPIIb/IIIa antibody or antigen-binding molecule thereof), an XTEN polypeptide, and a transferrin.

### II.D.9. PEG

In some embodiments, at least one heterologous moiety is a soluble polymer known in the art, including, but not limited to, polyethylene glycol, ethylene glycol/propylene glycol copolymers, carboxymethylcellulose, dextran, or polyvinyl alcohol. In some embodiments, the chimeric molecule comprising a PEG heterologous moiety further comprises a heterologous moiety selected from the group consisting of an immunoglobulin constant region or portion thereof (e.g., an Fc region), a PAS sequence, HES, and albumin, fragment, or variant thereof. In still other embodiments, the chimeric molecule comprises an activated clotting factor or fragment thereof and a PEG heterologous moiety, wherein the chimeric molecule further comprises a heterologous moiety selected from the group consisting of an immunoglobulin constant region or portion thereof (e.g., an Fc moiety), a PAS sequence, HES, and albumin, fragment, or variant thereof. In yet other embodiments, the chimeric molecule comprises a clotting factor or fragment thereof, a second clotting factor or fragment thereof, and a PEG heterologous moiety, wherein the chimeric molecule further comprises a heterologous moiety selected from the group consisting of an immunoglobulin constant region or portion thereof (e.g., an Fc moiety), a PAS sequence, HES, and albumin, fragment, or variant thereof.

In other embodiments, the chimeric molecule comprises a clotting factor or fragment thereof, a synthetic procoagulant polypeptide, and a PEG heterologous moiety, wherein the chimeric molecule further comprises a heterologous moiety selected from the group consisting of an immunoglobulin constant region or portion thereof (e.g., an Fc region), a PAS sequence, HES, and albumin, fragment, or variant thereof. In other embodiments, the chimeric molecule comprises two synthetic procoagulant peptides and a PEG heterologous moiety, wherein the chimeric molecule further comprises a heterologous moiety selected from the group consisting of an immunoglobulin constant region or portion thereof (e.g., an Fc region), a PAS sequence, HES, and albumin, fragment, or variant thereof. In yet another embodiment, the chimeric molecule comprises a clotting factor or fragment thereof, a clotting factor cofactor (e.g., Tissue Factor if the clotting factor is Factor VII), and a PEG heterologous moiety, wherein the chimeric molecule further comprises a heterologous moiety selected from the group consisting of an immunoglobulin constant region or portion thereof (e.g., an Fc region), a PAS sequence, HES, and albumin, fragment, or variant thereof.

The polymer can be of any molecular weight, and can be branched or unbranched. For polyethylene glycol, in one embodiment, the molecular weight is between about 1 kDa and about 100 kDa for ease in handling and manufacturing. Other sizes can be used, depending on the desired profile (e.g., the duration of sustained release desired, the effects, if any on biological activity, the ease in handling, the degree or lack of antigenicity and other known effects of the polyethylene glycol to a protein or analog). For example, the polyethylene glycol can have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

In some embodiments, the polyethylene glycol can have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), each of which is incorporated herein by reference in its entirety.

The number of polyethylene glycol moieties attached to each chimeric molecule of the invention (i.e., the degree of substitution) can also vary. For example, the PEGylated chimeric molecule can be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

In some embodiments, the chimeric molecule can be PEGylated. A PEGylated chimeric molecule comprises at least one polyethylene glycol (PEG) molecule. In other embodiments, the polymer can be water-soluble. Non-limiting examples of the polymer can be poly(alkylene oxide), poly(vinyl pyrrolidone), poly(vinyl alcohol), polyoxazoline, or poly(acryloylmorpholine). Additional types of polymer-conjugation to clotting factors are disclosed in U.S. Pat. No. 7,199,223. See also, Singh et al. Curr. Med. Chem. 15:1802-1826 (2008).

There are a number of PEG attachment methods available to those skilled in the art, for example Malik F et al., Exp. Hematol. 20:1028-35 (1992); Francis, Focus on Growth Factors 3(2):4-10 (1992); European Pat. Pub. Nos. EP0401384, EP0154316, and EP0401384; and International Pat. Appl. Pub. Nos. WO92/16221 and WO95/34326.

In some embodiments, the chimeric molecule comprises FVII, a targeting moiety (e.g., a GPIIb/IIIa antibody or antigen-binding molecule thereof), an XTEN polypeptide, and a PEG.

### II.D.10HES

In certain embodiments, at least one heterologous moiety is a polymer, e.g., hydroxyethyl starch (HES) or a derivative thereof. Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8(8), 271-278 (1987); and Weidler et al., Arzneim.-Forschung/Drug Res., 41, 494-498 (1991)).

Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. The degree of substitution, denoted as DS, relates to the molar substitution, is known to the skilled people. See Sommermeyer et al., Krankenhauspharmazie, 8(8), 271-278 (1987), as cited above, in particular p. 273.

In one embodiment, hydroxyethyl starch has a mean molecular weight (weight mean) of from 1 to 300 kD, from 2 to 200 kD, from 3 to 100 kD, or from 4 to 70 kD. Hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 1 l to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

In certain embodiments, the heterologous moiety can be a mixture of hydroxyethyl starches having different mean molecular weights and/or different degrees of substitution and/or different ratios of C2: C6 substitution. Therefore, mixtures of hydroxyethyl starches can be employed having different mean molecular weights and different degrees of substitution and different ratios of C2: C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2: C6 substitution, or having about the same mean molecular weight and about the same degree of substitution and about the same ratio of C2:C6 substitution.

In some embodiments, the chimeric molecule comprises FVII, a targeting moiety (e.g., a GPIIb/IIIa antibody or antigen-binding molecule thereof), an XTEN polypeptide, and a HES.

### II.D.11 PSA

In certain embodiments, at least one heterologous moiety is a polymer, e.g., polysialic acids (PSAs) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhäuser Verlag, Basel, Switzerland), pp 335-348. They can be produced in various degrees of polymerisation from n=about 80 or more sialic acid residues down to n=2 by limited acid hydrolysis or by digestion with neuraminidases, or by fractionation of the natural, bacterially derived forms of the polymer. The composition of different polysialic acids also varies such that there are homopolymeric forms i.e. the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist-such as the alternating alpha-2,8 alpha-2,9 polysialic acid of E. coli strain K92 and group C polysaccharides of N. meningitidis. Sialic acid can also be found in alternating copolymers with monomers other than sialic acid such as group W135 or group Y of N. meningitidis. Polysialic acids have important biological functions including the evasion of the immune and complement systems by pathogenic bacteria and the regulation of glial adhesiveness of immature neurons during foetal development (wherein the polymer has an anti-adhesive function) Cho and Troy, P.N.A.S., USA, 91 (1994) 11427-11431, although there are no known receptors for polysialic acids in mammals. The alpha-2,8-linked polysialic acid of E. coli strain K1 is also known as ‘colominic acid’ and is used (in various lengths) to exemplify the present invention. Various methods of attaching or conjugating polysialic acids to a polypeptide have been described (for example, see U.S. Pat. No. 5,846,951; WO-A-0187922, and US 2007/0191597 A1, which are incorporated herein by reference in their entireties.

In some embodiments, the chimeric molecule comprises FVII, a targeting moiety (e.g., a GPIIb/IIIa antibody or antigen-binding molecule thereof), an XTEN polypeptide, and a PSA.

### II.D.12 Clearance Receptors

In certain embodiments, the in vivo half-life of a chimeric molecule of the invention can be extended where the chimeric molecule comprises at least one heterologous molecule comprising a clearance receptor, fragment, variant, or derivative thereof. In specific embodiments wherein the chimeric molecule comprises Factor X, soluble forms of clearance receptors, such as the low density lipoprotein-related protein receptor LRP1, or fragments thereof, can block binding of Factor X to clearance receptors and thereby extend its in vivo half-life.

LRP1 is a 600 kDa integral membrane protein that is implicated in the receptor-mediate clearance of a variety of proteins, such as FVIII or X. See, e.g., Narita et al., Blood 91:555-560 (1998); Lenting et al., Haemophilia 16:6-16 (2010). Other suitable clearance receptors are, e.g., LDLR (low-density lipoprotein receptor), VLDLR (very low-density lipoprotein receptor), and megalin (LRP-2), or fragments thereof. See, e.g., Bovenschen et al., Blood 106:906-912 (2005); Bovenschen, Blood 116:5439-5440 (2010); Martinelli et al., Blood 116:5688-5697 (2010).

In some embodiments, the chimeric molecule comprises FVII, a targeting moiety (e.g., a GPIIb/IIIa antibody or antigen-binding molecule thereof), an XTEN polypeptide, and a clearance receptor, fragment, variant, or derivative thereof.

### II.E. Factor VII

The chimeric molecule comprises FVII, which is a mature form of Factor VII or a variant thereof. Factor VII (FVII, F7; also referred to as Factor 7, coagulation factor VII, serum factor VII, serum prothrombin conversion accelerator, SPCA, proconvertin and eptacog alpha) is a serine protease that is part of the coagulation cascade. FVII includes a Gla domain, two EGF domains (EGF-1 and EGF-2), and a serine protease domain (or peptidase S1 domain) that is highly conserved among all members of the peptidase S1 family of serine proteases, such as for example with chymotrypsin. FVII occurs as a single chain zymogen, an activated zymogen-like two-chain polypeptide (e.g., activatable FVII) and a fully activated two-chain form.

As used herein, a “zymogen-like” protein or polypeptide refers to a protein that has been activated by proteolytic cleavage, but still exhibits properties that are associated with a zymogen, such as, for example, low or no activity, or a conformation that resembles the conformation of the zymogen form of the protein. For example, when it is not bound to tissue factor, the two-chain activated form of FVII is a zymogen-like protein; it retains a conformation similar to the uncleaved FVII zymogen, and, thus, exhibits very low activity. Upon binding to tissue factor, the two-chain activated form of FVII undergoes conformational change and acquires its full activity as a coagulation factor.

Exemplary FVII variants include those with increased specific activity, e.g., mutations that increase the activity of FVII by increasing its enzymatic activity (Kcat or Km). Such variants have been described in the art and include, e.g., mutant forms of the molecule as described for example in Persson et al., Proc. Natl. Acad Sci. USA 98:13583 (2001); Petrovan and Ruf, J. Biol. Chem. 276:6616 (2001); Persson et al., J. Biol. Chem. 276:29195 (2001); Soejima et al., J. Biol. Chem. 276:17229 (2001); Soejima et al., J. Biol. Chem. 247:49027 (2002).

In one embodiment, a variant form of FVII includes mutations, e.g., V158D-E296V-M298Q. In another embodiment, a variant form of FVII includes a replacement of amino acids 608-619 (LQQSRKVGDSPN, corresponding to the 170-loop) from the FVII mature sequence with amino acids EASYPGK (SEQ ID NO: ______) from the 170-loop of trypsin. High specific activity variants of FVII are also known in the art. For example, Simioni et al. (N.E. Journal of Medicine 361:1671, 2009) describe an R338L mutation. Chang et al. (J. Biol. Chem. 273:12089, 1988) and Pierri et al. (Human Gene Therapy 20:479, 2009) describe an R338A mutation. Other mutations are known in the art and include those described, e.g., in Zogg and Brandstetter, Structure 17:1669 (2009); Sichler et al., J. Biol. Chem. 278:4121 (2003); and Sturzebecher et al., FEBS Lett. 412:295 (1997). The contents of these references are incorporated herein by reference.

Full activation, which occurs upon conformational change from a zymogen-like form, occurs upon binding to its co-factor, i.e., tissue factor. Also, mutations can be introduced that result in the conformation change in the absence of tissue factor. Hence, reference to FVIIa includes both two-chain forms thereof: the zymogen-like form, and the fully activated two-chain form.

In one embodiment, the heavy chain of FVII comprises an amino acid sequence at least about 60%, at least about 70%, at least about 80%, at least about 90%, at least about 95%, at least about 96%, at least about 97%, at least about 98%, at least about 99%, or at least about 100% identical to SEQ ID NO: 178, wherein FVII comprising the heavy chain has FVII clotting activity. In another embodiment, the light chain of FVII comprises an amino acid sequence at least about 60%, at least about 70%, at least about 80%, at least about 90%, at least about 95%, at least about 96%, at least about 97%, at least about 98%, at least about 99%, or at least about 100% identical to SEQ ID NO: 179, wherein FVII comprising the light chain has FVII clotting activity.

### II.F. Linkers

As used herein, the term “linker” or “linker moiety” (represented as L, L1, or L2 in the formulas disclosed herein) refers to a peptide or polypeptide sequence (e.g., a synthetic peptide or polypeptide sequence), or a non-peptide linker for which its main function is to connect two domains in a linear amino acid sequence of a polypeptide chain, for example, two heterologous moieties in a chimeric molecule of the invention. Accordingly, in some embodiments, linkers are interposed between two heterologous moieties, between a heterologous moiety and a targeting moiety, which binds to a platelet (e.g., an anti-GPIIb/IIIa antibody or antigen-binding molecule thereof disclosed in section II.A.1.), between FVII (either the heavy chain or the light chain) and a targeting moiety, which binds to a platelet (e.g., an anti-GPIIb/IIIa antibody or antigen-binding molecule thereof disclosed in section II.A.1.), or between FVII (either the heavy chain or the light chain) and a heterologous moiety.

When multiple linkers are present in a chimeric molecule of the invention, each of the linkers can be the same or different. Generally, linkers provide flexibility to the chimeric molecule. Linkers are not typically cleaved; however in certain embodiments, such cleavage can be desirable. Accordingly, in some embodiments a linker can comprise one or more protease-cleavable sites, which can be located within the sequence of the linker or flanking the linker at either end of the sequence of the linker.

In some embodiments, the chimeric molecule comprises one or more linkers, wherein one or more of the linkers comprise a peptide linker. In other embodiments, one or more of the linkers comprise a non-peptide linker. In some embodiments, the peptide linker can comprise at least one, at least two, at least three, at least four, at least five, at least 10, at least 20, at least 30, at least 40, at least 50, at least 60, at least 70, at least 80, at least 90, or at least 100 amino acids. In other embodiments, the peptide linker can comprise at least 200, at least 300, at least 400, at least 500, at least 600, at least 700, at least 800, at least 900, or at least 1,000 amino acids. In some embodiments, the peptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids.

The peptide linker can comprise 1-5 amino acids, 1-10 amino acids, 1-20 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, 900-1000, 1000-1100, 1100-1200, 1200-1300, 1300-1400, 1400-1500, 1500-1600, 1600-1700, 1700-1800, 1800-1900, or 1900-2000 amino acids.

Examples of peptide linkers are well known in the art, for example peptide linkers according to the formula [(Gly)ₓ-Ser_{y}]_{z} where x is from 1 to 4, y is 0 or 1, and z is from 1 to 50. In one embodiment, the peptide linker comprises the sequence Gₙ, where n can be an integer from 1 to 100. In a specific embodiment, the specific embodiment, the sequence of the peptide linker is GGGG. The peptide linker can comprise the sequence (GA)ₙ. The peptide linker can comprise the sequence (GGS)ₙ. In other embodiments, the peptide linker comprises the sequence (GGGS)ₙ (SEQ ID NO: 240). In still other embodiments, the peptide linker comprises the sequence (GGS)ₙ(GGGGS)ₙ (SEQ ID NO: 241). In these instances, n can be an integer from 1-100. In other instances, n can be an integer from 1-20, i.e., 1, 2, 3, 4, 5, 6, 7, 8, 9, 10, 11, 12, 13, 14, 15, 16, 17, 18, 19, or 20. Examples of linkers include, but are not limited to, GGG, SGGSGGS (SEQ ID NO: 242), GGSGGSGGSGGSGGG (SEQ ID NO: 243), GGSGGSGGGGSGGGGS (SEQ ID NO: 244), GGSGGSGGSGGSGGSGGS (SEQ ID NO: 245), or GGGGSGGGGSGGGGS (SEQ ID NO: 246). In other embodiments, the linker is a poly-G sequence (GGGG)ₙ, where n can be an integer from 1-100 (SEQ ID NO: 247).

An exemplary Gly/Ser peptide linker comprises the amino acid sequence (Gly₄Ser)ₙ (SEQ ID NO: 248), wherein n is an integer that is the same or higher than 1, 2, 3, 4, 5, 6, 7, 8, 9, 10, 15, 20, 25, 30, 35, 40, 46, 50, 55, 60, 70, 80, 90, or 100. In one embodiment, n=1, i.e., the linker is (Gly₄Ser) (SEQ ID NO: 249). In one embodiment, n=2, i.e., the linker is (Gly₄Ser)₂ (SEQ ID NO:250). In another embodiment, n=3, i.e., the linker is (Gly₄Ser)₃ (SEQ ID NO: 251). In another embodiment, n=4, i.e., the linker is (Gly₄Ser)₄ (SEQ ID NO: 252). In another embodiment, n=5, i.e., the linker is (Gly₄Ser)₅ (SEQ ID NO: 253). In yet another embodiment, n=6, i.e., the linker is (Gly₄Ser)₆ (SEQ ID NO: 254). In another embodiment, n=7, i.e., the linker is (Gly₄Ser)₇ (SEQ ID NO: 255). In yet another embodiment, n=8, i.e., the linker is (Gly₄Ser)₈ (SEQ ID NO: 256). In another embodiment, n=9, i.e., the linker is (Gly₄Ser)₉ (SEQ ID NO: 257). In yet another embodiment, n=10, i.e., the linker is (Gly₄Ser)₁₀ (SEQ ID NO: 258).

Another exemplary Gly/Ser peptide linker comprises the amino acid sequence Ser(Gly₄Ser)ₙ (SEQ ID NO: 248), wherein n is an integer that is the same or higher than 1, 2, 3, 4, 5, 6, 7, 8, 9, 10, 15, 20, 25, 30, 35, 40, 46, 50, 55, 60, 70, 80, 90, or 100. In one embodiment, n=1, i.e., the linker is Ser(Gly₄Ser) (SEQ ID NO: 259). In one embodiment, n=2, i.e., the linker is Ser(Gly₄Ser)₂ (SEQ ID NO: 260). In another embodiment, n=3, i.e., the linker is Ser(Gly₄Ser)₃ (SEQ ID NO: 261). In another embodiment, n=4, i.e., the linker is Ser(Gly₄Ser)₄ (SEQ ID NO: 262). In another embodiment, n=5, i.e., the linker is Ser(Gly₄Ser)₅ (SEQ ID NO: 263). In yet another embodiment, n=6, i.e., the linker is Ser(Gly₄Ser)₆ (SEQ ID NO: 264). In yet another embodiment, n=7, i.e., the linker is Ser(Gly₄Ser)₇ (SEQ ID NO: 265). In yet another embodiment, n=8, i.e., the linker is Ser(Gly₄Ser)₈ (SEQ ID NO: 266). In yet another embodiment, n=9, i.e., the linker is Ser(Gly₄Ser)₉ (SEQ ID NO: 267). In yet another embodiment, n=10, i.e., the linker is Ser(Gly₄Ser)₁₀ (SEQ ID NO: 268).

In certain embodiments, said Gly/Ser peptide linker can be inserted between two other sequences of the peptide linker (e.g., any of the peptide linker sequences described herein). In other embodiments, a Gly/Ser peptide linker is attached at one or both ends of another sequence of the peptide linker (e.g., any of the peptide linker sequences described herein). In yet other embodiments, two or more Gly/Ser linkers are incorporated in series in a peptide linker. In one embodiment, a peptide linker of the invention comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of Gly/Ser amino acid residues (e.g., a Gly/Ser linker such as (Gly₄Ser)ₙ).

Peptide linkers can be introduced into polypeptide sequences using techniques known in the art. Modifications can be confirmed by DNA sequence analysis. Plasmid DNA can be used to transform host cells for stable production of the polypeptides produced.

### II. G. Protease Cleavage Site

In some embodiments, a chimeric molecule comprises a protease cleavage site connecting any two components of the chimeric molecule, e.g., a light chain of FVII and a heavy chain of FVII. The protease-cleavage site can be an intracellular processing site for efficient cleavage and activation. For example, a chimeric molecule can comprise a single polypeptide chain, which comprises a light chain of FVII, an anti-GPIIb/IIIa antibody or antigen-binding molecule thereof, a protease cleavage site, a heavy chain of FVII, and an XTEN polypeptide or a light chain of FVII, an XTEN polypeptide, a protease cleavage site, and an anti-GPIIb/IIIa antibody or antigen-binding molecule thereof. The protease-cleavage site can be cleaved off by an intracellular processing enzyme in a host cell or by a protease at the site of coagulation.

Examples of the intracellular processing enzymes include furin, a yeast Kex2, PCSK1 (also known as PC1/Pc3), PCSK2 (also known as PC2), PCSK3 (also known as furin or PACE), PCSK4 (also known as PC4), PCSK5 (also known as PC5 or PC6), PCSK6 (also known as PACE4), or PCSK7 (also known as PC7/LPC, PC8, or SPC7). Other processing sites are known in the art. In constructs that include more than one processing or cleavage site, it will be understood that such sites can be the same or different.

In some embodiments, a chimeric molecule can comprise a protease cleavage site linking, for example, a light chain of FVII zymogen and a heavy chain of FVII zymogen. A protease-cleavage site linking a light chain of FVII zymogen and a heavy chain of FVII zymogen can be selected from any protease-cleavage site known in the art.

### III. Methods of Preparation

The present disclosure also provides a nucleic acid molecule or a set of nucleic acid molecules encoding any of the chimeric molecules disclosed herein or a complement thereof.

In one embodiment, the invention includes a nucleic acid molecule encoding a polypeptide chain, which comprises a light chain of FVII, an XTEN polypeptide, an intracellular processing site, a heavy chain of FVII, and a targeting moiety which binds to a platelet (e.g., an anti-GPIIb/IIIa antibody or antigen-binding molecule thereof disclosed in section II.A.1.). In another embodiment, the nucleic acid molecule of the invention encodes a polypeptide chain comprising a light chain of FVII, a targeting moiety which binds to a platelet (e.g., an anti-GPIIb/IIIa antibody or antigen-binding molecule thereof disclosed in section II.A.1.), an intracellular processing site, a heavy chain of FVII, and an XTEN polypeptide. In other embodiments, the nucleic acid molecule encodes a polypeptide chain comprising a light chain of FVII, an intracellular processing site, a heavy chain of FVII, an XTEN polypeptide, and a targeting moiety which binds to a platelet (e.g., an anti-GPIIb/IIIa antibody or antigen-binding molecule thereof disclosed in section II.A.1.). In some embodiments, the nucleic acid molecule encodes a polypeptide chain comprising a light chain of FVII, an intracellular processing site, a heavy chain of FVII, a targeting moiety which binds to a platelet (e.g., an anti-GPIIb/IIIa antibody or antigen-binding molecule thereof disclosed in section II.A.1.), and an XTEN polypeptide.

In some embodiments, the nucleic acid molecule comprises a set of nucleotide sequences, a first nucleotide sequence encoding a first polypeptide chain comprising a light chain of FVII, and an XTEN polypeptide and a second nucleotide sequence encoding a second polypeptide chain comprising a heavy chain of FVII, and a targeting moiety which binds to a platelet (e.g., an anti-GPIIb/IIIa antibody or antigen-binding molecule thereof disclosed in section II.A.1.). In other embodiments, the nucleic acid molecule comprises a set of nucleotide sequences, a first nucleotide sequence encoding a first polypeptide chain comprising a light chain of FVII, and a targeting moiety which binds to a platelet (e.g., an anti-GPIIb/IIIa antibody or antigen-binding molecule thereof disclosed in section II.A.1.) and a second nucleotide sequence encoding a second polypeptide chain comprising a heavy chain of FVII, and an XTEN polypeptide. In other embodiments, the nucleic acid molecule comprises a set of nucleotide sequences, a first nucleotide sequence encoding a light chain of FVII, and a second nucleotide sequence encoding a heavy chain of FVII, an XTEN polypeptide, and a targeting moiety which binds to a platelet (e.g., an anti-GPIIb/IIIa antibody or antigen-binding molecule thereof disclosed in section II.A.1.). In some embodiments, the nucleic acid molecule comprises a set of nucleotide sequences, a first nucleotide sequence encoding a light chain of FVII, and a second nucleotide sequence encoding a heavy chain of FVII, a targeting moiety which binds to a platelet (e.g., an anti-GPIIb/IIIa antibody or antigen-binding molecule thereof), and an XTEN polypeptide.

In some embodiments, the nucleotide sequence encoding a chimeric molecule comprises at least about 70%, 80%, 90%, 95%, 96%, 97%, 98%, 99%, or 100% identical to the nucleotide acid sequence of SEQ ID NO: 189, SEQ ID NO: 190, SEQ ID NO: 191, or SEQ ID NO: 192.

Also provided are a vector or a set of vectors comprising such nucleic acid molecule or the set of the nucleic acid molecules or a complement thereof, as well as a host cell comprising the vector.

The instant disclosure also provides a method for producing a chimeric molecule disclosed herein, such method comprising culturing the host cell disclosed herein and recovering the chimeric molecule from the culture medium.

In some embodiments, a chimeric molecule comprises a first amino acid sequence derived from a first source, bonded, covalently or non-covalently, to a second amino acid sequence derived from a second source, wherein the first and second source are not the same. A first source and a second source that are not the same can include two different biological entities, or two different proteins from the same biological entity, or a biological entity and a non-biological entity. A chimeric molecule can include for example, a protein derived from at least 2 different biological sources. A biological source can include any non-synthetically produced nucleic acid or amino acid sequence (e.g., a genomic or cDNA sequence, a plasmid or viral vector, a native virion or a mutant or analog, as further described herein, of any of the above). A synthetic source can include a protein or nucleic acid sequence produced chemically and not by a biological system (e.g., solid phase synthesis of amino acid sequences). A chimeric molecule can also include a protein derived from at least 2 different synthetic sources or a protein derived from at least one biological source and at least one synthetic source. A chimeric molecule can also comprise a first amino acid sequence derived from a first source, covalently or non-covalently linked to a nucleic acid, derived from any source or a small organic or inorganic molecule derived from any source. The chimeric molecule can also comprise a linker molecule between the first and second amino acid sequence or between the first amino acid sequence and the nucleic acid, or between the first amino acid sequence and the small organic or inorganic molecule.

A variety of methods are available for recombinantly producing a chimeric molecule disclosed herein. It will be understood that because of the degeneracy of the code, a variety of nucleic acid sequences will encode the amino acid sequence of the polypeptide. The desired polynucleotide can be produced by de novo solid-phase DNA synthesis or by PCR mutagenesis of an earlier prepared polynucleotide.

Oligonucleotide-mediated mutagenesis is one method for preparing a substitution, in-frame insertion, or alteration (e.g., altered codon) to introduce a codon encoding an amino acid substitution (e.g., into a chimeric molecule). For example, the starting polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide encoding any of the chimeric molecules disclosed herein.

For recombinant production, a polynucleotide sequence encoding a polypeptide (e.g., any of the chimeric molecules disclosed herein) is inserted into an appropriate expression vehicle, i.e., a vector which contains the necessary elements for the transcription and translation of the inserted coding sequence, or in the case of an RNA viral vector, the necessary elements for replication and translation.

The nucleic acid encoding any of the chimeric molecules disclosed herein is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14:725) and electroporation (Neumann et al. 1982, EMBO J. 1:841). A variety of host-expression vector systems can be utilized to express any of the chimeric molecules disclosed herein in eukaryotic cells. In one embodiment, the eukaryotic cell is an animal cell, including mammalian cells (e.g., 293 cells, PerC6, CHO, BHK, Cos, HeLa cells). When the polypeptide is expressed in a eukaryotic cell, the DNA encoding any of the chimeric molecules disclosed herein can also code for a signal sequence that will permit the polypeptide to be secreted. One skilled in the art will understand that while the polypeptide is translated, the signal sequence is cleaved by the cell to form the mature chimeric molecule. Various signal sequences are known in the art, e.g., native FVII signal sequence, native FIX signal sequence, native FX signal sequence, native GPIIb signal sequence, native GPIIIa signal sequence, and the mouse IgK light chain signal sequence. Alternatively, where a signal sequence is not included, the chimeric molecules disclosed herein can be recovered by lysing the cells.

The chimeric molecules disclosed herein can be synthesized in a transgenic animal, such as a rodent, goat, sheep, pig, or cow. The term “transgenic animals” refers to non-human animals that have incorporated a foreign gene into their genome. Because this gene is present in germline tissues, it is passed from parent to offspring. Exogenous genes are introduced into single-celled embryos (Brinster et al. 1985, Proc. Natl. Acad. Sci. USA 82:4438). Methods of producing transgenic animals are known in the art including transgenics that produce immunoglobulin molecules (Wagner et al. 1981, Proc. Natl. Acad Sci. USA 78:6376; McKnight et al. 1983, Cell 34:335; Brinster et al. 1983, Nature 306:332; Ritchie et al. 1984, Nature 312:517; Baldassarre et al. 2003, Theriogenology 59:831; Robl et al. 2003, Theriogenology 59:107; Malassagne et al. 2003, Xenotransplantation 10: 267).

The expression vectors can encode for tags that permit for easy purification or identification of the recombinantly produced polypeptide. Examples include, but are not limited to, vector pUR278 (Ruther et al. 1983, EMBO J. 2:1791) in which the chimeric molecules disclosed herein coding sequence can be ligated into the vector in frame with the lac z coding region so that a hybrid polypeptide is produced; pGEX vectors can be used to express proteins with a glutathione S-transferase (GST) tag. These proteins are usually soluble and can easily be purified from cells by adsorption to glutathione-agarose beads followed by elution in the presence of free glutathione. The vectors include cleavage sites, e.g., for PreCission Protease (Pharmacia, Peapack, N. J.) for easy removal of the tag after purification.

For the purposes of this invention, numerous expression vector systems can be employed. These expression vectors are typically replicable in the host organisms either as episomes or as an integral part of the host chromosomal DNA. Expression vectors can include expression control sequences including, but not limited to, promoters (e.g., naturally-associated or heterologous promoters), enhancers, signal sequences, splice signals, enhancer elements, and transcription termination sequences. Preferably, the expression control sequences are eukaryotic promoter systems in vectors capable of transforming or transfecting eukaryotic host cells. Expression vectors can also utilize DNA elements which are derived from animal viruses such as bovine papilloma virus, polyoma virus, adenovirus, vaccinia virus, baculovirus, retroviruses (RSV, MMTV or MOMLV), cytomegalovirus (CMV), or SV40 virus. Others involve the use of polycistronic systems with internal ribosome binding sites.

Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362). Cells which have integrated the DNA into their chromosomes can be selected by introducing one or more markers which allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation.

An exemplary expression vector is NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other embodiments, chimeric polypeptides of the invention can be expressed using polycistronic constructs. In these expression systems, multiple gene products of interest such as multiple polypeptides of multimer binding protein can be produced from a single polycistronic construct. These systems advantageously use an internal ribosome entry site (IRES) to provide relatively high levels of polypeptides of the invention in eukaryotic host cells. Compatible IRES sequences are disclosed in U.S. Pat. No. 6,193,980 which is also incorporated herein. Those skilled in the art will appreciate that such expression systems can be used to effectively produce the full range of polypeptides disclosed in the instant application.

More generally, once the vector or DNA sequence encoding a polypeptide has been prepared, the expression vector can be introduced into an appropriate host cell. That is, the host cells can be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), flow cytometry, immunohistochemistry, and the like.

As used herein, the term “transformation” refers in a broad sense to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of polypeptide unless it is clearly specified otherwise. In other words, recovery of polypeptide from the “cells” can mean either from spun down whole cells, or from the cell culture containing both the medium and the suspended cells.

In one embodiment, a host cell endogenously expresses an enzyme (or the enzymes) necessary to cleave a scFc linker (e.g., if such a linker is present and contains intracellular processing site(s)) during processing to form the mature polypeptide. During this processing, the scFc linker can be substantially removed to reduce the presence of extraneous amino acids. In another embodiment of the invention, a host cell is transformed to express one or more enzymes which are exogenous to the cell such that processing of a scFc linker occurs or is improved.

In one embodiment an enzyme which can be endogenously or exogenously expressed by a cell is a member of the furin family of enzymes. Complete cDNA and amino acid sequences of human furin (i.e., PACE) were published in 1990. Van den Ouweland A M et al. (1990) Nucleic Acids Res. 18:664; Erratum in: Nucleic Acids Res. 18:1332 (1990). U.S. Pat. No. 5,460,950, issued to Barr et al., describes recombinant PACE and the coexpression of PACE with a substrate precursor polypeptide of a heterologous protein to improve expression of active, mature heterologous protein. U.S. Pat. No. 5,935,815, likewise describes recombinant human furin (i.e., PACE) and the coexpression of furin with a substrate precursor polypeptide of a heterologous protein to improve expression of active, mature heterologous protein. Possible substrate precursors disclosed in this patent include a precursor of Factor IX. Other family members in the mammalian furin/subtilisin/Kex2p-like proprotein convertase (PC) family in addition to PACE are reported to include PCSK1 (also known as PC1/Pc3), PCSK2 (also known as PC2), PCSK3 (also known as furin or PACE), PCSK4 (also known as PC4), PCSK5 (also known as PC5 or PC6), PCSK6 (also known as PACE4), or PCSK7 (also known as PC7/LPC, PC8, or SPC7). While these various members share certain conserved overall structural features, they differ in their tissue distribution, subcellular localization, cleavage specificities, and preferred substrates. For a review, see Nakayama K (1997) Biochem J. 327:625-35. Similar to PACE, these proprotein convertases generally include, beginning from the amino terminus, a signal peptide, a propeptide (that can be autocatalytically cleaved), a subtilisin-like catalytic domain characterized by Asp, His, Ser, and Asn/Asp residues, and a Homo B domain that is also essential for catalytic activity and characterized by an Arg-Gly-Asp (RGD) sequence. PACE, PACE4, and PC5 also include a Cys-rich domain, the function of which is unknown. In addition, PC5 has isoforms with and without a transmembrane domain; these different isoforms are known as PC5B and PC5A, respectively. Comparison between the amino acid sequence of the catalytic domain of PACE and the amino acid sequences of the catalytic domains of other members of this family of proprotein convertases reveals the following degrees of identity: 70 percent for PC4; 65 percent for PACE4 and PC5; 61 percent for PC1/PC3; 54 percent for PC2; and 51 percent for LPC/PC7/PC8/SPC7. Nakayama K (1997) Biochem J. 327:625-35.

PACE and PACE4 have been reported to have partially overlapping but distinct substrates. In particular, PACE4, in striking contrast to PACE, has been reported to be incapable of processing the precursor polypeptide of FIX. Wasley et al. (1993) J. Biol. Chem. 268:8458-65; Rehemtulla et al. (1993) Biochemistry. 32:11586-90. U.S. Pat. No. 5,840,529, discloses nucleotide and amino acid sequences for human PC7 and the notable ability of PC7, as compared to other PC family members, to cleave HIV gp160 to gp120 and gp41.

Nucleotide and amino acid sequences of rodent PC5 were first described as PC5 by Lusson et al. (1993) Proc Natl Acad Sci USA 90:6691-5 and as PC6 by Nakagawa et al. (1993) J Biochem (Tokyo) 113:132-5. U.S. Pat. No. 6,380,171 discloses nucleotide and amino acid sequences for human PC5A, the isoform without the transmembrane domain. The sequences of these enzymes and method of cloning them are known in the art.

Genes encoding the polypeptides of the invention can also be expressed in non-mammalian cells such as bacteria or yeast or plant cells. In this regard it will be appreciated that various unicellular non-mammalian microorganisms such as bacteria can also be transformed; i.e., those capable of being grown in cultures or fermentation.

Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

In addition to prokaryates, eukaryotic microbes can also be used. Saccharomyces cerevisiae, or common baker's yeast, is the most commonly used among eukaryotic microorganisms although a number of other strains are commonly available.

For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

Other yeast hosts such Pichia can also be employed. Yeast expression vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Alternatively, polypeptide-coding nucleotide sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for polypeptides in operable linkage with a promoter and enhancer from a mammary gland specific gene, such as casein or beta lactoglobulin.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein. An affinity tag sequence (e.g. a His(6) tag) can optionally be attached or included within the polypeptide sequence to facilitate downstream purification.

Once expressed, the chimeric molecules can be purified according to standard procedures of the art, including ammonium sulfate precipitation, affinity column chromatography, HPLC purification, gel electrophoresis and the like (see generally Scopes, Protein Purification (Springer-Verlag, N.Y., (1982)) and see specifically the methods used in the instant Examples. Substantially pure proteins of at least about 90 to 95% homogeneity are preferred, and 98 to 99% or more homogeneity most preferred, for pharmaceutical uses.

### IV. Methods of Use

The present disclosure also provides is a pharmaceutical composition comprising

(i) a chimeric molecule disclosed herein;

(ii) a nucleic acid molecule or the set of nucleic acid molecules disclosed herein;

(iii) a vector or set of vectors disclosed herein; or

(iv) any combinations thereof,

and a pharmaceutically acceptable carrier.

In some embodiments, administering (i) a chimeric molecule disclosed herein, (ii) a nucleic acid molecule or a set of nucleic acid molecules disclosed herein, (iii) a vector or a set of vectors disclosed herein, or (iii) a pharmaceutical composition disclosed herein, can be used, for example, to reduce the frequency or degree of a bleeding episode in a subject in need, and/or reducing or preventing an occurrence of a bleeding episode in a subject in need thereof. In some embodiments, the subject has developed or has the capacity to develop an inhibitor against treatment with FVIII, FIX, or both. In some embodiments, the inhibitor against FVIII or FIX is a neutralizing antibody against FVIII, FIX, or both.

In some embodiments, the bleeding episode can be caused by a blood coagulation disorder, for example, hemophilia A or hemophilia B. In other embodiments, the bleeding episode can be derived from hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof. In certain embodiments, the subject is a human subject. In other embodiments, the subject is a mouse subject.

The instant disclosure also provides:

(a) a method to target FVII to the surface of platelets, wherein the method comprises fusing the agent to an XTEN polypeptide and one of the GPIIb/IIIa antibodies or antigen-binding molecules thereof disclosed in section II.A.1. or a targeting moiety, which binds to a platelet;

(b) a method to increase the activity of FVII comprising fusing FVII to an XTEN polypeptide and an anti-GPIIb/IIIa antibody or antigen-binding molecule thereof disclosed in section II.A.1. or a targeting moiety, which binds to a platelet; or,

(c) a method to improve the pharmacokinetic properties of FVII comprising fusing FVII to an XTEN polypeptide and an anti-GPIIb/IIIa antibody or antigen-binding molecule thereof disclosed in section II.A.1. or a targeting moiety, which binds to a platelet.

The invention also relates to a method of treating, ameliorating, or preventing a hemostatic disorder to a subject comprising administering a therapeutically effective amount of a chimeric molecule of the invention. The treatment, amelioration, and prevention by the chimeric molecule can be a bypass therapy. The subject in the bypass therapy can have already developed an inhibitor to a clotting factor, e.g., FVIII or FIX, or is subject to developing a clotting factor inhibitor. Compositions for administration to a subject include nucleic acid molecules which comprise a nucleotide sequence encoding a chimeric molecule the invention.

In one embodiment, a chimeric molecule composition of the invention is administered in combination with at least one other agent that promotes hemostasis. As an example, but not as a limitation, hemostatic agent can include FV, FVII, FVIII, FIX, FX, FXI, FXII, FXIII, prothrombin, or fibrinogen or activated forms of any of the preceding. The clotting factor or hemostatic agent can also include anti-fibrinolytic drugs, e.g., epsilon-amino-caproic acid, tranexamic acid.

The chimeric molecule of the invention can be administered intravenously, subcutaneously, intramuscularly, or via any mucosal surface, e.g., orally, sublingually, buccally, sublingually, nasally, rectally, vaginally or via pulmonary route. The chimeric molecule can be implanted within or linked to a biopolymer solid support that allows for the slow release of the chimeric molecule to the desired site.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle. For buccal and sublingual administration the composition can take the form of tablets, lozenges or fast dissolving films according to conventional protocols. For administration by inhalation, the chimeric molecules for use according to the present invention are conveniently delivered in the form of an aerosol spray from a pressurized pack or nebulizer (e.g., in PBS), with a suitable propellant.

In one embodiment, the route of administration of the polypeptides of the invention is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the invention, a form for administration would be a solution for injection, in particular for intravenous or intraarterial injection or drip. Usually, a suitable pharmaceutical composition for injection can comprise a buffer (e.g., acetate, phosphate or citrate buffer), a surfactant (e.g. polysorbate), optionally a stabilizer agent (e.g., human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to clotting disorders.

The pharmaceutical composition can also be formulated for rectal administration as a suppository or retention enema. e.g., containing conventional suppository bases such as cocoa butter or other glycerides.

Effective doses of the compositions of the present invention, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

In one embodiment, the dose of a biologically active moiety (e.g., comprising FVII), can range from about 90 to 270 μg/kg or 0.090 to 0.270 mg/kg. In another embodiment, the dose of a biologically active moiety (e.g., comprising FX), can range from about 1 μg/kg to 400 mg/kg.

Dosages can range from 1000 μg/kg to 0.1 ng/kg body weight. In one embodiment, the dosing range is 1 μg/kg to 100 μg/kg. The protein can be administered continuously or at specific timed intervals. In vitro assays can be employed to determine optimal dose ranges and/or schedules for administration. In vitro assays that measure clotting factor activity are known in the art, e.g., STA-CLOT Vlla-rTF clotting assay. Additionally, effective doses can be extrapolated from dose-response curves obtained from animal models, e g., a hemophiliac dog (Mount et al. 2002, Blood 99: 2670).

Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. In some methods, two or more polypeptides can be administered simultaneously, in which case the dosage of each polypeptide administered falls within the ranges indicated.

Polypeptides of the invention can be administered on multiple occasions. Intervals between single dosages can be daily, weekly, monthly or yearly. Intervals can also be irregular as indicated by measuring blood levels of modified polypeptide or antigen in the patient. Alternatively, polypeptides can be administered as a sustained release formulation, in which case less frequent administration is required. Dosage and frequency vary depending on the half-life of the polypeptide in the patient.

The dosage and frequency of administration can vary depending on whether the treatment is prophylactic or therapeutic. In prophylactic applications, compositions containing the polypeptides of the invention or a cocktail thereof are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

Polypeptides of the invention can optionally be administered in combination with other agents that are effective in treating the disorder or condition in need of treatment (e.g., prophylactic or therapeutic).

As used herein, the administration of polypeptides of the invention in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the polypeptides of the instant invention can be used in conjunction or combination with an agent or agents (e.g., to provide a combined therapeutic regimen). Exemplary agents with which a polypeptide of the invention can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the polypeptides of the instant invention can vary by subject or can be administered according to what is known in the art. See for example, Bruce A Chabner et al., Antineoplastic Agents, in Goodman & Gilman's The Pharmacological Basis of Therapeutics 1233-1287 ((Hardman et al., eds., 9th ed. 1996). In another embodiment, an amount of such an agent consistent with the standard of care is administered.

As previously discussed, the polypeptides of the present invention, can be administered in a pharmaceutically effective amount for the in vivo treatment of clotting disorders. In this regard, it will be appreciated that the polypeptides of the invention can be formulated to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. Of course, the pharmaceutical compositions of the present invention can be administered in single or multiple doses to provide for a pharmaceutically effective amount of the polypeptide.

In one embodiment, a chimeric molecule of the invention is administered as a nucleic acid molecule. Nucleic acid molecules can be administered using techniques known in the art, including via vector, plasmid, liposome, DNA injection, electroporation, gene gun, intravenously injection or hepatic artery infusion. Vectors for use in gene therapy embodiments are known in the art.

In keeping with the scope of the present disclosure, the chimeric molecule of the invention can be administered to a human or other animal in accordance with the aforementioned methods of treatment in an amount sufficient to produce a therapeutic or prophylactic effect.

The chimeric molecules of the invention have many uses as will be recognized by one skilled in the art, including, but not limited to methods of treating a subject with a disease or condition. The disease or condition can include, but is not limited to, hemostatic disorders.

In one embodiment, the invention relates to a method of treating a subject having a hemostatic disorder comprising administering a therapeutically effective amount of at least one chimeric molecule of the invention.

The chimeric molecules of the invention treat or prevent a hemostatic disorder by promoting the formation of a fibrin clot. The chimeric molecule of the invention can activate any member of a coagulation cascade. The clotting factor can be a participant in the extrinsic pathway, the intrinsic pathway or both. A chimeric molecule of the invention can be used to treat hemostatic disorders, e.g., those known to be treatable with the particular clotting factor present in the chimeric molecule. The hemostatic disorders that can be treated by administration of the chimeric molecule of the invention include, but are not limited to, hemophilia A, hemophilia B, von Willebrand's disease, Factor XI deficiency (PTA deficiency), Factor XII deficiency, as well as deficiencies or structural abnormalities in fibrinogen, prothrombin, Factor V, Factor VII, Factor X, or Factor XIII.

In one embodiment, the hemostatic disorder is an inherited disorder. In one embodiment, the subject has hemophilia A, and the chimeric molecule comprises activated FVII linked to or associated with a GPIIb/IIIa antibody or antigen-binding molecule thereof and an XTEN polypeptide. In another embodiment, the subject has hemophilia B and the chimeric molecule comprises activated FVII linked to or associated with a GPIIb/IIIa antibody or antigen-binding molecule thereof and an XTEN polypeptide. In some embodiments, the subject has inhibitory antibodies to FVIII or FVIIIa and the chimeric molecule comprises activated FVII linked to or associated with a GPIIb/IIIa antibody or antigen-binding molecule thereof and an XTEN polypeptide. In yet other embodiments, the subject has inhibitory antibodies against FIX or FIXa and the chimeric molecule comprises activated FVII linked to or associated with a GPIIb/IIIa antibody or antigen-binding molecule thereof and an XTEN polypeptide.

Chimeric molecules of the invention comprising FVII can be used to prophylactically treat a subject with a hemostatic disorder. Chimeric molecules of the invention comprising FVII can be used to treat an acute bleeding episode in a subject with a hemostatic disorder.

In one embodiment, the hemostatic disorder is the result of a deficiency in a clotting factor, e.g., FVII, FIX, or FVIII. In another embodiment, the hemostatic disorder can be the result of a defective clotting factor. In another embodiment, the hemostatic disorder can be an acquired disorder. The acquired disorder can result from an underlying secondary disease or condition. The unrelated condition can be, as an example, but not as a limitation, cancer, an autoimmune disease, or pregnancy. The acquired disorder can result from old age or from medication to treat an underlying secondary disorder (e.g. cancer chemotherapy).

The invention also relates to methods of treating a subject who does not have a hemostatic disorder or a secondary disease or condition resulting in acquisition of a hemostatic disorder. The invention thus relates to a method of treating a subject in need of a general hemostatic agent comprising administering a therapeutically effective amount of at least one chimeric molecule of the invention. For example, in one embodiment, the subject in need of a general hemostatic agent is undergoing, or is about to undergo, surgery. The chimeric molecule of the invention can be administered prior to or after surgery as a prophylactic. The chimeric molecule of the invention can be administered during or after surgery to control an acute bleeding episode. The surgery can include, but is not limited to, liver transplantation, liver resection, or stem cell transplantation. In another embodiment, the chimeric molecule of the invention can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding.

Having now described the present invention in detail, the same will be more clearly understood by reference to the following examples, which are included herewith for purposes of illustration only and are not intended to be limiting of the invention. All patents and publications referred to herein are expressly incorporated by reference in their entireties.

### EXAMPLES

### General Materials and Methods

In general, the practice of the present invention employs, unless otherwise indicated, conventional techniques of chemistry, biophysics, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., CS.H.L. Press, Pub. (1999); and Current Protocols in Molecular Biology, eds. Ausubel et al., John Wiley & Sons (1992).

### Example 1

### Identification and Characterization of Platelet-Targeted Antibodies

Hybridomas were generated from BALB/C mice immunized with plasmids containing DNA sequences encoding GPIIb/IIIa (SEQ ID NOs:183 and 184) according to methods known in the art. Hybridomas were then screened for binding to human and cynomolgus monkey platelets using flow cytometry, and for binding to GPIIb/IIIa using Enzyme-linked immunosorbent assays (ELISA). To determine binding to human and money platelets, gel-purified human or monkey (cynomolgus) platelets in Tyrode's buffer were incubated with hybridoma supernatant. Following a 30 minute incubation, cells were fixed in 1% formaldehyde. Following fixation, cells were washed in Tyrode's buffer and a detection antibody was added (Jackson Immunoresearch goat anti-mouse IgG-PE conjugated). Antibody binding was detected by flow cytometry.

The binding of supernatants from hybridomas to human GPIIb/IIIa (αIIbβ) was determined by using ELISA as follows, Costar plates (Cat. No. 3590) were coated with 100 μl/well of 5 μg/mL human GPIIb/IIIa (Calbiochem Cat No. 528240) in measuring buffer (20 mM Tris, pH 7.4, 150 mM NaCl, 1 mM MgCl₂, and 1 mM MnCl₂) and incubated for 1 hour at 37° C. with shaking. Wells were washed three times with TBST using a plate washer. Blocking was performed using 200 μl of measuring buffer containing 5% BSA (Bovine Serum albumin, Jackson Cat No 001 000 173) per well, and incubating 1 hour at 37° C. with shaking. 100 μL of hybridoma supernatant were added assay wells, incubated for 1 hour at 37° C. with shaking, and washed three times with TBST. A 1:10,000 dilution of goat anti mouse IgGHRP (Southern Biotech (Cat. No. 1010 05) in measuring buffer was added, incubated for 1 hour at 37° C. with shaking, and washed three times with TBST. HRP presence was developed using TMB and O.D. read at 450 nm using a Molecular Devices plate reader.

The supernatants from hybridomas which tested positive in the ELISA assays were mixed with platelets and screened for platelet activation using flow cytometry as follows.

(a) Reagents:

Citrated human whole blood; Sepharose 2B beads (GE Healthcare); Tyrode's buffer with 1 mg/mL BSA (no calcium); Tyrode's buffer with 5 mM CaCl₂ and 1 mg/mL BSA; 32% paraformaldehyde (PFA) (EM Sciences); PAC1 FITC antibody (BD Cat. No. 340507); CD62 PE antibody (BD Cat. No. 555524); ADP; SFFLRN peptide (Anaspec, Cat. No. 24191); IV.3 Fabb anti CD32 (StemCell, Cat. No. 01470).

b) Platelet Purification:

A 10 mL Sepharose 2B bead column was packed and equilibrated with 30 mL of Tyrode's buffer containing 1 mg/mL BSA. A volume of 1 to 1.5 mL of platelet-rich plasma (PRP) was loaded onto the equilibrated Sepharose column and allowed to enter the packed beads by gravity, followed with approximately 5 mL of Tyrode's buffer. The turbid drops, which contained the platelets, were collected.

(c) Assay:

First, 50 μL aliquots of hybridoma supernatant were added to assay wells of a 96 well round bottom plate. 10 μL of PAC1 FITC and 10 μL of CD62PE were added to all control and assay wells. 10 μL of ADP and 10 μL of SFFLRN were added to all control wells (no hybridoma supernatant). 10 μL of IV.3 inhibitor (antibody to FcγRIIA) were added wells to see if activation was Fc or antibody mediated. Next, a 50 μL aliquot of concentrated resting platelets, which was purified as described above, was added to all wells. Plates were incubated for 30 minutes in the dark and at room temperature. Cells were fixed with 1% PFA (final concentration) for 10 minutes at room temperature (a volume of 2% PFA equal to the content of each well was added). After fixation, samples were analyzed by flow cytometry.

The antibodies that did not activate platelets upon binding to GPIIb/IIIa were selected as candidates for clotting factor targeting moieties. The antibodies that activate platelets upon binding to GPIIb/IIIa were excluded from selection.

Antibodies can also activate platelets by binding to the FcγRIIA receptor via the Fc region, which were not excluded from the selection because their antigen-binding portion contain no Fc region and therefore not bind to the FcγRIIA receptor. These antibodies can be identified by blocking the FcγRIIA receptor with an inhibitor.

The supernatants from non-activating hybridomas were subject to additional characterization assays (i) to confirm antibody binding to human and cynomolgus platelets, (ii) to determine antibody binding specificity for the α and/or β subunit of GPIIb/IIIa, and (iii) to determine whether the antibodies can compete with fibrinogen for binding to platelets. Fibrinogen is the natural ligand of GPIIb/IIIa and its binding to GPIIbIIIa is essential to mediate platelet aggregation. Thus, the antibodies that compete with the binding of fibrinogen to GPIIb/IIIa were excluded from the selection.

Antibody binding to the α and/or β subunit of GPIIb/IIIa was assessed using ELISA, whereas antibody competition with fibrinogen was assessed using flow cytometry. Antibodies determined to be non-activating (e.g., clones 34D10, 2A2, 35D1, 36A8, 4B11, 1H6, 38G8, 21F10, 38A8, 18F7, 12B2, 38F6, 13C1, 5C4, 23C10, 37C7, 28C2, 9D6, 13A1) were clustered into 6 different groups according to the VH domain sequence similarity, α or β subunit specificity, ability to compete with fibrinogen, and relative strength of the signals measured via ELISA and flow cytometry (see TABLE 3).

TABLE 3
Platelet Specific Non-Activating Anti-GPIIb/IIIa Monoclonal Antibodies

| Group/Antibody | | | SEQ | CDR SEQ IDs | | |
|---|---|---|---|---|---|---|
| Characteristics | Antibody/Chain | | ID | CDR1 | CDR2 | CDR3 |
| 1/α subunit specific | 2A2 | HC | 3 | 25 | 26 | 27 |
| No fibrinogen competition | | LC | 4 | 28 | 29 | 30 |
| | 34D10^{#} | HC | 1 | 31 | 32 | 33 |
| | | LC | 2 | 34 | 35 | 36 |
| | 35D1 | HC | 97 | 111 | 112 | 113 |
| | | LC | 98 | 114 | 115 | 116 |
| | 36A8 | HC | 5 | 37 | 38 | 39 |
| | | LC | 6 | 40 | 41 | 42 |
| | 4B11 | HC | 7 | 43 | 44 | 45 |
| | | LC | 99 | 117 | 118 | 119 |
| 2/α subunit specific. | 1H6 | HC | 8 | 46 | 47 | 48 |
| Fibrinogen competition | | LC | 9 | 49 | 50 | 51 |
| | 38G8 | HC | 100 | 120 | 121 | 122 |
| | | LC | 101 | 123 | 124 | 125 |
| | 21F10 | HC | 102 | 126 | 127 | 128 |
| | | LC | 103 | 129 | 130 | 131 |
| | 38A8 | HC | 10 | 52 | 53 | 54 |
| | | LC | 11 | 55 | 56 | 57 |
| 3/α subunit specific. | | HC | 12 | 58 | 59 | 60 |
| Fibrinogen competition. Low | 18F7 | LC | 13 | 61 | 62 | 63 |
| ELISA signal v flow | | | | | | |
| cytometry | | | | | | |
| 4/β subunit specific. No | 12B2^{$} | HC | 14 | 64 | 65 | 66 |
| fibrinogen competition | | LC | 15 | 67 | 68 | 69 |
| | 38F6 | HC | 16 | 70 | 71 | 72 |
| | | LC* | 104 | 132 | 133 | 134 |
| | 13C1 | HC | 105 | 135 | 136 | 137 |
| | | LC* | 106 | 138 | 139 | 140 |
| 5/β subunit specific. | 5C4 | HC | 17 | 73 | 74 | 75 |
| Fibrinogen competition | | LC* | 107 | 141 | 142 | 143 |
| | 23C10 | HC | 18 | 76 | 77 | 78 |
| | | LC* | 108 | 144 | 145 | 146 |
| | 37C7 | HC | 109 | 147 | 148 | 149 |
| | | LC* | 110 | 150 | 151 | 152 |
| | 28C2 | HC | 19 | 79 | 80 | 81 |
| | | LC | 20 | 82 | 83 | 84 |
| | 9D6 | HC | 21 | 85 | 86 | 87 |
| | 9D6 | LC | 22 | 88 | 89 | 90 |
| 6/β subunit specific. | | HC | 23 | 91 | 92 | 93 |
| Fibrinogen competition. Low | 28F4 | LC | 24 | 94 | 95 | 96 |
| ELISA signal v flow | | | | | | |
| cytometry | | | | | | |

*Not sequenced.
HC = Heavy Chain (i.e., VH).
LC = Light Chain (i.e., VL)
^{#}α subunit or complex specific
^{$}β subunit specific

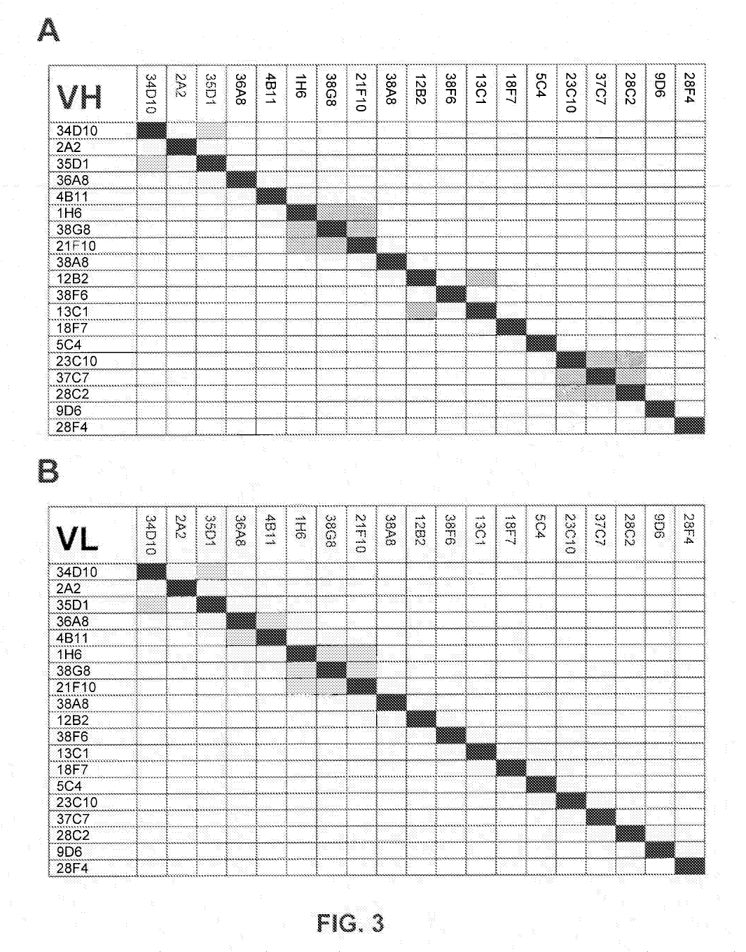

Several non-activating antibodies identified using the screening method described above shared the same VH or VL domains, as show in FIG. 3. For example, 35D1 and 34D10 shared the same VH domain.

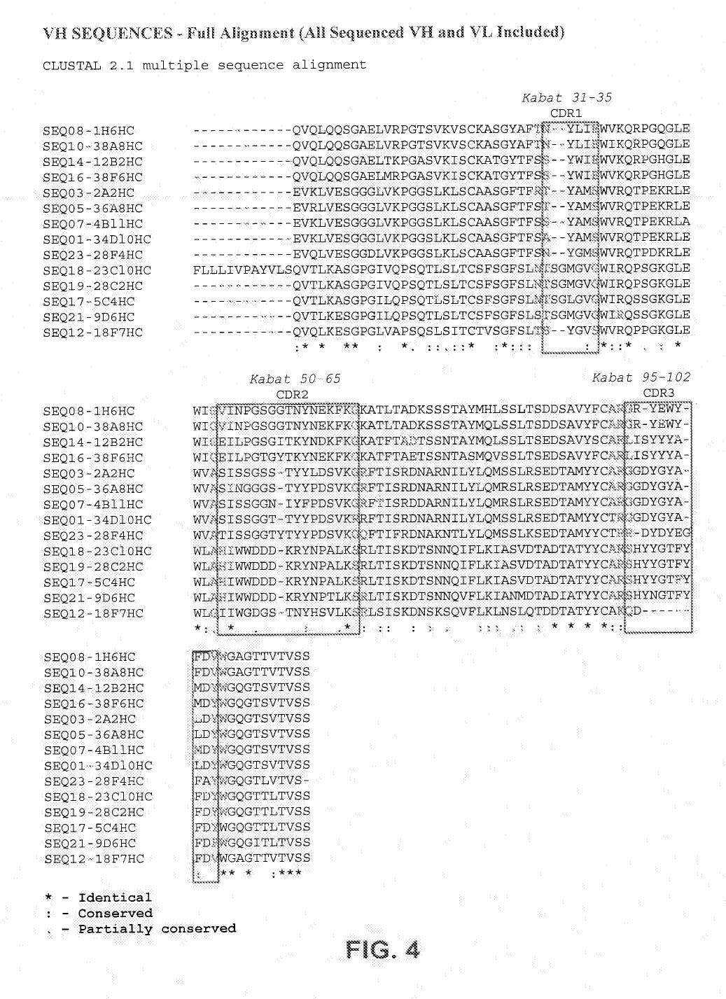

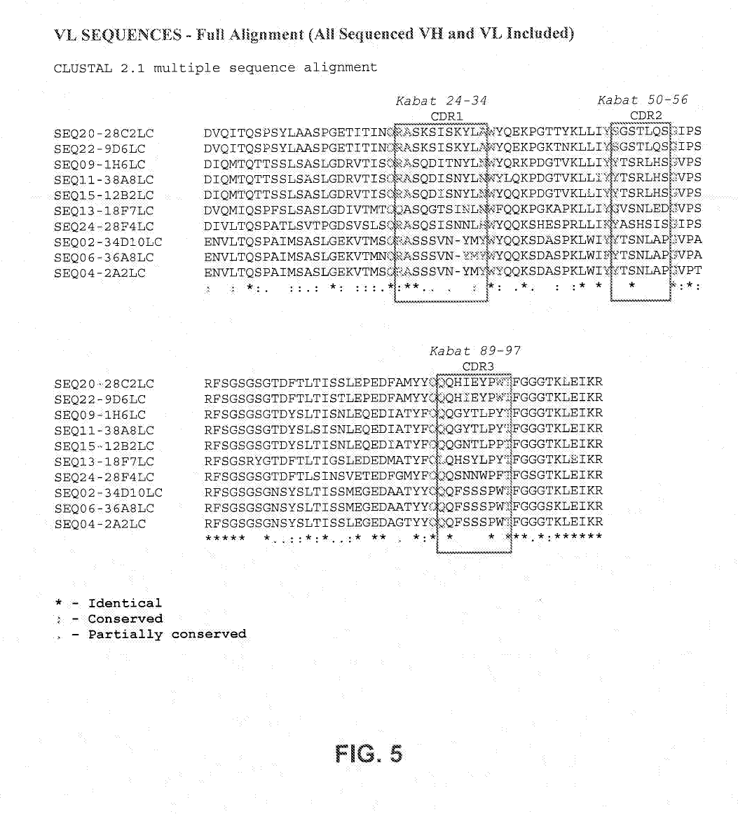

Multiple sequence alignments corresponding to the VH and VL domains of the above identified antibodies are shown in FIG. 4 and FIG. 5, respectively. The multiple sequence alignments show the location of the complementarity determining regions CDR1, CDR2, and CDR3 in the VH and VL domain and their location according to the EU numbering system (Kabat, E. A., Wu, T. T., Perry, H., Gottesman, K., and Foeller, C. (1991) “Sequences of Proteins of Immunological Interest,” 4th ed., U.S. Govt. Printing Off. No. 165-492, Nethesda, Md.).

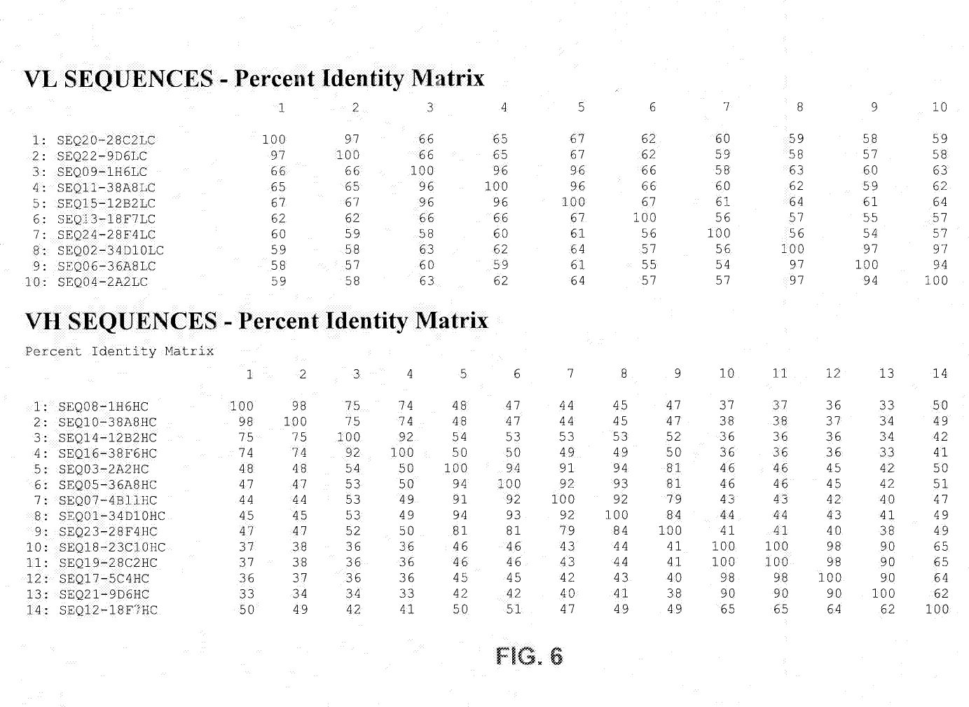

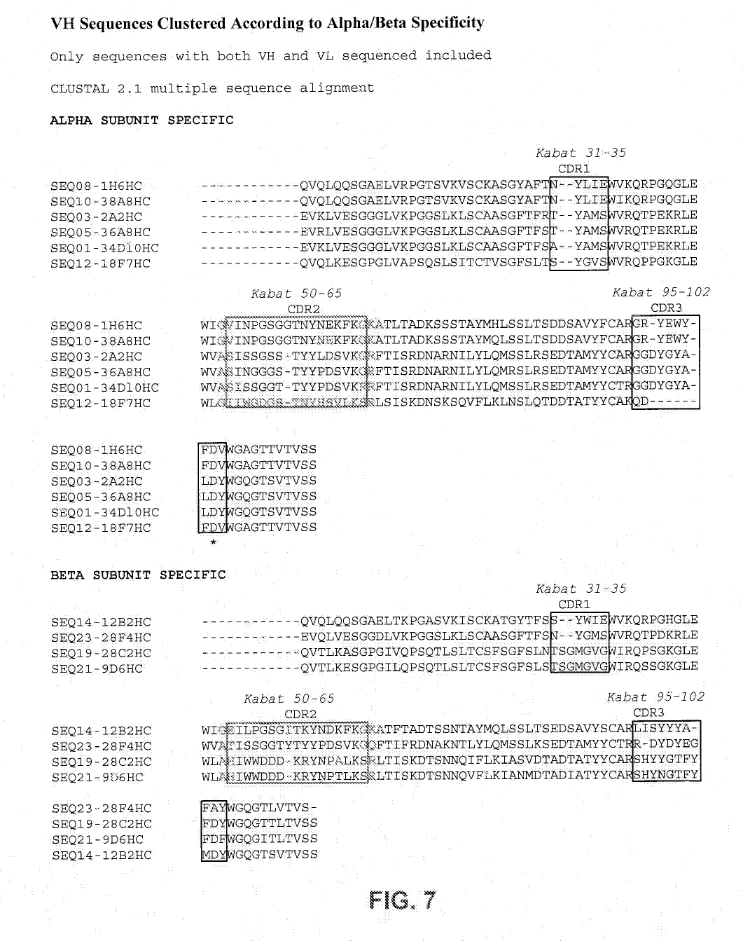

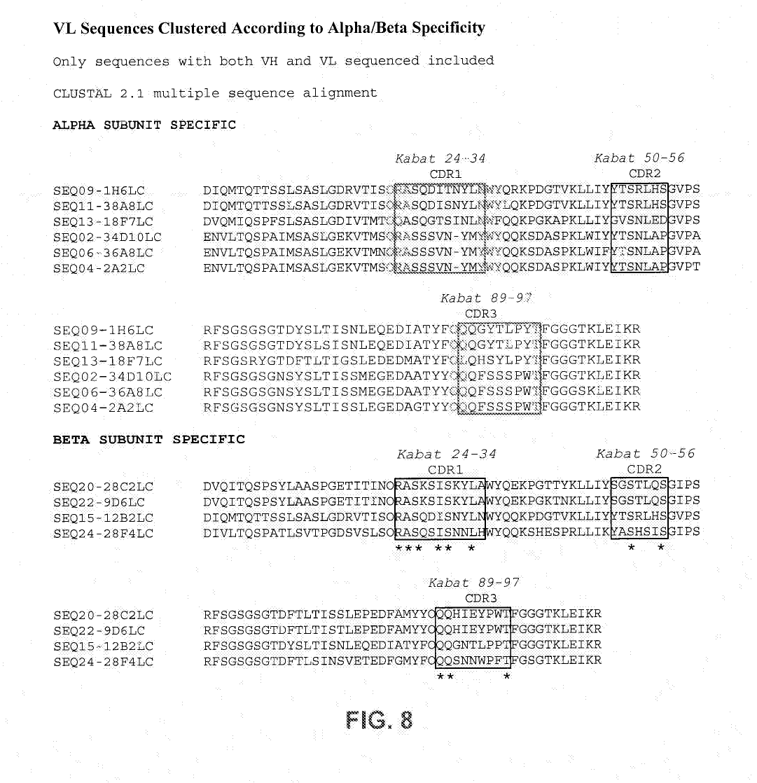

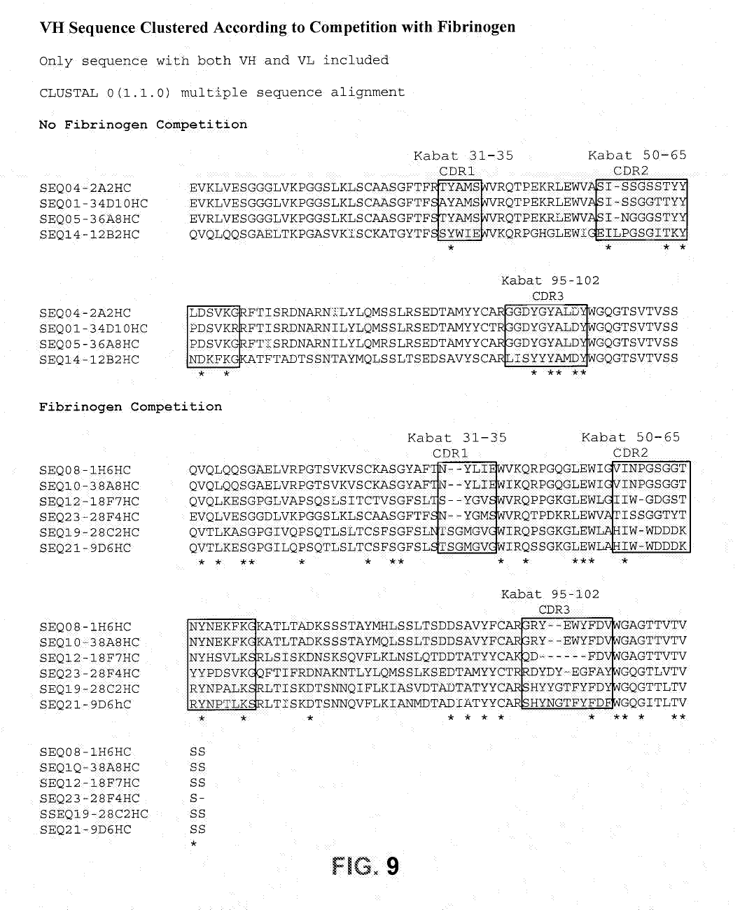

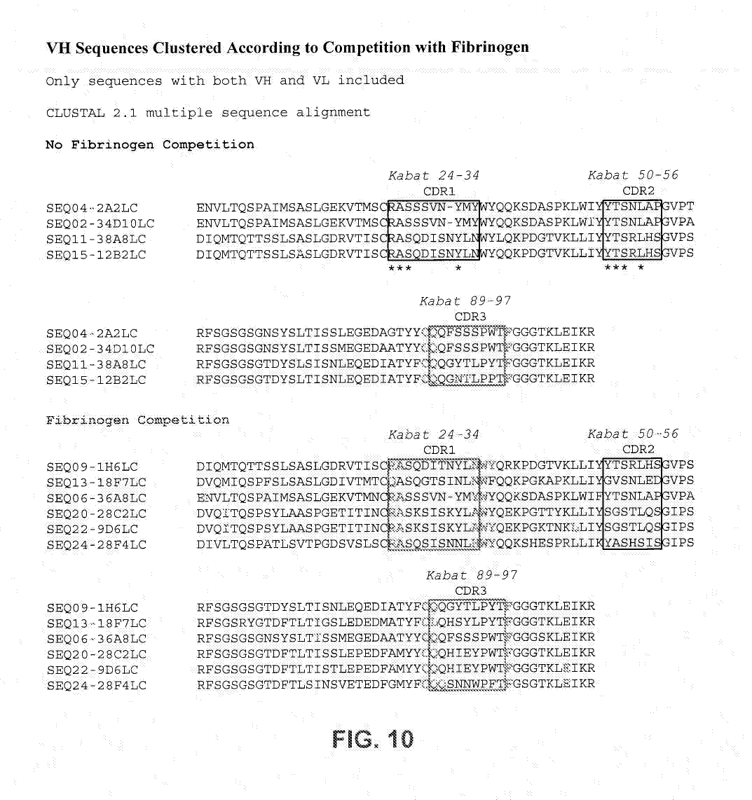

FIG. 6 shows percent identity matrices showing the percentage of sequence identity among each pair of the VH and VL sequences shown in the multiple sequence alignments of FIGS. 4 and 5. Column designations correspond to the designation applied to each row in the matrix. For example, column 2 corresponds to “2: SEQ 22-9D6LC.” The sequences aligned in FIG. 4 and FIG. 5 were clustered according to the specificity of the antibodies for the α or β subunits of GPIIb/IIIa (see FIG. 7 and FIG. 8), and according to their ability to compete with fibrinogen for binding to GPIIb/IIIa (see FIG. 9 and FIG. 10).

### Example 2

### Construction of Platelet-Targeted Chimeric Molecules Comprising Anti-GPIIb/IIIa Antibodies, XTEN, and FVIIa

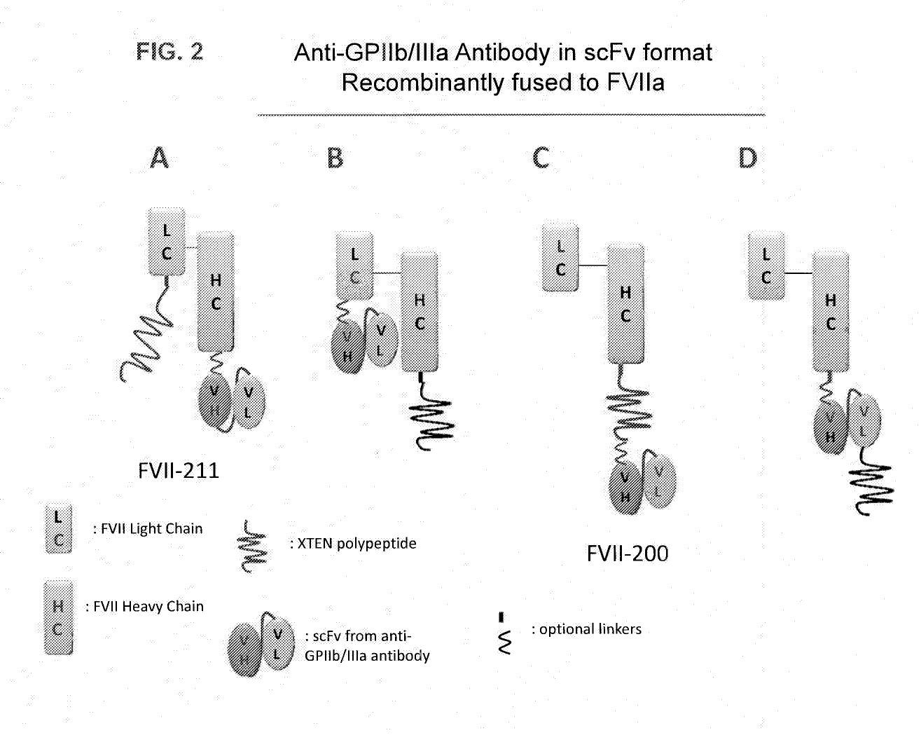

The monoclonal antibodies against GPIIb/IIIa were used to target the FVIIa clotting factor to the surface of platelets. Accordingly, scFvs derived from the platelet-specific monoclonal antibodies identified in section II.A.1. were fused to FVIIa using molecular biology methods known in the art. The constructs were transiently expressed in HEK 293 cell and purified by standard methods available in the art. In the resulting chimeric molecules, the C-terminus of the heavy chain of FVIIa was fused to the N-terminus of an scFv comprising a VH and a VL domain derived from non-activating platelet-targeting antibodies, and the C-terminus of the light chain of FVII was fused to the N-terminus of an XTEN polypeptide (see FIG. 2A). A chimeric molecule comprising an scFv derived from the GPIIb/IIIa α subunit-specific 34D10 antibody fused to the heavy chain of FVII and an XTEN polypeptide (i.e., ______) fused to the light chain of FVII was designated as “FVII-211”. A second chimeric molecule comprising a light chain of FVII and a fusion peptide consisting of the heavy chain of FVII, an XTEN polypeptide (i.e., AE288), and an scFv derived from the GPIIb/IIIa α subunit-specific 34D10 antibody was designated as “FVII-200.” A chimeric molecule comprising FVII and an scFv derived from the GPIIb/IIIa α subunit-specific 34D10 antibody, which was fused to the C-terminus of the heavy chain of FVII, was designated as “FVII-189”. These platelet-targeted FVIIa variants were characterized for their ability to bind to platelets, their procoagulant activity, and their pharmacokinetic properties in animals.

### Example 3

### XTEN Improves Pharmacokinetic Properties of rFVIIa but Reduces its Activity

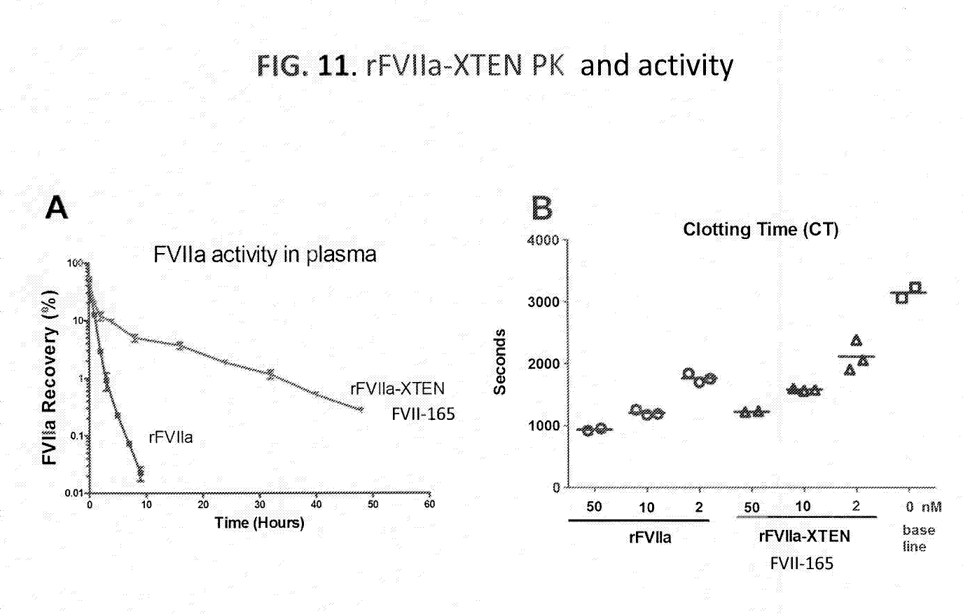

To assess the pharmacokinetic properties, HemA mice were administered with a single intravenous dose of either rFVIIa or FVIIa-XTEN at 20 nmol/kg. Plasma samples were collected at various times after dosing, and FVIIa activity was determined by a FVIIa specific, soluble tissue factor dependent prothrombin time (sTF-PT) assay (FIG. 11A). PK parameters were derived by using Phoenix 6 program (Pharsight).

As shown in FIG. 11A and TABLE 4, XTEN linked to the C-terminus of rFVIIa extended the circulating half-life of FVIIa by about 8-fold in HemA mice. Other PK parameters were improved as well, as indicated by an increase from 0.82 to 11 hours in mean residence time (MRT), and a roughly 5-fold increase in the dose-normalized area under the curve (AUC) in the plasma activity vs time chart (FIG. 11A).

TABLE 4
Pharmacokinetic Properties of rFVIIa and rFVIIa-XTEN

| | T_{1/2} | | CL | Vss | AUC/D |
|---|---|---|---|---|---|
| Treatment | (hr) | MRT (hr) | (mL/hr/kg) | (mL/kg) | (hr * kg/L) |
| rFVIIa-XTEN | 8.92 | 11 | 22 | 241 | 45.62 |
| rFVIIa | 1.17 | 0.82 | 109 | 89 | 9.18 |

The PK parameters were generated by Phoenix 6 (Parsight).

While XTEN improves the pharmacokinetics of rFVIIa, the clotting activity of rFVIIa-XTEN as measured in human hemophilia A blood by ROTEM was reduced, however, to about 25% of rFVIIa (FIG. 11B).

### Example 4

### Combining Platelet-Targeting with XTEN Increases its Activity and Improves PK

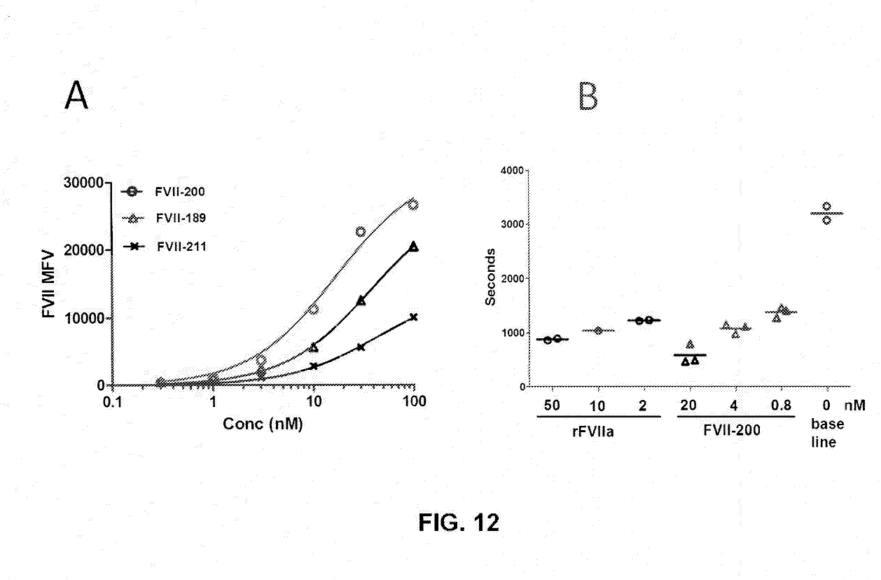

Targeting rFVIIa or rFVIIa-XTEN to platelets may result in an increase in the local concentration of the protein at the site of coagulation (platelets), thereby enhancing the clotting activities. The platelet-targeted chimeric molecules FVII-200, FVII-211, and FVII-189 were constructed to contain a scFv from monoclonal antibody 34D10 as the targeting moiety. The structure of the constructs was described above. All three molecules were capable of binding to human platelets (FIG. 12A) in a dose-dependent manner, and this binding relied on 34D10. However, rFVIIa by itself had no detectable platelet-binding activity in this assay. Therefore, the scFv from 34D10 could direct rFVIIa (FVII-189) or rFVIIa-XTEN (FVII-200 and FVII-211) to platelets.

As a result from the platelet-binding capability, the 34D10-targeted rFVIIa-XTEN chimeric molecules displayed enhanced clotting activity when assayed in human hemophilia A blood by ROTEM (FIG. 12B). Based on the clotting time recorded by ROTEM, FVII-200 was estimated to be 2 to 5-fold more active than rFVIIa. In comparison, rFVII-XTEN was only 25% as active as rFVIIa (FIG. 11B). Thus targeting rFVIIa-XTEN to platelets by 34D10 resulted in a substantial improvement in activity.

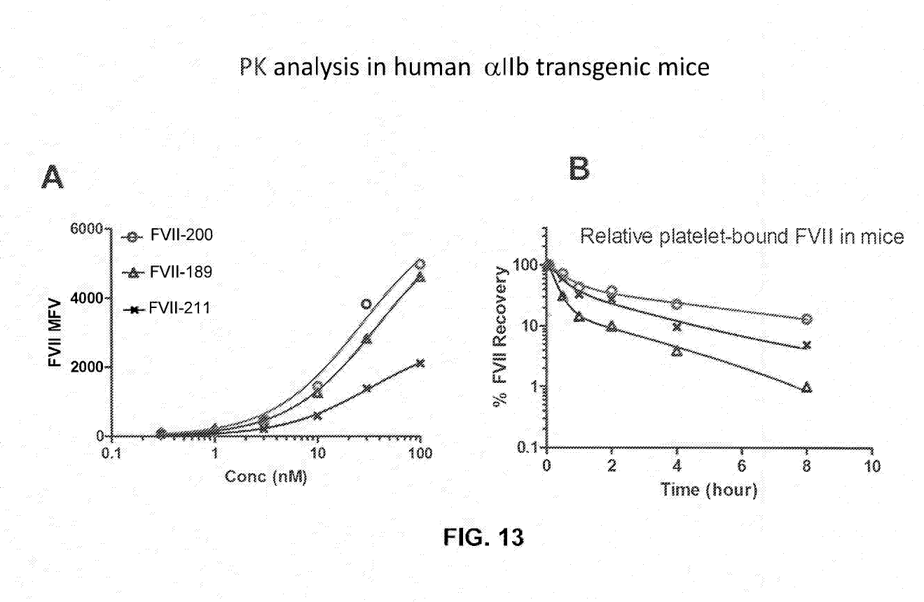

While the monoclonal antibody 34D10 does not bind to mouse platelets, it does bind to the platelets in blood from human αIIb transgenic (Tg) mice (hallb+/mallb−). Moreover, the 34D10-targeted FVIIa (FVII-189) and FVIIa-XTEN (FVII-200 and FVII-211) retained its ability to bind to platelets from human αIIb Tg mice (FIG. 13A), thus justifying the use of these Tg mice to assess the pharmacokinetic properties of the 34D10-containing rFVIIa chimeric molecules.

As shown in FIG. 13B, the clearance of platelet-bound FVIIa chimeric molecules in αIIb Tg mice fit with a two phase decay model. Compared to 34D10-targeted rFVIIa (FVII-189), addition of XTEN (FVII-200 and FVII-211) extended its terminal half-life, but also alleviated the initial c phase decay. Notably, the PK improvement was affected by the location of XTEN placement, as the PK profile of FVII-200 was markedly better than that of FVII-211. The improved PK property, together with the 2 to 5-fold better clotting activity than rFVIIa, shows the potential of this chimeric molecule for prolonged therapeutic efficacy.

### Example 5

### Platelet-Targeted FVIIa XTEN Chimeric Molecules with Alternative XTEN and the Targeting Moeity Placements

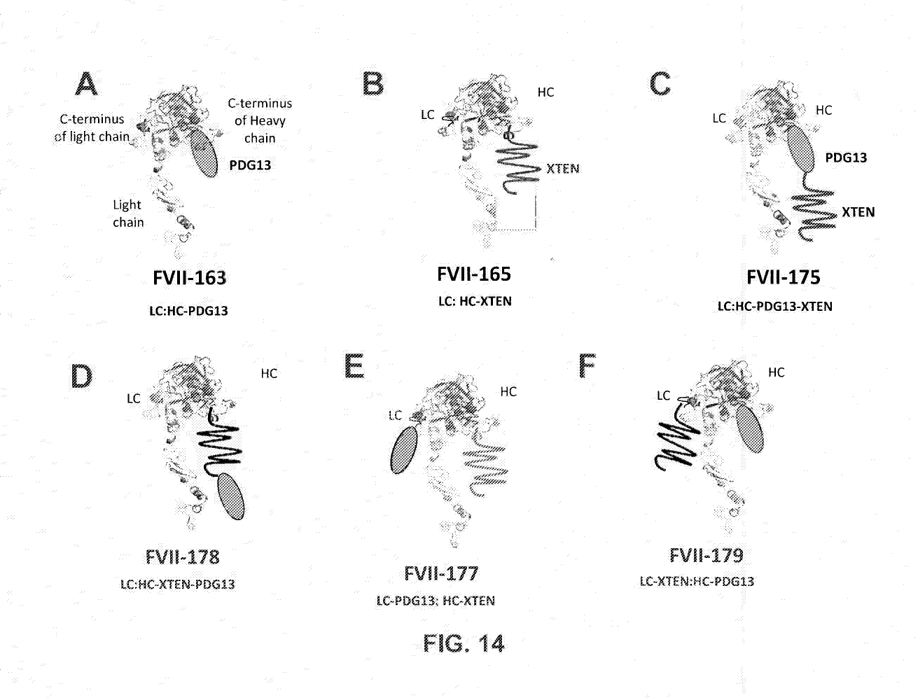

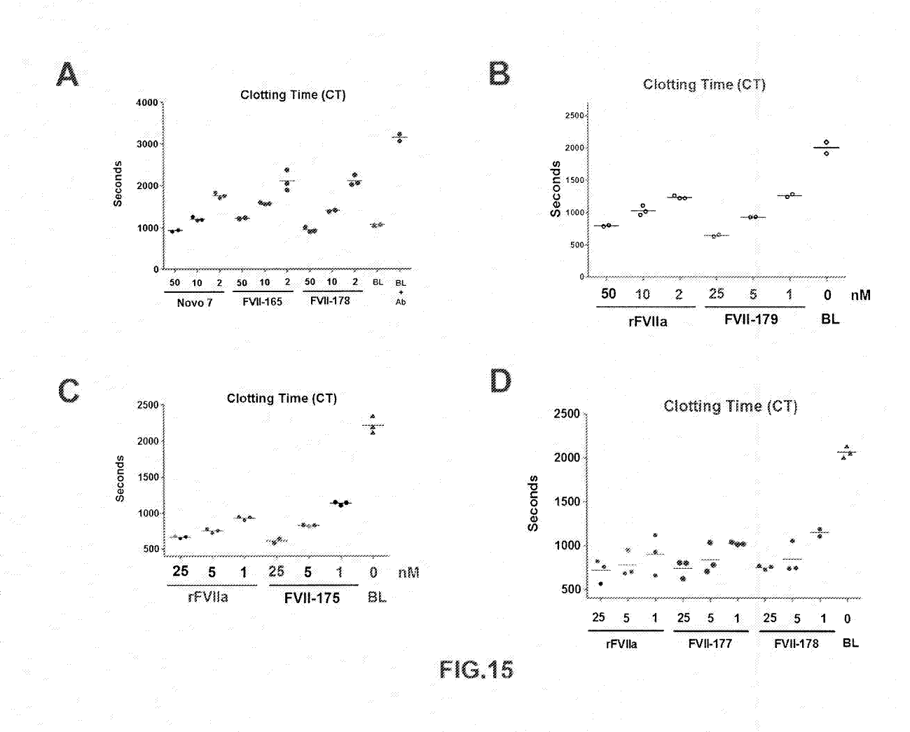

Using an scFv from monoclonal antibody PDG13, a class of chimeric molecules (FIG. 14) comprising FVIIa, PDG13, and XTEN were constructed, expressed in HEK 293 cells, and purified. Among the alternative placement of PDG13 and XTEN constructs (FIGS. 14C, 14D, 14E, 14F) tested, FVII-179, which contained XTEN at the light chain and PDG13 at the heavy chain of rFVIIa (FIG. 14F), was the most active. FVII-179 displayed about 5-fold increase in clotting activity (FIG. 15B) in human hemophilia A blood, when compared to that of rFVIIa. All other chimeric molecules, i.e., FVII-175 (FIGS. 14C, 15C), FVII-178 (FIGS. 14D,15A, 15D), FVII-177 (FIG. 14D, 15D) showed comparable clotting activities to that of rFVIIa, representing several fold improvement over FVII-165 (FIG. 14B), which is a chimeric molecule comprising the rFVIIa and XTEN fusion without the platelet-targeting moiety.

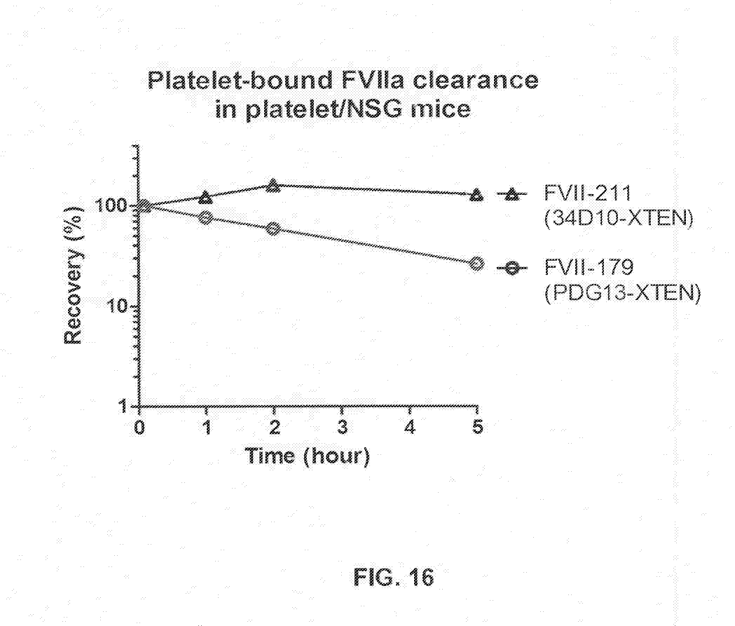

The effect of targeting moiety on PK was investigated. As shown in FIG. 16, when analyzed in NSG mice the recovery of administrated proteins on transfused human platelets over time, FVII-211 showed higher recovery than FVII-179 at all times following 5 min after protein administration, indicating a slower clearance on platelets of FVII-211 than that of FVII-179, although these two proteins were structurally identical except the platelet-targeting moiety, with FVII-211 being 34D10 and FVII-179 being PDG13. The result indicated that the targeting moiety can contribute to the PK improvement.

### Example 6

### Recombinant Factor FVIIa Fusion Proteins with Enhanced In Vitro Activity and Improved Half-Life in Mice

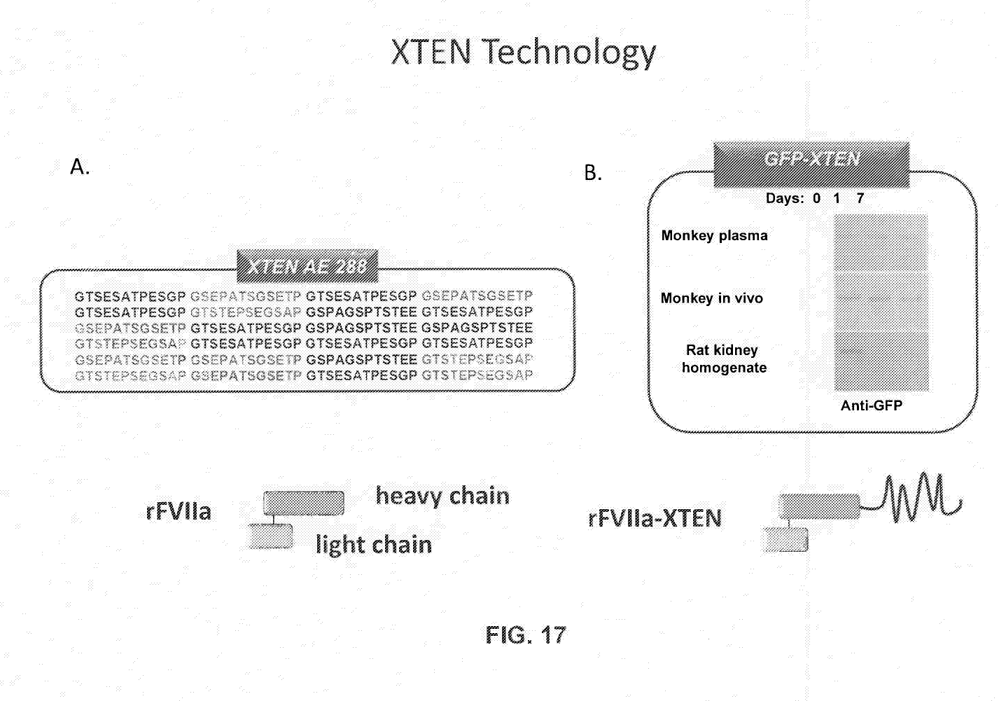

In order to achieve half-life extension to potentially enable prophylaxis, the use of XTEN technology was explored. XTEN sequences are unstructured protein sequences comprising repeated sequences of six different amino acids (G, S, E, T, A, P) that confer improved pharmacokinetic properties when appended to therapeutic peptides and proteins (FIG. 17). XTEN technology increases the hydrodynamic radius of fusion proteins, preventing renal clearance, and may shield from clearance receptors and/or protease cleavage. Recombinant nature allows precise control of composition, length, and placement, including multiple insertions, without chemical modification. XTEN contains natural amino acids that can be metabolized in vivo, as demonstrated in vitro with kidney homogenates. (FIG. 17B) An XTEN AE 288 sequence was recombinantly fused to the C-terminus of rFVIIa and characterized in vitro and in vivo. The goal is to utilize the XTEN technology to extend the half-life of FVIIa to potentially enable prophylaxis in people with hemophilia with inhibitors.

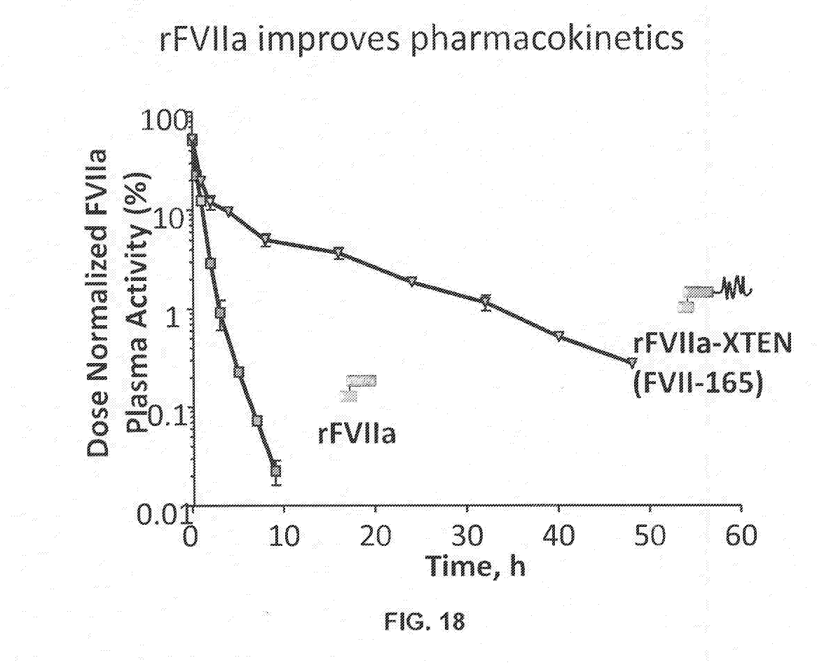

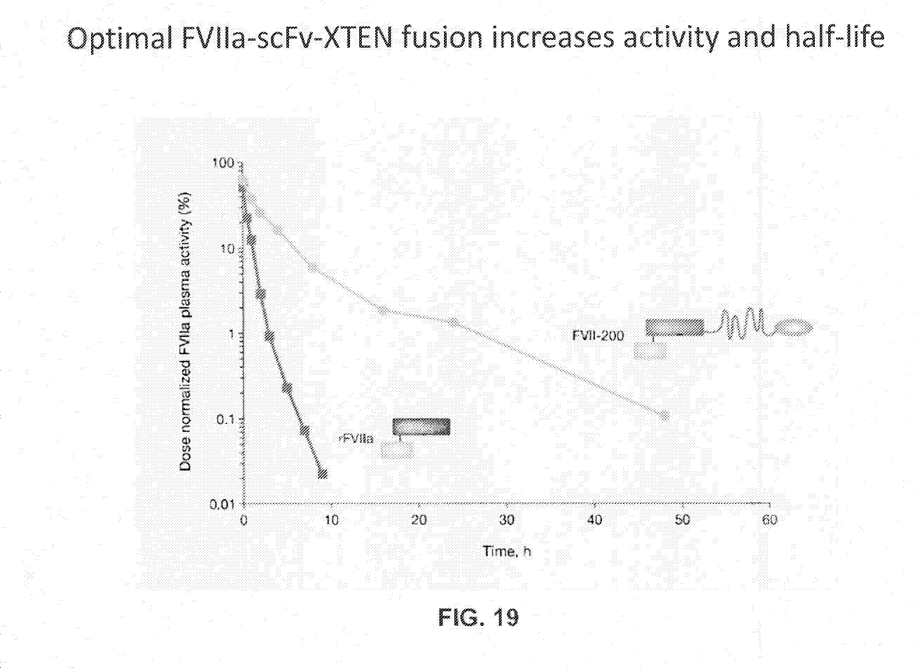

Linking an XTEN to the C-terminus of rFVIIa extended its circulating half-life by 8-fold in hemophilia A mice (FIG. 18), and resulted in a 5-fold increase in overall drug exposure (AUC). The activity of rFVIIa-XTEN, however, was reduced to approximately 25% of rFVIIa-based on ROTEM assays. A number of configurations of scFv and XTEN fusions with rFVIIa were tested for pharmacokinetics in hemophilia A mice and activity in ROTEM assays. The optimal configuration resulted in a significant increase in the half-life and overall exposure to rFVIIa (AUC) (FIG. 19), as well as 2- to 5-fold increase in activity over rFVIIa as measured by ROTEM.

In conclusion, the platelet-targeted rFVIIa molecules display a 25- to 50-fold increase in activity versus rFVIIa by ROTEM, do not activate or inhibit platelet function, and/or do not affect platelet clearance in vivo. Application of the XTEN technology to the platelet-targeted rFVIIa molecules enabled an 8-fold increase in half-life over rFVIIa, but with a 4-fold decrease in ROTEM activity. The combination of the platelet-targeting and XTEN technologies demonstrated that the optimal configuration of the XTEN and scFv moieties results in a 6-fold increase in AUC as well as a 2- to 5-fold increase in ROTEM activity. This combination of technologies may lead to improved bypass therapies utilizing a wild-type FVII sequence.

### Example 7

### Construction and Expression of Platelet-Targeted Constructs FVII-227, FVII-228, FVII-231, FVII-232, FVII-242, FVII-243 and FVII-238

Three (3) configurations of platelet-targeted rFVIIa-XTEN variants were generated: Configuration A (FVII-227, FVII-228, FVII-211), the XTEN and the targeting moiety were fused to the C-terminus of the light and heavy chain of FVIIa, respectively; Configuration B (FVII-231, FVII-232, FVII-200), both the XTEN and the platelet-targeting moiety were fused to the C-terminus of the heavy chain of rFVIIa; and Configuration C (FVII-242, FVII-243 and FVII-238), XTEN was fused to the C-terminus of both heavy and light chain, with the targeting moiety at the C-terminus of the heavy-chain XTEN. For each configuration, multiple XTEN lengths were tested ranging from 72 to 288 amino acids. The DNA encoding these proteins was generated using molecular biology methods known in the art. The constructs were transiently expressed in HEK 293 cell and purified by standard methods.

### Example 8

Characterization of the Activity of Proteins FVII-165, FVII-189, FVII-200, FVII-227, FVII-228, FVII-231, FVII-232, FVII-242, FVII-243 and FVII-238

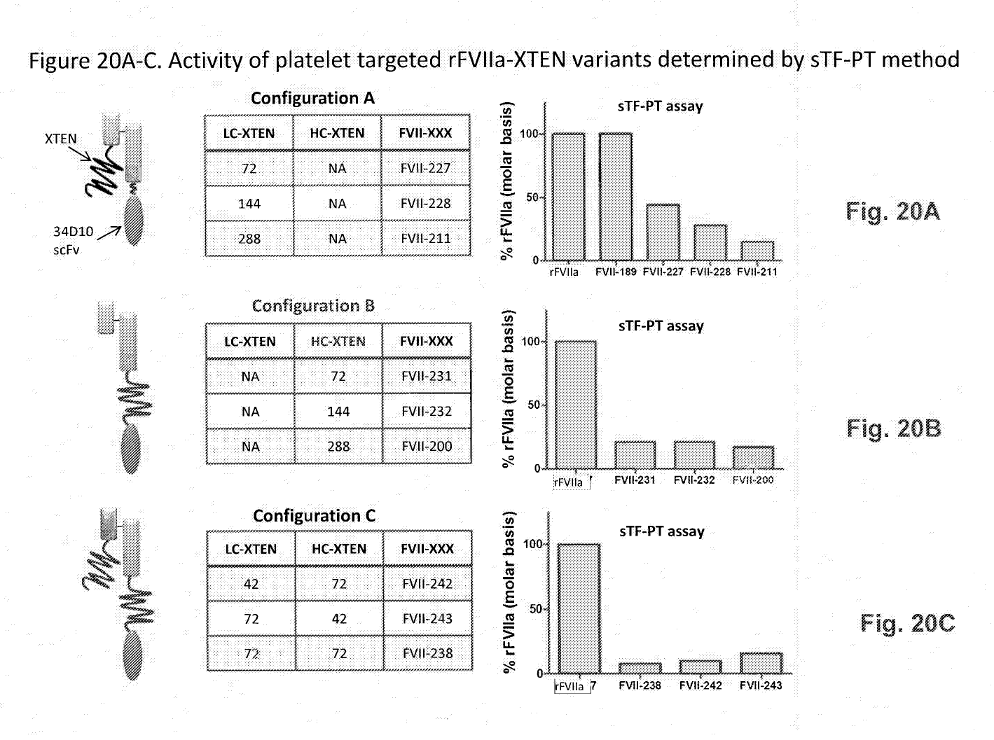

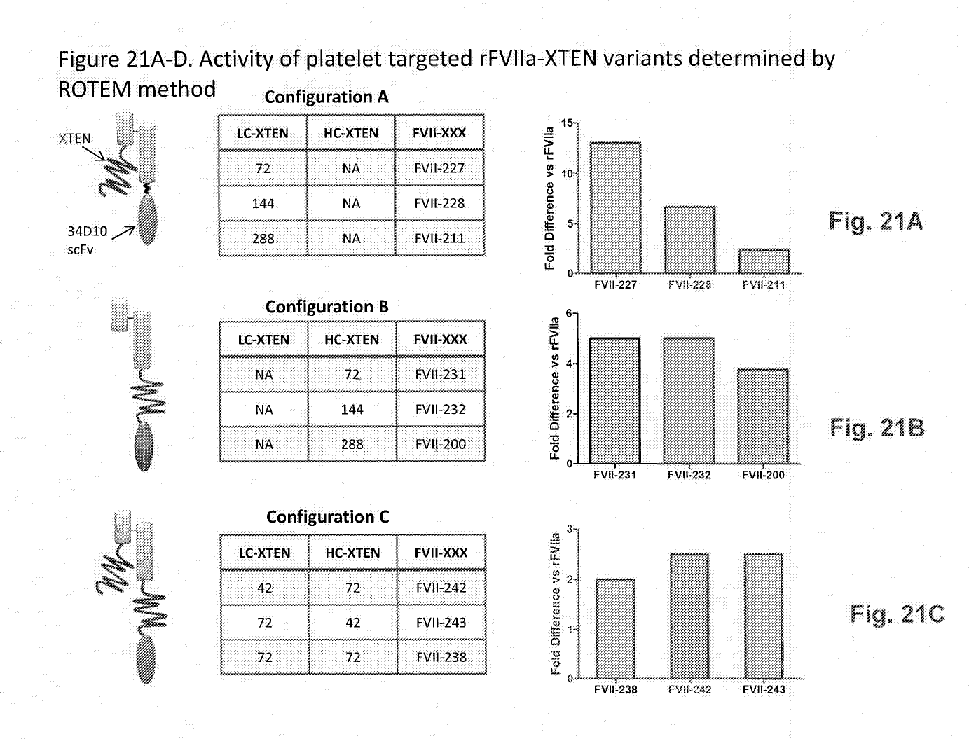

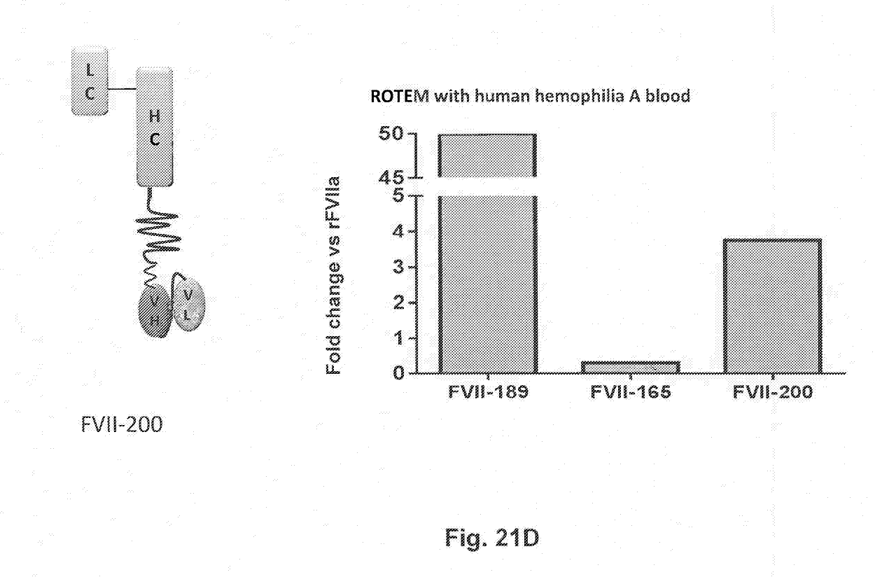

The activity of these variants was characterized by platelet-independent (sTF-PT) and platelet-dependent (ROTEM) methods. Based on the sTF-PT method, the specific activity of the platelet-targeted rFVIIa-XTEN variants in all configurations was lower than rFVIIa on a molar basis (FIG. 20A-C). Configuration A variants displayed the highest specific activity, ranging from 44% to 15% of rFVIIa, and activity was highly dependent and inversely correlated to XTEN length. The activity of Configuration B variants was lower and less dependent on XTEN length. Configuration C construct showed the lowest activity by this method. When tested in a platelet-dependent method (ROTEM) in human whole blood from hemophilia A donors, all configurations displayed an activity that was equal or greater than rFVIIa (FIG. 21A-C). Configuration A variants displayed the greatest activity by this method, reaching 15-fold higher activity than rFVIIa, and the activity was inversely correlated to XTEN length. The activity of Configuration B and C variants was less dependent on length of the XTEN. FIG. 21D shows that FVII-200 (LC:HC-XTEN-Targeting Moiety) demonstrates higher FVII activity than FVII-165 (LC:HC-XTEN) by ROTEM assay. This result indicates that adding a platelet-targeting moiety partially compensates for the loss of activity caused by the XTEN moiety.

### Example 9

### Effect of XTEN Length on PK

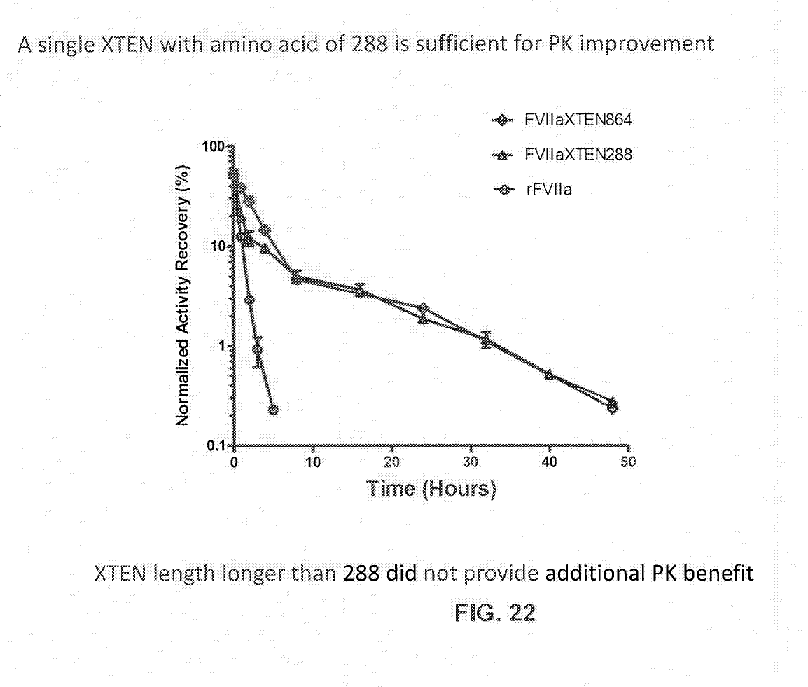

A single XTEN with amino acid of 288 was found to be sufficient for PK improvement. XTEN of 288 or 864 amino acids was fused to the C-terminus of FVIIa to generate FVIIa-XTEN288 and FVIIa-XTEN864, respectively. The proteins were produced and purified from the condition media of transiently transfected HEK293 cells. To assess the PK, the proteins were injected into HemA mice, and the plasma activity from various time after dosing were measured by a FVIIa dependent soluble tissue factor-prothrombin time (sTF-PT) assay. As shown in FIG. 22, fusing XTEN288 to FVIIa resulted in a slower clearance, but XTEN longer than 288 did not provide additional PK benefit, indicating that with a single XTEN, the length of 288 amino acids is long enough for PK improvement.

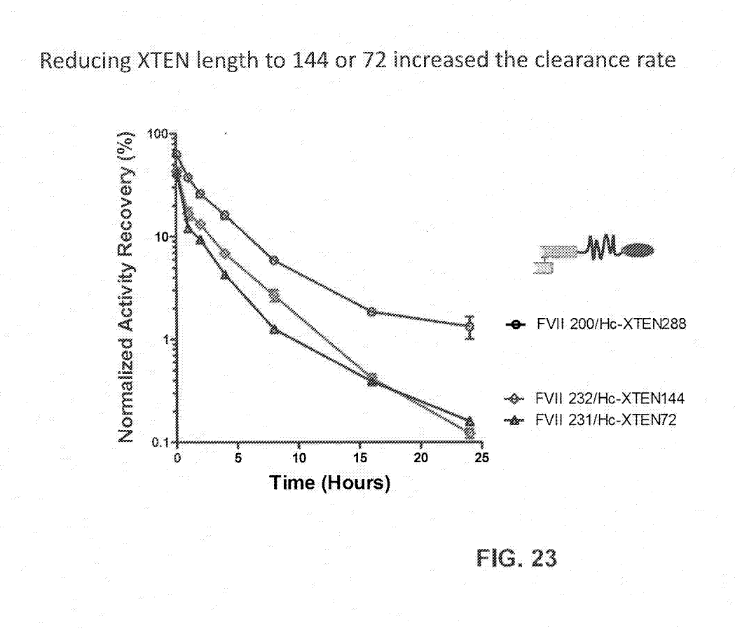

Next, XTEN with less than 288 amino acids was evaluated. In experiment of FIG. 23, XTEN of 288, 144, or 72 were fused to the C-terminus of the heavy chain of FVIIa, followed by the single chain variable region of monoclonal antibody 34D10. The proteins were administrated in hemA mice, and the plasma activities from dosed mice were measured by sTF-PT assay. As shown in FIG. 23, the activity recovery vs time chart indicated that the FVII232 and 231, which contained the XTEN of 144 and 72 amino acids respectively, cleared faster than that of FVII200, indicating that reducing XTEN length from 288 amino acids to 144 increased the clearance rate in mice.

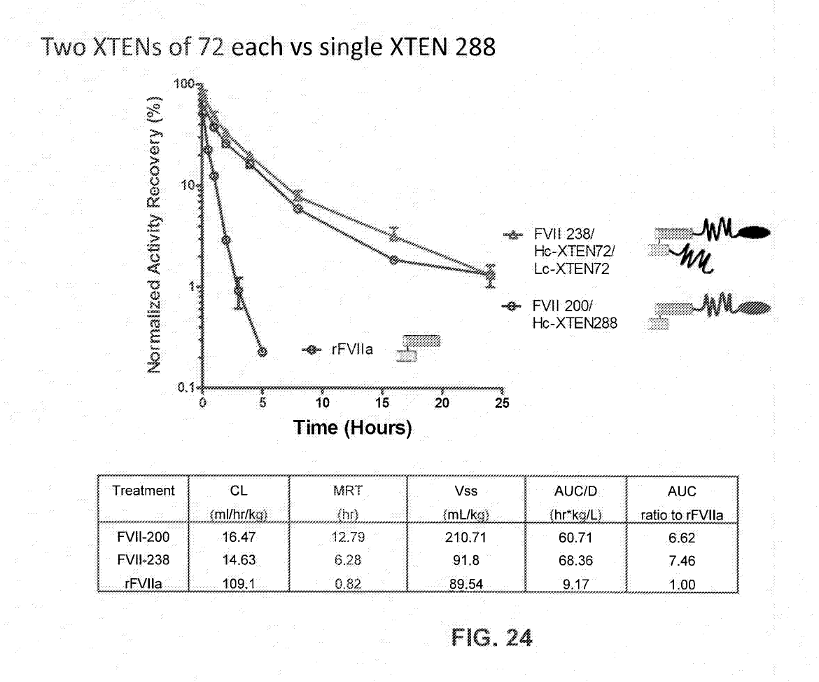

Because a single XTEN with length of less than 288 amino acids was not long enough to provide full PK benefit, the action of dual XTENs was investigated. In experiments of FIG. 24, Two XTENs of 72 amino acid were fused to FVIIa; one inserted between the C-terminus of the heavy chain of FVIIa and the N-terminus of 34D10, and one linked to the C-terminus of the light chain of FVIIa. The resulting protein, FVII238, was analyzed for the plasma activity PK in HemA mice. As shown in FIG. 24, the dual XTENs of only 72 amino acids each reduced the clearance rate from 109 ml/hr/kg to about 15 ml/hr/kg, resulting in a 7-fold increase in total protein exposure in HemA mice. This PK improvement was comparable to or better than did the single XTEN of 288 amino acids (FVII-200, FIG. 24).

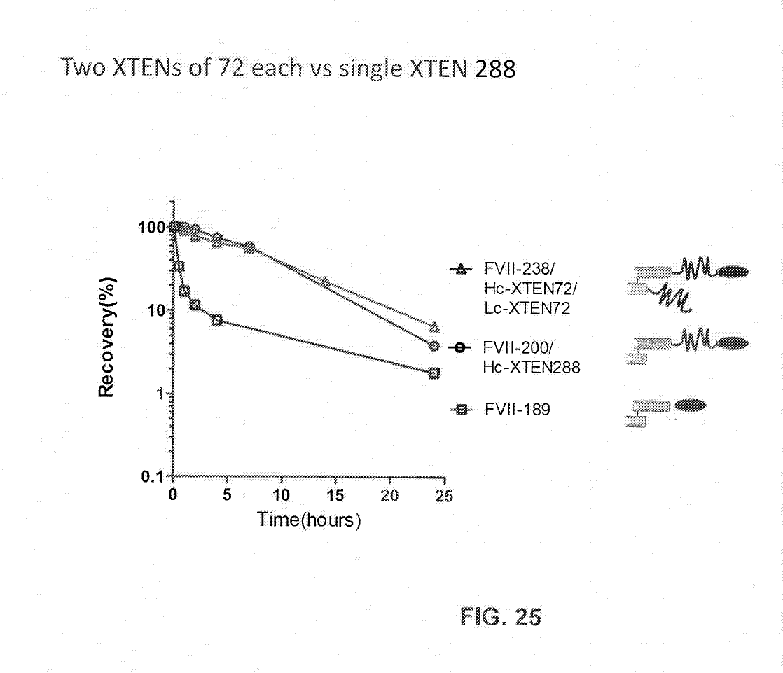

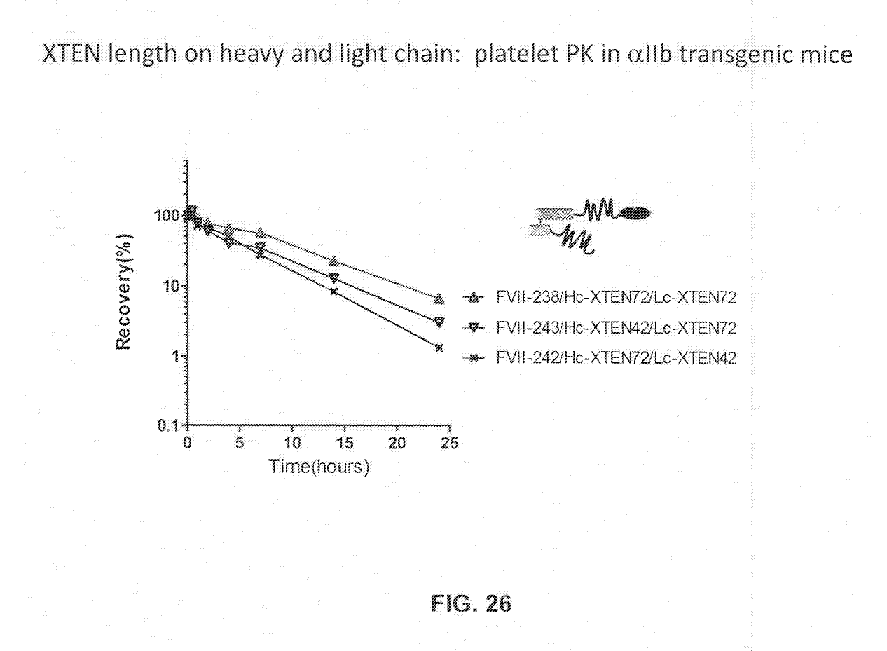

The PK of FVII-238 was also evaluated in the humanized αIIb transgenic mice. In the experiment of FIG. 25, FVII-238 was injected into αIIb mice and whole blood was collected via tail vein at various time after dosing. FVII protein concentration on platelets was measured by flow cytometry using fluorescently labeled antibody against human FVII. FVII-200 and FVII-189 were dosed and analyzed similarly. As shown in FIG. 25, FVII-189, which contains the platelet-binding single chain variable region from antibody 34D10, displayed a classic two phase decay: a rapid initial phase at a rate similar to that of rFVIIa in the circulation, and a terminal phase that was markedly slower than that of rFVIIa. Addition of either single XTEN of 288 amino acids (FVII-200), or dual XTENs of 72 each (FVII-238) dramatically reduced the initial clearance rate, resulting a similarly improved PK profile. Interestingly, with regards to the dual XTENs, reducing of the length of one XTEN from 72 to 42 at either the heavy chain (FVII-243) or light chain (FVII-242), led to increase in the clearance rate (FIG. 26), as measured by the platelet PK as described in FIG. 25.

Therefore, either a single XTEN of 288 amino acids that linked to the C-terminus of heavy chain of rFVIIa (i.e., FVII-200), or dual XTEN of 72 amino acids, with one fused to the heavy chain and one to the light chain (i.e., FVII-238), improved the pharmacokinetics of the platelet-targeted FVIIa, resulting in 6 to 7 fold increase in total exposure (AUC).

Examples 7-9 show, among other things, that it is possible to improve the activity and pharmacokinetic properties of rFVIIa by generating FVIIa variants with a platelet-targeting motif (a scFv directed to platelet receptor GPIIbIIIa) while fused to an XTEN moiety. To maximize the impact of XTEN and platelet targeting on PK and activity, respectively, 3 configurations of platelet-targeted rFVIIa-XTEN variants were generated: Configuration A, the XTEN and the targeting moiety were fused to the C-terminus of the light and heavy chain of FVIIa, respectively; Configuration B, both the XTEN was fused to the C-terminus of the heavy chain of rFVIIa while the targeting moiety was fused to the C-terminus of the XTEN; and Configuration C, XTEN was fused to the C-terminus of both heavy and light chain, with the targeting moiety at the C-terminus of the heavy-chain XTEN. For each configuration, multiple XTEN lengths were tested ranging from 72 to 288 amino acids.

Based on a platelet-independent method (sTF-PT), the specific activities of these variants in all configurations were lower than that of rFVIIa on a molar basis. Configuration A variants displayed the highest specific activity, as high as 44% of that of rFVIIa, while placement of the XTEN at the C-terminus of the heavy chain (Configuration B) had a higher impact on sTF-based activity. Configuration C variants displayed the lowest activities by this method. However, when tested in a platelet-dependent method in whole blood by ROTEM, all configurations displayed activities that were equal to or greater than that of rFVIIa. Configuration A variants displayed the greatest activity by this method, reaching 15-fold higher activity than rFVIIa. In general, the length of the XTEN element was inversely correlated with activity. To assess the impact of the XTEN moiety on platelet-targeting, binding of representative variants from each configuration to GPIIb/IIIa was measured by bio-layer interferometry. These assays revealed that the affinities of the platelet-targeted FVIIa-XTEN variants for GPIIb/IIIa were inversely correlated to the length of the XTEN. By combining platelet-targeting and XTEN technologies, rFVIIa variants that are more potent than rFVIIa in whole blood coagulation assays have been generated. In some cases, XTEN can affect the affinity of the rFVIIa variants to αIIbβ3. Therefore, while XTEN can improve the pharmacokinetic properties of rFVIIa, its placement and length must be optimized to maximize the impact of platelet-targeting technology.

Examples 5-9 further show XTEN can be integrated with different length. Based on the plasma activity PK in hemophilia A mice measured by a FVIIa specific soluble tissue factor-prothrombin time (sTF-PT) assay, for improving PK with single XTEN, a length of amino acid of 288 (XTEN₂₈₈) was found to be sufficient, as XTEN of 864 amino acids did not provide additional PK benefit. A single XTEN₂₈₈ was explored to improve the PK of αIIbβ3-targeted FVIIa. Two configurations that displayed equal or better hemostatic activity than FVIIa were investigated: 1) Heavy chain XTEN, where XTEN₂₈₈ was fused to the heavy chain and the αIIbβ3-scFv was further linked to XTEN₂₈₈, and 2) light chain XTEN, where the αIIbβ33-scFv was fused to the heavy chain and XTEN₂₈₈ to the light chain. The heavy chain XTEN cleared slower on platelets in circulation than that of the light chain XTEN, as judged by the platelet PK analysis in humanized αIIb transgenic mice where the platelet-bound proteins were quantified by flow cytometry using a fluorescently labeled FVII antibody. When compared to rFVIIa, plasma activity PK analysis of the heavy chain XTEN indicated about 7-fold reduction in clearance and 6-fold increase in total exposure. Although reducing XTEN length to 144 or 72 increased the clearance rate, attaching two XTENs of 72 amino acids, one to the heavy chain and one to the light chain, effectively improved the platelet and plasma PK to a degree similar to that of the heavy chain XTEN₂₈₈.

The present invention has been described above with the aid of functional building blocks illustrating the implementation of specified functions and relationships thereof. The boundaries of these functional building blocks have been arbitrarily defined herein for the convenience of the description. Alternative boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents. Other embodiments of the invention will be apparent to those skilled in the art from consideration of the specification and practice of the invention disclosed herein.

All patents and publications cited herein are incorporated by reference herein in their entirety.

### SEQUENCE LISTING

>SEQ_ID_NO:1 34D10 HC

EVKLVESEGGLVKPGGSLKLSCAASGFTFSAYAMSWVRQTPEKRLEWVA
SISSGGTTYYPDSVKRRFTIS RDNARNILYLQMSSLRSEDTAMYYCTRG
GDYGYALDYWGQGTSVTVSS

>SEQ_ID_NO:2 34D10 LC

ENVLTQSPAIMSASLGEKVTMSCRASSSVNYMYWYQQKSDASPKLWIYYT
SNLAPGVPARFSGSGSGNSYSLTISSMEGEDAATYYCQQFSSSPWTFGG
GTKLEIKR

>SEQ_ID_NO:3 2A2 NC

EVKLVESGGGLVKPGGSLKLSCAASGFTFRTYAMSWVRQTPEKRLEWVA
SISSGSSTYYLDSVKGRFTISRDNARNILYLQMSSLRSEDTAMYYCARGG
DYGYALDYWGQGTSVTVSS

>SEQ_ID_NO: 4 2A2 LC

ENVLTQSPAIMSASLGEKVTMSCRASSSVNYMYWYQQKSDASPKLWIYY
TSNLAPGVPTRFSGSGSGNSYSLTISSLEGEDAGTYYCQQFSSSPWTFG
GGTKLEIKR

>SEQ_ID_NO: 5 36A8 HC

EVRLVESGGGLVKPGGSLKLSCAASGFTFSTYAMSWVRQTPEKRLEWVA
SINGGGSTYYPDSVKGRFTISRDNARNILYLQMRSLRSEDTAMYYCARG
GDYGYALDYWGQGTSVTVSS

>SEQ_ID_NO: 6 36A8 LC

ENVLTQSPAIMSASLGEKVTMNCRASSSVNYMYWYQQKSDASPKLWIFY
TSNLAPGVPARFSGSGSGNSYSLTISSMEGEDAATYYCQQFSSSPWTF
GGGSKLEIKR

>SEQ_ID_NO:7 4B11 LC

EVKLVESGGGLVKPGGSLKLSCAASGFTFSSYAMSWVRQTPEKRLAWVA
SISSGGNIYFPDSVKGRFTISRDDARNILYLQMRSLRSEDTAMYYCARGG
DYGYAMDYWGQGTSVTVSS

>SEQ_ID_NO:8 1H6 HC

QVQLQQSGAELVRPGTSVKVSCKASGYAFTNYLIEWVKQRPGQGLEWIGV
INPGSGGTNYNEKFKGKATLTADKSSSTAYMHLSSLTSDDSAVYFCARGR
YEWYFDVWGAGTTVTVSS

>SEQ_ID_NO:9 1H6 LC

DIQMTQTTSSLSASLGDRVTISCRASQDITNYLNWYQRKPDGTVKLLIYY
TSRLHSGVPSRFSGSGSGTDYSLTISNLEQEDIATYFCQQGYTLPY
TFGGGTKLETKR

>SEQ_ID_NO:10 38A8 HC

QVQLQQSGAELVRPGTSVKVSCKASGYAFTNYLIEWIKQRPGQGLEWIGV
INPGSGGTNYNEKFKGKATLTADKSSSTAYMQLSSLTSDDSAVYFCARGR
YEWYFDVWGAGTTVTVSS

>SEQ_ID_NO:11 38A8 LC

DIQMTQTTSSLSASLGDRVTISCRASQDISNYLNWYLQKPDGTVKLLIYY
TSRLHSGVPSRFSGSGSGTDYSLSISNLEQEDIATYFCQQGYTLPYTEGG
GTKLEIKR

>SEQ_ID_NO: 12 18F7 HC

QVQLKESGPGLVAPSQSLSITCTVSGFSLTSYGVSWVRQPPGKGLEWLGI
IWGDGSTNYHSVLKSRLSISKDNSKSQVFLKLNSLQTDDTATYYCAKQDF
DVWGAGTTVTVSS

>SEQ_ID_NO:13 18F7 LC

DVQMIQSPFS [symbol] SASLGDIVTMTCQASQGTSINLNWFQQKPGKAPKLLIY
GVSNLEDGVPSRFSGSRYGTDFTLTIGSLEDEDMATYFCLQHSYLPYTFG
GGTKLEIKR [symbol]
indicates data missing or illegible when filed

>SEQ_ID_NO:14 12B2 HC

QVQLQQSGAELTKPGASVKISCKATGYTFSSYWIEWVKQRPGHGLEWIGE
ILPGSGITKYNDKFKGKATFTADTSSNTAYMQLSSLTSEDSAVYSCARLI
SYYYAMDYWGQGTSVTVSS

>SEQ_ID_NO:15 12B2 LC

DIQMTQTTSSLSASLGDRVTISCRASQDISNYLNWYQQKPDGTVKLLIYY
TSRLHSGVPSRFSGSGSGTDYSLTISNLEQEDIATYFCQQGNTLPPTFGG
GTKLEIKR

>SEQ_ID_NO:16 38F6 HC

QVQLQQSGAELMRPGASVKISCKATGYTFSSYWIEWVKQRPGHGLEWIGE
ILPGTGYTKYNEKFKGKATFTAETSSNTASMQVSSLTSEDSAVYFCARLI
SYYYAMDYWGQGTSVTVSS

>SEQ_ID_NO:17 5C4

QVTLKASGPGILQPSQTLSLTCSFSGFSLNTSGLGVGWIRQPSGKGLEWL
AHIWWDDDKRYNPALKSRLTISKDTSNNQIFLKIASVDTADTATYYCARS
HYYGTFYFDYWGQGTTLTVSS

>SEQ_ID_NO:18 23C10 HC

FLLLIVPAYVLSQVTLKASGPGIVQPSQTLSLTCSFSGFSLNTSGMGVGW
IRQPSGKGLEWLAHIWWDDDKRYNPALKSRLTISKDTSNNQIFLKIASVD
TADTATYYCARSHYYGTFYFDYWGQGTTLTVSS

>SEQ_ID_NO:19 28C2 HC

QVTLKASGPIVQPSQTLSLTCSFSGFSLNTSGMGVGWIRQPSGKGLEWLA
HIWWDDDKRYNPALKSRLTISKDTSNNQIFLKIASVDTADTATYYCARSH
YYGTFYFDYWGQGTTLTVSS

>SEQ_ID_NO:20 28C2 CL

DVQITQSPSYLAASPGETITINCRASKSISKYLAWYQEKPGTTYKLLIYS
GSTLQSGIPSRFSGSGSGTDFTLTISSLEPEDFAMYYCQQHIEYPWTFGG
GTKLEIKR

>SEQ_ID_NO:21 9D6 HC

QVTLKESGPGILQPSQTLSLTCSFSGFSLSTSGMGVGWIRQSSGKGLEWL
AHIWWDDDKRYNPTLKSRLTISKDTSNNQVFLKIANMDTADIATYYCARS
HYNGTFYFDFWGQGITLTVSS

>SEQ_ID_NO:22 9D6 LC

DVQITQSPSYLAASPGETITINCRASKSISKYLAWYQEKPGKTNKLLIYS
GSTLQSGIPSRFSGSGSGTDFTLTISTLEPEDFAMYYCQQHIEYPWTFGG
GTKLEIKR

>SEQ_ID_NO:23 28F4 HC

EVQLVESGGDLVKPGGSLKLSCAASGFTFSNYGMSWVRQTPDKRLEWVAT
ISSGGTYTYYPDSVKGQFTIFRDNAKNTLYLQMSSLKSEDTAMYYCTRRD
YDYEGFAYWGQGTLVTVS

>SEQ_ID_NO:24 28F4 LC

DIVLTQSPATLSVTPGDSVSLSCRASQSISNNLHWYQQKSHESPRLLIKY
ASHSISGIPSRFSGSGSGTDFTLSINSVETEDFGMYFCQQSNNWPFTFGS
GTKLEIKR

>SEQ_ID_NO:25 2A2 HC CDR1

TYAMS

>SEQ_ID_NO:26 2A2 HC CDR2

SISSGSSTYYLDSVKG

>SEQ_ID_NO:27 2A2 HC CDR3

GGDYGYALDY

>SEQ_ID_NO:28 2A2 LC CDR1

RASSSVNYMY

>SEQ_ID_NO:29 2A2 LC CDR2

YTSNLAP

>SEQ_ID_NO:30 2A2 LC CDR3

QQFSSPWT

>SEQ_ID_NO:31 34D10 HC CDR1

AYAMS

>SEQ_ID_NO:32 34D10 HC CDR2

SISSGGTTYYPDSVKR

>SEQ_ID_NO:33 34D10 HC CDR3

GGDYGYALDY

>SEQ_ID_NO:34 34D10 LC CDR1

RASSSVNYMY

>SEQ_ID_NO:35 34D10 LC CDR2

YTSNLAP

>SEQ_ID_NO:36 34D10 LC CDR3

QQFSSSPWT

>SEQ_ID_NO:37 36A8 HC CDR1

TYAMS

>SEQ_ID_NO:38 36A8 HC CDR2

SINGGGSTYYPDSVKG

>SEQ_ID_NO:39 36A8 HC CDR3

GGDYGYALDY

>SEQ_ID_NO:40 36A8 LC CDR1

RASSSVNYMY

>SEQ_ID_NO:41 36A8 LC CDR2

YTSNLAP

>SEQ_ID_NO:42 36A8 LC CDR3

QQFSSSPWT

>SEQ_ID_NO:43 4B11 HC CDR1

SYAMS

>SEQ_ID_NO:44 4B11 HC CDR2

SISSGGNIYFPDSVKG

>SEQ_ID_NO:45 4B11 HC CDR3

GGDYGYAMDY

>SEQ_ID_NO:46 1H6 HC CDR1

NYLIE

>SEQ_ID_NO:47 1H6 HC CDR2

VINPGSGGTNYNEKFKG

>SEQ_ID_NO:48 1H6 HC CDR3

GRYEWYFDV

>SEQ_ID_NO:49 1H6 LC CDR1

RASQDITNYLN

>SEQ_ID_NO:50 1H6 LC CDR2

YTSRLHS

>SEQ_ID_NO:51 1H6 LC CDR3

QQGYTLPYT

>SEQ_ID_NO:52 38A8 HC CDR1

NYLIE

>SEQ_ID_NO:53 38A8 HC CDR2

VINPGSGGTNYNEKFKG

>SEQ_ID_NO:54 38A8 HC CDR3

GRYEWYFDV

>SEQ_ID_NO:55 38A8 LC CDR1

RASQDISNYLN

>SEQ_ID_NO:56 38A8 LC CDR2

YTSRLHS

>SEQ_ID_NO:57 38A8 LC CDR3

QQGYTLPYT

>SEQ_ID_NO:58 18F7 HC CDR1

SYGVS

>SEQ_ID_NO:59 18F7 HC CDR2

IIWGDGSTNYHSVLKS

>SEQ_ID_NO:60 18F7 HC CDR3

QDFDV

>SEQ_ID_NO:61 18F7 LC CDR1

QASQGTSINLN

>SEQ_ID_NO:62 18F7 LC CDR2

GVSNLED

>SEQ_ID_NO:63 18F7 LC CDR3

LQHSYLPYT

>SEQ_ID_NO:64 12B2 HC CDR1

SYWIE

>SEQ_ID_NO:65 12B2 HC CDR2

EILPGSGITKYNDKFKG

>SEQ_ID_NO:66 12B2 HC CDR3

LISYYYAMDY

>SEQ_ID_NO:67 12B2 LC CDR1

RASQDISNYLN

>SEQ_ID_NO:68 12B2 LC CDR2

YTSRLHS

>SEQ_ID_NO:69 12B2 LC CDR3

QQGNTLPPT

>SEQ_ID_NO:70 38F6 HC CDR1

SYWIE

>SEQ_ID_NO:71 38F6 HC CDR2

EILPGTGYTKYNEKFKG

>SEQ_ID_NO:72 38F6 HC CDR3

LISYYYAMDY

>SEQ_ID_NO:73 5C4 HC CDR1

TSGLGVG

>SEQ_ID_NO:74 5C4 HC CDR2

HIWWDDDKRYNPALKS

>SEQ_ID_NO:75 5C4 HC CDR3

SHYYGTFYFDY

>SEQ_ID_NO:76 23C10 HC CDR1

TSGMGVG

>SEQ_ID_NO:77 23C10 HC CDR2

HIWWDDDKRYNPALKS

>SEQ_ID_NO:78 23C10 HC CDR3

SHYYGTFYFDY

>SEQ_ID_NO:79 28C2 HC CDR1

TSGMGVG

>SEQ_ID_NO:80 28C2 HC CDR2

HIWWDDDKRYNPALKS

>SEQ_ID_NO:81 28C2 HC CDR3

SHYYGTFYFDY

>SEQ_ID_NO:82 28C2 LC CDR1

RASKSISKYLA

>SEQ_ID_NO:83 28C2 LC CDR2

SGSTLQS

>SEQ_ID_NO:84 28C2 LC CDR3

QQHIEYPWT

>SEQ_ID_NO:85 9D6 HC CDR1

TSGMGVG

>SEQ_ID_NO:86 9D6 HC CDR2

HIWWDDDKRYNPTLKS

>SEQ_ID_NO:87 9D6 HC CDR3

SHYNGTFYFDF

>SEQ_ID_NO:88 9D6 LC CDR1

RASKSISKYLA

>SEQ_ID_NO:89 9D6 LC CDR2

SGSTLQS

>SEQ_ID_NO:90 9D6 LC CDR3

QQHIEYPWT

>SEQ_ID_NO:91 28F4 HC CDR1

NYGMS

>SEQ_ID_NO:92 28F4 HC CDR2

TISSGGTTYYPDSVKG

>SEQ_ID_NO:93 28F4 HC CDR3

RDYDYEGFAY

>SEQ_ID_NO:94 28F4 LC CDR1

RASQSISNNLH

>SEQ_ID_NO:95 28F4 LC CDR2

YASHSIS

>SEQ_ID_NO:96 28F4 LC CDR3

QQSNNWPFT

>SEQ_ID_NO:97 35D1 HC

EVKLVESGGGLVKPGGSLKLSCAASGFTFSAYAMSWVRQTPEKRLEWVAS
ISSGGTTYYPDSVKRRFTISRDNARNILYLQMSSLRSEDTAMYYCTRGGD
YGYALDYWGQGTSVTVSS

>SEQ_ID_NO:98 35D1 LC

ENVLTQSPAIMSASLGEKVTMSCRASSSVNYMYWYQQKSDASPKLWIYYT
SNLAPGVPARFSGSGSGNSYSLTISSMEGEDAATYYCQQFSSSPWTFGGG
TKLEIKR

>SEQ_ID_NO:99 4B11 LC

ENVLTQSPAIMSASLGEKVTMNCRASSSVNYMYWYQQKSDASPKLWIFYT
SNLAPGVPARFSGSGSGNSYSLTISSMEGEDAATYYCQQFSSSPWTFGGG
SKLEIKR

>SEQ_ID_NO:100 38G8 HC

QVQLQQSGAELVRPGTSVKVSCKASGYAFTNYLLEWVKQRPGQGLEWIGV
INPGSGGTNYNEKFKGKATLTADKSSSTAYMHLSSLTSDDSAVYFCARGR
YEWYFDVWGAGTTVTVSS

>SEQ_ID_NO:101 38G8 LC

DIQMTQTTSSLSASLGDRVTISCRASQDITNYLNWYQRKPDGTVKLLIYY
TSRLHSGVPSRFSGSGSGTDYSLTISNLEQEDIATYFCQQGYTLPYTFGG
GTKLEIKR

>SEQ_ID_NO:102 21F10 HC

DIQMTQTTSSLSASLGDRVTISCRASQDITNYLNWYQRKPDGTVKLLIYY
TSRLHSGVPSRFSGSGSGTDYSLTISNLEQEDIATYFCQQGYTLPYTEGG
GTKLEIKR

>SEQ_ID_NO:103 21F10 LC

DIQMTQTTSSLSASLGDRVTISCRASQDITNYLNWYQRKPDGTVKLLIYY
TSRLHSGVPSRFSGSGSGTDYSLTISNLEQEDIATYFCQQGYTLPYTFGG
GTKLEIKR

>SEQ_ID_NO:104 38F6 LC*
sequencing_pending
>SEQ_ID_NO:105 13C1 HC

QVQLQQSGAELTKPGASVKISCKATGYTFSSYWIEWVKQRPGEIGLEWIG
EILPGSGITKYNDKFKGKATFTADTSSNTAYMQLSSLTSEDSAVYSCARL
ISYYYANDYWGQGTSVTVSS

>SEQ_ID_NO:106 13C1 LC
sequencing_pending
>SEQ_ID_NO:107 5C4 LC*
sequencing_pending
>SEQ_ID_NO:108 23C10 LC*
sequencing_pending
>SEQ_ID_NO:109 37C7 HC
>SEQ_ID_NO:110 37C7 LC*
sequencing_pending
>SEQ_ID_NO:111 35D1 HC CDR1

AYAMS

>SEQ_ID_NO:112 35D1 HC CDR2

SISSGGTTYYPDSVKR

>SEQ_ID_NO:113 35D1 HC CDR3

GGDYGYALDY

>SEQ_ID_NO:114 35D2 LC CDR1

RASSSVNYMY

>SEQ_ID_NO:115 35D2 LC CDR2

YTSNLAP

>SEQ_ID_NO:116 35D2 LC CDR3

QQFSSSPWT

>SEQ_ID_NO:117 4B11 LC CDR1

RASSSVNYMY

>SEQ_ID_NO:118 4B11 LC CDR2

YTSNLAP

>SEQ_ID_NO:119 4B11 LC CDR3

QQFSSSPWT

>SEQ_ID_NO:120 38G8 HC CDR1

NYLIE

>SEQ_ID_NO:121 38G8 HC CDR2

VINPGSGGTNYNEKFKG

>SEQ_ID_NO:122 38G8 HC CDR3

GRYEWYFDV

>SEQ_ID_NO:123 38G8 LC CDR1

RASQDITNYLN

>SEQ_ID_NO:124 38G8 LC CDR2

YTSRLHS

>SEQ_ID_NO:125 38G8 LC CDR3

QQGYTLPYT

>SEQ_ID_NO:126 21F10 HC CDR1

NYLIE

>SEQ_ID_NO:127 21F10 HC CDR2

VINPGSGGTNYNEKFKG

>SEQ_ID_NO:128 21F10 HC CDR3

GRYEWYFDV

>SEQ_ID_NO:129 21F10 LC CDR1

RASQDITNYLN

>SEQ_ID_NO:130 21F10 LC CDR2

YTSRLHS

>SEQ_ID_NO:131 21F10 LC CDR3

QQGYTLPYT

>SEQ_ID_NO:132 38F6 LC* CDR1
sequencing_pending
>SEQ_ID_NO:133 38F6 LC* CDR2
sequencing_pending
>SEQ_ID_NO:134 38F6 LC* CDR3
sequencing_pending
>SEQ_ID_NO:135 13C1 HC CDR1

SYWIE

>SEQ_ID_NO:136 13C1 HC CDR2

EILPGSGITKYNDKFKG

>SEQ_ID_NO:137 13C1 HC CDR3

LISYYYAMDY

>SEQ_ID_NO:138 13C1 LC* CDR1
sequencing_pending
>SEQ_ID_NO:139 13C1 LC* CDR2
sequencing_pending
>SEQ_ID_NO:140 13C1 LC* CDR3
sequencing_pending
>SEQ_ID_NO:141 5C4 LC* CDR1
sequencing_pending
>SEQ_ID_NO:142 5C4 LC* CDR2
sequencing_pending
>SEQ_ID_NO:143 5C4 LC* CDR3
sequencing_pending
>SEQ_ID_NO:144 23C10 LC* CDR1
sequencing_pending
>SEQ_ID_NO:145 23C10 LC* CDR2
sequencing_pending
>SEQ_ID_NO:146 23C10 LC* CDR3
sequencing_pending
>SEQ_ID_NO:147 37C7 HC CDR1

TSGMGVG

>SEQ_ID_NO:148 37C7 HC CDR2

HIWWDDDKRYNPALKS

>SEQ_ID_NO:149 37C7 HC CDR3

SHYYGTFYFDY

>SEQ_ID_NO:150 37C7 LC* CDR1
sequencing_pending
>SEQ_ID_NO:151 37C7 LC* CDR2
sequencing_pending
>SEQ_ID_NO:152 37C7 LC* CDR3
sequencing_pending

### SEQ ID NO:153

>CTP peptide 1

DPRFQDSSSSKAPPPSLPSPSRLPGPSDTPIL

### SEQ ID NO:154

>CTP peptide 2

SSSSKAPPPSLPSPSRLPGPSDTPILPQ

### SEQ ID NO:155

>PAS peptide 1

ASPAAPAPASPAAPAPSAPA

### SEQ ID NO:156

>PAS peptide 2

AAPASPAPAAPSAPAPAAPS

### SEQ ID NO:157

>PAS peptide 3

APSSPSPSAPSSPSPASPSS

### SEQ ID NO:158

>PAS peptide 4

APSSPSPSAPSSPSPASPS

### SEQ ID NO:159

>PAS peptide 5

SSPSAPSPSSPASPSPSSPA

### SEQ ID NO:160

>PAS peptide 6

AASPAAPSAPPAAASPAAPSAPPA

### SEQ ID NO:161

>PAS peptide 7

ASAAAPAAASAAASAPSAAA

### SEQ ID NO:162

### >Albumin Binding Peptide Core Sequence

DICLPRWGCLW

### SEQ ID NO:163

>GFP protein, sequence (Genbank ID AAG34521.1)

MSKGEELFTGVVPILVELDGDVNGHKFSVSGEGEGDATYGKLTLKFICTT
GKLPVPWPTLVTTFGYGVQCFARYPDHMKQHDFFKSAMPEGYVQERTIFF
KDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNH
YLSTQSALSKDPNEKRDHMVLLEFVTAAGITHGMDELYKSRTSGSPGLQE
FDIKLIDTVDLESCN

### SEQ ID NO:164

>Example: Single-chain Human IgG1 Fc. (Fc sequences with Gly/Ser linker underlined.)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVF
SCSVMHEALHNHYTQKSLSLSPGK

### SEQ ID NO:165

>Mature human albumin protein sequence (derived from NCBI Ref. Sequence NP_000468):

RGVFRRDAHKSEVAHRFKDLGEENFKALVLIAFAQYLQQCPFEDHVKLVN
EVTEFAKTCVADESAENCDKSLHTLFGDKLCTVATLRETYGEMADCCAKQ
EPERNECFLQHKDDNPNLPRLVRPEVDVMCTAFHDNEETFLKKYLYETAR
RHPYFYAPELLFFAKRYKAAFTECCQAADKAACLLPKLDELRDEGKASSA
KQRLKCASLQKFGERAFKAWAVARLSQRFPKAEFAEVSKLVTDLTKVHTE
CCHGDLLECADDRADLAKYICENQDSISSKLKECCEKPLLEKSHCIAEVE
NDEMPADLPSLAADFVESKDVCKNYAEAKDVFLGMFLYEYARRHPDYSVV
LLLRLAKTYETTLEKCCAAADPHECYAKVFDEFKPLVEEPQNLIKQNCEL
FEQLGEYKFQNALLVRYTKKVPQVSTPTLVEVSRNLGKVGSKCCKHPEAK
RMPCAEDYLSVVLNQLCVLHEKTPVSDRVTKCCTESLVNRRPCFSALEVD
ETYVPKEFNAETFTFHADICTLSEKERQIKKQTALVELVKHKPKATKEQL
KAVMDDFAAFVEKCCKADDKETCFAEEGKKLVAASQAALGL

### SEQ ID NO:166

>Linker, n=0, 1, 2, 3, 4 or more

(GGGS)ₙ

### SEQ ID NO:167

>Albumin binding peptide 1

RLIEDICLPRWGCLWEDD

### SEQ ID NO:168

>Albumin binding peptide 2

QRLMEDICLPRWGCLWEDDF

### SEQ ID NO:169

>Albumin binding peptide 3

QGLIGDICLPRWGCLWGDSVK

### SEQ ID NO:170

>Albumin binding peptide 4

GEWWEDICLPRWGCLWEEED

### SEQ ID NO:171

>Cysteine-containing peptide

GGGSGCGGGS

### SEQ ID NO:172

>Human LRP1 sequence (signal peptide and transmembrane segment underlined; NCBI Reference Sequence: CAA32112)

MLTPPLLLLLPLLSALVAAAIDAPKTCSPKQFACRDQITCISKGWRCDGE
RDCPDGSDEAPEICPQSKAQRCQPNEHNCLGTELCVPMSRLCNGVQDCMD
GSDEGPHCRELQGNCSRLGCQHHCVPTLDGPTCYCNSSFQLQADGKTCKD
FDECSVYGTCSQLCTNTDGSFICGCVEGYLLQPDNRSCKAKNEPVDRPPV
LLIANSQNILATYLSGAQVSTITPTSTRQTTAMDFSYANETVCWVHVGDS
AAQTQLKCARMPGLKGFVDEHTINISLSLHHVEQMAIDWLTGNFYFVDDI
DDRIFVCNRNGDTCVTLLDLELYNPKGIALDPAMGKVFFTDYGQIPKVER
CDMDGQNRTKLVDSKIVFPHGITLDLVSRLVYWADAYLDYIEVVDYEGKG
RQTIIQGILIEHLYGLTVFENYLYATNSDNANAQQKTSVIRVNRFNSTEY
QVVTRVDKGGALHIYHQRRQPRVRSHACENDQYGKPGGCSDICLLANSHK
ARTCRCRSGFSLGSDGKSCKKPEHELFLVYGKGRPGIIRGMDMGAKVPDE
HMIPIENLMNPRALDFHAETGFIYFADTTSYLIGRQKIDGTERETILKDG
IHNVEGVAVDWMGDNLYWTDDGPKKTISVARLEKAAQTRKTLIEGKMTHP
RAIVVDPLNGWMYWTDWEEDPKDSRRGRLERAWMDGSHRDIFVTSKTVLW
PNGLSLDIPAGRLYWVDAFYDRIETILLNGTDRKIVYEGPELNHAFGLCH
HGNYLFWTEYRSGSVYRLERGVGGAPPTVTLLRSERPPIFEIRMYDAQQQ
QVGTNKCRVNNGGCSSLCLATPGSRQCACAEDQVLDADGVTCLANPSYVP
PPQCQPGEFACANSRCIQERWKCDGDNDCLDNSDEAPALCHQHTCPSDRF
KCENNRCIPNRWLCDGDNDCGNSEDESNATCSARTCPPNQFSCASGRCIP
ISWTCDLDDDCGDRSDESASCAYPTCFPLTQFTCNNGRCININWRCDNDN
DCGDNSDEAGCSHSCSSTQFKCNSGRCIPEHWTCDGDNDCGDYSDETHAN
CTNQATRPPGGCHTDEFQCRLDGLCIPLRWRCDGDTDCMDSSDEKSCEGV
THVCDPSVKFGCKDSARCISKAWVCDGDNDCEDNSDEENCESLACRPPSH
PCANNTSVCLPPDKLCDGNDDCGDGSDEGELCDQCSLNNGGCSHNCSVAP
GEGIVCSCPLGMELGPDNHTCQIQSYCAKHLKCSQKCDQNKFSVKCSCYE
GWVLEPDGESCRSLDPFKPFIIFSNRHEIRRIDLHKGDYSVLVPGLRNTI
ALDFHLSQSALYWTDVVEDKIYRGKLLDNGALTSFEVVIQYGLATPEGLA
VDWIAGNIYWVESNLDQIEVAKLDGTLRTTLLAGDIEHPRAIALDPRDGI
LFWTDWDASLPRIEAASMSGAGRRTVHRETGSGGWPNGLTVDYLEKRILW
IDARSDAIYSARYDGSGHMEVLRGHEFLSHPFAVTLYGGEVYWTDWRTNT
LAKANKWTGHNVTVVQRTNTQPFDLQVYHPSRQPMAPNPCEANGGQGPCS
HLCLINYNRTVSCACPHLMKLHKDNTTCYEFKKFLLYARQMEIRGVDLDA
PYYNYIISFTVPDIDNVTVLDYDAREQRVYWSDVRTQAIKRAFINGTGVE
TVVSADLPNAHGLAVDWVSRNLFWTSYDTNKKQINVARLDGSFKNAVVQG
LEQPHGLVVHPLRGKLYWTDGDNISMANMDGSNRTLLFSGQKGPVGLAID
FPESKLYWISSGNHTINRCNLDGSGLEVIDAMRSQLGKATALAIMGDKLW
WADQVSEKMGTCSKADGSGSVVLRNSTTLVMHMKVYDESIQLDHKGTNPC
SVNNGDCSQLCLPTSETTRSCMCTAGYSLRSGQQACEGVGSFLLYSVHEG
IRGIPLDPNDKSDALVPVSGTSLAVGIDFHAENDTIYWVDMGLSTISRAK
RDQTWREDVVTNGIGRVEGIAVDWIAGNIYWTDQGFDVIEVARLNGSFRY
VVISQGLDKPRAITVHPEKGYLFWTEWGQYPRIERSRLDGTERVVLVNVS
ISWPNGISVDYQDGKLYWCDARTDKIERIDLETGENREVVLSSNNMDMFS
VSVFEDFIYWSDRTHANGSIKRGSKDNATDSVPLRTGIGVQLKDIKVFNR
DRQKGTNVCAVANGGCQQLCLYRGRGQRACACAHGMLAEDGASCREYAGY
LLYSERTILKSIHLSDERNLNAPVQPFEDPEHMKNVIALAFDYRAGTSPG
TPNRIFFSDIHFGNIQQINDDGSRRITIVENVGSVEGLAYHRGWDTLYWT
SYTTSTITRHTVDQTRPGAFERETVITMSGDDHPRAFVLDECQNLMFWTN
WNEQHPSIMRAALSGANVLTLIEKDIRTPNGLAIDHRAEKLYFSDATLDK
IERCEYDGSHRYVILKSEPVHPFGLAVYGEHIFWTDWVRRAVQRANKHVG
SNMKLLRVDIPQQPMGIIAVANDTNSCELSPCRINNGGCQDLCLLTHQGH
VNCSCRGGRILQDDLTCRAVNSSCRAQDEFECANGECINFSLTCDGVPHC
KDKSDEKPSYCNSRRCKKTFRQCSNGRCVSNMLWCNGADDCGDGSDEIPC
NKTACGVGEFRCRDGTCIGNSSRCNQFVDCEDASDEMNCSATDCSSYFRL
GVKGVLFQPCERTSLCYAPSWVCDGANDCGDYSDERDCPGVKRPRCPLNY
FACPSGRCIPMSWTCDKEDDCEHGEDETHCNKFCSEAQFECQNHRCISKQ
WLCDGSDDCGDGSDEAAHCEGKTCGPSSFSCPGTHVCVPERWLCDGDKDC
ADGADESIAAGCLYNSTCDDREFMCQNRQCIPKHFVCDHDRDCADGSDES
PECEYPTCGPSEFRCANGRCLSSRQWECDGENDCHDQSDEAPKNPHCTSP
EHKCNASSQFLCSSGRCVAEALLCNGQDDCGDSSDERGCHINECLSRKLS
GCSQDCEDLKIGFKCRCRPGFRLKDDGRTCADVDECSTTFPCSQRCINTH
GSYKCLCVEGYAPRGGDPHSCKAVTDEEPFLIFANRYYLRKLNLDGSNYT
LLKQGLNNAVALDFDYREQMIYWTDVTTQGSMIRRMHLNGSNVQVLHRTG
LSNPDGLAVDWVGGNLYWCDKGRDTIEVSKLNGAYRTVLVSSGLREPRAL
VVDVQNGYLYWTDWGDHSLIGRIGMDGSSRSVIVDTKITWPNGLTLDYVT
ERIYWADAREDYIEFASLDGSNRHVVLSQDIPHIFALTLFEDYVYWTDWE
TKSINRAHKTTGTNKTLLISTLHRPMDLHVFHALRQPDVPNHPCKVNNGG
CSNLCLLSPGGGHKCACPTNFYLGSDGRTCVSNCTASQFVCKNDKCIPFW
WKCDTEDDCGDHSDEPPDCPEFKCRPGQFQCSTGICTNPAFICDGDNDCQ
DNSDEANCDIHVCLPSQFKCTNTNRCIPGIFRCNGQDNCGDGEDERDCPE
VTCAPNQFQCSITKRCIPRVWVCDRDNDCVDGSDEPANCTQMTCGVDEFR
CKDSGRCIPARWKCDGEDDCGDGSDEPKEECDERTCEPYQFRCKNNRCVP
GRWQCDYDNDCGDNSDEESCTPRPCSESEFSCANGRCIAGRWKCDGDHDC
ADGSDEKDCTPRCDMDQFQCKSGHCIPLRWRCDADADCMDGSDEEACGTG
VRTCPLDEFQCNNTLCKPLAWKCDGEDDCGDNSDENPEECARFVCPPNRP
FRCKNDRVCLWIGRQCDGTDNCGDGTDEEDCEPPTAHTTHCKDKKEFLCR
NQRCLSSSLRCNMFDDCGDGSDEEDCSIDPKLTSCATNASICGDEARCVR
TEKAAYCACRSGFHTVPGQPGCQDINECLRFGTCSQLCNNTKGGHLCSCA
RNFMKTHNTCKAEGSEYQVLYIADDNEIRSLFPGHPHSAYEQAFQGDESV
RIDAMDVHVKAGRVYWTNWHTGTISYRSLPPAAPPTTSNRHRRQIDRGVT
HLNISGLKMPRGIAIDWVAGNVYWTDSGRDVIEVAQMKGENRKTLISGMI
DEPHAIVVDPLRGTMYWSDWGNHPKIETAAMDGTLRETLVQDNIQWPTGL
AVDYHNERLYWADAKLSVIGSIRLNGTDPIVAADSKRGLSHPFSIDVFED
YIYGVTYINNRVFKIHKFGHSPLVNLTGGLSHASDVVLYHQHKQPEVTNP
CDRKKCEWLCLLSPSGPVCTCPNGKRLDNGTCVPVPSPTPPPDAPRPGTC
NLQCFNGGSCFLNARRQPKCRCQPRYTGDKCELDQCWEHCRNGGTCAASP
SGMPTCRCPTGFTGPKCTQQVCAGYCANNSTCTVNQGNQPQCRCLPGFLG
DRCQYRQCSGYCENFGTCQMAADGSRQCRCTAYFEGSRCEVNKCSRCLEG
ACVVNKQSGDVTCNCTDGRVAPSCLTCVGHCSNGGSCTMNSKMMPECQCP
PHMTGPRCEEHVESQQQPGHIASILIPLLLLLLLVLVAGVVFWYKRRVQG
AKGFQHQRMTNGAMNVEIGNPTYKMYEGGEPDDVGGLLDADFALDPDKPT
NFTNPVYATLYMGGHGSRHSLASTDEKRELLGRGPEDEIGDPLA

### SEQ ID NO:173

### >Biotin Acceptor Peptide (BAP)

LNDIFEAQKIEWH

### SEQ ID NO:174

### >Lipoate Acceptor Peptide 2 (LAP2)

GFEIDKVWYDLDA

### SEQ ID NO:175

>HAPylation motif, n=1 to 400

(Gly4Ser)n

### SEQ ID NO:176

>Alternative linker

PEAPTDPEAPTD

### SEQ ID NO:177

### >CTP

DSSSSKAPPPSLPSPSRLPGPSDTPILPQ

### SEQ ID NO:178

### >FVII-HC PPT

| | |
|---|---|
| 1 | IVGGKVCPKG ECPWQVLLLV NGAQLCGGTL INTIWVVSAA HCFDKIKNWR |
| 51 | NLIAVLGEHD LSEHDGDEQS RRVAQVIIPS TYVPGTTNHD IALLRLHQPV |
| 101 | VLTDHVVPLC LPERTFSERT LAFVRFSLVS GWGQLLDRGA TALELMVLNV |
| 151 | PRLMTQDCLQ QSRKVGDSPN ITEYMFCAGY SDGSKDSCKG DSGGPHATHY |
| 201 | RGTWYLTGIV SWGQGCATVG HFGVYTRVSQ YIEWLQKLMR SEPRPGVLLR |
| 251 | APFP |

### SEQ ID NO:179

### >FVII-LC PPT

| | |
|---|---|
| 1 | ANAFLEELRP GSLERECKEE QCSFEEAREI FKDAERTKLF WISYSDGDQC |
| 51 | ASSPCQNGGS CKDQLQSYIC FCLPAFEGRN CETHKDDQLI CVNENGGCEQ |
| 101 | YCSDHTGTKR SCRCHEGYSL LADGVSCTPT VEYPCGKIPI LEKRNASKPQ |
| 151 | GR |

### SEQ ID NO:180

### >FVII HC DNA.

| | |
|---|---|
| 1 | ATTGTG GGGGGC AAGGTG TGCCCC AAAGGG GAGTGT CCATGG CAGGTC CTGTTG TTGGTG |
| 61 | AATGGA GCTCAG TTGTGT GGGGGG ACCCTG ATCAAC ACCATC TGGGTG GTCTCC GCGGCC |
| 121 | CACTGT TTCGAC AAAATC AAGAAC TGGAGG AACCTG ATCGCG GTGCTG GGCGAG CACGAC |
| 181 | CTCAGC GAGCAC GACGGG GATGAG CAGAGC CGGCGG GTGGCG CAGGTC ATCATC CCCAGC |
| 241 | ACGTAC GTCCCG GGCACC ACCAAC CACGAC ATCGCG CTGCTC CGCCTG CACCAG CCCGTG |
| 301 | GTCCTC ACTGAC CATGTG GTGCCC CTCTGC CTGCCC GAACGG ACGTTC TCTGAG AGGACG |
| 361 | CTGGCC TTCGTG CGCTTC TCATTG GTCAGC GGCTGG GGCCAG CTGCTG GACCGT GGCGCC |
| 421 | ACGGCC CTGGAG CTCATG GTCCTC AACGTG CCCCGG CTGATG ACCCAG GACTGC CTGCAG |
| 481 | CAGTCA CGGAAG GTGGGA GACTCC CCAAAT ATCACG GAGTAC ATGTTC TGTGCC GGCTAC |
| 541 | TCGGAT GGCAGC AAGGAC TCCTGC AAGGGG GACAGT GGAGGC CCACAT GCCACC CACTAC |
| 601 | CGGGGC ACGTGG TACCTG ACGGGC ATCGTC AGCTGG GGCCAG GGCTGC GCAACC GTGGGC |
| 661 | CACTTT GGGGTG TACACC AGGGTC TCCCAG TACATC GAGTGG CTGCAA AAGCTC ATGCGC |
| 721 | TCAGAG CCACGC CCAGGA GTCCTC CTGCGA GCCCCA TTTCCC |

### SEQ ID NO:181

### >FVII LC DNA

| | |
|---|---|
| 1 | GCCAAC GCGTTC CTGGAG GAGCTG CGGCCG GGCTCC CTGGAG AGGGAG TGCAAG GAGGAG |
| 61 | CAGTGC TCCTTC GAGGAG GCCCGG GAGATC TTCAAG GACGCG GAGAGG ACGAAG CTGTTC |
| 121 | TGGATT TCTTAC AGTGAT GGGGAC CAGTGT GCCTCA AGTCCA TGCCAG AATGGG GGCTCC |
| 181 | TGCAAG GACCAG CTCCAG TCCTAT ATCTGC TTCTGC CTCCCT GCCTTC GAGGGC CGGAAC |
| 241 | TGTGAG ACGCAC AAGGAT GACCAG CTGATC TGTGTG AACGAG AACGGC GGCTGT GAGCAG |
| 301 | TACTGC AGTGAC CACACG GGCACC AAGCGC TCCTGT CGGTGC CACGAG GGGTAC TCTCTG |
| 361 | CTGGCA GACGGG GTGTCC TGCACA CCCACA GTTGAA TATCCA TGTGGA AAAATA CCTATT |
| 421 | CTAGAA AAAAGA AATGCC AGCAAA CCCCAA GGCCGA |

### SEQ ID NO:182

### >Insert

### SEQ ID NO:183

>Human GPIIb. Signal sequence (1-31). Transmembrane (981-1019). Cytoplasmic (1020-1039)

MARALCPLQALWLLEWVLLLLGPCAAPPAWALNLDPVQLTFYAGPNGSQF
GFSLDFHKDSHGRVAIVVGAPRTLGPSQEETGGVFLCPWRAEGGQCPSLL
FDLRDETRNVGSQTLQTFKARQGLGASVVSWSDVIVACAPWQHWNVLEKT
EEREKTPVGSCFLAQPESGRRAEYSPCRGNTLSRIYVENDFSWDKRYCEA
GFSSVVTQAGELVLGAPGGYYFLGLLAQAPVADIFSSYRPGILLWHVSSQ
SLSFDSSNPEYFDGYWGYSVAVGEFDGDLNTTEYVVGAPTWSWTLGAVEI
LDSYYQRLHRLRGEQMASYFGHSVAVTDVNGDGRHDLLVGAPLYMESRAD
RKLAEVGRVYLFTQPRGPHALGAPSLLLTGTQLYGRFGSAIAPLGDLDRD
GYNDIAVAAPYGGPSGRGQVLVFLGQSEGLRSRPSQVLDSPFPTGSAFGF
SLRGAVDIDDNGYPDLIVGAYGANQVAVYRAQPVVKASVQLLVQDSLNPA
VKSCVLPQTKTPVSCFNIQMCVGATGHNIPQKLSLNAELQLDRQKPRQGR
RVLLLGSQQAGTTLNLDLGGKHSPICHTTMAFLRDEADFRDKLSPIVLSL
NVSLPPTEAGMAPAVVLHGDTHVQEQTRIVLDCGEDDVCVPQLQLTASVT
GSPLLVGADNVLELQMDAANEGEGAYEAELAVHLPQGAHYMRALSNVEGF
ERLICNQKKENETRVVLCELGNPMKKNAQIGIAMLVSVGNLEEAGESVSF
QLQIRSKNSQNPNSKIVLLDVPVRAEAQVELRGNSFPASLVVAAEEGERE
QNSLDSWGPKVEHTYELHNNGPGTVNGLHLSIHLPGQSQPSDLLYILDIQ
PQGGLQCFPQPPVNPLKVDWGLPIPSPSPIHPAHHKRDRRQIFLPEPEQP
SRLQDPVLVSCDSAPCTVVQCDLQEMARGQRAMVTVLAFLWLPSLYQRPL
DQFVLQSHAWFNVSSLPYAVPPLSLPRGEAQVWTQLLRALEERAIPIWWV
LVGVLGGLLLLTILVLAMWKVGFFKRNRPPLEEDDEEGE

### SEQ ID NO:184

>Human GPIIIa. Signal sequence (1-26), Transmembrane (719-747). Cytoplasmic (748-788)

MRARPRPRPLWATVLALGALAGVGVGGPNICTTRGVSSCQQCLAVSPMCA
WCSDEALPLGSPRCDLKENLLKDNCAPESIEFPVSEARVLEDRPLSDKGS
GDSSQVTQVSPQRIALRLRPDDSKNFSIQVRQVEDYPVDIYYLMDLSYSM
KDDLWSIQNLGTKLATQMRKLTSNLRIGFGAFVDKPVSPYMYISPPEALE
NPCYDMKTTCLPMFGYKHVLTLTDQVTRFNEEVKKQSVSRNRDAPEGGFD
AIMQATVCDEKIGWRNDASHLLVFTTDAKTHIALDGRLAGIVQPNDGQCH
VGSDNHYSASTTMDYPSLGLMTEKLSQKNINLIFAVTENVVNLYQNYSEL
IPGTTVGVLSMDSSNVLQLIVDAYGKIRSKVELEVRDLPEELSLSFNATC
LNNEVIPGLKSCMGLKIGDTVSFSIEAKVRGCPQEKEKSFTIKPVGFKDS
LIVQVTFDCDCACQAQAEPNSHRCNNGNGTFECGVCRCGPGWLGSQCECS
EEDYRPSQQDECSPREGQPVCSQRGECLCGQCVCHSSDFGKITGKYCECD
DFSCVRYKGEMCSGHGQCSCGDCLCDSDWTGYYCNCTTRTDTCMSSNGLL
CSGRGKCECGSCVCIQPGSYGDTCEKCPTCPDACTFKKECVECKKFDRGA
LHDENTCNRYCRDEIESVKELKDTGKDAVNCTYKNEDDCVVRFQYYEDSS
GKSILYVVEEPECPKGPDILVVLLSVMGAILLIGLAALLIWKLLITIHDR
KEFAKFEEERARAKWDTANNPLYKEATSTFTNITYRGT

### SEQ ID NO:185

### >Human GPIIb DNA.

ATGGCCAGAGCTTTGTGTCCACTGCAAGCCCTCTGGCTTCTGGAGTGGGT
GCTGCTGCTCTACCGGTCTCGAAACACAGGTGACGTTCGGGAGACCGAAG
ACCTCACCCACGACGACGAGTTGGGACCTTGTGCTGCCCCTCCAGCCTGG
GCCTTGAACCTGGACCCAGTGCAGCTCACCAACCCTGGAACACGACGGGG
AGGTCGGACCCGGAACTTGGACCTGGGTCACGTCGAGTGGTTCTATGCAG
GCCCCAATGGCAGCCAGTTTGGATTTTCACTGGACTTCCACAAGGACAGC
AAGATACGTCCGGGGTTACCGTCGGTCAAACCTAAAAGTGACCTGAAGGT
GTTCCTGTCGCATGGGAGAGTGGCCATCGTGGTGGGCGCCCCGCGGACCC
TGGGCCCCAGCCAGGAGGAGGTACCCTCTCACCGGTAGCACCACCCGCGG
GGCGCCTGGGACCCGGGGTCGGTCCTCCTCACGGGCGGCGTGTTCCTGTG
CCCCTGGAGGGCCGAGGGCGGCCAGTGCCCCTCGCTGCTCTGCCCGCCGC
ACAAGGACACGGGGACCTCCCGGCTCCCGCCGGTCACGGGGAGCGACGAG
TTTGACCTCCGTGATGAGACCCGAAATGTAGGCTCCCAAACTTTACAAAC
CTTCAAGGCCAAACTGGAGGCACTACTCTGGGCTTTACATCCGAGGGTTT
GAAATGTTTGGAAGTTCCGGCGCCAAGGACTGGGGGCGTCGGTCGTCAGC
TGGAGCGACGTCATTGTGGCCTGCGCCCCCGCGGTTCCTGACCCCCGCAG
CCAGCAGTCGACCTCGCTGCAGTAACACCGGACGCGGGGGTGGCAGCACT
GGAACGTCCTAGAAAAGACTGAGGAGGCTGAGAAGACGCCCGTAGGTAGC
ACCGTCGTGACCTTGCAGGATCTTTTCTGACTCCTCCGACTCTTCTGCGG
GCATCCATCGTGCTTTTTGGCTCAGCCAGAGAGCGGCCGGCGCGCCGAGT
ACTCCCCCTGTCGCGGGAACACGAAAAACCGAGTCGGTCTCTCGCCGGCC
GCGCGGCTCATGAGGGGGACAGCGCCCTTGACCCTGAGCCGCATTTACGT
GGAAAATGATTTTAGCTGGGACAAGCGTTACTGTGAAGCGTGGGACTCGG
CGTAAATGCACCTTTTACTAAAATCGACCCTGTTCGCAATGACACTTCGC
GGCTTCAGCTCCGTGGTCACTCAGGCCGGAGAGCTGGTGCTTGGGGCTCC
TGGCGGCTATCCGAAGTCGAGGCACCAGTGAGTCCGGCCTCTCGACCACG
AACCCCGAGGACCGCCGATATATTTCTTAGGTCTCCTGGCCCAGGCTCCA
GTTGCGGATATTTTCTCGAGTTACCGCCCAATAAAGAATCCAGAGGACCG
GGTCCGAGGTCAACGCCTATAAAAGAGCTCAATGGCGGGTGGCATCCTTT
TGTGGCACGTGTCCTCCCAGAGCCTCTCCTTTGACTCCAGCAACCCAGAG
CCGTAGGAAAACACCGTGCACAGGAGGGTCTCGGAGAGGAAACTGAGGTC
GTTGGGTCTCTACTTCGACGGCTACTGGGGGTACTCGGTGGCCGTGGGCG
AGTTCGACGGGGATCTCAACATGAAGCTGCCGATGACCCCCATGAGCCAC
CGGCACCCGCTCAAGCTGCCCCTAGAGTTGACTACAGAATATGTCGTCGG
TGCCCCCACTTGGAGCTGGACCCTGGGAGCGGTGGAAATTTGATGTCTTA
TACAGCAGCCACGGGGGTGAACCTCGACCTGGGACCCTCGCCACCTTTAA
TTGGATTCCTACTACCAGAGGCTGCATCGGCTGCGCGGAGAGCAGATGGC
GTCGTATTTTAACCTAAGGATGATGGTCTCCGACGTAGCCGACGCGCCTC
TCGTCTACCGCAGCATAAAAGGGCATTCAGTGGCTGTCACTGACGTCAAC
GGGGATGGGAGGCATGATCTGCTGGTGGGCCCCGTAAGTCACCGACAGTG
ACTGCAGTTGCCCCTACCCTCCGTACTAGACGACCACCCGGCTCCACTGT
ATATGGAGAGCCGGGCAGACCGAAAACTGGCCGAAGTGGGGCGTGTGTAT
CGAGGTGACATATACCTCTCGGCCCGTCTGGCTTTTGACCGGCTTCACCC
CGCACACATATTGTTCCTGCAGCCGCGAGGCCCCCACGCGCTGGGTGCCC
CCAGCCTCCTGCTGACTGGCAACAAGGACGTCGGCGCTCCGGGGGTGCGC
GACCCACGGGGGTCGGAGGACGACTGACCGACACAGCTCTATGGGCGATT
CGGCTCTGCCATCGCACCCCTGGGCGACCTCGACCGGGATTGTGTCGAGA
TACCCGCTAAGCCGAGACGGTAGCGTGGGGACCCGCTGGAGCTGGCCCTA
GGCTACAATGACATTGCAGTGGCTGCCCCCTACGGGGGTCCCAGTGGCCG
GGGCCAAGTGCCGATGTTACTGTAACGTCACCGACGGGGGATGCCCCCAG
GGTCACCGGCCCCGGTTCACCTGGTGTTCCTGGGTCAGAGTGAGGGGCTG
AGGTCACGTCCCTCCCAGGTCCTGGACAGCGACCACAAGGACCCAGTCTC
ACTCCCCGACTCCAGTGCAGGGAGGGTCCAGGACCTGTCGCCCTTCCCCA
CAGGCTCTGCCTTTGGCTTCTCCCTTCGAGGTGCCGTAGACATCGATGAC
GGGAAGGGGTGTCCGAGACGGAAACCGAAGAGGGAAGCTCCACGGCATCT
GTAGCTACTGAACGGATACCCAGACCTGATCGTGGGAGCTTACGGGGCCA
ACCAGGTGGCTGTGTACAGATTGCCTATGGGTCTGGACTAGCACCCTCGA
ATGCCCCGGTTGGTCCACCGACACATGTCTGCTCAGCCAGTGGTGAAGGC
CTCTGCCAGCTACTGGTGCAAGATTCACTGAATCCTGCTCGAGTCGGTCA
CCACTTCCGGAGACAGGTCGATGACCACGTTCTAAGTGACTTAGGACGAG
TGAAGAGCTGTGTCCTACCTCAGACCAAGACACCCGTGAGCTGCTTCAAC
ATCCAGATGCACTTCTCGACACAGGATGGAGTCTGGTTCTGTGGGCACTC
GACGAAGTTGTAGGTCTACTGTGTTGGAGCCACTGGGCACAACATTCCTC
AGAAGCTATCCCTAAATGCCGAGCTGCAGACACAACCTCGGTGACCCGTG
TTGTAAGGAGTCTTCGATAGGGATTTACGGCTCGACGTCCTGGACCGGCA
GAAGCCCCGCCAGGGCCGGCGGGTGCTGCTGCTGGGCTCTCAACAGGCAG
ACCTGGCCGTCTTCGGGGCGGTCCCGGCCGCCCACGACGACGACCCGAGA
GTTGTCCGTGGCACCACCCTGAACCTGGATCTGGGCGGAAAGCACAGCCC
CATCTGCCACACCACCATGCCGTGGTGGGACTTGGACCTAGACCCGCCTT
TCGTGTCGGGGTAGACGGTGTGGTGGTACGCCTTCCTTCGAGATGAGGCA
GACTTCCGGGACAAGCTGAGCCCCATTGTGCTCAGCCTCCGGAAGGAAGC
TCTACTCCGTCTGAAGGCCCTGTTCGACTCGGGGTAACACGAGTCGGAGA
ATGTGTCCCTACCGCCCACGGAGGCTGGAATGGCCCCTGCTGTCGTGCTG
CATGGAGACTTACACAGGGATGGCGGGTGCCTCCGACCTTACCGGGGACG
ACAGCACGACGTACCTCTGACCCATGTGCAGGAGCAGACACGAATCGTCC
TGGACTGTGGGGAAGATGACGTATGTGTGTGGGTACACGTCCTCGTCTGT
GCTTAGCAGGACCTGACACCCCTTCTACTGCATACACACCCCCAGCTTCA
GCTCACTGCCAGCGTGACGGGCTCCCCGCTCCTAGTTGGGGCAGATAATG
GGGTCGAAGTCGAGTGACGGTCGCACTGCCCGAGGGGCGAGGATCAACCC
CGTCTATTAGTCCTGGAGCTGCAGATGGACGCAGCCAACGAGGGCGAGGG
GGCCTATGAAGCAGAGCTGCAGGACCTCGACGTCTACCTGCGTCGGTTGC
TCCCGCTCCCCCGGATACTTCGTCTCGACGCCGTGCACCTGCCCCAGGGC
GCCCACTACATGCGGGCCCTAAGCAATGTCGAGGGCTTTCGGCACGTGGA
CGGGGTCCCGCGGGTGATGTACGCCCGGGATTCGTTACAGCTCCCGAAAG
AGAGACTCATCTGTAATCAGAAGAAGGAGAATGAGACCAGGGTGGTGCTG
TGTGAGCTGCTCTCTGAGTAGACATTAGTCTTCTTCCTCTTACTCTGGTC
CCACCACGACACACTCGACGGCAACCCCATGAAGAAGAACGCCCAGATAG
GAATCGCGATGTTGGTGAGCGTGGGGAATCCGTTGGGGTACTTCTTCTTG
CGGGTCTATCCTTAGCGCTACAACCACTCGCACCCCTTACTGGAAGAGGC
TGGGGAGTCTGTGTCCTTCCAGCTGCAGATACGGAGCAAGAACAGCCAGG
ACCTTCTCCGACCCCTCAGACACAGGAAGGTCGACGTCTATGCCTCGTTC
TTGTCGGTCAATCCAAACAGCAAGATTGTGCTGCTGGACGTGCCGGTCCG
GGCAGAGGCCCAAGTGGAGTTAGGTTTGTCGTTCTAACACGACGACCTGC
ACGGCCAGGCCCGTCTCCGGGTTCACCTCCTGCGAGGGAACTCCTTTCCA
GCCTCCCTGGTGGTGGCAGCAGAAGAAGGTGAGAGGGAGGACGCTCCCTT
GAGGAAAGGTCGGAGGGACCACCACCGTCGTCTTCTTCCACTCTCCCTCC
AGAACAGCTTGGACAGCTGGGGACCCAAAGTGGAGCACACCTATGAGCTC
CACAACAATGTCTTGTCGAACCTGTCGACCCCTGGGTTTCACCTCGTGTG
GATACTCGAGGTGTTGTTAGGCCCTGGGACTGTGAATGGTCTTCACCTCA
GCATCCACCTTCCGGGACAGTCCCAGCCCCCGGGACCCTGACACTTACCA
GAAGTGGAGTCGTAGGTGGAAGGCCCTGTCAGGGTCGGGTCCGACCTGCT
CTACATCCTGGATATACAGCCCCAGGGGGGCCTTCAGTGCTTCCCACAGA
GGCTGGACGAGATGTAGGACCTATATGTCGGGGTCCCCCCGGAAGTCACG
AAGGGTGTCCCTCCTGTCAACCCTCTCAAGGTGGACTGGGGGCTGCCCAT
CCCCAGCCCCTCCCCCATTGGAGGACAGTTGGGAGAGTTCCACCTGACCC
CCGACGGGTAGGGGTCGGGGAGGGGGTAACACCCGGCCCATCACAAGCGG
GATCGCAGACAGATCTTCCTGCCAGAGCCCGAGCAGCCCGTGGGCCGGGT
AGTGTTCGCCCTAGCGTCTGTCTAGAAGGACGGTCTCGGGCTCGTCGGGT
CGAGGCTTCAGGATCCAGTTCTCGTAAGCTGCGACTCGGCGCCCTGTACT
GTGGTGCAGAGCTCCGAAGTCCTAGGTCAAGAGCATTCGACGCTGAGCCG
CGGGACATGACACCACGTCTGTGACCTGCAGGAGATGGCGCGCGGGCAGC
GGGCCATGGTCACGGTGCTGGCCTTCCTGACACTGGACGTCCTCTACCGC
GCGCCCGTCGCCCGGTACCAGTGCCACGACCGGAAGGACTGGCTGCCCAG
CCTCTACCAGAGGCCTCTGGATCAGTTTGTGCTGCAGTCGCACGCATGGA
CCGACGGGTCGGAGATGGTCTCCGGAGACCTAGTCAAACACGACGTCAGC
GTGCGTACCTTCAACGTGTCCTCCCTCCCCTATGCGGTGCCCCCGCTCAG
CCTGCCCCGAGGGGAAGCTAAGTTGCACAGGAGGGAGGGGATACGCCACG
GGGGCGAGTCGGACGGGCTCCCCTTCGACAGGTGTGGACACAGCTGCTCC
GGGCCTTGGAGGAGAGGGCCATTCCAATCTGGTGGGTGGTCCACACCTGT
GTCGACGAGGCCCGGAACCTCCTCTCCCGGTAAGGTTAGACCACCCACCT
GGTGGGTGTGCTGGGTGGCCTGCTGCTGCTCACCATCCTGGTCCTGGCCA
TGTGGAAGGACCACCCACACGACCCACCGGACGACGACGAGTGGTAGGAC
CAGGACCGGTACACCTTCGTCGGCTTCTTCAAGCGGAACCGGCCACCCCT
GGAAGAAGATGATGAAGAGGGGGAGTGACAGCCGAAGAAGTTCGCCTTGG
CCGGTGGGGACCTTCTTCTACTACTTCTCCCCCTCACT

### SEQ ID NO:186

### >Human GPIIIa DNA

ATGCGAGCGCGCCCGCGGCCCCGGCCGCTCTGGGCGACTGTGCTGGCGCT
GGGGGCGCTGTACGCTCGCGCGGGCGCCGGGGCCGGCGAGACCCGCTGAC
ACGACCGCGACCCCCGCGACGCGGGCGTTGGCGTAGGAGGGCCCAACATC
TGTACCACGCGAGGTGTGAGCTCCTGCCAGCGCCCGCAACCGCATCCTCC
CGGGTTGTAGACATGGTGCGCTCCACACTCGAGGACGGTCCAGTGCCTGG
CTGTGAGCCCCATGTGTGCCTGGTGCTCTGATGAGGCCCTGCCTCTGGGC
GTCACGGACCGACACTCGGGGTACACACGGACCACGAGACTACTCCGGGA
CGGAGACCCGTCACCTCGCTGTGACCTGAAGGAGAATCTGCTGAAGGATA
ACTGTGCCCCAGAATCCATCAGTGGAGCGACACTGGACTTCCTCTTAGAC
GACTTCCTATTGACACGGGGTCTTAGGTAGGAGTTCCCAGTGAGTGAGGC
CCGAGTACTAGAGGACAGGCCCCTCAGCGACAAGGGCTCTCTCAAGGGTC
ACTCACTCCGGGCTCATGATCTCCTGTCCGGGGAGTCGCTGTTCCCGAGA
GGAGACAGCTCCCAGGTCACTCAAGTCAGTCCCCAGAGGATTGCACTCCG
GCTCCGGCCACCTCTGTCGAGGGTCCAGTGAGTTCAGTCAGGGGTCTCCT
AACGTGAGGCCGAGGCCGGTGATGATTCGAAGAATTTCTCCATCCAAGTG
CGGCAGGTGGAGGATTACCCTGTGGACATCCTACTAAGCTTCTTAAAGAG
GTAGGTTCACGCCGTCCACCTCCTAATGGGACACCTGTAGTACTACTTGA
TGGACCTGTCTTACTCCATGAAGGATGATCTGTGGAGCATCCAGAACCTG
ATGATGAACTACCTGGACAGAATGAGGTACTTCCTACTAGACACCTCGTA
GGTCTTGGACGGTACCAAGCTGGCCACCCAGATGCGAAAGCTCACCAGTA
ACCTGCGGATTGGCTTCGGGCCATGGTTCGACCGGTGGGTCTACGCTTTC
GAGTGGTCATTGGACGCCTAACCGAAGCCCGCATTTGTGGACAAGCCTGT
GTCACCATACATGTATATCTCCCCACCAGAGGCCCTCGAACGTAAACACC
TGTTCGGACACAGTGGTATGTACATATAGAGGGGTGGTCTCCGGGAGCTT
AACCCCTGCTATGATATGAAGACCACCTGCTTGCCCATGTTTGGCTACAA
ACACGTGCTGTTGGGGACGATACTATACTTCTGGTGGACGAACGGGTACA
AACCGATGTTTGTGCACGACACGCTAACTGACCAGGTGACCCGCTTCAAT
GAGGAAGTGAAGAAGCAGAGTGTGTCACGGTGCGATTGACTGGTCCACTG
GGCGAAGTTACTCCTTCACTTCTTCGTCTCACACAGTGCCAACCGAGATG
CCCCAGAGGGTGGCTTTGATGCCATCATGCAGGCTACAGTCTGTGATGAA
TTGGCTCTACGGGGTCTCCCACCGAAACTACGGTAGTACGTCCGATGTCA
GACACTACTTAAGATTGGCTGGAGGAATGATGCATCCCACTTGCTGGTGT
TTACCACTGATGCCAAGACTTTCTAACCGACCTCCTTACTACGTAGGGTG
AACGACCACAAATGGTGACTACGGTTCTGACATATAGCATTGGACGGAAG
GCTGGCAGGCATTGTCCAGCCTAATGACGGGCAGTGTCATGTATATCGTA
ACCTGCCTTCCGACCGTCCGTAACAGGTCGGATTACTGCCCGTCACAGTA
GTTGGTAGTGACAATCATTACTCTGCCTCCACTACCATGGATTATCCCTC
TTTGGGGCTGCAACCATCACTGTTAGTAATGAGACGGAGGTGATGGTACC
TAATAGGGAGAAACCCCGACATGACTGAGAAGCTATCCCAGAAAAACATC
AATTTGATCTTTGCAGTGACTGAAAATGTATACTGACTCTTCGATAGGGT
CTTTTTGTAGTTAAACTAGAAACGTCACTGACTTTTACATGTCAATCTCT
ATCAGAACTATAGTGAGCTCATCCCAGGGACCACAGTTGGGGTTCTGTCC
CAGTTAGAGATAGTCTTGATATCACTCGAGTAGGGTCCCTGGTGTCAACC
CCAAGACAGGATGGATTCCAGCAATGTCCTCCAGCTCATTGTTGATGCTT
ATGGGAAAATCCGTTCTAAATACCTAAGGTCGTTACAGGAGGTCGAGTAA
CAACTACGAATACCCTTTTAGGCAAGATTTGTAGAGCTGGAAGTGCGTGA
CCTCCCTGAAGAGTTGTCTCTATCCTTCAATGCCACCTGCCATCTCGACC
TTCACGCACTGGAGGGACTTCTCAACAGAGATAGGAAGTTACGGTGGACG
CTCAACAATGAGGTCATCCCTGGCCTCAAGTCTTGTATGGGACTCAAGAT
TGGAGACACGGAGTTGTTACTCCAGTAGGGACCGGAGTTCAGAACATACC
CTGAGTTCTAACCTCTGTGCGTGAGCTTCAGCATTGAGGCCAAGGTGCGA
GGCTGTCCCCAGGAGAAGGAGAAGTCCTTTCACTCGAAGTCGTAACTCCG
GTTCCACGCTCCGACAGGGGTCCTCTTCCTCTTCAGGAAAACCATAAAGC
CCGTGGGCTTCAAGGACAGCCTGATCGTCCAGGTCACCTTTGATTGTGAC
TGGTATTTCGGGCACCCGAAGTTCCTGTCGGACTAGCAGGTCCAGTGGAA
ACTAACACTGTGTGCCTGCCAGGCCCAAGCTGAACCTAATAGCCATCGCT
GCAACAATGGCAATGGGACCACACGGACGGTCCGGGTTCGACTTGGATTA
TCGGTAGCGACGTTGTTACCGTTACCCTGGTTTGAGTGTGGGGTATGCCG
TTGTGGGCCTGGCTGGCTGGGATCCCAGTGTGAGTGCTCAAAACTCACAC
CCCATACGGCAACACCCGGACCGACCGACCCTAGGGTCACACTCACGAGT
GAGGAGGACTATCGCCCTTCCCAGCAGGACGAATGCAGCCCCCGGGAGGG
TCAGCCCGTCCTCCTCCTGATAGCGGGAAGGGTCGTCCTGCTTACGTCGG
GGGCCCTCCCAGTCGGGCAGTGCAGCCAGCGGGGCGAGTGCCTCTGTGGT
CAATGTGTCTGCCACAGCAGTGACTTTGGCACGTCGGTCGCCCCGCTCAC
GGAGACACCAGTTACACAGACGGTGTCGTCACTGAAACCGAAGATCACGG
GCAAGTACTGCGAGTGTGACGACTTCTCCTGTGTCCGCTACAAGGGGGAG
TTCTAGTGCCCGTTCATGACGCTCACACTGCTGAAGAGGACACAGGCGAT
GTTCCCCCTCATGTGCTCAGGCCATGGCCAGTGCAGCTGTGGGGACTGCC
TGTGTGACTCCGACTGGACCTACACGAGTCCGGTACCGGTCACGTCGACA
CCCCTGACGGACACACTGAGGCTGACCTGGGGCTACTACTGCAACTGTAC
CACGCGTACTGACACCTGCATGTCCAGCAATGGGCTGCTGCCGATGATGA
CGTTGACATGGTGCGCATGACTGTGGACGTACAGGTCGTTACCCGACGAC
TGCAGCGGCCGGGGCAAGTGTGAATGTGGCAGCTGTGTCTGTATCCAGCC
GGGCTCCTATACGTCGCCGGCCCCGTTCACACTTACACCGTCGACACAGA
CATAGGTCGGCCCGAGGATAGGGGACACCTGTGAGAAGTGCCCCACCTGC
CCAGATGCCTGCACCTTTAAGAAAGAATGTCCCCTGTGGACACTCTTCAC
GGGGTGGACGGGTCTACGGACGTGGAAATTCTTTCTTACAGTGGAGTGTA
AGAAGTTTGACCGGGGAGCCCTACATGACGAAAATACCTGCAACCGTTAC
CACCTCACATTCTTCAAACTGGCCCCTCGGGATGTACTGCTTTTATGGAC
GTTGGCAATGTGCCGTGACGAGATTGAGTCAGTGAAAGAGCTTAAGGACA
CTGGCAAGGATGCAGTGAATACGGCACTGCTCTAACTCAGTCACTTTCTC
GAATTCCTGTGACCGTTCCTACGTCACTTATGTACCTATAAGAATGAGGA
TGACTGTGTCGTCAGATTCCAGTACTATGAAGATTCTAGTACATGGATAT
TCTTACTCCTACTGACACAGCAGTCTAAGGTCATGATACTTCTAAGATCA
GGAAAGTCCATCCTGTATGTGGTAGAAGAGCCAGAGTGTCCCAAGGGCCC
TGACATCCTGCCTTTCAGGTAGGACATACACCATCTTCTCGGTCTCACAG
GGTTCCCGGGACTGTAGGACGTGGTCCTGCTCTCAGTGATGGGGGCCATT
CTGCTCATTGGCCTTGCCGCCCTGCTCATCCACCAGGACGAGAGTCACTA
CCCCCGGTAAGACGAGTAACCGGAACGGCGGGACGAGTAGTGGAAACTCC
TCATCACCATCCACGACCGAAAAGAGTTCGCTAAATTTGAGGAAGAACGC
ACCTTTGAGGAGTAGTGGTAGGTGCTGGCTTTTCTCAAGCGATTTAAACT
CCTTCTTGCGGCCAGAGCAAAATGGGACACAGCCAACAACCCACTGTATA
AAGAGGCCACGTCTACCTTCCGGTCTCGTTTTACCCTGTGTCGGTTGTTG
GGTGACATATTTCTCCGGTGCAGATGGAAGACCAATATCACGTACCGGGG
CACTTAATGGTTATAGTGCATGGCCCCGTGAATT

### SEQ ID NO:187

>DNA sequence of FVII-165

| | |
|---|---|
| 1 | ATGGTC TCCCAG GCCCTC AGGCTC CTCTGC CTTCTG CTTGGG CTTCAG GGCTGC CTGGCT |
| 61 | GCAGTC TTCGTA ACCCAG GAGGAA GCCCAC GGCGTC CTGCAC CGGCGC CGGCGC GCCAAC |
| 121 | GCGTTC CTGGAG GAGCTG CGGCCG GGCTCC CTGGAG AGGGAG TGCAAG GAGGAG CAGTGC |
| 181 | TCCTTC GAGGAG GCCCGG GAGATC TTCAAG GACGCG GAGAGG ACGAAG CTGTTC TGGATT |
| 241 | TCTTAC AGTGAT GGGGAC CAGTGT GCCTCA AGTCCA TGCCAG AATGGG GGCTCC TGCAAG |
| 301 | GACCAG CTCCAG TCCTAT ATCTGC TTCTGC CTCCCT GCCTTC GAGGGC CGGAAC TGTGAG |
| 361 | ACGCAC AAGGAT GACCAG CTGATC TGTGTG AACGAG AACGGC GGCTGT GAGCAG TACTGC |
| 421 | AGTGAC CACACG GGCACC AAGCGC TCCTGT CGGTGC CACGAG GGGTAC TCTCTG CTGGCA |
| 481 | GACGGG GTGTCC TGCACA CCCACA GTTGAA TATCCA TGTGGA AAAATA CCTATT CTAGAA |
| 541 | AAAAGA AATGCC AGCAAA CCCCAA GGCCGA ATTGTG GGGGGC AAGGTG TGCCCC AAAGGG |
| 601 | GAGTGT CCATGG CAGGTC CTGTTG TTGGTG AATGGA GCTCAG TTGTGT GGGGGG ACCCTG |
| 661 | ATCAAC ACCATC TGGGTG GTCTCC GCGGCC CACTGT TTCGAC AAAATC AAGAAC TGGAGG |
| 721 | AACCTG ATCGCG GTGCTG GGCGAG CACGAC CTCAGC GAGCAC GACGGG GATGAG CAGAGC |
| 781 | CGGCGG GTGGCG CAGGTC ATCATC CCCAGC ACGTAC GTCCCG GGCACC ACCAAC CACGAC |
| 841 | ATCGCG CTGCTC CGCCTG CACCAG CCCGTG GTCCTC ACTGAC CATGTG GTGCCC CTCTGC |
| 901 | CTGCCC GAACGG ACGTTC TCTGAG AGGACG CTGGCC TTCGTG CGCTTC TCATTG GTCAGC |
| 961 | GGCTGG GGCCAG CTGCTG GACCGT GGCGCC ACGGCC CTGGAG CTCATG GTCCTC AACGTG |
| 1021 | CCCCGG CTGATG ACCCAG GACTGC CTGCAG CAGTCA CGGAAG GTGGGA GACTCC CCAAAT |
| 1081 | ATCACG GAGTAC ATGTTC TGTGCC GGCTAC TCGGAT GGCAGC AAGGAC TCCTGC AAGGGG |
| 1141 | GACAGT GGAGGC CCACAT GCCACC CACTAC CGGGGC ACGTGG TACCTG ACGGGC ATCGTC |
| 1201 | AGCTGG GGCCAG GGCTGC GCAACC GTGGGC CACTTT GGGGTG TACACC AGGGTG TCCCAG |
| 1261 | TACATC GAGTGG CTGCAA AAGCTC ATGCGC TCAGAG CCACGC CCAGGA GTCCTC CTGCGA |
| 1321 | GCCCCA TTTCCC GGGTCT CCAGGT ACCTCA GAGTCT GCTACC CCCGAG TCAGGG CCAGGA |
| 1381 | TCAGAG CCAGCC ACCTCC GGGTCT GAGACA CCCGGG ACTTCC GAGAGT GCCACC CCTGAG |
| 1441 | TCCGGA CCCGGG TCCGAG CCCGCC ACTTCC GGCTCC GAAACT CCCGGC ACAAGC GAGAGC |
| 1501 | GCTACC CCAGAG TCAGGA CCAGGA ACATCT ACAGAG CCCTCT GAAGGC TCCGCT CCAGGG |
| 1561 | TCCCCA GCCGGC AGTCCC ACTAGC ACCGAG GAGGGA ACCTCT GAAAGC GCCACA CCCGAA |
| 1621 | TCAGGG CCAGGG TCTGAG CCTGCT ACCAGC GGCAGC GAGACA CCAGGC ACCTCT GAGTCC |
| 1681 | GCCACA CCAGAG TCCGGA CCCGGA TCTCCC GCTGGG AGCCCC ACCTCC ACTGAG GAGGGA |
| 1741 | TCTCCT GCTGGC TCTCCA ACATCT ACTGAG GAAGGT ACCTCA ACCGAG CCATCC GAGGGA |
| 1801 | TCAGCT CCCGGC ACCTCA GAGTCG GCAACC CCGGAG TCTGGA CCCGGA ACTTCC GAAAGT |
| 1861 | GCCACA CCAGAG TCCGGT CCCGGG ACTTCA GAATCA GCAACA CCCGAG TCCGGC CCTGGG |
| 1921 | TCTGAA CCCGCC ACAAGT GGTAGT GAGACA CCAGGA TCAGAA CCTGCT ACCTCA GGGTCA |
| 1981 | GAGACA CCCGGA TCTCCG GCAGGC TCACCA ACCTCC ACTGAG GAGGGC ACCAGC ACAGAA |
| 2041 | CCAAGC GAGGGC TCCGCA CCCGGA ACAAGC ACTGAA CCCAGT GAGGGT TCAGCA CCCGGC |
| 2101 | TCTGAG CCGGCC ACAAGT GGCAGT GAGACA CCCGGC ACTTCA GAGAGT GCCACC CCCGAG |
| 2161 | AGTGGC CCAGGC ACTAGT ACCGAG CCCTCT GAAGGC AGTGCG CCA |

### SEQ ID NO:188

>DNA sequence for FVII-175

| | |
|---|---|
| 1 | ATGGTC TCCCAG GCCCTC AGGCTC CTCTGC CTTCTG CTTGGG CTTCAG GGCTGC CTGGCT |
| 61 | GCAGTC TTCGTA ACCCAG GAGGAA GCCCAC GGCGTC CTGCAC CGGCGC CGGCGC GCCAAC |
| 121 | GCGTTC CTGGAG GAGCTG CGGCCG GGCTCC CTGGAG AGGGAG TGCAAG GAGGAG CAGTGC |
| 181 | TCCTTC GAGGAG GCCCGG GAGATC TTCAAG GACGCG GAGAGG ACGAAG CTGTTC TGGATT |
| 241 | TCTTAC AGTGAT GGGGAC CAGTGT GCCTCA AGTCCA TGCCAG AATGGG GGCTCC TGCAAG |
| 301 | GACCAG CTCCAG TCCTAT ATCTGC TTCTGC CTCCCT GCCTTC GAGGGC CGGAAC TGTGAG |
| 361 | ACGCAC AAGGAT GACCAG CTGATC TGTGAG AACGAG AACGGC GGCTGT GAGCAG TACTGC |
| 421 | AGTGAC CACACG GGCACC AAGCGC TCCTGT CGGTGC CACGAG GGGTAC TCTCTG CTGGCA |
| 481 | GACGGG GTGTCC TGCACA CCCACA GTTGAA TATCCA TGTGGA AAAATA CCTATT CTAGAA |
| 541 | AAAAGA AATGCC AGCAAA CCCCAA GGCCGA ATTGTG GGGGGC AAGGTG TGCCCC AAAGGG |
| 601 | GAGTGT CCATGG CAGGTC CTGTTG TTGGTG AATGGA GCTCAG TTGTGT GGGGGG ACCCTG |
| 661 | ATCAAC ACCATC TGGGTG GTCTCC GCGGCC CACTGT TTCGAC AAAATC AAGAAC TGGAGG |
| 721 | AACCTG ATCGCG GTGCTG GGCGAG CACGAC CTCAGC GAGCAC GACGGG GATGAG CAGAGC |
| 781 | CGGCGG GTGGCG CAGGTC ATCATC CCCAGC ACGTAC GTCCCG GGCACC ACCAAC CACGAC |
| 841 | ATCGCG CTGCTC CGCCTG CACCAG CCCGTG GTCCTC ACTGAC CATGTG GTGCCC CTCTGC |
| 901 | CTGCCC GAACGG ACGTTC TCTGAG AGGACG CTGGCC TTCGTG CGCTTC TCATTG GTCAGC |
| 961 | GGCTGG GGCCAG CTGCTG GACCGT GGCGCC ACGGCC CTGGAG CTCATG GTCCTC AACGTG |
| 1021 | CCCCGG CTGATG ACCCAG GACTGC CTGCAG CAGTCA CGGAAG GTGGGA GACTCC CCAAAT |
| 1081 | ATCACG GAGTAC ATGTTC TGTGCC GGCTAC TCGGAT GGCAGC AAGGAC TCCTGC AAGGGG |
| 1141 | GACAGT GGAGGC CCACAT GCCACC CACTAC CGGGGC ACGTGG TACCTG ACGGGC ATCGTC |
| 1201 | AGCTGG GGCCAG GGCTGC GCAACC GTGGGC CACTTT GGGGTG TACACC AGGGTC TCCCAG |
| 1261 | TACATC GAGTGG CTGCAA AAGCTC ATGCGC TCAGAG CCACGC CCAGGA GTCCTC CTGCGA |
| 1321 | GCCCCA TTTCCC GGTGGC GGTGGC TCCGGC GGAGGT GGGTCC GGTGGC GGCGGA TCAGGT |
| 1381 | GGGGGT GGATCA GGCGGT GGAGGT TCCGGT GGCGGG GGATCC CAGGTG AAACTG CTCGAG |
| 1441 | TCTGGG GGAGGC GTGGTC CAGCCT GGGAGG TCCCTG AGACTC TCCTGT GCAGCC TCTGGA |
| 1501 | TTCACC TTCAGT AGCTAT GCTATG CACTGG GTCCGC CAGGCT CCAGGC AAGGGG CTGGAG |
| 1561 | TGGGTG GCAGTT ATATCA TATGAT GGAAGC AATAAA TACTAC GCAGAC TCCGTG AAGGGC |
| 1621 | CGATTC GCCATC TCCAGA GACAAT TCCAAG AACACG CTGTAT CTGCAA ATGAAC AGCCTG |
| 1681 | AGAGCT GAGGAC ACGGCT GTGTAT TACTGT GCGAGA GCGCTG GGGAGC TGGGGG GGTTGG |
| 1741 | GACCAC TACATG GACGTC TGGGGC AAAGGG ACCACG GTCACC GTCTCC TCAGGT GGCGGC |
| 1801 | GGATCA GGTGGG GGTGGA TCAGGT GGCGGT GGCTCC GGTGGC GGGGGA TCAGTG GTGACT |
| 1861 | CAGCCA CCCTCA GCGTCT GGGACC CCCGGG CAGAGG GTCACC ATCTCT TGTTCT GGAAGC |
| 1921 | AGCTCC AACATC GGAAGT AATACT GTAAAC TGGTAC CAGCAG CTCCCA GGAACG GCCCCC |
| 1981 | AAACTC CTCATC TATAGT AATAAT CAGCGG CCCTCA GGGGTC CCTGAC CGATTC TCTGGC |
| 2041 | TCCAAG TCTGGC ACCTCA GCCTCC CTGGCC ATCAGT GGGCTC CAGTCT GAGGAT GAGGCT |
| 2101 | GATTAT TACTGT GCAGCA TGGGAT GACAGC CTGAAT GGTTGG GTGTTC GGCGGA GGGACC |
| 2161 | AAGCTG ACCGTC CTAGGT CAGCCC GGTGGC GGCGGA TCAGGT GGGGGT GGATCA GGCGGT |
| 2221 | GGAGGT TCCGGT GGCGGG GGATCC GGCGGT GGAGGT TCCGGT GGGGGT GGATCA GGCTCG |
| 2281 | AGTGGT ACCTCA GAGTCT GCTACC CCCGAG TCAGGG CCAGGA TCAGAG CCAGCC ACCTCC |
| 2341 | GGGTCT GAGACA CCCGGG ACTTCC GAGAGT GCCACC CCTGAG TCCGGA CCCGGG TCCGAG |
| 2401 | CCCGCC ACTTCC GGCTCC GAAACT CCCGGC ACAAGC GAGAGC GCTACC CCAGAG TCAGGA |
| 2461 | CCAGGA ACATCT ACAGAG CCCTCT GAAGGC TCCGCT CCAGGG TCCCCA GCCGGC AGTCCC |
| 2521 | ACTAGC ACCGAG GAGGGA ACCTCT GAAAGC GCCACA CCCGAA TCAGGG CCAGGG TCTGAG |
| 2581 | CCTGCT ACCAGC GGCAGC GAGACA CCAGGC ACCTCT GAGTCC GCCACA CCAGAG TCCGGA |
| 2641 | CCCGGA TCTCCC GCTGGG AGCCCC ACCTCC ACTGAG GAGGGA TCTCCT GCTGGC TCTCCA |
| 2701 | ACATCT ACTGAG GAAGGT ACCTCA ACCGAG CCATCC GAGGGA TCAGCT CCCGGC ACCTCA |
| 2761 | GAGTCG GCAACC CCGGAG TCTGGA CCCGGA ACTTCC GAAAGT GCCACA CCAGAG TCCGGT |
| 2821 | CCCGGG ACTTCA GAATCA GCAACA CCCGAG TCCGGC CCTGGG TCTGAA CCCGCC ACAAGT |
| 2881 | GGTAGT GAGACA CCAGGA TCAGAA CCTGCT ACCTCA GGGTCA GAGACA CCCGGA TCTCCG |
| 2941 | GCAGGC TCACCA ACCTCC ACTGAG GAGGGC ACCAGC ACAGAA CCAAGC GAGGGC TCCGCA |
| 3001 | CCCGGA ACAAGC ACTGAA CCCAGT GAGGGT TCAGCA CCCGGC TCTGAG CCGGCC ACAAGT |
| 3061 | GGCAGT GAGACA CCCGGC ACTTCA GAGAGT GCCACC CCCGAG AGTGGC CCAGGC ACTAGT |
| 3121 | ACCGAG CCCTCT GAAGGC AGTGCG CCA |

### SEQ ID NO:189

>DNA sequence for FVII-177

| | |
|---|---|
| 1 | ATGGTC TCCCAG GCCCTC AGGCTC CTCTGC CTTCTG CTTGGG CTTCAG GGCTGC CTGGCT |
| 61 | GCAGTC TTCGTA ACCCAG GAGGAA GCCCAC GGCGTC CTGCAC CGGCGC CGGCGC GCCAAC |
| 121 | GCGTTC CTGGAG GAGCTG CGGCCG GGCTCC CTGGAG AGGGAG TGCAAG GAGGAG CAGTGC |
| 181 | TCCTTC GAGGAG GCCCGG GAGATC TTCAAG GACGCG GAGAGG ACGAAG CTGTTC TGGATT |
| 241 | TCTTAC AGTGAT GGGGAC CAGTGT GCCTCA AGTCCA TGCCAG AATGGG GGCTCC TGCAAG |
| 301 | GACCAG CTCCAG TCCTAT ATCTGC TTCTGC CTCCCT GCCTTC GAGGGC CGGAAC TGTGAG |
| 361 | ACGCAC AAGGAT GACCAG CTGATC TGTGTG AACGAG AACGGC GGCTGT GAGCAG TACTGC |
| 421 | AGTGAC CACACG GGCACC AAGCGC TCCTGT CGGTGC CACGAG GGGTAC TCTCTG CTGGCA |
| 481 | GACGGG GTGTCC TGCACA CCCACA GTTGAA TATCCA TGTGGA AAAATA CCTATT CTAGAA |
| 541 | AAAAGA AATGCC AGCAAA CCCCAA GGCCGA GGTGGC GGTGGC TCCGGC GGAGGT GGGTCC |
| 601 | GGTGGC GGCGGA TCAGGT GGGGGT GGATCA GGCGGT GGAGGT TCCGGT GGCGGG GGATCC |
| 661 | CAGGTG AAACTG CTCGAG TCTGGG GGAGGC GTGGTC CAGCCT GGGAGG TCCCTG AGACTC |
| 721 | TCCTGT GCAGCC TCTGGA TTCACC TTCAGT AGCTAT GCTATG CACTGG GTCCGC CAGGCT |
| 781 | CCAGGC AAGGGG CTGGAG TGGGTG GCAGTT ATATCA TATGAT GGAAGC AATAAA TACTAC |
| 841 | GCAGAC TCCGTG AAGGGC CGATTC GCCATC TCCAGA GACAAT TCCAAG AACACG CTGTAT |
| 901 | CTGCAA ATGAAC AGCCTG AGAGCT GAGGAC ACGGCT GTGTAT TACTGT GCGAGA GCGCTG |
| 961 | GGGAGC TGGGGG GGTTGG GACCAC TACATG GACGTC TGGGGC AAAGGG ACCACG GTCACC |
| 1021 | GTCTCC TCAGGT GGCGGC GGATCA GGTGGG GGTGGA TCAGGT GGCGGT GGCTCC GGTGGC |
| 1081 | GGGGGA TCAGTG GTGACT CAGCCA CCCTCA GCGTCT GGGACC CCCGGG CAGAGG GTCACC |
| 1141 | ATCTCT TGTTCT GGAAGC AGCTCC AACATC GGAAGT AATACT GTAAAC TGGTAC CAGCAG |
| 1201 | CTCCCA GGAACG GCCCCC AAACTC CTCATC TATAGT AATAAT CAGCGG CCCTCA GGGGTC |
| 1261 | CCTGAC CGATTC TCTGGC TCCAAG TCTGGC ACCTCA GCCTCC CTGGCC ATCAGT GGGCTC |
| 1321 | CAGTCT GAGGAT GAGGCT GATTAT TACTGT GCAGCA TGGGAT GACAGC CTGAAT GGTTGG |
| 1381 | GTGTTC GGCGGA GGGACC AAGCTG ACCGTC CTAGGT CAGCCC GGTGGC GGTGGC TCCGGC |
| 1441 | GGAGGT GGGTCC GGTGGC GGCGGA TCAGGT GGGGGT GGATCA GGCGGT GGAGGT TCCGGT |
| 1501 | GGCGGG GGAAGG AAGAGG AGGAAG AGGATT GTGGGG GGCAAG GTGTGC CCCAAA GGGGAG |
| 1561 | TGTCCA TGGCAG GTCCTG TTGTTG GTGAAT GGAGCT CAGTTG TGTGGG GGGACC CTGATC |
| 1621 | AACACC ATCTGG GTGGTC TCCGCG GCCCAC TGTTTC GACAAA ATCAAG AACTGG AGGAAC |
| 1681 | CTGATC GCGGTG CTGGGC GAGCAC GACCTC AGCGAG CACGAC GGGGAT GAGCAG AGCCGG |
| 1741 | CGGGTG GCGCAG GTCATC ATCCCC AGCACG TACGTC CCGGGC ACCACC AACCAC GACATC |
| 1801 | GCGCTG CTCCGC CTGCAC CAGCCC GTGGTC CTCACT GACCAT GTGGTG CCCCTC TGCCTG |
| 1861 | CCCGAA CGGACG TTCTCT GAGAGG ACGCTG GCCTTC GTGCGC TTCTCA TTGGTC AGCGGC |
| 1921 | TGGGGC CAGCTG CTGGAC CGTGGC GCCACG GCCCTG GAGCTC ATGGTC CTCAAC GTGCCC |
| 1981 | CGGCTG ATGACC CAGGAC TGCCTG CAGCAG TCACGG AAGGTG GGAGAC TCCCCA AATATC |
| 2041 | ACGGAG TACATG TTCTGT GCCGGC TACTCG GATGGC AGCAAG GACTCC TGCAAG GGGGAC |
| 2101 | AGTGGA GGCCCA CATGCC ACCCAC TACCGG GGCACG TGGTAC CTGACG GGCATC GTCAGC |
| 2161 | TGGGGC CAGGGC TGCGCA ACCGTG GGCCAC TTTGGG GTGTAC ACCAGG GTGTCC CAGTAC |
| 2221 | ATCGAG TGGCTG CAAAAG CTCATG CGCTCA GAGCCA CGCCCA GGAGTC CTCCTG CGAGCC |
| 2281 | CCATTT CCCGGG TCTCCA GGTACC TCAGAG TCTGCT ACCCCC GAGTCA GGGCCA GGATCA |
| 2341 | GAGCCA GCCACC TCCGGG TCTGAG ACACCC GGGACT TCCGAG AGTGCC ACCCCT GAGTCC |
| 2401 | GGACCC GGGTCC GAGCCC GCCACT TCCGGC TCCGAA ACTCCC GGCACA AGCGAG AGCGCT |
| 2461 | ACCCCA GAGTCA GGACCA GGAACA TCTACA GAGCCC TCTGAA GGCTCC GCTCCA GGGTCC |
| 2521 | CCAGCC GGCAGT CCCACT AGCACC GAGGAG GGAACC TCTGAA AGCGCC ACACCC GAATCA |
| 2581 | GGGCCA GGGTCT GAGCCT GCTACC AGCGGC AGCGAG ACACCA GGCACC TCTGAG TCCGCC |
| 2641 | ACACCA GAGTCC GGACCC GGATCT CCCGCT GGGAGC CCCACC TCCACT GAGGAG GGATCT |
| 2701 | CCTGCT GGCTCT CCAACA TCTACT GAGGAA GGTACC TCAACC GAGCCA TCCGAG GGATCA |
| 2761 | GCTCCC GGCACC TCAGAG TCGGCA ACCCCG GAGTCT GGACCC GGAACT TCCGAA AGTGCC |
| 2821 | ACACCA GAGTCC GGTCCC GGGACT TCAGAA TCAGCA ACACCC GAGTCC GGCCCT GGGTCT |
| 2881 | GAACCC GCCACA AGTGGT AGTGAG ACACCA GGATCA GAACCT GCTACC TCAGGG TCAGAG |
| 2941 | ACACCC GGATCT CCGGCA GGCTCA CCAACC TCCACT GAGGAG GGCACC AGCACA GAACCA |
| 3001 | AGCGAG GGCTCC GCACCC GGAACA AGCACT GAACCC AGTGAG GGTTCA GCACCC GGCTCT |
| 3061 | GAGCCG GCCACA AGTGGC AGTGAG ACACCC GGCACT TCAGAG AGTGCC ACCCCC GAGAGT |
| 3121 | GGCCCA GGCACT AGTACC GAGCCC TCTGAA GGCAGT GCGCCA |

### SEQ ID NO:190

>DNA sequence for FVII-178

| | |
|---|---|
| 1 | ATGGTC TCCCAG GCCCTC AGGCTC CTCTGC CTTCTG CTTGGG CTTCAG GGCTGC CTGGCT |
| 61 | GCAGTC TTCGTA ACCCAG GAGGAA GCCCAC GGCGTC CTGCAC CGGCGC CGGCGC GCCAAC |
| 121 | GCGTTC CTGGAG GAGCTG CGGCCG GGCTCC CTGGAG AGGGAG TGCAAG GAGGAG CAGTGC |
| 181 | TCCTTC GAGGAG GCCCGG GAGATC TTCAAG GACGCG GAGAGG ACGAAG CTGTTC TGGATT |
| 241 | TCTTAC AGTGAT GGGGAC CAGTGT GCCTCA AGTCCA TGCCAG AATGGG GGCTCC TGCAAG |
| 301 | GACCAG CTCCAG TCCTAT ATCTGC TTCTGC CTCCCT GCCTTC GAGGGC CGGAAC TGTGAG |
| 361 | ACGCAC AAGGAT GACCAG CTGATC TGTGTG AACGAG AACGGC GGCTGT GAGCAG TACTGC |
| 421 | AGTGAC CACACG GGCACC AAGCGC TCCTGT CGGTGC CACGAG GGGTAC TCTCTG CTGGCA |
| 481 | GACGGG GTGTCC TGCACA CCCACA GTTGAA TATCCA TGTGGA AAAATA CCTATT CTAGAA |
| 541 | AAAAGA AATGCC AGCAAA CCCCAA GGCCGA ATTGTG GGGGGC AAGGTG TGCCCC AAAGGG |
| 601 | GAGTGT CCATGG CAGGTC CTGTTG TTGGTG AATGGA GCTCAG TTGTGT GGGGGG ACCCTG |
| 661 | ATCAAC ACCATC TGGGTG GTCTCC GCGGCC CACTGT TTCGAC AAAATC AAGAAC TGGAGG |
| 721 | AACCTG ATCGCG GTGCTG GGCGAG CACGAC CTCAGC GAGCAC GACGGG GATGAG CAGAGC |
| 781 | CGGCGG GTGGCG CAGGTC ATCATC CCCAGC ACGTAC GTCCCG GGCACC ACCAAC CACGAC |
| 841 | ATCGCG CTGCTC CGCCTG CACCAG CCCGTG GTCCTC ACTGAC CATGTG GTGCCC CTCTGC |
| 901 | CTGCCC GAACGG ACGTTC TCTGAG AGGACG CTGGCC TTCGTG CGCTTC TCATTG GTCAGC |
| 961 | GGCTGG GGCCAG CTGCTG GACCGT GGCGCC ACGGCC CTGGAG CTCATG GTCCTC AACGTG |
| 1021 | CCCCGG CTGATG ACCCAG GACTGC CTGCAG CAGTCA CGGAAG GTGGGA GACTCC CCAAAT |
| 1081 | ATCACG GAGTAC ATGTTC TGTGCC GGCTAC TCGGAT GGCAGC AAGGAC TCCTGC AAGGGG |
| 1141 | GACAGT GGAGGC CCACAT GCCACC CACTAC CGGGGC ACGTGG TACCTG ACGGGC ATCGTC |
| 1201 | AGCTGG GGCCAG GGCTGC GCAACC GTGGGC CACTTT GGGGTG TACACC AGGGTG TCCCAG |
| 1261 | TACATC GAGTGG CTGCAA AAGCTC ATGCGC TCAGAG CCACGC CCAGGA GTCCTC CTGCGA |
| 1321 | GCCCCA TTTCCC GGGTCT CCAGGT ACCTCA GAGTCT GCTACC CCCGAG TCAGGG CCAGGA |
| 1381 | TCAGAG CCAGCC ACCTCC GGGTCT GAGACA CCCGGG ACTTCC GAGAGT GCCACC CCTGAG |
| 1441 | TCCGGA CCCGGG TCCGAG CCCGCC ACTTCC GGCTCC GAAACT CCCGGC ACAAGC GAGAGC |
| 1501 | GCTACC CCAGAG TCAGGA CCAGGA ACATCT ACAGAG CCCTCT GAAGGC TCCGCT CCAGGG |
| 1561 | TCCCCA GCCGGC AGTCCC ACTAGC ACCGAG GAGGGA ACCTCT GAAAGC GCCACA CCCGAA |
| 1621 | TCAGGG CCAGGG TCTGAG CCTGCT ACCAGC GGCAGC GAGACA CCAGGC ACCTCT GAGTCC |
| 1681 | GCCACA CCAGAG TCCGGA CCCGGA TCTCCC GCTGGG AGCCCC ACCTCC ACTGAG GAGGGA |
| 1741 | TCTCCT GCTGGC TCTCCA ACATCT ACTGAG GAAGGT ACCTCA ACCGAG CCATCC GAGGGA |
| 1801 | TCAGCT CCCGGC ACCTCA GAGTCG GCAACC CCGGAG TCTGGA CCCGGA ACTTCC GAAAGT |
| 1861 | GCCACA CCAGAG TCCGGT CCCGGG ACTTCA GAATCA GCAACA CCCGAG TCCGGC CCTGGG |
| 1921 | TCTGAA CCCGCC ACAAGT GGTAGT GAGACA CCAGGA TCAGAA CCTGCT ACCTCA GGGTCA |
| 1981 | GAGACA CCCGGA TCTCCG GCAGGC TCACCA ACCTCC ACTGAG GAGGGC ACCAGC ACAGAA |
| 2041 | CCAAGC GAGGGC TCCGCA CCCGGA ACAAGC ACTGAA CCCAGT GAGGGT TCAGCA CCCGGC |
| 2101 | TCTGAG CCGGCC ACAAGT GGCAGT GAGACA CCCGGC ACTTCA GAGAGT GCCACC CCCGAG |
| 2161 | AGTGGC CCAGGC ACTAGT ACCGAG CCCTCT GAAGGC AGTGCG CCAGGT TCGTCT TCAGGT |
| 2221 | GGCGGT GGCTCC GGCGGA GGTGGG TCCGGT GGCGGC GGATCA GGTGGG GGTGGA TCAGGC |
| 2281 | GGTGGA GGTTCC GGTGGC GGGGGA TCCCAG GTGAAA CTGCTC GAGTCT GGGGGA GGCGTG |
| 2341 | GTCCAG CCTGGG AGGTCC CTGAGA CTCTCC TGTGCA GCCTCT GGATTC ACCTTC AGTAGC |
| 2401 | TATGCT ATGCAC TGGGTC CGCCAG GCTCCA GGCAAG GGGCTG GAGTGG GTGGCA GTTATA |
| 2461 | TCATAT GATGGA AGCAAT AAATAC TACGCA GACTCC GTGAAG GGCCGA TTCGCC ATCTCC |
| 2521 | AGAGAC AATTCC AAGAAC ACGCTG TATCTG CAAATG AACAGC CTGAGA GCTGAG GACACG |
| 2581 | GCTGTG TATTAC TGTGCG AGAGCG CTGGGG AGCTGG GGGGGT TGGGAC CACTAC ATGGAC |
| 2641 | GTCTGG GGCAAA GGGACC ACGGTC ACCGTC TCCTCA GGTGGC GGCGGA TCAGGT GGGGGT |
| 2701 | GGATCA GGTGGC GGTGGC TCCGGT GGCGGG GGATCA GTGGTG ACTCAG CCACCC TCAGCG |
| 2761 | TCTGGG ACCCCC GGGCAG AGGGTC ACCATC TCTTGT TCTGGA AGCAGC TCCAAC ATCGGA |
| 2821 | AGTAAT ACTGTA AACTGG TACCAG CAGCTC CCAGGA ACGGCC CCCAAA CTCCTC ATCTAT |
| 2881 | AGTAAT AATCAG CGGCCC TCAGGG GTCCCT GACCGA TTCTCT GGCTCC AAGTCT GGCACC |
| 2941 | TCAGCC TCCCTG GCCATC AGTGGG CTCCAG TCTGAG GATGAG GCTGAT TATTAC TGTGCA |
| 3001 | GCATGG GATGAC AGCCTG AATGGT TGGGTG TTCGGC GGAGGG ACCAAG CTGACC GTCCTA |
| 3061 | GGTCAG CCC |

### SEQ ID NO:191

>DNA sequence for FVII-179

| | |
|---|---|
| 1 | GCCGCC ACCATG GTCTCC CAGGCC CTCAGG CTCCTC TGCCTT CTGCTT GGGCTT CAGGGC |
| 61 | TGCCTG GCTGCA GTCTTC GTAACC CAGGAG GAAGCC CACGGC GTCCTG CACCGG CGCCGG |
| 121 | CGCGCC AACGCG TTCCTG GAGGAG CTGCGG CCGGGC TCCCTG GAGAGG GAGTGC AAGGAG |
| 181 | GAGCAG TGCTCC TTCGAG GAGGCC CGGGAG ATCTTC AAGGAC GCGGAG AGGACG AAGCTG |
| 241 | TTCTGG ATTTCT TACAGT GATGGG GACCAG TGTGCC TCAAGT CCATGC CAGAAT GGGGGC |
| 301 | TCCTGC AAGGAC CAGCTC CAGTCC TATATC TGCTTC TGCCTC CCTGCC TTCGAG GGCCGG |
| 361 | AACTGT GAGACG CACAAG GATGAC CAGCTG ATCTGT GTGAAC GAGAAC GGCGGC TGTGAG |
| 421 | CAGTAC TGCAGT GACCAC ACGGGC ACCAAG CGCTCC TGTCGG TGCCAC GAGGGG TACTCT |
| 481 | CTGCTG GCAGAC GGGGTG TCCTGC ACACCC ACAGTT GAATAT CCATGT GGAAAA ATACCT |
| 541 | ATTCTA GAAAAA AGAAAT GCCAGC AAACCC CAAGGT GGCGGC GGATCA GGTGGG GGTGGA |
| 601 | TCAGGC GGTGGA GGTTCC GGTGGC GGGGGA TCCGGC GGTGGA GGTTCC GGTGGG GGTGGA |
| 661 | TCAGGC TCGAGT GGTACC TCAGAG TCTGCT ACCCCC GAGTCA GGGCCA GGATCA GAGCCA |
| 721 | GCCACC TCCGGG TCTGAG ACACCC GGGACT TCCGAG AGTGCC ACCCCT GAGTCC GGACCC |
| 781 | GGGTCC GAGCCC GCCACT TCCGGC TCCGAA ACTCCC GGCACA AGCGAG AGCGCT ACCCCA |
| 841 | GAGTCA GGACCA GGAACA TCTACA GAGCCC TCTGAA GGCTCC GCTCCA GGGTCC CCAGCC |
| 901 | GGCAGT CCCACT AGCACC GAGGAG GGAACC TCTGAA AGCGCC ACACCC GAATCA GGGCCA |
| 961 | GGGTCT GAGCCT GCTACC AGCGGC AGCGAG ACACCA GGCACC TCTGAG TCCGCC ACACCA |
| 1021 | GAGTCC GGACCC GGATCT CCCGCT GGGAGC CCCACC TCCACT GAGGAG GGATCT CCTGCT |
| 1081 | GGCTCT CCAACA TCTACT GAGGAA GGTACC TCAACC GAGCCA TCCGAG GGATCA GCTCCC |
| 1141 | GGCACC TCAGAG TCGGCA ACCCCG GAGTCT GGACCC GGAACT TCCGAA AGTGCC ACACCA |
| 1201 | GAGTCC GGTCCC GGGACT TCAGAA TCAGCA ACACCC GAGTCC GGCCCT GGGTCT GAACCC |
| 1261 | GCCACA AGTGGT AGTGAG ACACCA GGATCA GAACCT GCTACC TCAGGG TCAGAG ACACCC |
| 1321 | GGATCT CCGGCA GGCTCA CCAACC TCCACT GAGGAG GGCACC AGCACA GAACCA AGCGAG |
| 1381 | GGCTCC GCACCC GGAACA AGCACT GAACCC AGTGAG GGTTCA GCACCC GGCTCT GAGCCG |
| 1441 | GCCACA AGTGGC AGTGAG ACACCC GGCACT TCAGAG AGTGCC ACCCCC GAGAGT GGCCCA |
| 1501 | GGCACT AGTACC GAGCCC TCTGAA GGCAGT GCGCCA ACCGGT GGTGGC GGTGGC TCCGGC |
| 1561 | GGAGGT GGGTCC GGTGGC GGCGGA TCAGGT GGGGGT GGATCA GGCGGT GGAGGT TCCGGT |
| 1621 | GGCGGG GGATCA AGAAAG AGAAGG AAAAGA ATTGTG GGGGGC AAGGTG TGCCCC AAAGGG |
| 1681 | GAGTGT CCATGG CAGGTC CTGTTG TTGGTG AATGGA GCTCAG TTGTGT GGGGGG ACCCTG |
| 1741 | ATCAAC ACCATC TGGGTG GTGTCC GCGGCC CACTGT TTCGAC AAAATC AAGAAC TGGAGG |
| 1801 | AACCTG ATCGCG GTGCTG GGCGAG CACGAC CTCAGC GAGCAC GACGGG GATGAG CAGAGC |
| 1861 | CGGCGG GTGGCG CAGGTC ATCATC CCCAGC ACGTAC GTCCCG GGCACC ACCAAC CACGAC |
| 1921 | ATCGCG CTGCTC CGCCTG CACCAG CCCGTG GTCCTC ACTGAC CATGTG GTGCCC CTCTGC |
| 1981 | CTGCCC GAACGG ACGTTC TCTGAG AGGACG CTGGCC TTCGTG CGCTTC TCATTG GTCAGC |
| 2041 | GGCTGG GGCCAG CTGCTG GACCGT GGCGCC ACGGCC CTGGAG CTCATG GTCCTC AACGTG |
| 2101 | CCCCGG CTGATG ACCCAG GACTGC CTGCAG CAGTCA CGGAAG GTGGGA GACTCC CCAAAT |
| 2161 | ATCACG GAGTAC ATGTTC TGTGCC GGCTAC TCGGAT GGCAGC AAGGAC TCCTGC AAGGGG |
| 2221 | GACAGT GGAGGC CCACAT GCCACC CACTAC CGGGGC ACGTGG TACCTG ACGGGC ATCGTC |
| 2281 | AGCTGG GGCCAG GGCTGC GCAACC GTGGGC CACTTT GGGGTG TACACC AGGGTC TCCCAG |
| 2341 | TACATC GAGTGG CTGCAA AAGCTC ATGCGC TCAGAG CCACGC CCAGGA GTCCTC CTGCGA |
| 2401 | GCCCCA TTTCCC GGTGGC GGTGGC TCCGGC GGAGGT GGGTCC GGTGGC GGCGGA TCAGGT |
| 2461 | GGGGGT GGATCA GGCGGT GGAGGT TCCGGT GGCGGG GGATCC CAGGTG AAACTG CTCGAG |
| 2521 | TCTGGG GGAGGC GTGGTC CAGCCT GGGAGG TCCCTG AGACTC TCCTGT GCAGCC TCTGGA |
| 2581 | TTCACC TTCAGT AGCTAT GCTATG CACTGG GTCCGC CAGGCT CCAGGC AAGGGG CTGGAG |
| 2641 | TGGGTG GCAGTT ATATCA TATGAT GGAAGC AATAAA TACTAC GCAGAC TCCGTG AAGGGC |
| 2701 | CGATTC GCCATC TCCAGA GACAAT TCCAAG AACACG CTGTAT CTGCAA ATGAAC AGCCTG |
| 2761 | AGAGCT GAGGAC ACGGCT GTGTAT TACTGT GCGAGA GCGCTG GGGAGC TGGGGG GGTTGG |
| 2821 | GACCAC TACATG GACGTC TGGGGC AAAGGG ACCACG GTCACC GTCTCC TCAGGT GGCGGC |
| 2881 | GGATCA GGTGGG GGTGGA TCAGGT GGCGGT GGCTCC GGTGGC GGGGGA TCAGTG GTGACT |
| 2941 | CAGCCA CCCTCA GCGTCT GGGACC CCCGGG CAGAGG GTCACC ATCTCT TGTTCT GGAAGC |
| 3001 | AGCTCC AACATC GGAAGT AATACT GTAAAC TGGTAC CAGCAG CTCCCA GGAACG GCCCCC |
| 3061 | AAACTC CTCATC TATAGT AATAAT CAGCGG CCCTCA GGGGTC CCTGAC CGATTC TCTGGC |
| 3121 | TCCAAG TCTGGC ACCTCA GCCTCC CTGGCC ATCAGT GGGCTC CAGTCT GAGGAT GAGGCT |
| 3181 | GATTAT TACTGT GCAGCA TGGGAT GACAGC CTGAAT GGTTGG GTGTTC GGCGGA GGGACC |
| 3241 | AAGCTG ACCGTC CTAGGT CAGCCC |

### SEQ ID NO:192

>DNA sequence for FVII-200

| | |
|---|---|
| 1 | ATGGTC TCCCAG GCCCTC AGGCTC CTCTGC CTTCTG CTTGGG CTTCAG GGCTGC CTGGCT |
| 61 | GCAGTC TTCGTA ACCCAG GAGGAA GCCCAC GGCGTC CTGCAC CGGCGC CGGCGC GCCAAC |
| 121 | GCGTTC CTGGAG GAGCTG CGGCCG GGCTCC CTGGAG AGGGAG TGCAAG GAGGAG CAGTGC |
| 181 | TCCTTC GAGGAG GCCCGG GAGATC TTCAAG GACGCG GAGAGG ACGAAG CTGTTC TGGATT |
| 241 | TCTTAC AGTGAT GGGGAC CAGTGT GCCTCA AGTCCA TGCCAG AATGGG GGCTCC TGCAAG |
| 301 | GACCAG CTCCAG TCCTAT ATCTGC TTCTGC CTCCCT GCCTTC GAGGGC CGGAAC TGTGAG |
| 361 | ACGCAC AAGGAT GACCAG CTGATC TGTGTG AACGAG AACGGC GGCTGT GAGCAG TACTGC |
| 421 | AGTGAC CACACG GGCACC AAGCGC TCCTGT CGGTGC CACGAG GGGTAC TCTCTG CTGGCA |
| 481 | GACGGG GTGTCC TGCACA CCCACA GTTGAA TATCCA TGTGGA AAAATA CCTATT CTAGAA |
| 541 | AAAAGA AATGCC AGCAAA CCCCAA GGCCGA ATTGTG GGGGGC AAGGTG TGCCCC AAAGGG |
| 601 | GAGTGT CCATGG CAGGTC CTGTTG TTGGTG AATGGA GCTCAG TTGTGT GGGGGG ACCCTG |
| 661 | ATCAAC ACCATC TGGGTG GTCTCC GCGGCC CACTGT TTCGAC AAAATC AAGAAC TGGAGG |
| 721 | AACCTG ATCGCG GTGCTG GGCGAG CACGAC CTCAGC GAGCAC GACGGG GATGAG CAGAGC |
| 781 | CGGCGG GTGGCG CAGGTC ATCATC CCCAGC ACGTAC GTCCCG GGCACC ACCAAC CACGAC |
| 841 | ATCGCG CTGCTC CGCCTG CACCAG CCCGTG GTCCTC ACTGAC CATGTG GTGCCC CTCTGC |
| 901 | CTGCCC GAACGG ACGTTC TCTGAG AGGACG CTGGCC TTCGTG CGCTTC TCATTG GTCAGC |
| 961 | GGCTGG GGCCAG CTGCTG GACCGT GGCGCC ACGGCC CTGGAG CTCATG GTCCTC AACGTG |
| 1021 | CCCCGG CTGATG ACCCAG GACTGC CTGCAG CAGTCA CGGAAG GTGGGA GACTCC CCAAAT |
| 1081 | ATCACG GAGTAC ATGTTC TGTGCC GGCTAC TCGGAT GGCAGC AAGGAC TCCTGC AAGGGG |
| 1141 | GACAGT GGAGGC CCACAT GCCACC CACTAC CGGGGC ACGTGG TACCTG ACGGGC ATCGTC |
| 1201 | AGCTGG GGCCAG GGCTGC GCAACC GTGGGC CACTTT GGGGTG TACACC AGGGTG TCCCAG |
| 1261 | TACATC GAGTGG CTGCAA AAGCTC ATGCGC TCAGAG CCACGC CCAGGA GTCCTC CTGCGA |
| 1321 | GCCCCA TTTCCC GGGTCT CCAGGT ACCTCA GAGTCT GCTACC CCCGAG TCAGGG CCAGGA |
| 1381 | TCAGAG CCAGCC ACCTCC GGGTCT GAGACA CCCGGG ACTTCC GAGAGT GCCACC CCTGAG |
| 1441 | TCCGGA CCCGGG TCCGAG CCCGCC ACTTCC GGCTCC GAAACT CCCGGC ACAAGC GAGAGC |
| 1501 | GCTACC CCAGAG TCAGGA CCAGGA ACATCT ACAGAG CCCTCT GAAGGC TCCGCT CCAGGG |
| 1561 | TCCCCA GCCGGC AGTCCC ACTAGC ACCGAG GAGGGA ACCTCT GAAAGC GCCACA CCCGAA |
| 1621 | TCAGGG CCAGGG TCTGAG CCTGCT ACCAGC GGCAGC GAGACA CCAGGC ACCTCT GAGTCC |
| 1681 | GCCACA CCAGAG TCCGGA CCCGGA TCTCCC GCTGGG AGCCCC ACCTCC ACTGAG GAGGGA |
| 1741 | TCTCCT GCTGGC TCTCCA ACATCT ACTGAG GAAGGT ACCTCA ACCGAG CCATCC GAGGGA |
| 1801 | TCAGCT CCCGGC ACCTCA GAGTCG GCAACC CCGGAG TCTGGA CCCGGA ACTTCC GAAAGT |
| 1861 | GCCACA CCAGAG TCCGGT CCCGGG ACTTCA GAATCA GCAACA CCCGAG TCCGGC CCTGGG |
| 1921 | TCTGAA CCCGCC ACAAGT GGTAGT GAGACA CCAGGA TCAGAA CCTGCT ACCTCA GGGTCA |
| 1981 | GAGACA CCCGGA TCTCCG GCAGGC TCACCA ACCTCC ACTGAG GAGGGC ACCAGC ACAGAA |
| 2041 | CCAAGC GAGGGC TCCGCA CCCGGA ACAAGC ACTGAA CCCAGT GAGGGT TCAGCA CCCGGC |
| 2101 | TCTGAG CCGGCC ACAAGT GGCAGT GAGACA CCCGGC ACTTCA GAGAGT GCCACC CCCGAG |
| 2161 | AGTGGC CCAGGC ACTAGT ACCGAG CCCTCT GAAGGC AGTGCG CCAGGT TCGTCT TCAGGT |
| 2221 | GGCGGT GGCTCC GGCGGA GGTGGG TCCGGT GGCGGC GGATCA GGTGGG GGTGGA TCAGGC |
| 2281 | GGTGGA GGTTCC GGTGGC GGGGGA TCCGAA AATGTG CTCACC CAGTCT CCAGCA ATCATG |
| 2341 | TCTGCA TCTCTA GGGGAG AAGGTC ACCATG AGCTGC AGGGCC AGCTCA AGTGTA AATTAC |
| 2401 | ATGTAC TGGTAC CAGCAG AAGTCA GATGCC TCCCCC AAACTA TGGATT TATTAC ACATCC |
| 2461 | AACCTG GCTCCT GGAGTC CCAGCT CGCTTC AGTGGC AGTGGG TCTGGG AACTCT TATTCT |
| 2521 | CTCACA ATCAGC AGCATG GAGGGT GAAGAT GCTGCC ACTTAT TACTGC CAGCAG TTTAGT |
| 2581 | AGTTCC CCGTGG ACGTTC GGTGGA GGCACC AAGCTG GAAATC AAACGC GGTGGC GGCGGA |
| 2641 | TCAGGT GGAGGT GGATCA GGTGGC GGTGGC AGTGGT GGCGGT GGATCA GAAGTG AAGCTG |
| 2701 | GTGGAG TCTGGG GGAGGC TTAGTG AAGCCT GGAGGG TCCCTG AAACTC TCCTGT GCAGCC |
| 2761 | TCTGGA TTCACT TTCAGT GCCTAT GCCATG TCTTGG GTTCGC CAGACT CCAGAG AAGAGG |
| 2821 | CTGGAG TGGGTC GCATCC ATTAGT AGTGGT GGTACC ACCTAC TATCCA GACAGT GTGAAA |
| 2881 | CGCCGA TTCACC ATCTCC AGAGAT AATGCC AGGAAC ATCCTG TACCTG CAAATG AGCAGT |
| 2941 | CTGAGG TCTGAG GACACG GCCATG TATTAC TGTACA AGAGGC GGGGAC TACGGC TACGCT |
| 3001 | CTGGAC TACTGG GGTCAA GGAACC TCAGTC ACCGTC TCCTCA |

### SEQ ID NO:193

>DNA sequence for FVII-211

| | |
|---|---|
| 1 | ATGGTC TCCCAG GCCCTC AGGCTC CTCTGC CTTCTG CTTGGG CTTCAG GGCTGC CTGGCT |
| 61 | GCAGTC TTCGTA ACCCAG GAGGAA GCCCAC GGCGTC CTGCAC CGGCGC CGGCGC GCCAAC |
| 121 | GCGTTC CTGGAG GAGCTG CGGCCG GGCTCC CTGGAG AGGGAG TGCAAG GAGGAG CAGTGC |
| 181 | TCCTTC GAGGAG GCCCGG GAGATC TTCAAG GACGCG GAGAGG ACGAAG CTGTTC TGGATT |
| 241 | TCTTAC AGTGAT GGGGAC CAGTGT GCCTCA AGTCCA TGCCAG AATGGG GGCTCC TGCAAG |
| 301 | GACCAG CTCCAG TCCTAT ATCTGC TTCTGC CTCCCT GCCTTC GAGGGC CGGAAC TGTGAG |
| 361 | ACGCAC AAGGAT GACCAG CTGATC TGTGTG AACGAG AACGGC GGCTGT GAGCAG TACTGC |
| 421 | AGTGAC CACACG GGCACC AAGCGC TCCTGT CGGTGC CACGAG GGGTAC TCTCTG CTGGCA |
| 481 | GACGGG GTGTCC TGCACA CCCACA GTTGAA TATCCA TGTGGA AAAATA CCTATT CTAGAA |
| 541 | AAAAGA AATGCC AGCAAA CCCCAA GGTGGC GGCGGA TCAGGT GGGGGT GGATCA GGCGGT |
| 601 | GGAGGT TCCGGT GGCGGG GGATCC GGCGGT GGAGGT TCCGGT GGGGGT GGATCA GGCTCG |
| 661 | AGTGGT ACCTCA GAGTCT GCTACC CCCGAG TCAGGG CCAGGA TCAGAG CCAGCC ACCTCC |
| 721 | GGGTCT GAGACA CCCGGG ACTTCC GAGAGT GCCACC CCTGAG TCCGGA CCCGGG TCCGAG |
| 781 | CCCGCC ACTTCC GGCTCC GAAACT CCCGGC ACAAGC GAGAGC GCTACC CCAGAG TCAGGA |
| 841 | CCAGGA ACATCT ACAGAG CCCTCT GAAGGC TCCGCT CCAGGG TCCCCA GCCGGC AGTCCC |
| 901 | ACTAGC ACCGAG GAGGGA ACCTCT GAAAGC GCCACA CCCGAA TCAGGG CCAGGG TCTGAG |
| 961 | CCTGCT ACCAGC GGCAGC GAGACA CCAGGC ACCTCT GAGTCC GCCACA CCAGAG TCCGGA |
| 1021 | CCCGGA TCTCCC GCTGGG AGCCCC ACCTCC ACTGAG GAGGGA TCTCCT GCTGGC TCTCCA |
| 1081 | ACATCT ACTGAG GAAGGT ACCTCA ACCGAG CCATCC GAGGGA TCAGCT CCCGGC ACCTCA |
| 1141 | GAGTCG GCAACC CCGGAG TCTGGA CCCGGA ACTTCC GAAAGT GCCACA CCAGAG TCCGGT |
| 1201 | CCCGGG ACTTCA GAATCA GCAACA CCCGAG TCCGGC CCTGGG TCTGAA CCCGCC ACAAGT |
| 1261 | GGTAGT GAGACA CCAGGA TCAGAA CCTGCT ACCTCA GGGTCA GAGACA CCCGGA TCTCCG |
| 1321 | GCAGGC TCACCA ACCTCC ACTGAG GAGGGC ACCAGC ACAGAA CCAAGC GAGGGC TCCGCA |
| 1381 | CCCGGA ACAAGC ACTGAA CCCAGT GAGGGT TCAGCA CCCGGC TCTGAG CCGGCC ACAAGT |
| 1441 | GGCAGT GAGACA CCCGGC ACTTCA GAGAGT GCCACC CCCGAG AGTGGC CCAGGC ACTAGT |
| 1501 | ACCGAG CCCTCT GAAGGC AGTGCG CCAACC GGTGGT GGCGGT GGCTCC GGCGGA GGTGGG |
| 1561 | TCCGGT GGCGGC GGATCA GGTGGG GGTGGA TCAGGC GGTGGA GGTTCC GGTGGC GGGGGA |
| 1621 | TCAAGA AAGAGA AGGAAA AGAATT GTGGGG GGCAAG GTGTGC CCCAAA GGGGAG TGTCCA |
| 1681 | TGGCAG GTCCTG TTGTTG GTGAAT GGAGCT CAGTTG TGTGGG GGGACC CTGATC AACACC |
| 1741 | ATCTGG GTGGTG TCCGCG GCCCAC TGTTTC GACAAA ATCAAG AACTGG AGGAAC CTGATC |
| 1801 | GCGGTG CTGGGC GAGCAC GACCTC AGCGAG CACGAC GGGGAT GAGCAG AGCCGG CGGGTG |
| 1861 | GCGCAG GTCATC ATCCCC AGCACG TACGTC CCGGGC ACCACC AACCAC GACATC GCGCTG |
| 1921 | CTCCGC CTGCAC CAGCCC GTGGTC CTCACT GACCAT GTGGTG CCCCTC TGCCTG CCCGAA |
| 1981 | CGGACG TTCTCT GAGAGG ACGCTG GCCTTC GTGCGC TTCTCA TTGGTC AGCGGC TGGGGC |
| 2041 | CAGCTG CTGGAC CGTGGC GCCACG GCCCTG GAGCTC ATGGTC CTCAAC GTGCCC CGGCTG |
| 2101 | ATGACC CAGGAC TGCCTG CAGCAG TCACGG AAGGTG GGAGAC TCCCCA AATATC ACGGAG |
| 2161 | TACATG TTCTGT GCCGGC TACTCG GATGGC AGCAAG GACTCC TGCAAG GGGGAC AGTGGA |
| 2221 | GGCCCA CATGCC ACCCAC TACCGG GGCACG TGGTAC CTGACG GGCATC GTCAGC TGGGGC |
| 2281 | CAGGGC TGCGCA ACCGTG GGCCAC TTTGGG GTGTAC ACCAGG GTCTCC CAGTAC ATCGAG |
| 2341 | TGGCTG CAAAAG CTCATG CGCTCA GAGCCA CGCCCA GGAGTC CTCCTG CGAGCC CCATTT |
| 2401 | CCCGGT GGCGGT GGCTCC GGCGGA GGTGGG TCCGGT GGCGGC GGATCA GGTGGG GGTGGA |
| 2461 | TCAGGC GGTGGA GGTTCC GGTGGC GGGGGA TCCGAA AATGTG CTCACC CAGTCT CCAGCA |
| 2521 | ATCATG TCTGCA TCTCTA GGGGAG AAGGTC ACCATG AGCTGC AGGGCC AGCTCA AGTGTA |
| 2581 | AATTAC ATGTAC TGGTAC CAGCAG AAGTCA GATGCC TCCCCC AAACTA TGGATT TATTAC |
| 2641 | ACATCC AACCTG GCTCCT GGAGTC CCAGCT CGCTTC AGTGGC AGTGGG TCTGGG AACTCT |
| 2701 | TATTCT CTCACA ATCAGC AGCATG GAGGGT GAAGAT GCTGCC ACTTAT TACTGC CAGCAG |
| 2761 | TTTAGT AGTTCC CCGTGG ACGTTC GGTGGA GGCACC AAGCTG GAAATC AAACGC GGTGGC |
| 2821 | GGCGGA TCAGGT GGAGGT GGATCA GGTGGC GGTGGC AGTGGT GGCGGT GGATCA GAAGTG |
| 2881 | AAGCTG GTGGAG TCTGGG GGAGGC TTAGTG AAGCCT GGAGGG TCCCTG AAACTC TCCTGT |
| 2941 | GCAGCC TCTGGA TTCACT TTCAGT GCCTAT GCCATG TCTTGG GTTCGC CAGACT CCAGAG |
| 3001 | AAGAGG CTGGAG TGGGTC GCATCC ATTAGT AGTGGT GGTACC ACCTAC TATCCA GACAGT |
| 3061 | GTGAAA CGCCGA TTCACC ATCTCC AGAGAT AATGCC AGGAAC ATCCTG TACCTG CAAATG |
| 3121 | AGCAGT CTGAGG TCTGAG GACACG GCCATG TATTAC TGTACA AGAGGC GGGGAC TACGGC |
| 3181 | TACGCT CTGGAC TACTGG GGTCAA GGAACC TCAGTC ACCGTC TCCTCA |

### SEQ ID NO:269

FVII-227 DNA sequence

| | |
|---|---|
| 1 | ATGGTCTCCC AGGCCCTCAG GCTCCTCTGC CTTCTGCTTG GGCTTCAGGG CTGCCTGGCT |
| 61 | GCAGTCTTCG TAACCCAGGA GGAAGCCCAC GGCGTCCTGC ACCGGCGCCG GCGCGCCAAC |
| 121 | GCGTTCCTGG AGGAGCTACG GCCGGGCTCC CTGGAGAGGG AGTGCAAGGA GGAGCAGTGC |
| 181 | TCCTTCGAGG AGGCCCGGGA GATCTTCAAG GACGCGGAGA GGACGAAGCT GTTCTGGATT |
| 241 | TCTTACAGTG ATGGGGACCA GTGTGCCTCA AGTCCATGCC AGAATGGGGG CTCCTGCAAG |
| 301 | GACCAGCTCC AGTCCTATAT CTGCTTCTGC CTCCCTGCCT TCGAGGGCCG GAACTGTGAG |
| 361 | ACGCACAAGG ATGACCAGCT GATCTGTGTG AACGAGAACG GCGGCTGTGA GCAGTACTGC |
| 421 | AGTGACCACA CGGGCACCAA GCGCTCCTGT CGGTGCCACG AGGGGTACTC TCTGCTGGCA |
| 481 | GACGGGGTGT CCTGCACACC CACAGTTGAA TATCCATGTG GAAAAATACC TATTCTAGAA |
| 541 | AAAAGAAATG CCAGCAAACC CCAAGGTGGC GGCGGATCAG GTGGGGGTGG ATCAGGCGGT |
| 601 | GGAGGTTCCG GTGGCGGGGG ATCCGGCGGT GGAGGTTCCG GTGGGGGTGG ATCAGGCTCG |
| 661 | AGTAGTCCCG CTGGAAGCCC AACTAGCACC GAAGAGGGGA CCTCAGAGTC CGCCACCCCC |
| 721 | GAGTCCGGCC CTGGCTCTGA GCCTGCCACT AGCGGCTCCG AGACTCCTGG CACATCCGAA |
| 781 | AGCGCTACAC CCGAGAGTGG ACCCGGCACC TCTACCGAGC CCAGTGAGGG CTCCGCCCCT |
| 841 | GGAACAAGCA CCGAGCCCAG CGAAGGCAGC GCCCCAGGGA CCGGTGGTGG CGGTGGCTCC |
| 901 | GGCGGAGGTG GGTCCGGTGG CGGCGGATCA GGTGGGGGTG GATCAGGCGG TGGAGGTTCC |
| 961 | GGTGGCGGGG GATCAAGAAA GAGAAGGAAA AGAATTGTGG GGGGCAAGGT GTGCCCCAAA |
| 1021 | GGGGAGTGTC CATGGCAGGT CCTGTTGTTG GTGAATGGAG CTCAGTTGTG TGGGGGGACC |
| 1081 | CTGATCAACA CCATCTGGGT GGTGTCCGCG GCCCACTGTT TCGACAAAAT CAAGAACTGG |
| 1141 | AGGAACCTGA TCGCGGTGCT GGGCGAGCAC GACCTCAGCG AGCACGACGG GGATGAGCAG |
| 1201 | AGCCGGCGGG TGGCGCAGGT CATCATCCCC AGCACGTACG TCCCGGGCAC CACCAACCAC |
| 1261 | GACATCGCGC TGCTCCGCCT GCACCAGCCC GTGGTCCTCA CTGACCATGT GGTGCCCCTC |
| 1321 | TGCCTGCCCG AACGGACGTT CTCTGAGAGG ACGCTGGCCT TCGTGCGCTT CTCATTGGTC |
| 1381 | AGCGGCTGGG GCCAGCTGCT GGACCGTGGC GCCACGGCCC TGGAGCTCAT GGTCCTCAAC |
| 1441 | GTGCCCCGGC TGATGACCCA GGACTGCCTG CAGCAGTCAC GGAAGGTGGG AGACTCCCCA |
| 1501 | AATATCACGG AGTACATGTT CTGTGCCGGC TACTCGGATG GCAGCAAGGA CTCCTGCAAG |
| 1561 | GGGGACAGTG GAGGCCCACA TGCCACCCAC TACCGGGGCA CGTGGTACCT GACGGGCATC |
| 1621 | GTCAGCTGGG GCCAGGGCTG CGCAACCGTG GGCCACTTTG GGGTGTACAC CAGGGTCTCC |
| 1681 | CAGTACATCG AGTGGCTGCA AAAGCTCATG CGCTCAGAGC CACGCCCAGG AGTCCTCCTG |
| 1741 | CGAGCCCCAT TTCCCGGTGG CGGTGGCTCC GGCGGAGGTG GGTCCGGTGG CGGCGGATCA |
| 1801 | GGTGGGGGTG GATCAGGCGG TGGAGGTTCC GGTGGCGGGG GATCCGAAAA TGTGCTCACC |
| 1861 | CAGTCTCCAG CAATCATGTC TGCATCTCTA GGGGAGAAGG TCACCATGAG CTGCAGGGCC |
| 1921 | AGCTCAAGTG TAAATTACAT GTACTGGTAC CAGCAGAAGT CAGATGCCTC CCCCAAACTA |
| 1981 | TGGATTTATT ACACATCCAA CCTGGCTCCT GGAGTCCCAG CTCGCTTCAG TGGCAGTGGG |
| 2041 | TCTGGGAACT CTTATTCTCT CACAATCAGC AGCATGGAGG GTGAAGATGC TGCCACTTAT |
| 2101 | TACTGCCAGC AGTTTAGTAG TTCCCCGTGG ACGTTCGGTG GAGGCACCAA GCTGGAAATC |
| 2161 | AAACGCGGTG GCGGCGGATC AGGTGGAGGT GGATCAGGTG GCGGTGGCAG TGGTGGCGGT |
| 2221 | GGATCAGAAG TGAAGCTGGT GGAGTCTGGG GGAGGCTTAG TGAAGCCTGG AGGGTCCCTG |
| 2281 | AAACTCTCCT GTGCAGCCTC TGGATTCACT TTCAGTGCCT ATGCCATGTC TTGGGTTCGC |
| 2341 | CAGACTCCAG AGAAGAGGCT GGAGTGGGTC GCATCCATTA GTAGTGGTGG TACCACCTAC |
| 2401 | TATCCAGACA GTGTGAAACG CCGATTCACC ATCTCCAGAG ATAATGCCAG GAACATCCTG |
| 2461 | TACCTGCAAA TGAGCAGTCT GAGGTCTGAG GACACGGCCA TGTATTACTG TACAAGAGGC |
| 2521 | GGGGACTACG GCTACGCTCT GGACTACTGG GGTCAAGGAA CCTCAGTCAC CGTCTCCTCA |
| 2581 | GGTGGCGGAG GTTCCCATCA TCACCATCAC CATTGA |

### SEQ ID NO:270

FVII-228 DNA sequence

| | |
|---|---|
| 1 | ATGGTCTCCC AGGCCCTCAG GCTCCTCTGC CTTCTGCTTG GGCTTCAGGG CTGCCTGGCT |
| 61 | GCAGTCTTCG TAACCCAGGA GGAAGCCCAC GGCGTCCTGC ACCGGCGCCG GCGCGCCAAC |
| 121 | GCGTTCCTGG AGGAGCTACG GCCGGGCTCC CTGGAGAGGG AGTGCAAGGA GGAGCAGTGC |
| 181 | TCCTTCGAGG AGGCCCGGGA GATCTTCAAG GACGCGGAGA GGACGAAGCT GTTCTGGATT |
| 241 | TCTTACAGTG ATGGGGACCA GTGTGCCTCA AGTCCATGCC AGAATGGGGG CTCCTGCAAG |
| 301 | GACCAGCTCC AGTCCTATAT CTGCTTCTGC CTCCCTGCCT TCGAGGGCCG GAACTGTGAG |
| 361 | ACGCACAAGG ATGACCAGCT GATCTGTGTG AACGAGAACG GCGGCTGTGA GCAGTACTGC |
| 421 | AGTGACCACA CGGGCACCAA GCGCTCCTGT CGGTGCCACG AGGGGTACTC TCTGCTGGCA |
| 481 | GACGGGGTGT CCTGCACACC CACAGTTGAA TATCCATGTG GAAAAATACC TATTCTAGAA |
| 541 | AAAAGAAATG CCAGCAAACC CCAAGGTGGC GGCGGATCAG GTGGGGGTGG ATCAGGCGGT |
| 601 | GGAGGTTCCG GTGGCGGGGG ATCCGGCGGT GGAGGTTCCG GTGGGGGTGG ATCAGGCTCG |
| 661 | AGTGGTTCTC CAGCCGGGTC CCCAACTTCG ACCGAGGAAG GGACCTCCGA GTCAGCTACC |
| 721 | CCGGAGTCCG GTCCTGGCAC CTCCACCGAA CCATCGGAGG GCAGCGCCCC TGGGAGCCCT |
| 781 | GCCGGGAGCC CTACAAGCAC CGAAGAGGGC ACCAGTACAG AGCCAAGTGA GGGGAGCGCC |
| 841 | CCTGGTACTA GTACTGAACC ATCCGAGGGG TCAGCTCCAG GCACGAGTGA GTCCGCTACC |
| 901 | CCCGAGAGCG GACCGGGCTC AGAGCCCGCC ACGAGTGGCA GTGAAACTCC AGGCTCAGAA |
| 961 | CCCGCCACTA GTGGGTCAGA GACTCCAGGC AGCCCTGCCG GATCCCCTAC GTCCACCGAG |
| 1021 | GAGGGAACAT CTGAGTCCGC AACACCCGAA TCCGGTCCAG GCACCTCCAC GGAACCTAGT |
| 1081 | GAAGGCTCGG CACCAACCGG TGGTGGCGGT GGCTCCGGCG GAGGTGGGTC CGGTGGCGGC |
| 1141 | GGATCAGGTG GGGGTGGATC AGGCGGTGGA GGTTCCGGTG GCGGGGGATC AAGAAAGAGA |
| 1201 | AGGAAAAGAA TTGTGGGGGG CAAGGTGTGC CCCAAAGGGG AGTGTCCATG GCAGGTCCTG |
| 1261 | TTGTTGGTGA ATGGAGCTCA GTTGTGTGGG GGGACCCTGA TCAACACCAT CTGGGTGGTG |
| 1321 | TCCGCGGCCC ACTGTTTCGA CAAAATCAAG AACTGGAGGA ACCTGATCGC GGTGCTGGGC |
| 1381 | GAGCACGACC TCAGCGAGCA CGACGGGGAT GAGCAGAGCC GGCGGGTGGC GCAGGTCATC |
| 1441 | ATCCCCAGCA CGTACGTCCC GGGCACCACC AACCACGACA TCGCGCTGCT CCGCCTGCAC |
| 1501 | CAGCCCGTGG TCCTCACTGA CCATGTGGTG CCCCTCTGCC TGCCCGAACG GACGTTCTCT |
| 1561 | GAGAGGACGC TGGCCTTCGT GCGCTTCTCA TTGGTCAGCG GCTGGGGCCA GCTGCTGGAC |
| 1621 | CGTGGCGCCA CGGCCCTGGA GCTCATGGTC CTCAACGTGC CCCGGCTGAT GACCCAGGAC |
| 1681 | TGCCTGCAGC AGTCACGGAA GGTGGGAGAC TCCCCAAATA TCACGGAGTA CATGTTCTGT |
| 1741 | GCCGGCTACT CGGATGGCAG CAAGGACTCC TGCAAGGGGG ACAGTGGAGG CCCACATGCC |
| 1801 | ACCCACTACC GGGGCACGTG GTACCTGACG GGCATCGTCA GCTGGGGCCA GGGCTGCGCA |
| 1861 | ACCGTGGGCC ACTTTGGGGT GTACACCAGG GTCTCCCAGT ACATCGAGTG GCTGCAAAAG |
| 1921 | CTCATGCGCT CAGAGCCACG CCCAGGAGTC CTCCTGCGAG CCCCATTTCC CGGTGGCGGT |
| 1981 | GGCTCCGGCG GAGGTGGGTC CGGTGGCGGC GGATCAGGTG GGGGTGGATC AGGCGGTGGA |
| 2041 | GGTTCCGGTG GCGGGGGATC CGAAAATGTG CTCACCCAGT CTCCAGCAAT CATGTCTGCA |
| 2101 | TCTCTAGGGG AGAAGGTCAC CATGAGCTGC AGGGCCAGCT CAAGTGTAAA TTACATGTAC |
| 2161 | TGGTACCAGC AGAAGTCAGA TGCCTCCCCC AAACTATGGA TTTATTACAC ATCCAACCTG |
| 2221 | GCTCCTGGAG TCCCAGCTCG CTTCAGTGGC AGTGGGTCTG GGAACTCTTA TTCTCTCACA |
| 2281 | ATCAGCAGCA TGGAGGGTGA AGATGCTGCC ACTTATTACT GCCAGCAGTT TAGTAGTTCC |
| 2341 | CCGTGGACGT TCGGTGGAGG CACCAAGCTG GAAATCAAAC GCGGTGGCGG CGGATCAGGT |
| 2401 | GGAGGTGGAT CAGGTGGCGG TGGCAGTGGT GGCGGTGGAT CAGAAGTGAA GCTGGTGGAG |
| 2461 | TCTGGGGGAG GCTTAGTGAA GCCTGGAGGG TCCCTGAAAC TCTCCTGTGC AGCCTCTGGA |
| 2521 | TTCACTTTCA GTGCCTATGC CATGTCTTGG GTTCGCCAGA CTCCAGAGAA GAGGCTGGAG |
| 2581 | TGGGTCGCAT CCATTAGTAG TGGTGGTACC ACCTACTATC CAGACAGTGT GAAACGCCGA |
| 2641 | TTCACCATCT CCAGAGATAA TGCCAGGAAC ATCCTGTACC TGCAAATGAG CAGTCTGAGG |
| 2701 | TCTGAGGACA CGGCCATGTA TTACTGTACA AGAGGCGGGG ACTACGGCTA CGCTCTGGAC |
| 2761 | TACTGGGGTC AAGGAACCTC AGTCACCGTC TCCTCAGGTG GCGGAGGTTC CCATCATCAC |
| 2821 | CATCACCATT GA |

### SEQ ID NO:271

FVII-231 DNA sequence

| | |
|---|---|
| 1 | ATGGTCTCCC AGGCCCTCAG GCTCCTCTGC CTTCTGCTTG GGCTTCAGGG CTGCCTGGCT |
| 61 | GCAGTCTTCG TAACCCAGGA GGAAGCCCAC GGCGTCCTGC ACCGGCGCCG GCGCGCCAAC |
| 121 | GCGTTCCTGG AGGAGCTGCG GCCGGGCTCC CTGGAGAGGG AGTGCAAGGA GGAGCAGTGC |
| 181 | TCCTTCGAGG AGGCCCGGGA GATCTTCAAG GACGCGGAGA GGACGAAGCT GTTCTGGATT |
| 241 | TCTTACAGTG ATGGGGACCA GTGTGCCTCA AGTCCATGCC AGAATGGGGG CTCCTGCAAG |
| 301 | GACCAGCTCC AGTCCTATAT CTGCTTCTGC CTCCCTGCCT TCGAGGGCCG GAACTGTGAG |
| 361 | ACGCACAAGG ATGACCAGCT GATCTGTGTG AACGAGAACG GCGGCTGTGA GCAGTACTGC |
| 421 | AGTGACCACA CGGGCACCAA GCGCTCCTGT CGGTGCCACG AGGGGTACTC TCTGCTGGCA |
| 481 | GACGGGGTGT CCTGCACACC CACAGTTGAA TATCCATGTG GAAAAATACC TATTCTAGAA |
| 541 | AAAAGAAATG CCAGCAAACC CCAAGGCCGA ATTGTGGGGG GCAAGGTGTG CCCCAAAGGG |
| 601 | GAGTGTCCAT GGCAGGTCCT GTTGTTGGTG AATGGAGCTC AGTTGTGTGG GGGGACCCTG |
| 661 | ATCAACACCA TCTGGGTGGT CTCCGCGGCC CACTGTTTCG ACAAAATCAA GAACTGGAGG |
| 721 | AACCTGATCG CGGTGCTGGG CGAGCACGAC CTCAGCGAGC ACGACGGGGA TGAGCAGAGC |
| 781 | CGGCGGGTGG CGCAGGTCAT CATCCCCAGC ACGTACGTCC CGGGCACCAC CAACCACGAC |
| 841 | ATCGCGCTGC TCCGCCTGCA CCAGCCCGTG GTCCTCACTG ACCATGTGGT GCCCCTCTGC |
| 901 | CTGCCCGAAC GGACGTTCTC TGAGAGGACG CTGGCCTTCG TGCGCTTCTC ATTGGTCAGC |
| 961 | GGCTGGGGCC AGCTGCTGGA CCGTGGCGCC ACGGCCCTGG AGCTCATGGT CCTCAACGTG |
| 1021 | CCCCGGCTGA TGACCCAGGA CTGCCTGCAG CAGTCACGGA AGGTGGGAGA CTCCCCAAAT |
| 1081 | ATCACGGAGT ACATGTTCTG TGCCGGCTAC TCGGATGGCA GCAAGGACTC CTGCAAGGGG |
| 1141 | GACAGTGGAG GCCCACATGC CACCCACTAC CGGGGCACGT GGTACCTGAC GGGCATCGTC |
| 1201 | AGCTGGGGCC AGGGCTGCGC AACCGTGGGC CACTTTGGGG TGTACACCAG GGTGTCCCAG |
| 1261 | TACATCGAGT GGCTGCAAAA GCTCATGCGC TCAGAGCCAC GCCCAGGAGT CCTCCTGCGA |
| 1321 | GCCCCATTTC CCGGGTCTCC AGGTACCTCA GAGTCTGCTA CCCCCGAGTC AGGGCCAGGA |
| 1381 | TCAGAGCCAG CCACCTCCGG GTCTGAGACA CCCGGGACTT CCGAGAGTGC CACCCCTGAG |
| 1441 | TCCGGACCCG GGTCCGAGCC CGCCACTTCC GGCTCCGAAA CTCCCGGCAC AAGCGAGAGC |
| 1501 | GCTACCCCAG AGTCAGGACC AGGAACATCT ACAGAGCCCT CTGAAGGCTC CGCTCCAGGT |
| 1561 | GGCGGTGGCT CCGGCGGAGG TGGGTCCGGT GGCGGCGGAT CAGGTGGGGG TGGATCAGGC |
| 1621 | GGTGGAGGTT CCGGTGGCGG GGGATCCGAA AATGTGCTCA CCCAGTCTCC AGCAATCATG |
| 1681 | TCTGCATCTC TAGGGGAGAA GGTCACCATG AGCTGCAGGG CCAGCTCAAG TGTAAATTAC |
| 1741 | ATGTACTGGT ACCAGCAGAA GTCAGATGCC TCCCCCAAAC TATGGATTTA TTACACATCC |
| 1801 | AACCTGGCTC CTGGAGTCCC AGCTCGCTTC AGTGGCAGTG GGTCTGGGAA CTCTTATTCT |
| 1861 | CTCACAATCA GCAGCATGGA GGGTGAAGAT GCTGCCACTT ATTACTGCCA GCAGTTTAGT |
| 1921 | AGTTCCCCGT GGACGTTCGG TGGAGGCACC AAGCTGGAAA TCAAACGCGG TGGCGGCGGA |
| 1981 | TCAGGTGGAG GTGGATCAGG TGGCGGTGGC AGTGGTGGCG GTGGATCAGA AGTGAAGCTG |
| 2041 | GTGGAGTCTG GGGGAGGCTT AGTGAAGCCT GGAGGGTCCC TGAAACTCTC CTGTGCAGCC |
| 2101 | TCTGGATTCA CTTTCAGTGC CTATGCCATG TCTTGGGTTC GCCAGACTCC AGAGAAGAGG |
| 2161 | CTGGAGTGGG TCGCATCCAT TAGTAGTGGT GGTACCACCT ACTATCCAGA CAGTGTGAAA |
| 2221 | CGCCGATTCA CCATCTCCAG AGATAATGCC AGGAACATCC TGTACCTGCA AATGAGCAGT |
| 2281 | CTGAGGTCTG AGGACACGGC CATGTATTAC TGTACAAGAG GCGGGGACTA CGGCTACGCT |
| 2341 | CTGGACTACT GGGGTCAAGG AACCTCAGTC ACCGTCTCCT CAGGTGGCGG AGGTTCCCAT |
| 2401 | CATCACCATC ACCATTGA |

### SEQ ID NO:272

FVII-232 DNA sequence

| | |
|---|---|
| 1 | ATGGTCTCCC AGGCCCTCAG GCTCCTCTGC CTTCTGCTTG GGCTTCAGGG CTGCCTGGCT |
| 61 | GCAGTCTTCG TAACCCAGGA GGAAGCCCAC GGCGTCCTGC ACCGGCGCCG GCGCGCCAAC |
| 121 | GCGTTCCTGG AGGAGCTGCG GCCGGGCTCC CTGGAGAGGG AGTGCAAGGA GGAGCAGTGC |
| 181 | TCCTTCGAGG AGGCCCGGGA GATCTTCAAG GACGCGGAGA GGACGAAGCT GTTCTGGATT |
| 241 | TCTTACAGTG ATGGGGACCA GTGTGCCTCA AGTCCATGCC AGAATGGGGG CTCCTGCAAG |
| 301 | GACCAGCTCC AGTCCTATAT CTGCTTCTGC CTCCCTGCCT TCGAGGGCCG GAACTGTGAG |
| 361 | ACGCACAAGG ATGACCAGCT GATCTGTGTG AACGAGAACG GCGGCTGTGA GCAGTACTGC |
| 421 | AGTGACCACA CGGGCACCAA GCGCTCCTGT CGGTGCCACG AGGGGTACTC TCTGCTGGCA |
| 481 | GACGGGGTGT CCTGCACACC CACAGTTGAA TATCCATGTG GAAAAATACC TATTCTAGAA |
| 541 | AAAAGAAATG CCAGCAAACC CCAAGGCCGA ATTGTGGGGG GCAAGGTGTG CCCCAAAGGG |
| 601 | GAGTGTCCAT GGCAGGTCCT GTTGTTGGTG AATGGAGCTC AGTTGTGTGG GGGGACCCTG |
| 661 | ATCAACACCA TCTGGGTGGT CTCCGCGGCC CACTGTTTCG ACAAAATCAA GAACTGGAGG |
| 721 | AACCTGATCG CGGTGCTGGG CGAGCACGAC CTCAGCGAGC ACGACGGGGA TGAGCAGAGC |
| 781 | CGGCGGGTGG CGCAGGTCAT CATCCCCAGC ACGTACGTCC CGGGCACCAC CAACCACGAC |
| 841 | ATCGCGCTGC TCCGCCTGCA CCAGCCCGTG GTCCTCACTG ACCATGTGGT GCCCCTCTGC |
| 901 | CTGCCCGAAC GGACGTTCTC TGAGAGGACG CTGGCCTTCG TGCGCTTCTC ATTGGTCAGC |
| 961 | GGCTGGGGCC AGCTGCTGGA CCGTGGCGCC ACGGCCCTGG AGCTCATGGT CCTCAACGTG |
| 1021 | CCCCGGCTGA TGACCCAGGA CTGCCTGCAG CAGTCACGGA AGGTGGGAGA CTCCCCAAAT |
| 1081 | ATCACGGAGT ACATGTTCTG TGCCGGCTAC TCGGATGGCA GCAAGGACTC CTGCAAGGGG |
| 1141 | GACAGTGGAG GCCCACATGC CACCCACTAC CGGGGCACGT GGTACCTGAC GGGCATCGTC |
| 1201 | AGCTGGGGCC AGGGCTGCGC AACCGTGGGC CACTTTGGGG TGTACACCAG GGTGTCCCAG |
| 1261 | TACATCGAGT GGCTGCAAAA GCTCATGCGC TCAGAGCCAC GCCCAGGAGT CCTCCTGCGA |
| 1321 | GCCCCATTTC CCGGGTCTCC AGGTACCTCA GAGTCTGCTA CCCCCGAGTC AGGGCCAGGA |
| 1381 | TCAGAGCCAG CCACCTCCGG GTCTGAGACA CCCGGGACTT CCGAGAGTGC CACCCCTGAG |
| 1441 | TCCGGACCCG GGTCCGAGCC CGCCACTTCC GGCTCCGAAA CTCCCGGCAC AAGCGAGAGC |
| 1501 | GCTACCCCAG AGTCAGGACC AGGAACATCT ACAGAGCCCT CTGAAGGCTC CGCTCCAGGG |
| 1561 | TCCCCAGCCG GCAGTCCCAC TAGCACCGAG GAGGGAACCT CTGAAAGCGC CACACCCGAA |
| 1621 | TCAGGGCCAG GGTCTGAGCC TGCTACCAGC GGCAGCGAGA CACCAGGCAC CTCTGAGTCC |
| 1681 | GCCACACCAG AGTCCGGACC CGGATCTCCC GCTGGGAGCC CCACCTCCAC TGAGGAGGGA |
| 1741 | TCTCCTGCTG GCTCTCCAAC ATCTACTGAG GAAGGTGGCG GTGGCTCCGG CGGAGGTGGG |
| 1801 | TCCGGTGGCG GCGGATCAGG TGGGGGTGGA TCAGGCGGTG GAGGTTCCGG TGGCGGGGGA |
| 1861 | TCCGAAAATG TGCTCACCCA GTCTCCAGCA ATCATGTCTG CATCTCTAGG GGAGAAGGTC |
| 1921 | ACCATGAGCT GCAGGGCCAG CTCAAGTGTA AATTACATGT ACTGGTACCA GCAGAAGTCA |
| 1981 | GATGCCTCCC CCAAACTATG GATTTATTAC ACATCCAACC TGGCTCCTGG AGTCCCAGCT |
| 2041 | CGCTTCAGTG GCAGTGGGTC TGGGAACTCT TATTCTCTCA CAATCAGCAG CATGGAGGGT |
| 2101 | GAAGATGCTG CCACTTATTA CTGCCAGCAG TTTAGTAGTT CCCCGTGGAC GTTCGGTGGA |
| 2161 | GGCACCAAGC TGGAAATCAA ACGCGGTGGC GGCGGATCAG GTGGAGGTGG ATCAGGTGGC |
| 2221 | GGTGGCAGTG GTGGCGGTGG ATCAGAAGTG AAGCTGGTGG AGTCTGGGGG AGGCTTAGTG |
| 2281 | AAGCCTGGAG GGTCCCTGAA ACTCTCCTGT GCAGCCTCTG GATTCACTTT CAGTGCCTAT |
| 2341 | GCCATGTCTT GGGTTCGCCA GACTCCAGAG AAGAGGCTGG AGTGGGTCGC ATCCATTAGT |
| 2401 | AGTGGTGGTA CCACCTACTA TCCAGACAGT GTGAAACGCC GATTCACCAT CTCCAGAGAT |
| 2461 | AATGCCAGGA ACATCCTGTA CCTGCAAATG AGCAGTCTGA GGTCTGAGGA CACGGCCATG |
| 2521 | TATTACTGTA CAAGAGGCGG GGACTACGGC TACGCTCTGG ACTACTGGGG TCAAGGAACC |
| 2581 | TCAGTCACCG TCTCCTCAGG TGGCGGAGGT TCCCATCATC ACCATCACCA TTGA |

### SEQ ID NO:273

FVII-242 DNA sequence

| | |
|---|---|
| 1 | ATGGTCTCCC AGGCCCTCAG GCTCCTCTGC CTTCTGCTTG GGCTTCAGGG CTGCCTGGCT |
| 61 | GCAGTCTTCG TAACCCAGGA GGAAGCCCAC GGCGTCCTGC ACCGGCGCCG GCGCGCCAAC |
| 121 | GCGTTCCTGG AGGAGCTACG GCCGGGCTCC CTGGAGAGGG AGTGCAAGGA GGAGCAGTGC |
| 181 | TCCTTCGAGG AGGCCCGGGA GATCTTCAAG GACGCGGAGA GGACGAAGCT GTTCTGGATT |
| 241 | TCTTACAGTG ATGGGGACCA GTGTGCCTCA AGTCCATGCC AGAATGGGGG CTCCTGCAAG |
| 301 | GACCAGCTCC AGTCCTATAT CTGCTTCTGC CTCCCTGCCT TCGAGGGCCG GAACTGTGAG |
| 361 | ACGCACAAGG ATGACCAGCT GATCTGTGTG AACGAGAACG GCGGCTGTGA GCAGTACTGC |
| 421 | AGTGACCACA CGGGCACCAA GCGCTCCTGT CGGTGCCACG AGGGGTACTC TCTGCTGGCA |
| 481 | GACGGGGTGT CCTGCACACC CACAGTTGAA TATCCATGTG GAAAAATACC TATTCTAGAA |
| 541 | AAAAGAAATG CCAGCAAACC CCAAGGTGGC GGCGGATCAG GTGGGGGTGG ATCAGGCGGT |
| 601 | GGAGGTTCCG GTGGCGGGGG ATCCGGCGGT GGAGGTTCCG GTGGGGGTGG ATCAGGCTCG |
| 661 | AGTGGTACCT CAGAGTCTGC TACCCCCGAG TCAGGGCCAG GATCAGAGCC AGCCACCTCC |
| 721 | GGGTCTGAGA CACCCGGGAC TTCCGAGAGT GCCACCCCTG AGTCCGGACC CGGGTCCGAG |
| 781 | CCCGCCACTA CCGGTGGTGG CGGTGGCTCC GGCGGAGGTG GGTCCGGTGG CGGCGGATCA |
| 841 | GGTGGGGGTG GATCAGGCGG TGGAGGTTCC GGTGGCGGGG GATCAAGAAA GAGAAGGAAA |
| 901 | AGAATTGTGG GGGGCAAGGT GTGCCCCAAA GGGGAGTGTC CATGGCAGGT CCTGTTGTTG |
| 961 | GTGAATGGAG CTCAGTTGTG TGGGGGGACC CTGATCAACA CCATCTGGGT GGTGTCCGCG |
| 1021 | GCCCACTGTT TCGACAAAAT CAAGAACTGG AGGAACCTGA TCGCGGTGCT GGGCGAGCAC |
| 1081 | GACCTCAGCG AGCACGACGG GGATGAGCAG AGCCGGCGGG TGGCGCAGGT CATCATCCCC |
| 1141 | AGCACGTACG TCCCGGGCAC CACCAACCAC GACATCGCGC TGCTCCGCCT GCACCAGCCC |
| 1201 | GTGGTCCTCA CTGACCATGT GGTGCCCCTC TGCCTGCCCG AACGGACGTT CTCTGAGAGG |
| 1261 | ACGCTGGCCT TCGTGCGCTT CTCATTGGTC AGCGGCTGGG GCCAGCTGCT GGACCGTGGC |
| 1321 | GCCACGGCCC TGGAGCTCAT GGTCCTCAAC GTGCCCCGGC TGATGACCCA GGACTGCCTG |
| 1381 | CAGCAGTCAC GGAAGGTGGG AGACTCCCCA AATATCACGG AGTACATGTT CTGTGCCGGC |
| 1441 | TACTCGGATG GCAGCAAGGA CTCCTGCAAG GGGGACAGTG GAGGCCCACA TGCCACCCAC |
| 1501 | TACCGGGGCA CGTGGTACCT GACGGGCATC GTCAGCTGGG GCCAGGGCTG CGCAACCGTG |
| 1561 | GGCCACTTTG GGGTGTACAC CAGGGTGTCC CAGTACATCG AGTGGCTGCA AAAGCTCATG |
| 1621 | CGCTCAGAGC CACGCCCAGG AGTCCTCCTG CGAGCCCCAT TTCCCGGGTC TCCAGGTACC |
| 1681 | TCAGAGTCTG CTACCCCCGA GTCAGGGCCA GGATCAGAGC CAGCCACCTC CGGGTCTGAG |
| 1741 | ACACCCGGGA CTTCCGAGAG TGCCACCCCT GAGTCCGGAC CCGGGTCCGA GCCCGCCACT |
| 1801 | TCCGGCTCCG AAACTCCCGG CACAAGCGAG AGCGCTACCC CAGAGTCAGG ACCAGGAACA |
| 1861 | TCTACAGAGC CCTCTGAAGG CTCCGCTCCA GGTGGCGGTG GCTCCGGCGG AGGTGGGTCC |
| 1921 | GGTGGCGGCG GATCAGGTGG GGGTGGATCA GGCGGTGGAG GTTCCGGTGG CGGGGGATCC |
| 1981 | GAAAATGTGC TCACCCAGTC TCCAGCAATC ATGTCTGCAT CTCTAGGGGA GAAGGTCACC |
| 2041 | ATGAGCTGCA GGGCCAGCTC AAGTGTAAAT TACATGTACT GGTACCAGCA GAAGTCAGAT |
| 2101 | GCCTCCCCCA AACTATGGAT TTATTACACA TCCAACCTGG CTCCTGGAGT CCCAGCTCGC |
| 2161 | TTCAGTGGCA GTGGGTCTGG GAACTCTTAT TCTCTCACAA TCAGCAGCAT GGAGGGTGAA |
| 2221 | GATGCTGCCA CTTATTACTG CCAGCAGTTT AGTAGTTCCC CGTGGACGTT CGGTGGAGGC |
| 2281 | ACCAAGCTGG AAATCAAACG CGGTGGCGGC GGATCAGGTG GAGGTGGATC AGGTGGCGGT |
| 2341 | GGCAGTGGTG GCGGTGGATC AGAAGTGAAG CTGGTGGAGT CTGGGGGAGG CTTAGTGAAG |
| 2401 | CCTGGAGGGT CCCTGAAACT CTCCTGTGCA GCCTCTGGAT TCACTTTCAG TGCCTATGCC |
| 2461 | ATGTCTTGGG TTCGCCAGAC TCCAGAGAAG AGGCTGGAGT GGGTCGCATC CATTAGTAGT |
| 2521 | GGTGGTACCA CCTACTATCC AGACAGTGTG AAACGCCGAT TCACCATCTC CAGAGATAAT |
| 2581 | GCCAGGAACA TCCTGTACCT GCAAATGAGC AGTCTGAGGT CTGAGGACAC GGCCATGTAT |
| 2641 | TACTGTACAA GAGGCGGGGA CTACGGCTAC GCTCTGGACT ACTGGGGTCA AGGAACCTCA |
| 2701 | GTCACCGTCT CCTCAGGTGG CGGAGGTTCC CATCATCACC ATCACCATTG A |

### SEQ ID NO:274

FVII-243 DNA sequence

| | |
|---|---|
| 1 | ATGGTCTCCC AGGCCCTCAG GCTCCTCTGC CTTCTGCTTG GGCTTCAGGG CTGCCTGGCT |
| 61 | GCAGTCTTCG TAACCCAGGA GGAAGCCCAC GGCGTCCTGC ACCGGCGCCG GCGCGCCAAC |
| 121 | GCGTTCCTGG AGGAGCTACG GCCGGGCTCC CTGGAGAGGG AGTGCAAGGA GGAGCAGTGC |
| 181 | TCCTTCGAGG AGGCCCGGGA GATCTTCAAG GACGCGGAGA GGACGAAGCT GTTCTGGATT |
| 241 | TCTTACAGTG ATGGGGACCA GTGTGCCTCA AGTCCATGCC AGAATGGGGG CTCCTGCAAG |
| 301 | GACCAGCTCC AGTCCTATAT CTGCTTCTGC CTCCCTGCCT TCGAGGGCCG GAACTGTGAG |
| 361 | ACGCACAAGG ATGACCAGCT GATCTGTGTG AACGAGAACG GCGGCTGTGA GCAGTACTGC |
| 421 | AGTGACCACA CGGGCACCAA GCGCTCCTGT CGGTGCCACG AGGGGTACTC TCTGCTGGCA |
| 481 | GACGGGGTGT CCTGCACACC CACAGTTGAA TATCCATGTG GAAAAATACC TATTCTAGAA |
| 541 | AAAAGAAATG CCAGCAAACC CCAAGGTGGC GGCGGATCAG GTGGGGGTGG ATCAGGCGGT |
| 601 | GGAGGTTCCG GTGGCGGGGG ATCCGGCGGT GGAGGTTCCG GTGGGGGTGG ATCAGGCTCG |
| 661 | AGTAGTCCCG CTGGAAGCCC AACTAGCACC GAAGAGGGGA CCTCAGAGTC CGCCACCCCC |
| 721 | GAGTCCGGCC CTGGCTCTGA GCCTGCCACT AGCGGCTCCG AGACTCCTGG CACATCCGAA |
| 781 | AGCGCTACAC CCGAGAGTGG ACCCGGCACC TCTACCGAGC CCAGTGAGGG CTCCGCCCCT |
| 841 | GGAACAAGCA CCGAGCCCAG CGAAGGCAGC GCCCCAGGGA CCGGTGGTGG CGGTGGCTCC |
| 901 | GGCGGAGGTG GGTCCGGTGG CGGCGGATCA GGTGGGGGTG GATCAGGCGG TGGAGGTTCC |
| 961 | GGTGGCGGGG GATCAAGAAA GAGAAGGAAA AGAATTGTGG GGGGCAAGGT GTGCCCCAAA |
| 1021 | GGGGAGTGTC CATGGCAGGT CCTGTTGTTG GTGAATGGAG CTCAGTTGTG TGGGGGGACC |
| 1081 | CTGATCAACA CCATCTGGGT GGTGTCCGCG GCCCACTGTT TCGACAAAAT CAAGAACTGG |
| 1141 | AGGAACCTGA TCGCGGTGCT GGGCGAGCAC GACCTCAGCG AGCACGACGG GGATGAGCAG |
| 1201 | AGCCGGCGGG TGGCGCAGGT CATCATCCCC AGCACGTACG TCCCGGGCAC CACCAACCAC |
| 1261 | GACATCGCGC TGCTCCGCCT GCACCAGCCC GTGGTCCTCA CTGACCATGT GGTGCCCCTC |
| 1321 | TGCCTGCCCG AACGGACGTT CTCTGAGAGG ACGCTGGCCT TCGTGCGCTT CTCATTGGTC |
| 1381 | AGCGGCTGGG GCCAGCTGCT GGACCGTGGC GCCACGGCCC TGGAGCTCAT GGTCCTCAAC |
| 1441 | GTGCCCCGGC TGATGACCCA GGACTGCCTG CAGCAGTCAC GGAAGGTGGG AGACTCCCCA |
| 1501 | AATATCACGG AGTACATGTT CTGTGCCGGC TACTCGGATG GCAGCAAGGA CTCCTGCAAG |
| 1561 | GGGGACAGTG GAGGCCCACA TGCCACCCAC TACCGGGGCA CGTGGTACCT GACGGGCATC |
| 1621 | GTCAGCTGGG GCCAGGGCTG CGCAACCGTG GGCCACTTTG GGGTGTACAC CAGGGTGTCC |
| 1681 | CAGTACATCG AGTGGCTGCA AAAGCTCATG CGCTCAGAGC CACGCCCAGG AGTCCTCCTG |
| 1741 | CGAGCCCCAT TTCCCGGGTC TCCAGGTACC TCAGAGTCTG CTACCCCCGA GTCAGGGCCA |
| 1801 | GGATCAGAGC CAGCCACCTC CGGGTCTGAG ACACCCGGGA CTTCCGAGAG TGCCACCCCT |
| 1861 | GAGTCCGGAC CCGGGTCCGA GCCCGCCACT GGTGGCGGTG GCTCCGGCGG AGGTGGGTCC |
| 1921 | GGTGGCGGCG GATCAGGTGG GGGTGGATCA GGCGGTGGAG GTTCCGGTGG CGGGGGATCC |
| 1981 | GAAAATGTGC TCACCCAGTC TCCAGCAATC ATGTCTGCAT CTCTAGGGGA GAAGGTCACC |
| 2041 | ATGAGCTGCA GGGCCAGCTC AAGTGTAAAT TACATGTACT GGTACCAGCA GAAGTCAGAT |
| 2101 | GCCTCCCCCA AACTATGGAT TTATTACACA TCCAACCTGG CTCCTGGAGT CCCAGCTCGC |
| 2161 | TTCAGTGGCA GTGGGTCTGG GAACTCTTAT TCTCTCACAA TCAGCAGCAT GGAGGGTGAA |
| 2221 | GATGCTGCCA CTTATTACTG CCAGCAGTTT AGTAGTTCCC CGTGGACGTT CGGTGGAGGC |
| 2281 | ACCAAGCTGG AAATCAAACG CGGTGGCGGC GGATCAGGTG GAGGTGGATC AGGTGGCGGT |
| 2341 | GGCAGTGGTG GCGGTGGATC AGAAGTGAAG CTGGTGGAGT CTGGGGGAGG CTTAGTGAAG |
| 2401 | CCTGGAGGGT CCCTGAAACT CTCCTGTGCA GCCTCTGGAT TCACTTTCAG TGCCTATGCC |
| 2461 | ATGTCTTGGG TTCGCCAGAC TCCAGAGAAG AGGCTGGAGT GGGTCGCATC CATTAGTAGT |
| 2521 | GGTGGTACCA CCTACTATCC AGACAGTGTG AAACGCCGAT TCACCATCTC CAGAGATAAT |
| 2581 | GCCAGGAACA TCCTGTACCT GCAAATGAGC AGTCTGAGGT CTGAGGACAC GGCCATGTAT |
| 2641 | TACTGTACAA GAGGCGGGGA CTACGGCTAC GCTCTGGACT ACTGGGGTCA AGGAACCTCA |
| 2701 | GTCACCGTCT CCTCAGGTGG CGGAGGTTCC CATCATCACC ATCACCATTG A |

### SEQ ID NO:275

FVII-238 DNA sequence

| | |
|---|---|
| 1 | ATGGTCTCCC AGGCCCTCAG GCTCCTCTGC CTTCTGCTTG GGCTTCAGGG CTGCCTGGCT |
| 61 | GCAGTCTTCG TAACCCAGGA GGAAGCCCAC GGCGTCCTGC ACCGGCGCCG GCGCGCCAAC |
| 121 | GCGTTCCTGG AGGAGCTACG GCCGGGCTCC CTGGAGAGGG AGTGCAAGGA GGAGCAGTGC |
| 181 | TCCTTCGAGG AGGCCCGGGA GATCTTCAAG GACGCGGAGA GGACGAAGCT GTTCTGGATT |
| 241 | TCTTACAGTG ATGGGGACCA GTGTGCCTCA AGTCCATGCC AGAATGGGGG CTCCTGCAAG |
| 301 | GACCAGCTCC AGTCCTATAT CTGCTTCTGC CTCCCTGCCT TCGAGGGCCG GAACTGTGAG |
| 361 | ACGCACAAGG ATGACCAGCT GATCTGTGTG AACGAGAACG GCGGCTGTGA GCAGTACTGC |
| 421 | AGTGACCACA CGGGCACCAA GCGCTCCTGT CGGTGCCACG AGGGGTACTC TCTGCTGGCA |
| 481 | GACGGGGTGT CCTGCACACC CACAGTTGAA TATCCATGTG GAAAAATACC TATTCTAGAA |
| 541 | AAAAGAAATG CCAGCAAACC CCAAGGTGGC GGCGGATCAG GTGGGGGTGG ATCAGGCGGT |
| 601 | GGAGGTTCCG GTGGCGGGGG ATCCGGCGGT GGAGGTTCCG GTGGGGGTGG ATCAGGCTCG |
| 661 | AGTAGTCCCG CTGGAAGCCC AACTAGCACC GAAGAGGGGA CCTCAGAGTC CGCCACCCCC |
| 721 | GAGTCCGGCC CTGGCTCTGA GCCTGCCACT AGCGGCTCCG AGACTCCTGG CACATCCGAA |
| 781 | AGCGCTACAC CCGAGAGTGG ACCCGGCACC TCTACCGAGC CCAGTGAGGG CTCCGCCCCT |
| 841 | GGAACAAGCA CCGAGCCCAG CGAAGGCAGC GCCCCAGGGA CCGGTGGTGG CGGTGGCTCC |
| 901 | GGCGGAGGTG GGTCCGGTGG CGGCGGATCA GGTGGGGGTG GATCAGGCGG TGGAGGTTCC |
| 961 | GGTGGCGGGG GATCAAGAAA GAGAAGGAAA AGAATTGTGG GGGGCAAGGT GTGCCCCAAA |
| 1021 | GGGGAGTGTC CATGGCAGGT CCTGTTGTTG GTGAATGGAG CTCAGTTGTG TGGGGGGACC |
| 1081 | CTGATCAACA CCATCTGGGT GGTGTCCGCG GCCCACTGTT TCGACAAAAT CAAGAACTGG |
| 1141 | AGGAACCTGA TCGCGGTGCT GGGCGAGCAC GACCTCAGCG AGCACGACGG GGATGAGCAG |
| 1201 | AGCCGGCGGG TGGCGCAGGT CATCATCCCC AGCACGTACG TCCCGGGCAC CACCAACCAC |
| 1261 | GACATCGCGC TGCTCCGCCT GCACCAGCCC GTGGTCCTCA CTGACCATGT GGTGCCCCTC |
| 1321 | TGCCTGCCCG AACGGACGTT CTCTGAGAGG ACGCTGGCCT TCGTGCGCTT CTCATTGGTC |
| 1381 | AGCGGCTGGG GCCAGCTGCT GGACCGTGGC GCCACGGCCC TGGAGCTCAT GGTCCTCAAC |
| 1441 | GTGCCCCGGC TGATGACCCA GGACTGCCTG CAGCAGTCAC GGAAGGTGGG AGACTCCCCA |
| 1501 | AATATCACGG AGTACATGTT CTGTGCCGGC TACTCGGATG GCAGCAAGGA CTCCTGCAAG |
| 1561 | GGGGACAGTG GAGGCCCACA TGCCACCCAC TACCGGGGCA CGTGGTACCT GACGGGCATC |
| 1621 | GTCAGCTGGG GCCAGGGCTG CGCAACCGTG GGCCACTTTG GGGTGTACAC CAGGGTGTCC |
| 1681 | CAGTACATCG AGTGGCTGCA AAAGCTCATG CGCTCAGAGC CACGCCCAGG AGTCCTCCTG |
| 1741 | CGAGCCCCAT TTCCCGGGTC TCCAGGTACC TCAGAGTCTG CTACCCCCGA GTCAGGGCCA |
| 1801 | GGATCAGAGC CAGCCACCTC CGGGTCTGAG ACACCCGGGA CTTCCGAGAG TGCCACCCCT |
| 1861 | GAGTCCGGAC CCGGGTCCGA GCCCGCCACT TCCGGCTCCG AAACTCCCGG CACAAGCGAG |
| 1921 | AGCGCTACCC CAGAGTCAGG ACCAGGAACA TCTACAGAGC CCTCTGAAGG CTCCGCTCCA |
| 1981 | GGTGGCGGTG GCTCCGGCGG AGGTGGGTCC GGTGGCGGCG GATCAGGTGG GGGTGGATCA |
| 2041 | GGCGGTGGAG GTTCCGGTGG CGGGGGATCC GAAAATGTGC TCACCCAGTC TCCAGCAATC |
| 2101 | ATGTCTGCAT CTCTAGGGGA GAAGGTCACC ATGAGCTGCA GGGCCAGCTC AAGTGTAAAT |
| 2161 | TACATGTACT GGTACCAGCA GAAGTCAGAT GCCTCCCCCA AACTATGGAT TTATTACACA |
| 2221 | TCCAACCTGG CTCCTGGAGT CCCAGCTCGC TTCAGTGGCA GTGGGTCTGG GAACTCTTAT |
| 2281 | TCTCTCACAA TCAGCAGCAT GGAGGGTGAA GATGCTGCCA CTTATTACTG CCAGCAGTTT |
| 2341 | AGTAGTTCCC CGTGGACGTT CGGTGGAGGC ACCAAGCTGG AAATCAAACG CGGTGGCGGC |
| 2401 | GGATCAGGTG GAGGTGGATC AGGTGGCGGT GGCAGTGGTG GCGGTGGATC AGAAGTGAAG |
| 2461 | CTGGTGGAGT CTGGGGGAGG CTTAGTGAAG CCTGGAGGGT CCCTGAAACT CTCCTGTGCA |
| 2521 | GCCTCTGGAT TCACTTTCAG TGCCTATGCC ATGTCTTGGG TTCGCCAGAC TCCAGAGAAG |
| 2581 | AGGCTGGAGT GGGTCGCATC CATTAGTAGT GGTGGTACCA CCTACTATCC AGACAGTGTG |
| 2641 | AAACGCCGAT TCACCATCTC CAGAGATAAT GCCAGGAACA TCCTGTACCT GCAAATGAGC |
| 2701 | AGTCTGAGGT CTGAGGACAC GGCCATGTAT TACTGTACAA GAGGCGGGGA CTACGGCTAC |
| 2761 | GCTCTGGACT ACTGGGGTCA AGGAACCTCA GTCACCGTCT CCTCAGGTGG CGGAGGTTCC |
| 2821 | CATCATCACC ATCACCATTG A |

### SEQ ID NO:______

FVII-189 DNA sequence

| | |
|---|---|
| 1 | ATGGT CTCCC AGGCC CTCAG GCTCC TCTGC CTTCT GCTTG GGCTT CAGGG |
| | TACCA GAGGG TCCGG GAGTC CGAGG AGACG GAAGA CGAAC CCGAA GTCCC |
| 51 | CTGCC TGGCT GCAGT CTTCG TAACC CAGGA GGAAG CCCAC GGCGT CCTGC |
| | GACGG ACCGA CGTCA GAAGC ATTGG GTCCT CCTTC GGGTG CCGCA GGACG |
| 101 | ACCGG CGCCG GCGCG CCAAC GCGTT CCTGG AGGAG CTGCG GCCGG GCTCC |
| | TGGCC GCGGC CGCGC GGTTG CGCAA GGACC TCCTC GACGC CGGCC CGAGG |
| 151 | CTGGA GAGGG AGTGC AAGGA GGAGC AGTGC TCCTT CGAGG AGGCC CGGGA |
| | GACCT CTCCC TCACG TTCCT CCTCG TCACG AGGAA GCTCC TCCGG GCCCT |
| 201 | GATCT TCAAG GACGC GGAGA GGACG AAGCT GTTCT GGATT TCTTA CAGTG |
| | CTAGA AGTTC CTGCG CCTCT CCTGC TTCGA CAAGA CCTAA AGAAT GTCAC |
| 251 | ATGGG GACCA GTGTG CCTCA AGTCC ATGCC AGAAT GGGGG CTCCT GCAAG |
| | TACCC CTGGT CACAC GGAGT TCAGG TACGG TCTTA CCCCC GAGGA CGTTC |
| 301 | GACCA GCTCC AGTCC TATAT CTGCT TCTGC CTCCC TGCCT TCGAG GGCCG |
| | CTGGT CGAGG TCAGG ATATA GACGA AGACG GAGGG ACGGA AGCTC CCGGC |
| 351 | GAACT GTGAG ACGCA CAAGG ATGAC CAGCT GATCT GTGTG AACGA GAACG |
| | CTTGA CACTC TGCGT GTTCC TACTG GTCGA CTAGA CACAC TTGCT CTTGC |
| 401 | GCGGC TGTGA GCAGT ACTGC AGTGA CCACA CGGGC ACCAA GCGCT CCTGT |
| | CGCCG ACACT CGTCA TGACG TCACT GGTGT GCCCG TGGTT CGCGA GGACA |
| 451 | CGGTG CCACG AGGGG TACTC TCTGC TGGCA GACGG GGTGT CCTGC ACACC |
| | GCCAC GGTGC TCCCC ATGAG AGACG ACCGT CTGCC CCACA GGACG TGTGG |
| 501 | CACAG TTGAA TATCC ATGTG GAAAA ATACC TATTC TAGAA AAAAG AAATG |
| | GTGTC AACTT ATAGG TACAC CTTTT TATGG ATAAG ATCTT TTTTC TTTAC |
| 551 | CCAGC AAACC CCAAG GCCGA ATTGT GGGGG GCAAG GTGTG CCCCA AAGGG |
| | GGTCG TTTGG GGTTC CGGCT TAACA CCCCC CGTTC CACAC GGGGT TTCCC |
| 601 | GAGTG TCCAT GGCAG GTCCT GTTGT TGGTG AATGG AGCTC AGTTG TGTGG |
| | CTCAC AGGTA CCGTC CAGGA CAACA ACCAC TTACC TCGAG TCAAC ACACC |
| 651 | GGGGA CCCTG ATCAA CACCA TCTGG GTGGT CTCCG CGGCC CACTG TTTCG |
| | CCCCT GGGAC TAGTT GTGGT AGACC CACCA GAGGC GCCGG GTGAC AAAGC |
| 701 | ACAAA ATCAA GAACT GGAGG AACCT GATCG CGGTG CTGGG CGAGC ACGAC |
| | TGTTT TAGTT CTTGA CCTCC TTGGA CTAGC GCCAC GACCC GCTCG TGCTG |
| 751 | CTCAG CGAGC ACGAC GGGGA TGAGC AGAGC CGGCG GGTGG CGCAG GTCAT |
| | GAGTC GCTCG TGCTG CCCCT ACTCG TCTCG GCCGC CCACC GCGTC CAGTA |
| 801 | CATCC CCAGC ACGTA CGTCC CGGGC ACCAC CAACC ACGAC ATCGC GCTGC |
| | GTAGG GGTCG TGCAT GCAGG GCCCG TGGTG GTTGG TGCTG TAGCG CGACG |
| 851 | TCCGC CTGCA CCAGC CCGTG GTCCT CACTG ACCAT GTGGT GCCCC TCTGC |
| | AGGCG GACGT GGTCG GGCAC CAGGA GTGAC TGGTA CACCA CGGGG AGACG |
| 901 | CTGCC CGAAC GGACG TTCTC TGAGA GGACG CTGGC CTTCG TGCGC TTCTC |
| | GACGG GCTTG CCTGC AAGAG ACTCT CCTGC GACCG GAAGC ACGCG AAGAG |
| 951 | ATTGG TCAGC GGCTG GGGCC AGCTG CTGGA CCGTG GCGCC ACGGC CCTGG |
| | TAACC AGTCG CCGAC CCCGG TCGAC GACCT GGCAC CGCGG TGCCG GGACC |
| 1001 | AGCTC ATGGT CCTCA ACGTG CCCCG GCTGA TGACC CAGGA CTGCC TGCAG |
| | TCGAG TACCA GGAGT TGCAC GGGGC CGACT ACTGG GTCCT GACGG ACGTC |
| 1051 | CAGTC ACGGA AGGTG GGAGA CTCCC CAAAT ATCAC GGAGT ACATG TTCTG |
| | GTCAG TGCCT TCCAC CCTCT GAGGG GTTTA TAGTG CCTCA TGTAC AAGAC |
| 1101 | TGCCG GCTAC TCGGA TGGCA GCAAG GACTC CTGCA AGGGG GACAG TGGAG |
| | ACGGC CGATG AGCCT ACCGT CGTTC CTGAG GACGT TCCCC CTGTC ACCTC |
| 1151 | GCCCA CATGC CACCC ACTAC CGGGG CACGT GGTAC CTGAC GGGCA TCGTC |
| | CGGGT GTACG GTGGG TGATG GCCCC GTGCA CCATG GACTG CCCGT AGCAG |
| 1201 | AGCTG GGGCC AGGGC TGCGC AACCG TGGGC CACTT TGGGG TGTAC ACCAG |
| | TCGAC CCCGG TCCCG ACGCG TTGGC ACCCG GTGAA ACCCC ACATG TGGTC |
| 1251 | GGTCT CCCAG TACAT CGAGT GGCTG CAAAA GCTCA TGCGC TCAGA GCCAC |
| | CCAGA GGGTC ATGTA GCTCA CCGAC GTTTT CGAGT ACGCG AGTCT CGGTG |
| 1301 | GCCCA GGAGT CCTCC TGCGA GCCCC ATTTC CCGGT GGCGG TGGCT CCGGC |
| | CGGGT CCTCA GGAGG ACGCT CGGGG TAAAG GGCCA CCGCC ACCGA GGCCG |
| 1351 | GGAGG TGGGT CCGGT GGCGG CGGAT CAGGT GGGGG TGGAT CAGGC GGTGG |
| | CCTCC ACCCA GGCCA CCGCC GCCTA GTCCA CCCCC ACCTA GTCCG CCACC |
| 1401 | AGGTT CCGGT GGCGG GGGAT CCGAA AATGT GCTCA CCCAG TCTCC AGCAA |
| | TCCAA GGCCA CCGCC CCCTA GGCTT TTACA CGAGT GGGTC AGAGG TCGTT |
| 1451 | TCATG TCTGC ATCTC TAGGG GAGAA GGTCA CCATG AGCTG CAGGG CCAGC |
| | AGTAC AGACG TAGAG ATCCC CTCTT CCAGT GGTAC TCGAC GTCCC GGTCG |
| 1501 | TCAAG TGTAA ATTAC ATGTA CTGGT ACCAG CAGAA GTCAG ATGCC TCCCC |
| | AGTTC ACATT TAATG TACAT GACCA TGGTC GTCTT CAGTC TACGG AGGGG |
| 1551 | CAAAC TATGG ATTTA TTACA CATCC AACCT GGCTC CTGGA GTCCC AGCTC |
| | GTTTG ATACC TAAAT AATGT GTAGG TTGGA CCGAG GACCT CAGGG TCGAG |
| 1601 | GCTTC AGTGG CAGTG GGTCT GGGAA CTCTT ATTCT CTCAC AATCA GCAGC |
| | CGAAG TCACC GTCAC CCAGA CCCTT GAGAA TAAGA GAGTG TTAGT CGTCG |
| 1651 | ATGGA GGGTG AAGAT GCTGC CACTT ATTAC TGCCA GCAGT TTAGT AGTTC |
| | TACCT CCCAC TTCTA CGACG GTGAA TAATG ACGGT CGTCA AATCA TCAAG |
| 1701 | CCCGT GGACG TTCGG TGGAG GCACC AAGCT GGAAA TCAAA CGCGG TGGCG |
| | GGGCA CCTGC AAGCC ACCTC CGTGG TTCGA CCTTT AGTTT GCGCC ACCGC |
| 1751 | GCGGA TCAGG TGGAG GTGGA TCAGG TGGCG GTGGC AGTGG TGGCG GTGGA |
| | CGCCT AGTCC ACCTC CACCT AGTCC ACCGC CACCG TCACC ACCGC CACCT |
| 1801 | TCAGA AGTGA AGCTG GTGGA GTCTG GGGGA GGCTT AGTGA AGCCT GGAGG |
| | AGTCT TCACT TCGAC CACCT CAGAC CCCCT CCGAA TCACT TCGGA CCTCC |
| 1851 | GTCCC TGAAA CTCTC CTGTG CAGCC TCTGG ATTCA CTTTC AGTGC CTATG |
| | CAGGG ACTTT GAGAG GACAC GTCGG AGACC TAAGT GAAAG TCACG GATAC |
| 1901 | CCATG TCTTG GGTTC GCCAG ACTCC AGAGA AGAGG CTGGA GTGGG TCGCA |
| | GGTAC AGAAC CCAAG CGGTC TGAGG TCTCT TCTCC GACCT CACCC AGCGT |
| 1951 | TCCAT TAGTA GTGGT GGTAC CACCT ACTAT CCAGA CAGTG TGAAA CGCCG |
| | AGGTA ATCAT CACCA CCATG GTGGA TGATA GGTCT GTCAC ACTTT GCGGC |
| 2001 | ATTCA CCATC TCCAG AGATA ATGCC AGGAA CATCC TGTAC CTGCA AATGA |
| | TAAGT GGTAG AGGTC TCTAT TACGG TCCTT GTAGG ACATG GACGT TTACT |
| 2051 | GCAGT CTGAG GTCTG AGGAC ACGGC CATGT ATTAC TGTAC AAGAG GCGGG |
| | CGTCA GACTC CAGAC TCCTG TGCCG GTACA TAATG ACATG TTCTC CGCCC |
| 2101 | GACTA CGGCT ACGCT CTGGA CTACT GGGGT CAAGG AACCT CAGTC ACCGT |
| | CTGAT GCCGA TGCGA GACCT GATGA CCCCA GTTCC TTGGA GTCAG TGGCA |
| 2151 | CTCCT CATGA |
| | GAGGA GTACT |

What is claimed is:

1. A chimeric molecule comprising Factor VII (“FVII”), an XTEN polypeptide, and an anti-GPIIb/IIIa antibody or antigen-binding molecule thereof, wherein the anti-GPIIb/IIIa antibody or antigen-binding molecule thereof exhibits one or more of the following characteristics:
(a) the anti-GPIIb/IIIa antibody or antigen-binding molecule thereof specifically binds to the same GPIIb/IIIa epitope as an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4;
(b) the anti-GPIIb/IIIa antibody or antigen-binding molecule thereof competitively inhibits GPIIb/IIIa binding to an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4; or
(c) the anti-GPIIb/IIIa antibody or antigen-binding molecule thereof comprises at least one, at least two, at least three, at least four, at least five, or at least six complementarity determining regions (CDR) or variants thereof selected from the CDRs of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, or 28F4.

2. The chimeric molecule of claim 1, wherein the anti-GPIIb/IIIa antibody or antigen-binding molecule thereof comprises six CDRs or variants thereof of an antibody selected from the group consisting of 34D10, 12B2, 2A2, 35D1, 36A8, 4B11, 1H6, 38G8, 21F10, 38A8, 18F7, 38F6, 13C1, 5C4, 23C10, 37C7, 28C2, 9D6, and 28F4.

3. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence at least about 60%, 70%, 80%, 90%, 95%, or 100% identical to any one of SEQ ID NOS: 25, 31, 37, 43 or 111;
(ii) a variable heavy chain CDR-2 (VH-CDR2) sequence at least about 60%, 70%, 80%, 90%, 95%, or 100% identical to any one of SEQ ID NOS:26, 32, 38, 44, or 112;
(iii) a variable heavy chain CDR-3 (VH-CDR3) sequence at least about 60%, 70%, 80%, 90%, 95%, or 100% identical to any one of SEQ ID NOS: 27, 33, 39, 45, or 113;
(iv) a variable light chain CDR-1 (VL-CDR1) sequence at least about 60%, 70%, 80%, 90%, 95%, or 100% identical to any one of SEQ ID NOS: 28, 34, 40, 117, or 114;
(v) a variable light chain CDR-2 (VL-CDR2) sequence at least about 60%, 70%, 80%, 90%, 95%, or 100% identical to any one of SEQ ID NOS: 29, 35, 41, 118, or 115; and,
(vi) a variable light chain CDR-3 (VL-CDR3) sequence at least about 60, 70, 80, 90, or 95% identical to any one of SEQ ID NOS: 30, 36, 42, 119, or 116.

4. A chimeric molecule comprising FVII, an XTEN polypeptide, and an anti-GPIIb/IIIa antibody or antigen-binding molecule thereof which comprises:
(i) a VH-CDR1 comprising the consensus sequence X₁YAMS wherein X₁ represents amino acid residues Thr (T), Ser (S), or Ala (A);
(ii) a VH-CDR2 comprising the consensus sequence SIX₂X₃GX₄X₅TYX₆X₇DSVKX₈ wherein X₂ represents amino acid residues Ser (S) or Asn (N), X₃ represents amino acid residues Ser (S) or Gly (G), X₄ represents amino acid residues Ser (S) or Gly (G), X₅ represents amino acid residues Ser (S), Asn (N), or Thr (T), X₆ represents amino acid residues Tyr (Y) or Phe (F), X₇ represents amino acid residues Leu (L) or Pro (P), and X₈ represents amino acids Gly (G) or Arg (R);
(iii) a VH-CDR3 comprising the consensus sequence GGDYGYAX₉DY,
wherein X₉ represents amino acid residues Leu (L) or Met (M);
(iv) a VL-CDR1 comprising the sequence RASSSVNYMY (SEQ ID NO: 28);
(v) a VL-CDR2 comprising the sequence YTSNLAP (SEQ ID NO: 29); and,
(vi) a VL-CDR3 comprising the sequence QQFSSSPWT (SEQ ID NO: 30).

5. The chimeric molecule of claim 4, wherein the anti-GPIIb/IIIa antibody or antigen-binding molecule thereof comprises:
(i) a VH-CDR1 sequence selected from the group consisting of SEQ ID NOS: 25, 31, 37, 43, and 111;
(ii) a VH-CDR2 sequence selected from the group consisting of SEQ ID NOS: 26, 32, 38, 44, and 112;
(iii) a VH-CDR3 sequence selected from the group consisting of SEQ ID NOS: 27, 33, 39, 45, and 113;
(iv) a VL-CDR1 sequence selected from the group consisting of SEQ ID NOS: 28, 34, 40, 117, and 114;
(v) a VL-CDR2 sequence selected from the group consisting of SEQ ID NOS: 29, 35, 41, 118, and 115; and,
(vi) a VL-CDR3 sequence selected from the group consisting of SEQ ID NOS: 30, 36, 42, 119, and 116.

6. A chimeric molecule comprising FVII, an XTEN polypeptide, and an anti-GPIIb/IIIa antibody or antigen-binding molecule thereof which comprises a VH comprising an amino acid sequence at least about 80%, 85%, 90%, 95%, or 100% identical to any one of SEQ ID NOS: 1, 3, 5, 7, or 97 and a VL comprising an amino acid sequence at least about 80%, 85%, 90%, 95%, or 100% identical to any one of SEQ ID NOS: 2, 4, 6, 99, or 98.

7. The chimeric molecule of claim 6, wherein the VH comprises the amino acid sequence of SEQ ID NO: 1 and the VL comprises the amino acid sequence of SEQ ID NO: 2 (34D10 antibody).

8. The chimeric molecule of claim 6, wherein the VH comprises the amino acid sequence of SEQ ID NO: 3 and the VL comprises the amino acid sequence of SEQ ID NO: 4 (2A2 antibody).

9. The chimeric molecule of claim 6, wherein the VH comprises the amino acid sequence of SEQ ID NO: 5 and the VL comprises the amino acid sequence of SEQ ID NO: 6 (36A8 antibody).

10. The chimeric molecule of claim 6, wherein the VH comprises the amino acid sequence of SEQ ID NO: 7 and the VL comprises the amino acid sequence of SEQ ID NO: 99 (4B11 antibody).

11. The chimeric molecule of claim 6, wherein the VH comprises the amino acid sequence of SEQ ID NO: 97 and the VL comprises the amino acid sequence of SEQ ID NO: 98 (35D1 antibody).

12. The chimeric molecule of any one of claims 1 to 11, the anti-GPIIb/IIIa antibody or antigen-binding molecule thereof binds to an epitope located in the extracellular domain of the alpha subunit of GPIIb/IIIa or the extracellular domain of the GPIIb/IIIa complex.

13. The chimeric molecule of any one of claims 1 to 12, wherein the anti-GPIIb/IIIa antibody or antigen-binding molecule thereof does not compete with fibrinogen for binding to GPIIb/IIIa.

14. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence at least about 60%, 70%, 80%, 90%, 95%, or 100% identical to any one of SEQ ID NOS: 46, 52, 120, or 126;
(ii) a variable heavy chain CDR-2 (VH-CDR2) sequence at least about 60%, 70%, 80%, 90%, 95%, or 100% identical to any one of SEQ ID NOS: 47, 53, 121, or 127;
(iii) a variable heavy chain CDR-3 (VH-CDR3) sequence at least about 60%, 70%, 80%, 90%, 95%, or 100% identical to any one of SEQ ID NOS: 48, 54, 122, or 128;
(iv) a variable light chain CDR-1 (VL-CDR1) sequence at least about 60%, 70%, 80%, 90%, 95%, or 100% identical to any one of SEQ ID NOS: 49, 55, 123, or 129;
(v) a variable light chain CDR-2 (VL-CDR2) sequence at least about 60%, 70%, 80%, 90%, 95%, or 100% identical to any one of SEQ ID NOS: 50, 56, 124, or 130; and,
(vi) a variable light chain CDR-3 (VL-CDR3) sequence at least about 60%, 70%, 80%, 90%, 95%, or 100% identical to any one of SEQ ID NO: 51, 57, 125, or 131.

15. The chimeric molecule of any one of claims 1, 2 and 14, wherein the anti-GPIIb/IIIa antibody or antigen-binding molecule thereof comprises a VH comprising an amino acid sequence at least about 80%, 85%, 90%, 95%, or 100% identical to any one of SEQ ID NOS: 8, 10, 100, or 102, and a VL comprising an amino acid sequence at least about 80%, 85%, 90%, 95%, or 100% identical to any one of SEQ ID NOS: 9, 11, 101, or 103.

16. The chimeric molecule of claim 15, wherein the VH comprises the amino acid sequence of SEQ ID NO: 8 and the VL comprises the amino acid sequence of SEQ ID NO: 9 (1H6 antibody).

17. The chimeric molecule of claim 15, wherein the VH comprises the amino acid sequence of SEQ ID NO: 10 and the VL comprises the amino acid sequence of SEQ ID NO: 11 (38A8 antibody).

18. The chimeric molecule of claim 15, wherein the VH comprises the amino acid sequence of SEQ ID NO: 100 and the VL comprises the amino acid sequence of SEQ ID NO: 101 (38G8 antibody).

19. The chimeric molecule of claim 15, wherein the VH comprises the amino acid sequence of SEQ ID NO: 102 and the VL comprises the amino acid sequence of SEQ ID NO: 103 (21F10 antibody).

20. The chimeric molecule of any one of claims 1, 2 and 14 to 19, wherein the anti-GPIIb/IIIa antibody or antigen-binding molecule thereof binds to an epitope located in the extracellular domain of the alpha subunit of GPIIb/IIIa.

21. The chimeric molecule of any one of claims 1, 2 and 14 to 20, wherein the anti-GPIIb/IIIa antibody or antigen-binding molecule thereof competes with fibrinogen for binding to GPIIb/IIIa.

22. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence at least about 60%, 70%, 80%, 90%, 95%, or 100% identical to SEQ ID NO: 58;
(ii) a variable heavy chain CDR-2 (VH-CDR2) sequence at least about 60%, 70%, 80%, 90%, 95%, or 100% identical to SEQ ID NO: 59;
(iii) a variable heavy chain CDR-3 (VH-CDR3) sequence at least about 60%, 70%, 80%, 90%, 95%, or 100% identical to SEQ ID NO: 60;
(iv) a variable light chain CDR-1 (VL-CDR1) sequence at least about 60%, 70%, 80%, 90%, 95%, or 100% identical to SEQ ID NO: 61;
(v) a variable light chain CDR-2 (VL-CDR2) sequence at least about 60%, 70%, 80%, 90%, 95%, or 100% identical to SEQ ID NO: 62; and,
(vi) a variable light chain CDR-3 (VL-CDR3) sequence at least about 60%, 70%, 80%, 90%, 95%, or 100% identical to SEQ ID NO: 63.

23. The chimeric molecule of claim 1, 2, and 22, wherein the anti-GPIIb/IIIa antibody or antigen-binding molecule thereof comprises a VH comprising an amino acid sequence at least about 80%, 85%, 90%, 95%, or 100% identical to SEQ ID NO: 12 and a VL comprising an amino acid sequence at least about 80%, 85%, 90%, 95%, or 100% identical to SEQ ID NO: 13 (18F7 antibody).

24. The chimeric molecule of claim 22 or 23, wherein the anti-GPIIb/IIIa antibody or antigen-binding molecule thereof binds to an epitope located in the extracellular domain of the alpha subunit of GPIIb/IIIa.

25. The chimeric molecule of any one of claims 22 to 24, wherein the anti-GPIIb/IIIa antibody or antigen-binding molecule thereof competes with fibrinogen for binding to GPIIb/IIIa.

26. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence at least about 60%, 70%, 80%, 90%, 95%, or 100% identical to any one of SEQ ID NOS: 64, 70, or 135;
(ii) a variable heavy chain CDR-2 (VH-CDR2) sequence at least about 60%, 70%, 80%, 90%, 95%, or 100% identical to any one of SEQ ID NOS: 65, 71, or 136;
(iii) a variable heavy chain CDR-3 (VH-CDR3) sequence at least about 60%, 70%, 80%, 90%, 95%, or 100% identical to any one of SEQ ID NOS: 66, 72, or 137;
(iv) a variable light chain CDR-1 (VL-CDR1) sequence at least about 60%, 70%, 80%, 90%, 95%, or 100% identical to any one of SEQ ID NOS: 67, 132, or 138;
(v) a variable light chain CDR-2 (VL-CDR2) sequence at least about 60%, 70%, 80%, 90%, 95%, or 100% identical to any one of SEQ ID NOS: 68, 133, or 139; and,
(vi) a variable light chain CDR-3 (VL-CDR3) sequence at least about 60%, 70%, 80%, 90%, 95%, or 100% identical to any one of SEQ ID NOS: 69, 134, or 140.

27. A chimeric molecule comprising FVII, an XTEN polypeptide, and an anti-GPIIb/IIIa antibody or antigen-binding molecule thereof which comprises:
(i) a VH-CDR1 comprising the sequence SYWIE (SEQ ID NO: 64);
(ii) a VH-CDR2 comprising the consensus sequence EILPGX₁₄GX₁₅TKYNX₁₆KFKG (SEQ ID NO: ______) wherein X₁₄ represents amino acid residues Ser (S) or Thr (T), X₁₅ represents amino acid residues Ile (I) or Tyr (Y), and X₁₆ represents amino acid residues Asp (D) or Glu (E);
(iii) a VH-CDR3 comprising the sequence LISYYYAMDY (SEQ ID NO: 66);
(iv) a VL-CDR1 comprising the sequence RASQDISNYLN (SEQ ID NO: 67);
(v) a VL-CDR2 comprising the sequence YTSRLHS (SEQ ID NO: 68); and,
(vi) a VL-CDR3 comprising the sequence QQGNTLPPT (SEQ ID NO: 69).

28. The chimeric molecule of any one of claims 1, 2, 26, and 27, wherein the anti-GPIIb/IIIa antibody or antigen-binding molecule thereof comprises a VH comprising an amino acid sequence at least about 80%, 85%, 90%, 95%, or 100% identical to any one of SEQ ID NOS: 14, 16, or 105 and a VL comprising an amino acid sequence at least about 80%, 85%, 90%, 95%, or 100% identical to any one of SEQ ID NOS: 15, 104, or 106.

29. The chimeric molecule of claim 28, wherein the VH comprises the amino acid sequence of SEQ ID NO: 14 and the VL comprises the amino acid sequence of SEQ ID NO: 15 (12B2 antibody).

30. The chimeric molecule of claim 28, wherein the VH comprises the amino acid sequence of SEQ ID NO: 16 and the VL comprises the amino acid sequence of SEQ ID NO: 104 (38F6 antibody).

31. The chimeric molecule of claim 28, wherein the VH comprises the amino acid sequence of SEQ ID NO: 105 and the VL comprises the amino acid sequence of SEQ ID NO: 106 (13C1 antibody).

32. The chimeric molecule of any one of claims 26 to 31, wherein the anti-GPIIb/IIIa antibody or antigen-binding molecule thereof binds to an epitope located in the extracellular domain of the beta subunit of GPIIb/IIIa.

33. The chimeric molecule of any one of claims 26 to 32, wherein the GPIIb/IIIa antibody or antigen-binding molecule thereof does not compete with fibrinogen for binding to GPIIb/IIIa.

34. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence at least about 60%, 70%, 80%, 90%, 95%, or 100% identical to any one of SEQ ID NOS: 73, 76, 79, 85, or 147;
(ii) a variable heavy chain CDR-2 (VH-CDR2) sequence at least about 60%, 70%, 80%, 90%, 95%, or 100% identical to any one of SEQ ID NOS: 74, 77, 80, 86, or 148;
(iii) a variable heavy chain CDR-3 (VH-CDR3) sequence at least about 60%, 70%, 80%, 90%, 95%, or 100% identical to any one of SEQ ID NOS: 75, 78, 81, 87, or 149;
(iv) a variable light chain CDR-1 (VL-CDR1) sequence at least about 60%, 70%, 80%, 90%, 95%, or 100% identical to any one of SEQ ID NOS: 141, 144, 82, 88, or 150;
(v) a variable light chain CDR-2 (VL-CDR2) sequence at least about 60%, 70%, 80%, 90%, 95%, or 100% identical to any one of SEQ ID NOS: 142, 145, 83, 89, or 151; and,
(vi) a variable light chain CDR-3 (VL-CDR3) sequence at least about 60%, 70%, 80%, 90%, 95%, or 100% identical to any one of SEQ ID NO: 143, 146, 84, 90, or 152.

35. The chimeric molecule of any one of claims 1, 2, and 34, wherein the anti-GPIIb/IIIa antibody or antigen-binding molecule thereof comprises a VH comprising an amino acid sequence at least 80%, 85%, 90%, 95%, or 100% identical to any one of SEQ ID NOS: 17, 18, 19, 21, or 109 and a VL comprising an amino acid sequence at least 80%, 85%, 90%, 95%, or 100% identical to any one of SEQ ID NOS: 107, 108, 20, 22, or 110.

36. The chimeric molecule of claim 35, wherein the VH comprises the amino acid sequence of SEQ ID NO: 17 and the VL comprising the amino acid sequence of SEQ ID NO: 107 (5C4 antibody).

37. The chimeric molecule of claim 35, wherein the VH comprises the amino acid sequence of SEQ ID NO: 18 and the VL comprising the amino acid sequence of SEQ ID NO: 108 (23C10 antibody).

38. The chimeric molecule of claim 35, wherein the VH comprises the amino acid sequence of SEQ ID NO: 109 and the VL comprising the amino acid sequence of SEQ ID NO: 110 (37C7 antibody).

39. The chimeric molecule of claim 35, wherein the VH comprises the amino acid sequence of SEQ ID NO: 19 and the VL comprising the amino acid sequence of SEQ ID NO: 20 (28C2 antibody).

40. The chimeric molecule of claim 35, wherein the VH comprises the amino acid sequence of SEQ ID NO: 21 and the VL comprising the amino acid sequence of SEQ ID NO: 22 (9D6 antibody).

41. The chimeric molecule of any one of any one of claims 1, 2, and 34 to 40, wherein the anti-GPIIb/IIIa antibody or antigen-binding molecule thereof binds to an epitope located in the extracellular domain of the beta subunit of GPIIb/IIIa.

42. The chimeric molecule of any one of claims 1, 2, and 34 to 41, wherein the anti-GPIIb/IIIa antibody or antigen-binding molecule thereof competes with fibrinogen for binding to GPIIb/IIIa.

43. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence at least about 60%, 70%, 80%, 90%, 95%, or 100% identical to SEQ ID NO: 91;
(ii) a variable heavy chain CDR-2 (VH-CDR2) sequence at least about 60%, 70%, 80%, 90%, 95%, or 100% identical to SEQ ID NO: 92;
(iii) a variable heavy chain CDR-3 (VH-CDR3) sequence at least about 60%, 70%, 80%, 90%, 95%, or 100% identical to SEQ ID NO: 93;
(iv) a variable light chain CDR-1 (VL-CDR1) sequence at least about 60%, 70%, 80%, 90%, 95%, or 100% identical to SEQ ID NO: 94;
(v) a variable light chain CDR-2 (VL-CDR2) sequence at least about 60%, 70%, 80%, 90%, 95%, or 100% identical to SEQ ID NO: 95; and,
(vi) a variable light chain CDR-3 (VL-CDR3) sequence at least about 60%, 70%, 80%, 90%, 95%, or 100% identical to SEQ ID NO: 96.

44. The chimeric molecule of any one of claims 1, 2, and 43, wherein the anti-GPIIb/IIIa antibody or antigen-binding molecule thereof comprises a VH comprising an amino acid sequence at least about 80%, 85%, 90%, 95%, or 100% identical to SEQ ID NO: 23 and a VL comprising an amino acid sequence at least about 80%, 85%, 90%, 95%, or 100% identical to SEQ ID NO: 24 (28F4 antibody).

45. The chimeric molecule of claim 44, wherein the anti-GPIIb/IIIa antibody or antigen-binding molecule thereof binds to an epitope located in the extracellular domain of the beta subunit of GPIIb/IIIa.

46. The chimeric molecule of any one of claims 43 to 45, wherein the anti-GPIIb/IIIa antibody or antigen-binding molecule thereof competes with fibrinogen for binding to GPIIb/IIIa.

47. The chimeric molecule of any one of claims 1 to 46, wherein the anti-GPIIb/IIIa antibody or antigen-binding molecule thereof comprises:
(a) a single chain Fv (“scFv”);
(b) a diabody;
(c) a minibody;
(d) a polypeptide chain of an antibody;
(e) F(ab′)2; or
(f) F(ab).

48. The chimeric molecule of any one of claims 1 to 47, wherein the anti-GPIIb/IIIa antibody or antigen-binding molecule thereof does not inhibit platelet function.

49. The chimeric molecule of any one of claims 1 to 48, wherein the anti-GPIIb/IIIa antibody or antigen-binding molecule thereof does not activate platelet.

50. The chimeric molecule of any one of claims 1 to 49, wherein the chimeric molecule does not induce thrombocytopenia.

51. The chimeric molecule of any one of claims 1 to 50, wherein FVII is activated FVII (“FVIIa”).

52. The chimeric molecule of any one of claims 1 to 51, further comprising an optional linker between FVII and the XTEN polypeptide, between FVII and the anti-GPIIb/IIIa antibody or antigen-binding molecule thereof, or between the XTEN polypeptide and the anti-GPIIb/IIIa antibody or antigen-binding molecule thereof.

53. The chimeric molecule of any one of claims 1 to 52, which comprises a formula selected from the group consisting of:
(a) FVII-(L1)-X-(L2)-Tm;
(b) FVII-(L1)-Tm-(L2)-X;
(c) Tm-(L1)-X-(L2)-FVII;
(d) Tm-(L1)-FVII-(L2)-X;
(e) X-(L1)-Tm-(L2)-FVII; and
(f) X-(L1)-FVII-(L2)-Tm; wherein
FVII IS FVIIA;
X IS THE XTEN POLYPEPTIDE;
TM IS THE ANTI-GPIIB/IIIA ANTIBODY OR ANTIGEN-BINDING MOLECULE THEREOF;
L1 IS A FIRST OPTIONAL LINKER, AND
L2 IS A SECOND OPTIONAL LINKER.

54. The chimeric molecule of any one of claims 1 to 50, which comprises a first polypeptide chain and a second polypeptide chain, which are associated with each other,
(a) wherein the first polypeptide chain comprises a light chain of FVII and the XTEN polypeptide and the second polypeptide chain comprises a heavy chain of FVII and the anti-GPIIb/IIIa antibody or antigen-binding molecule thereof;
(b) wherein the first polypeptide chain comprises a light chain of FVII and the anti-GPIIb/IIIa antibody or antigen-binding molecule thereof and the second polypeptide chain comprises a heavy chain of FVII and the XTEN polypeptide;
(c) wherein the first polypeptide chain comprises a light chain of FVII, the XTEN polypeptide, and the anti-GPIIb/IIIa antibody or antigen-binding molecule thereof, in any order, and the second chain comprises a heavy chain of FVII; or
(d) wherein the first polypeptide chain comprises a light chain of FVII and the second chain comprises a heavy chain of FVII, the XTEN polypeptide, and the anti-GPIIb/IIIa antibody or antigen-binding molecule thereof, in any order.

55. The chimeric molecule of any one of claims 1 to 50, which comprises a first polypeptide chain and a second polypeptide chain, which are associated with each other,
(g) wherein the first polypeptide chain comprises the formula of FVIIL-X or X-FVIIL and the second polypeptide chain comprises the formula of FVIIH-Tm or Tm-FVIIH,
(h) wherein the first polypeptide chain comprise the formula of FVIIL-Tm or Tm-FVIIL and the second polypeptide chain comprises the formula of FVIIH-X or X-FVIIH;
(i) wherein the first polypeptide chain comprise the formula of FVIIL and the second polypeptide chain comprises the formula of FVIIH-X-Tm or Tm-X-FVIIH;
(j) wherein the first polypeptide chain comprise the formula of FVIIL and the second polypeptide chain comprises the formula of FVIIH-Tm-X or X-Tm-FVIIH;
(k) wherein the first polypeptide chain comprise the formula of FVIIL-Tm-X or X-Tm-FVIIL or and the second polypeptide chain comprises the formula of FVIIH; or
(l) wherein the first polypeptide chain comprise the formula of FVIIL-X-Tm or Tm-X-FVIIL and the second polypeptide chain comprises the formula of FVIIH, wherein
FVII_{H} is a heavy chain of FVII;
Tm is the anti-GPIIb/IIIa antibody or antigen-binding molecule thereof;
FVII_{L} is a light chain of FVII; and
X is the XTEN polypeptide.

56. The chimeric molecule of any one of claims 1 to 50, comprising a formula selected from the group consisting of:
(f) X-FVII_{L}:FVII_{H}-Tm;
(g) Tm-FVII_{L}:FVII_{H}-X;
(h) FVII_{L}:FVII_{H}-X-Tm or Tm-X-FVII_{H}:FVII_{L};
(i) FVII_{L}:FVII_{H}-Tm-X or X-Tm-FVII_{H}:FVII_{L};
(j) FVII_{L}-X-Tm:FVII_{H} or FVII_{H}:Tm-X-FVII_{L}; and
(k) FVII_{L}-Tm-X:FVII_{H} or FVII_{H}:Tm-X-FVII_{L},
wherein FVII_{H} is a heavy chain of FVII;
Tm is the anti-GPIIb/IIIa antibody or antigen-binding molecule thereof;
FVII_{L} is a light chain of FVII;
X is the XTEN polypeptide; and
(:) is an association between two polypeptide chains.

57. The chimeric molecule of any one of claims 54 to 56, wherein the association between the first polypeptide chain and the second polypeptide chain is a covalent bond or a non-covalent bond.

58. The chimeric molecule of any one of claims 54 to 57, wherein the association between the first polypeptide chain and the second polypeptide chain is a covalent bond between the heavy chain and the light chain of FVII.

59. The chimeric molecule of claim 58, wherein the covalent bond is a disulfide bond.

60. The chimeric molecule of claim 1 to 50, which comprises a single polypeptide chain, which comprises, from N terminus to C terminus,
(a) a light chain of FVII, the XTEN polypeptide, a protease cleavage site, a heavy chain of FVII, and the anti-GPIIb/IIIa antibody or antigen-binding molecule thereof; or
(b) a light chain of FVII, the anti-GPIIb/IIIa antibody or antigen-binding molecule thereof, a protease cleavage site, a heavy chain of FVII, and the XTEN polypeptide.

61. The chimeric molecule of claim 60, wherein the protease cleavage site is an intracellular processing site.

62. The chimeric molecule of claim 61, wherein the intracellular processing site is processed by a proprotein convertase.

63. The chimeric molecule of claim 62, wherein the proprotein convertase is selected from the group consisting of PC5, PACE, PC7, and any combinations thereof.

64. A chimeric molecule comprising a first polypeptide chain and a second polypeptide chain, which are associated with each other,
(e) wherein the first polypeptide chain comprises a light chain of FVII and an XTEN polypeptide and the second chain polypeptide chain comprises a heavy chain of FVII and a targeting moiety, which binds to a platelet;
(f) wherein the first polypeptide chain comprises a light chain of FVII and a targeting moiety, which binds to a platelet, and the second polypeptide chain comprises a heavy chain of FVII and an XTEN polypeptide;
(g) wherein the first polypeptide chain comprises a light chain of FVII and the second polypeptide chain comprises a heavy chain of FVII, an XTEN polypeptide, and a targeting moiety, which binds to a platelet; or
(h) wherein the first polypeptide chain comprises a light chain of FVII and the second polypeptide chain comprises a heavy chain of FVII, a targeting moiety, which binds to a platelet, or an XTEN polypeptide.

65. The chimeric molecule of claim 64,
(e) wherein the first polypeptide chain comprises a formula of FVII_{L}-Tm or Tm-FVII_{L} and the second polypeptide chain comprises FVII_{H}-X or X-FVII_{H};
(f) wherein the first polypeptide chain comprises a formula of FVII_{L}-X or X-FVII_{L} and the second polypeptide chain comprises a formula of FVII_{H}-Tm or Tm-FVII_{H};
(g) wherein the first polypeptide chain comprises the formula of FVII_{L} and the second polypeptide chain comprises a formula of FVII_{H}-X-Tm or Tm-X-FVII_{H}; or
(h) wherein the first polypeptide chain comprises the formula of FVII_{L} and the second polypeptide chain comprises a formula of FVII_{H}-Tm-X or X-Tm-FVII_{H},
wherein FVII_{H} is the heavy chain of FVII;
Tm is the targeting moiety, which binds to a platelet;
FVII_{L} is the light chain of FVII; and
X is the XTEN polypeptide.

66. The chimeric molecule of claim 64, comprising a formula selected from the group consisting of:
(e) X-FVIIL:FVIIH-Tm or Tm-FVIIH: FVIIL-X;
(f) Tm-FVIIL:FVIIH-X or X-FVIIH: FVIIL-Tm;
(g) FVIIL:FVIIH-X-Tm or Tm-X-FVIIH:FVIIL; and
(h) FVIIL:FVIIH-Tm-X or X-Tm-FVIIH:FVIIL;
wherein FVII_{H} is the heavy chain of FVII;
Tm is the targeting moiety, which binds to a platelet;
FVII_{L} is the light chain of FVII;
X is the XTEN polypeptide; and
(:) is an association between two polypeptide chains.

67. The chimeric molecule of any one of claims 64 to 66, wherein the association between the first polypeptide chain and the second polypeptide chain is a covalent bond or a non-covalent bond.

68. The chimeric molecule of any one of claims 64 to 67, wherein the association between the first polypeptide chain and the second polypeptide chain is a covalent bond between the heavy chain and the light chain of FVII.

69. The chimeric molecule of claim 68, wherein the covalent bond is a disulfide bond.

70. A chimeric molecule comprising a single polypeptide chain, which comprises, from N terminus to C terminus,
(a) a light chain of FVII, an XTEN polypeptide, a protease cleavage site, a heavy chain of FVII, and a targeting moiety which binds to a platelet;
(b) a light chain of FVII, a targeting moiety which binds to a platelet, a protease cleavage site, a heavy chain of FVII, and an XTEN polypeptide;
(c) a light chain of FVII, a protease cleavage site, a heavy chain of FVII, an XTEN polypeptide, and a targeting moiety which binds to a platelet; or
(d) a light chain of FVII, a protease cleavage site, a heavy chain of FVII, a targeting moiety which binds to a platelet, and an XTEN polypeptide.

71. The chimeric molecule of claim 70, wherein the protease cleavage site is an intracellular processing site.

72. The chimeric molecule of claim 71, wherein the intracellular processing site is processed by a proprotein convertase.

73. The chimeric molecule of claim 72, wherein the proprotein convertase is selected from the group consisting of PC5, PACE, PC7, and any combinations thereof.

74. The chimeric molecule of any one of claims 64 to 73, wherein the targeting moiety is selected from the group consisting of: an antibody or antigen-binding molecule thereof, a receptor binding portion of a receptor, and a peptide.

75. The chimeric molecule of any one of claims 64 to 74, wherein the targeting moiety selectively binds to a resting platelet or an activated platelet.

76. The chimeric molecule of any one of claims 64 to 75, wherein the targeting moiety selectively binds to a target selected from the group consisting of: GPIba, GPVI, GPIX, a nonactive form of glycoprotein IIb/IIIa (“GPIIb/IIIa”), an active form of GPIIb/IIIa, P selectin, GMP-33, LAMP-1, LAMP-2, CD40L, LOX-1, and any combinations thereof.

77. The chimeric molecule of claim 76, wherein the targeting moiety is an antibody or antigen-binding molecule thereof, which binds to a GPIIb/IIIa epitope.

78. The chimeric molecule of any one of claims 1 to 77, wherein the half-life of FVII is increased compared to FVIIa consisting of the heavy chain and the light chain.

79. The chimeric molecule of claim 78, wherein the half-life of FVII is extended at least by about 1.5 fold, about 2.0 fold, about 2.5 fold, about 3.0 fold, about 3.5 fold, about 4 fold, about 4.5 fold, about 5 fold, about 6 fold, about 7 fold, about 8 fold, about 9 fold, about 10 fold, about 11 fold, about 12 fold, about 13 fold, about 14 fold, or about 15 fold compared to FVIIa consisting of the heavy chain and the light chain.

80. The chimeric molecule of any one of claims 1 to 79, wherein the clotting activity of FVII is equal to or greater than FVIIa consisting of the heavy chain and the light chain.

81. The chimeric molecule of claim 80, wherein the clotting activity is measured by a ROTEM assay.

82. The chimeric molecule of claim 81, wherein the clotting activity is measured by an aPTT assay.

83. The chimeric molecule of any one of claims 1 to 82, wherein the XTEN polypeptide comprises an AE motif, an AG motif, an AD motif, an AM motif, an AQ motif, an AF motif, a BC motif, a BD motif, or any combinations thereof.

84. The chimeric molecule of any one of claim 83, wherein the XTEN polypeptide comprises about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, about 2004 amino acids, or any combinations thereof.

85. The chimeric molecule of any one of claim 84, wherein the XTEN polypeptide is selected from the group consisting of: AE42, AE72, AE864, AE576, AE288, AE144, AG864, AG576, AG288, AG144, and any combinations thereof.

86. The chimeric molecule of claim 85, wherein the XTEN polypeptide is selected from the group consisting of SEQ ID NOs: 224-239, and any combinations thereof.

87. The chimeric molecule of any one of claims 54 to 59, 64 to 69, and 74 to 97, further comprising a linker, wherein the linker connects the light chain of FVII with the XTEN polypeptide, the heavy chain of FVII with the targeting moiety, or both.

88. The chimeric molecule of any one of claims 54 to 59, 64 to 69, and 74 to 97, further comprising a linker, wherein the linker connects the light chain of FVII with the targeting moiety, the light chain of FVII with the XTEN polypeptide, or both.

89. The chimeric molecule of claim 87 or 88, wherein the linker comprises at least about 1 amino acid, about 10 amino acids, about 20 amino acids, about 30 amino acids, about 40 amino acids, about 50 amino acids, about 60 amino acids, about 70 amino acids, about 80 amino acids, about 90 amino acids, about 100 amino acids, about 110 amino acids, abut 120 amino acids, about 130 amino acids, about 140 amino acids, about 150 amino acids, about 160 amino acids, or any combinations thereof.

90. The chimeric molecule of any one of claims 87 to 89, wherein the linker comprises a peptide having the formula [(Gly)ₓ-Ser_{y}]_{z}, where x is from 1 to 4, y is 0 or 1, and z is from 1 to 50.

91. The chimeric molecule of any one of claims 1 to 90, wherein the heavy chain of FVII comprises at least about 80%, 85%, 90%, 95%, 96%, 97%, 98%, 99%, or 100% identical to SEQ ID NO: 178.

92. The chimeric molecule of any one of claims 1 to 91, wherein the light chain of FVII comprises at least about 80%, 85%, 90%, 95%, 96%, 97%, 98%, 99%, or 100% identical to SEQ ID NO: 179.

93. The chimeric molecule of any one of claims 1 to 92, which comprises an amino acid sequence at least about 70%, 80%, 90%, 95%, 96%, 97%, 98%, or 99% identical to the amino acid sequence encoded by SEQ ID NO: 192 or SEQ ID NO: 193.

94. The chimeric molecule of claim 93, wherein the chimeric molecule comprises the amino acid sequence encoded by SEQ ID NO: 192 or SEQ ID NO: 193.

95. The chimeric molecule of any one of claims 1 to 94, further comprising a heterologous moiety fused to the heavy chain of FVII, the light chain of FVII, the XTEN polypeptide, the targeting moiety, or any combinations thereof.

96. The chimeric molecule of claim 95, wherein the heterologous moiety is a polypeptide moiety or a non-polypeptide moiety.

97. The chimeric molecule of claim 96, wherein the heterologous moiety extends the half-life of FVII.

98. The chimeric molecule of claim 97, wherein the heterologous moiety is selected from the group consisting of albumin, albumin binding polypeptide or fatty acid, Fc, transferrin, PAS, the C-terminal peptide (CTP) of the β subunit of human chorionic gonadotropin, polyethylene glycol (PEG), hydroxyethyl starch (HES), albumin-binding small molecules, vWF, an additional XTEN polypeptide, and any combinations thereof.

99. A pharmaceutical composition comprising the chimeric molecule of any one of claims 1 to 98 and a pharmaceutically acceptable carrier.

100. A polynucleotide encoding the chimeric molecule of any one of claims 1 to 98 or the complement thereof.

101. A set of polynucleotides comprising a first polynucleotide encoding the first polypeptide chain of the chimeric molecule of any one of claims 54 to 59, 64 to 69, and 74 to 98 or the complement thereof and a second polynucleotide encoding the second polypeptide chain of said chimeric molecule or the complement thereof.

102. A vector comprising the polynucleotide of claim 100 or the complement thereof or the set of polynucleotides of claim 101 or the complement thereof.

103. A set of vectors comprising a first vector comprising the first polynucleotide of claim 101 or the complement thereof, and a second vector comprising the second polynucleotide or the complement thereof.

104. The vector of claim 102 or the set of vectors of claim 103, further comprising a nucleotide sequence encoding an enzyme which processes the intracellular processing site.

105. A host cell comprising the vector of claim 102 or 104 or the set of vectors of claim 103 or 104.

106. The host cell of claim 105, further comprising a nucleotide sequence encoding an enzyme which processes the intracellular processing site.

107. A method of making a chimeric molecule comprising transfecting a host cell with the vector of claim 102 or 104 or the set of vectors of claim 103 or 104 and culturing the cell in a medium under a suitable condition.

108. The method of claim 107, further comprising isolating the chimeric molecule.

109. A method of reducing a frequency or degree of a bleeding episode in a subject in need thereof comprising administering the chimeric molecule of any one of claims 1 to 98, the composition of claim 99, the polynucleotide of claim 100 or the set of polynucleotides of claim 101, the vector of claim 102 or 104 or the set of vectors of claim 103 or 104, or the host cell of claim 105 or 106.

110. A method of preventing an occurrence of a bleeding episode in a subject in need thereof comprising administering the chimeric molecule of any one of claims 1 to 98, the composition of claim 99, the polynucleotide of claim 100 or the set of polynucleotides of claim 101, the vector of claim 102 or 104 or the set of vectors of claim 103 or 104, or the host cell of claim 105 or 106.

111. The method of claim 109 or 110, wherein the subject has developed or has the capacity to develop an inhibitor against Factor VIII (“FVIII”), Factor IX (“FIX”), or both.

112. The method of claim 111, wherein the inhibitor against FVIII or FIX is a neutralizing antibody against FVIII, FIX, or both.

113. The method of any one of claims 109 to 112, wherein the bleeding episode is caused by a blood coagulation disorder.

114. The method of claim 113, wherein the blood coagulation disorder is hemophilia A or hemophilia B.

115. The method of any one of claims 109 to 114, wherein the bleeding episode is derived from hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof.

116. The method of any one of claims 109 to 115, wherein the subject is a human subject.

117. The chimeric molecule of any one of claims 1 to 98, the composition of claim 99, the polynucleotide of claim 100 or the set of polynucleotides of claim 101, the vector of claim 102 or 104 or the set of vectors of claims 103 or 104, or the host cell of claim 105 or 106 for use in reducing a frequency or degree of a bleeding episode or reducing or preventing an occurrence of a bleeding episode in a subject in need thereof.

118. Use of the chimeric molecule of any one of claims 1 to 98, the composition of claim 99, the polynucleotide of claim 100 or the set of polynucleotides of claim 101, the vector of claim 102 or 104 or the set of vectors of claims 103 or 104, or the host cell of claim 105 or 106 for the manufacture of a medicament for reducing a frequency or degree of a bleeding episode or reducing or preventing an occurrence of a bleeding episode in a subject in need thereof.

119. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence at least about 60%, 70%, 80%, 90%, 95%, or 100% identical to SEQ ID NO: 31;
(ii) a variable heavy chain CDR-2 (VH-CDR2) sequence at least about 60%, 70%, 80%, 90%, 95%, or 100% identical to SEQ ID NO: 32;
(iii) a variable heavy chain CDR-3 (VH-CDR3) sequence at least about 60%, 70%, 80%, 90%, 95%, or 100% identical to SEQ ID NO: 33;
(iv) a variable light chain CDR-1 (VL-CDR1) sequence at least about 60%, 70%, 80%, 90%, 95%, or 100% identical to SEQ ID NO: 34;
(v) a variable light chain CDR-2 (VL-CDR2) sequence at least about 60%, 70%, 80%, 90%, 95%, or 100% identical to SEQ ID NO: 35; and
(vi) a variable light chain CDR-3 (VL-CDR3) sequence at least about 60, 70, 80, 90, or 95% identical to SEQ ID NO: 36.

120. A chimeric molecule comprising FVII, an XTEN polypeptide, and an anti-GPIIb/IIIa antibody or antigen-binding molecule thereof which comprises:
(i) a variable heavy chain CDR-1 (VH-CDR1) sequence of SEQ ID NO: 31;
(ii) a variable heavy chain CDR-2 (VH-CDR2) sequence of SEQ ID NO: 32;
(iii) a variable heavy chain CDR-3 (VH-CDR3) sequence of SEQ ID NO: 33;
(iv) a variable light chain CDR-1 (VL-CDR1) sequence of SEQ ID NO: 34;
(v) a variable light chain CDR-2 (VL-CDR2) sequence of SEQ ID NO: 35; and
(vi) a variable light chain CDR-3 (VL-CDR3) sequence of SEQ ID NO: 36.

121. A chimeric molecule comprising FVII, an XTEN polypeptide, and an anti-GPIIb/IIIa antibody or antigen-binding molecule thereof which comprises a VH and a VL, wherein the VH comprises the amino acid sequence of SEQ ID NO: 1.

122. A chimeric molecule comprising FVII, an XTEN polypeptide, and an anti-GPIIb/IIIa antibody or antigen-binding molecule thereof which comprises a VH and a VL, wherein the VL comprises an amino acid sequence of SEQ ID NO: 2.


SEQUENCE LISTING (275 sequences)

SEQ ID NO: 1 - protein, 118 aa, Artificial Sequence
  34D10 HC
EVKLVESGGGLVKPGGSLKLSCAASGFTFSAYAMSWVRQTPEKRLEWVASISSGGTTYYPDSVKRRFTISRDNARNILYLQMSSLRSEDTAMYYCTRGGDYGYALDYWGQGTSVTVSS

SEQ ID NO: 2 - protein, 107 aa, Artificial Sequence
  34D10 LC
ENVLTQSPAIMSASLGEKVTMSCRASSSVNYMYWYQQKSDASPKLWIYYTSNLAPGVPARFSGSGSGNSYSLTISSMEGEDAATYYCQQFSSSPWTFGGGTKLEIKR

SEQ ID NO: 3 - protein, 118 aa, Artificial Sequence
  2A2 HC
EVKLVESGGGLVKPGGSLKLSCAASGFTFRTYAMSWVRQTPEKRLEWVASISSGSSTYYLDSVKGRFTISRDNARNILYLQMSSLRSEDTAMYYCARGGDYGYALDYWGQGTSVTVSS

SEQ ID NO: 4 - protein, 107 aa, Artificial Sequence
  2A2 LC
ENVLTQSPAIMSASLGEKVTMSCRASSSVNYMYWYQQKSDASPKLWIYYTSNLAPGVPTRFSGSGSGNSYSLTISSLEGEDAGTYYCQQFSSSPWTFGGGTKLEIKR

SEQ ID NO: 5 - protein, 118 aa, Artificial Sequence
  36A8 HC
EVRLVESGGGLVKPGGSLKLSCAASGFTFSTYAMSWVRQTPEKRLEWVASINGGGSTYYPDSVKGRFTISRDNARNILYLQMRSLRSEDTAMYYCARGGDYGYALDYWGQGTSVTVSS

SEQ ID NO: 6 - protein, 107 aa, Artificial Sequence
  36A8 LC
ENVLTQSPAIMSASLGEKVTMNCRASSSVNYMYWYQQKSDASPKLWIFYTSNLAPGVPARFSGSGSGNSYSLTISSMEGEDAATYYCQQFSSSPWTFGGGSKLEIKR

SEQ ID NO: 7 - protein, 118 aa, Artificial Sequence
  4B11 LC
EVKLVESGGGLVKPGGSLKLSCAASGFTFSSYAMSWVRQTPEKRLAWVASISSGGNIYFPDSVKGRFTISRDDARNILYLQMRSLRSEDTAMYYCARGGDYGYAMDYWGQGTSVTVSS

SEQ ID NO: 8 - protein, 118 aa, Artificial Sequence
  1H6 HC
QVQLQQSGAELVRPGTSVKVSCKASGYAFTNYLIEWVKQRPGQGLEWIGVINPGSGGTNYNEKFKGKATLTADKSSSTAYMHLSSLTSDDSAVYFCARGRYEWYFDVWGAGTTVTVSS

SEQ ID NO: 9 - protein, 108 aa, Artificial Sequence
  1H6 LC
DIQMTQTTSSLSASLGDRVTISCRASQDITNYLNWYQRKPDGTVKLLIYYTSRLHSGVPSRFSGSGSGTDYSLTISNLEQEDIATYFCQQGYTLPYTFGGGTKLEIKR

SEQ ID NO: 10 - protein, 118 aa, Artificial Sequence
  38A8 HC
QVQLQQSGAELVRPGTSVKVSCKASGYAFTNYLIEWIKQRPGQGLEWIGVINPGSGGTNYNEKFKGKATLTADKSSSTAYMQLSSLTSDDSAVYFCARGRYEWYFDVWGAGTTVTVSS

SEQ ID NO: 11 - protein, 108 aa, Artificial Sequence
  38A8 LC
DIQMTQTTSSLSASLGDRVTISCRASQDISNYLNWYLQKPDGTVKLLIYYTSRLHSGVPSRFSGSGSGTDYSLSISNLEQEDIATYFCQQGYTLPYTFGGGTKLEIKR

SEQ ID NO: 12 - protein, 113 aa, Artificial Sequence
  18F7 HC
QVQLKESGPGLVAPSQSLSITCTVSGFSLTSYGVSWVRQPPGKGLEWLGIIWGDGSTNYHSVLKSRLSISKDNSKSQVFLKLNSLQTDDTATYYCAKQDFDVWGAGTTVTVSS

SEQ ID NO: 13 - protein, 108 aa, Artificial Sequence
  18F7 LC
DVQMIQSPFSLSASLGDIVTMTCQASQGTSINLNWFQQKPGKAPKLLIYGVSNLEDGVPSRFSGSRYGTDFTLTIGSLEDEDMATYFCLQHSYLPYTFGGGTKLEIKR

SEQ ID NO: 14 - protein, 119 aa, Artificial Sequence
  12B2 HC
QVQLQQSGAELTKPGASVKISCKATGYTFSSYWIEWVKQRPGHGLEWIGEILPGSGITKYNDKFKGKATFTADTSSNTAYMQLSSLTSEDSAVYSCARLISYYYAMDYWGQGTSVTVSS

SEQ ID NO: 15 - protein, 108 aa, Artificial Sequence
  12B2 LC
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEIKR

SEQ ID NO: 16 - protein, 119 aa, Artificial Sequence
  38F6 HC
QVQLQQSGAELMRPGASVKISCKATGYTFSSYWIEWVKQRPGHGLEWIGEILPGTGYTKYNEKFKGKATFTAETSSNTASMQVSSLTSEDSAVYFCARLISYYYAMDYWGQGTSVTVSS

SEQ ID NO: 17 - protein, 121 aa, Artificial Sequence
  5C4 HC
QVTLKASGPGILQPSQTLSLTCSFSGFSLNTSGLGVGWIRQPSGKGLEWLAHIWWDDDKRYNPALKSRLTISKDTSNNQIFLKIASVDTADTATYYCARSHYYGTFYFDYWGQGTTLTVSS

SEQ ID NO: 18 - protein, 133 aa, Artificial Sequence
  23C10 HC
FLLLIVPAYVLSQVTLKASGPGIVQPSQTLSLTCSFSGFSLNTSGMGVGWIRQPSGKGLEWLAHIWWDDDKRYNPALKSRLTISKDTSNNQIFLKIASVDTADTATYYCARSHYYGTFYFDYWGQGTTLTVSS

SEQ ID NO: 19 - protein, 121 aa, Artificial Sequence
  28C2 HC
QVTLKASGPGIVQPSQTLSLTCSFSGFSLNTSGMGVGWIRQPSGKGLEWLAHIWWDDDKRYNPALKSRLTISKDTSNNQIFLKIASVDTADTATYYCARSHYYGTFYFDYWGQGTTLTVSS

SEQ ID NO: 20 - protein, 108 aa, Artificial Sequence
  28C2 LC
DVQITQSPSYLAASPGETITINCRASKSISKYLAWYQEKPGTTYKLLIYSGSTLQSGIPSRFSGSGSGTDFTLTISSLEPEDFAMYYCQQHIEYPWTFGGGTKLEIKR

SEQ ID NO: 21 - protein, 121 aa, Artificial Sequence
  9D6 HC
QVTLKESGPGILQPSQTLSLTCSFSGFSLSTSGMGVGWIRQSSGKGLEWLAHIWWDDDKRYNPTLKSRLTISKDTSNNQVFLKIANMDTADIATYYCARSHYNGTFYFDFWGQGITLTVSS

SEQ ID NO: 22 - protein, 108 aa, Artificial Sequence
  9D6 LC
DVQITQSPSYLAASPGETITINCRASKSISKYLAWYQEKPGKTNKLLIYSGSTLQSGIPSRFSGSGSGTDFTLTISTLEPEDFAMYYCQQHIEYPWTFGGGTKLEIKR

SEQ ID NO: 23 - protein, 118 aa, Artificial Sequence
  28F4 HC
EVQLVESGGDLVKPGGSLKLSCAASGFTFSNYGMSWVRQTPDKRLEWVATISSGGTYTYYPDSVKGQFTIFRDNAKNTLYLQMSSLKSEDTAMYYCTRRDYDYEGFAYWGQGTLVTVS

SEQ ID NO: 24 - protein, 108 aa, Artificial Sequence
  28F4 LC
DIVLTQSPATLSVTPGDSVSLSCRASQSISNNLHWYQQKSHESPRLLIKYASHSISGIPSRFSGSGSGTDFTLSINSVETEDFGMYFCQQSNNWPFTFGSGTKLEIKR

SEQ ID NO: 25 - protein, 5 aa, Artificial Sequence
  2A2 HC CDR1
TYAMS

SEQ ID NO: 26 - protein, 16 aa, Artificial Sequence
  2A2 HC CDR2
SISSGSSTYYLDSVKG

SEQ ID NO: 27 - protein, 10 aa, Artificial Sequence
  2A2 HC CDR3
GGDYGYALDY

SEQ ID NO: 28 - protein, 10 aa, Artificial Sequence
  2A2 LC CDR1
RASSSVNYMY

SEQ ID NO: 29 - protein, 7 aa, Artificial Sequence
  2A2 LC CDR2
YTSNLAP

SEQ ID NO: 30 - protein, 9 aa, Artificial Sequence
  2A2 LC CDR3
QQFSSSPWT

SEQ ID NO: 31 - protein, 5 aa, Artificial Sequence
  34D10 HC CDR1
AYAMS

SEQ ID NO: 32 - protein, 16 aa, Artificial Sequence
  34D10 HC CDR2
SISSGGTTYYPDSVKR

SEQ ID NO: 33 - protein, 10 aa, Artificial Sequence
  34D10 HC CDR3
GGDYGYALDY

SEQ ID NO: 34 - protein, 10 aa, Artificial Sequence
  34D10 LC CDR1
RASSSVNYMY

SEQ ID NO: 35 - protein, 7 aa, Artificial Sequence
  34D10 LC CDR2
YTSNLAP

SEQ ID NO: 36 - protein, 9 aa, Artificial Sequence
  34D10 LC CDR3
QQFSSSPWT

SEQ ID NO: 37 - protein, 5 aa, Artificial Sequence
  36A8 HC CDR1
TYAMS

SEQ ID NO: 38 - protein, 16 aa, Artificial Sequence
  36A8 HC CDR2
SINGGGSTYYPDSVKG

SEQ ID NO: 39 - protein, 10 aa, Artificial Sequence
  36A8 HC CDR3
GGDYGYALDY

SEQ ID NO: 40 - protein, 10 aa, Artificial Sequence
  36A8 LC CDR1
RASSSVNYMY

SEQ ID NO: 41 - protein, 7 aa, Artificial Sequence
  36A8 LC CDR2
YTSNLAP

SEQ ID NO: 42 - protein, 9 aa, Artificial Sequence
  36A8 LC CDR3
QQFSSSPWT

SEQ ID NO: 43 - protein, 5 aa, Artificial Sequence
  4B11 HC CDR1
SYAMS

SEQ ID NO: 44 - protein, 16 aa, Artificial Sequence
  4B11 HC CDR2
SISSGGNIYFPDSVKG

SEQ ID NO: 45 - protein, 10 aa, Artificial Sequence
  4B11 HC CDR3
GGDYGYAMDY

SEQ ID NO: 46 - protein, 5 aa, Artificial Sequence
  1H6 HC CDR1
NYLIE

SEQ ID NO: 47 - protein, 17 aa, Artificial Sequence
  1H6 HC CDR2
VINPGSGGTNYNEKFKG

SEQ ID NO: 48 - protein, 9 aa, Artificial Sequence
  1H6 HC CDR3
GRYEWYFDV

SEQ ID NO: 49 - protein, 11 aa, Artificial Sequence
  1H6 LC CDR1
RASQDITNYLN

SEQ ID NO: 50 - protein, 7 aa, Artificial Sequence
  1H6 LC CDR2
YTSRLHS

SEQ ID NO: 51 - protein, 9 aa, Artificial Sequence
  1H6 LC CDR3
QQGYTLPYT

SEQ ID NO: 52 - protein, 5 aa, Artificial Sequence
  38A8 HC CDR1
NYLIE

SEQ ID NO: 53 - protein, 17 aa, Artificial Sequence
  38A8 HC CDR2
VINPGSGGTNYNEKFKG

SEQ ID NO: 54 - protein, 9 aa, Artificial Sequence
  38A8 HC CDR3
GRYEWYFDV

SEQ ID NO: 55 - protein, 11 aa, Artificial Sequence
  38A8 LC CDR1
RASQDISNYLN

SEQ ID NO: 56 - protein, 7 aa, Artificial Sequence
  38A8 LC CDR2
YTSRLHS

SEQ ID NO: 57 - protein, 9 aa, Artificial Sequence
  38A8 LC CDR3
QQGYTLPYT

SEQ ID NO: 58 - protein, 5 aa, Artificial Sequence
  18F7 HC CDR1
SYGVS

SEQ ID NO: 59 - protein, 16 aa, Artificial Sequence
  18F7 HC CDR2
IIWGDGSTNYHSVLKS

SEQ ID NO: 60 - protein, 5 aa, Artificial Sequence
  18F7 HC CDR3
QDFDV

SEQ ID NO: 61 - protein, 11 aa, Artificial Sequence
  18F7 LC CDR1
QASQGTSINLN

SEQ ID NO: 62 - protein, 7 aa, Artificial Sequence
  18F7 LC CDR2
GVSNLED

SEQ ID NO: 63 - protein, 9 aa, Artificial Sequence
  18F7 LC CDR3
LQHSYLPYT

SEQ ID NO: 64 - protein, 5 aa, Artificial Sequence
  12B2 HC CDR1
SYWIE

SEQ ID NO: 65 - protein, 17 aa, Artificial Sequence
  12B2 HC CDR2
EILPGSGITKYNDKFKG

SEQ ID NO: 66 - protein, 10 aa, Artificial Sequence
  12B2 HC CDR3
LISYYYAMDY

SEQ ID NO: 67 - protein, 11 aa, Artificial Sequence
  12B2 LC CDR1
RASQDISNYLN

SEQ ID NO: 68 - protein, 7 aa, Artificial Sequence
  12B2 LC CDR2
YTSRLHS

SEQ ID NO: 69 - protein, 9 aa, Artificial Sequence
  12B2 LC CDR3
QQGNTLPPT

SEQ ID NO: 70 - protein, 5 aa, Artificial Sequence
  38F6 HC CDR1
SYWIE

SEQ ID NO: 71 - protein, 17 aa, Artificial Sequence
  38F6 HC CDR2
EILPGTGYTKYNEKFKG

SEQ ID NO: 72 - protein, 10 aa, Artificial Sequence
  38F6 HC CDR3
LISYYYAMDY

SEQ ID NO: 73 - protein, 7 aa, Artificial Sequence
  5C4 HC CDR1
TSGLGVG

SEQ ID NO: 74 - protein, 16 aa, Artificial Sequence
  5C4 HC CDR2
HIWWDDDKRYNPALKS

SEQ ID NO: 75 - protein, 11 aa, Artificial Sequence
  5C4 HC CDR3
SHYYGTFYFDY

SEQ ID NO: 76 - protein, 7 aa, Artificial Sequence
  23C10 HC CDR1
TSGMGVG

SEQ ID NO: 77 - protein, 16 aa, Artificial Sequence
  23C10 HC CDR2
HIWWDDDKRYNPALKS

SEQ ID NO: 78 - protein, 11 aa, Artificial Sequence
  23C10 HC CDR3
SHYYGTFYFDY

SEQ ID NO: 79 - protein, 7 aa, Artificial Sequence
  28C2 HC CDR1
TSGMGVG

SEQ ID NO: 80 - protein, 16 aa, Artificial Sequence
  28C2 HC CDR2
HIWWDDDKRYNPALKS

SEQ ID NO: 81 - protein, 11 aa, Artificial Sequence
  28C2 HC CDR3
SHYYGTFYFDY

SEQ ID NO: 82 - protein, 11 aa, Artificial Sequence
  28C2 LC CDR1
RASKSISKYLA

SEQ ID NO: 83 - protein, 7 aa, Artificial Sequence
  28C2 LC CDR2
SGSTLQS

SEQ ID NO: 84 - protein, 9 aa, Artificial Sequence
  28C2 LC CDR3
QQHIEYPWT

SEQ ID NO: 85 - protein, 7 aa, Artificial Sequence
  9D6 HC CDR1
TSGMGVG

SEQ ID NO: 86 - protein, 16 aa, Artificial Sequence
  9D6 HC CDR2
HIWWDDDKRYNPTLKS

SEQ ID NO: 87 - protein, 11 aa, Artificial Sequence
  9D6 HC CDR3
SHYNGTFYFDF

SEQ ID NO: 88 - protein, 11 aa, Artificial Sequence
  9D6 LC CDR1
RASKSISKYLA

SEQ ID NO: 89 - protein, 7 aa, Artificial Sequence
  9D6 LC CDR2
SGSTLQS

SEQ ID NO: 90 - protein, 9 aa, Artificial Sequence
  9D6 LC CDR3
QQHIEYPWT

SEQ ID NO: 91 - protein, 5 aa, Artificial Sequence
  28F4 HC CDR1
NYGMS

SEQ ID NO: 92 - protein, 17 aa, Artificial Sequence
  28F4 HC CDR2
TISSGGTYTYYPDSVKG

SEQ ID NO: 93 - protein, 10 aa, Artificial Sequence
  28F4 HC CDR3
RDYDYEGFAY

SEQ ID NO: 94 - protein, 11 aa, Artificial Sequence
  28F4 LC CDR1
RASQSISNNLH

SEQ ID NO: 95 - protein, 7 aa, Artificial Sequence
  28F4 LC CDR2
YASHSIS

SEQ ID NO: 96 - protein, 9 aa, Artificial Sequence
  28F4 LC CDR3
QQSNNWPFT

SEQ ID NO: 97 - protein, 118 aa, Artificial Sequence; identical to SEQ ID NO: 1
  35D1 HC

SEQ ID NO: 98 - protein, 107 aa, Artificial Sequence; identical to SEQ ID NO: 2
  35D1 LC

SEQ ID NO: 99 - protein, 107 aa, Artificial Sequence; identical to SEQ ID NO: 6
  4B11 LC

SEQ ID NO: 100 - protein, 118 aa, Artificial Sequence; identical to SEQ ID NO: 8
  38G8 HC

SEQ ID NO: 101 - protein, 108 aa, Artificial Sequence; identical to SEQ ID NO: 9
  38G8 LC

SEQ ID NO: 102 - protein, 108 aa, Artificial Sequence; identical to SEQ ID NO: 9
  21F10 HC

SEQ ID NO: 103 - protein, 108 aa, Artificial Sequence; identical to SEQ ID NO: 9
  21F10 LC

SEQ ID NO: 104 - skipped

SEQ ID NO: 105 - protein, 119 aa, Artificial Sequence; identical to SEQ ID NO: 14
  13C1 HC

SEQ ID NO: 106-110 - skipped (5 entries)

SEQ ID NO: 111 - protein, 5 aa, Artificial Sequence
  35D1 HC CDR1
AYAMS

SEQ ID NO: 112 - protein, 16 aa, Artificial Sequence
  35D1 HC CDR2
SISSGGTTYYPDSVKR

SEQ ID NO: 113 - protein, 10 aa, Artificial Sequence
  35D1 HC CDR3
GGDYGYALDY

SEQ ID NO: 114 - protein, 10 aa, Artificial Sequence
  35D2 LC CDR1
RASSSVNYMY

SEQ ID NO: 115 - protein, 7 aa, Artificial Sequence
  35D2 LC CDR2
YTSNLAP

SEQ ID NO: 116 - protein, 9 aa, Artificial Sequence
  35D2 LC CDR3
QQFSSSPWT

SEQ ID NO: 117 - protein, 10 aa, Artificial Sequence
  4B11 LC CDR1
RASSSVNYMY

SEQ ID NO: 118 - protein, 7 aa, Artificial Sequence
  4B11 LC CDR2
YTSNLAP

SEQ ID NO: 119 - protein, 9 aa, Artificial Sequence
  4B11 LC CDR3
QQFSSSPWT

SEQ ID NO: 120 - protein, 5 aa, Artificial Sequence
  38G8 HC CDR1
NYLIE

SEQ ID NO: 121 - protein, 17 aa, Artificial Sequence
  38G8 HC CDR2
VINPGSGGTNYNEKFKG

SEQ ID NO: 122 - protein, 9 aa, Artificial Sequence
  38G8 HC CDR3
GRYEWYFDV

SEQ ID NO: 123 - protein, 11 aa, Artificial Sequence
  38G8 LC CDR1
RASQDITNYLN

SEQ ID NO: 124 - protein, 7 aa, Artificial Sequence
  38G8 LC CDR2
YTSRLHS

SEQ ID NO: 125 - protein, 9 aa, Artificial Sequence
  38G8 LC CDR3
QQGYTLPYT

SEQ ID NO: 126 - protein, 5 aa, Artificial Sequence
  21F10 HC CDR1
NYLIE

SEQ ID NO: 127 - protein, 17 aa, Artificial Sequence
  21F10 HC CDR2
VINPGSGGTNYNEKFKG

SEQ ID NO: 128 - protein, 9 aa, Artificial Sequence
  21F10 HC CDR3
GRYEWYFDV

SEQ ID NO: 129 - protein, 11 aa, Artificial Sequence
  21F10 LC CDR1
RASQDITNYLN

SEQ ID NO: 130 - protein, 7 aa, Artificial Sequence
  21F10 LC CDR2
  MISC_FEATURE (1)..(7): 21F10 LC CDR2
YTSRLHS

SEQ ID NO: 131 - protein, 9 aa, Artificial Sequence
  21F10 LC CDR3
QQGYTLPYT

SEQ ID NO: 132-134 - skipped (3 entries)

SEQ ID NO: 135 - protein, 5 aa, Artificial Sequence
  13C1 HC CDR1
SYWIE

SEQ ID NO: 136 - protein, 17 aa, Artificial Sequence
  13C1 HC CDR2
EILPGSGITKYNDKFKG

SEQ ID NO: 137 - protein, 10 aa, Artificial Sequence
  13C1 HC CDR3
LISYYYAMDY

SEQ ID NO: 138-146 - skipped (9 entries)

SEQ ID NO: 147 - protein, 7 aa, Artificial Sequence
  37C7 HC CDR1
TSGMGVG

SEQ ID NO: 148 - protein, 16 aa, Artificial Sequence
  37C7 HC CDR2
HIWWDDDKRYNPALKS

SEQ ID NO: 149 - protein, 11 aa, Artificial Sequence
  37C7 HC CDR3
SHYYGTFYFDY

SEQ ID NO: 150-152 - skipped (3 entries)

SEQ ID NO: 153 - protein, 32 aa, Artificial Sequence
  CTP peptide 1
DPRFQDSSSSKAPPPSLPSPSRLPGPSDTPIL

SEQ ID NO: 154 - protein, 28 aa, Artificial Sequence
  CTP peptide 2
SSSSKAPPPSLPSPSRLPGPSDTPILPQ

SEQ ID NO: 155 - protein, 20 aa, Artificial Sequence
  PAS peptide 1
ASPAAPAPASPAAPAPSAPA

SEQ ID NO: 156 - protein, 20 aa, Artificial Sequence
  PAS peptide 2
AAPASPAPAAPSAPAPAAPS

SEQ ID NO: 157 - protein, 20 aa, Artificial Sequence
  PAS peptide 3
APSSPSPSAPSSPSPASPSS

SEQ ID NO: 158 - protein, 19 aa, Artificial Sequence
  PAS peptide 4
APSSPSPSAPSSPSPASPS

SEQ ID NO: 159 - protein, 20 aa, Artificial Sequence
  PAS peptide 5
SSPSAPSPSSPASPSPSSPA

SEQ ID NO: 160 - protein, 24 aa, Artificial Sequence
  PAS peptide 6
AASPAAPSAPPAAASPAAPSAPPA

SEQ ID NO: 161 - protein, 20 aa, Artificial Sequence
  PAS peptide 7
ASAAAPAAASAAASAPSAAA

SEQ ID NO: 162 - protein, 11 aa, Artificial Sequence
  Albumin Binding Peptide Core Sequence
DICLPRWGCLW

SEQ ID NO: 163 - protein, 264 aa, Artificial Sequence
  GFP protein sequence
MSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKSRTSGSPGLQEFDIKLIDTVDLESCN

SEQ ID NO: 164 - protein, 474 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 591 aa, Homo sapiens
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 166 - protein, 4 aa, Artificial Sequence
  Linker
GGGS

SEQ ID NO: 167 - protein, 18 aa, Artificial Sequence
  Albumin binding peptide 1
RLIEDICLPRWGCLWEDD

SEQ ID NO: 168 - protein, 20 aa, Artificial Sequence
  Albumin binding peptide 2
QRLMEDICLPRWGCLWEDDF

SEQ ID NO: 169 - protein, 21 aa, Artificial Sequence
  Albumin binding peptide 3
QGLIGDICLPRWGCLWGDSVK

SEQ ID NO: 170 - protein, 20 aa, Artificial Sequence
  Albumin binding peptide 4
GEWWEDICLPRWGCLWEEED

SEQ ID NO: 171 - protein, 10 aa, Artificial Sequence
  Cysteine-containing peptide
GGGSGCGGGS

SEQ ID NO: 172 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 173 - protein, 13 aa, Artificial Sequence
  Biotin Acceptor Peptide (BAP)
LNDIFEAQKIEWH

SEQ ID NO: 174 - protein, 13 aa, Artificial Sequence
  Lipoate Acceptor Peptide 2 (LAP2)
GFEIDKVWYDLDA

SEQ ID NO: 175 - protein, 5 aa, Artificial Sequence
  HAPylation motif (can be repeated up to 400 times)
GGGGS

SEQ ID NO: 176 - protein, 12 aa, Artificial Sequence
  Alternative linker
PEAPTDPEAPTD

SEQ ID NO: 177 - protein, 29 aa, Artificial Sequence
  CTP
DSSSSKAPPPSLPSPSRLPGPSDTPILPQ

SEQ ID NO: 178 - protein, 254 aa, Homo sapiens [sequence omitted]

SEQ ID NO: 179 - protein, 152 aa, Homo sapiens [sequence omitted]

SEQ ID NO: 180 - DNA, 762 bp, Homo sapiens
attgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccc

SEQ ID NO: 181 - DNA, 456 bp, Homo sapiens
gccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccga

SEQ ID NO: 182 - skipped

SEQ ID NO: 183 - protein, 1039 aa, Homo sapiens
  MISC_FEATURE (1)..(31): (Human GPIIb) Signal sequence
  MISC_FEATURE (981)..(1019): (Human GPIIb) Transmembrane
  MISC_FEATURE (1020)..(1039): (Human GPIIb) Cytoplasmic
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

SEQ ID NO: 184 - protein, 788 aa, Homo sapiens
  MISC_FEATURE (1)..(26): (Human GPIIIa) Signal Sequence
  MISC_FEATURE (719)..(747): (Human GPIIIa) Transmembrane
  MISC_FEATURE (748)..(788): (Human GPIIIa) Cytoplasmic
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 185 - DNA, 6238 bp, Homo sapiens
atggccagagctttgtgtccactgcaagccctctggcttctggagtgggtgctgctgctctaccggtctcgaaacacaggtgacgttcgggagaccgaagacctcacccacgacgacgagttgggaccttgtgctgcccctccagcctgggccttgaacctggacccagtgcagctcaccaaccctggaacacgacggggaggtcggacccggaacttggacctgggtcacgtcgagtggttctatgcaggccccaatggcagccagtttggattttcactggacttccacaaggacagcaagatacgtccggggttaccgtcggtcaaacctaaaagtgacctgaaggtgttcctgtcgcatgggagagtggccatcgtggtgggcgccccgcggaccctgggccccagccaggaggaggtaccctctcaccggtagcaccacccgcggggcgcctgggacccggggtcggtcctcctcacgggcggcgtgttcctgtgcccctggagggccgagggcggccagtgcccctcgctgctctgcccgccgcacaaggacacggggacctcccggctcccgccggtcacggggagcgacgagtttgacctccgtgatgagacccgaaatgtaggctcccaaactttacaaaccttcaaggccaaactggaggcactactctgggctttacatccgagggtttgaaatgtttggaagttccggcgccaaggactgggggcgtcggtcgtcagctggagcgacgtcattgtggcctgcgcccccgcggttcctgacccccgcagccagcagtcgacctcgctgcagtaacaccggacgcgggggtggcagcactggaacgtcctagaaaagactgaggaggctgagaagacgcccgtaggtagcaccgtcgtgaccttgcaggatcttttctgactcctccgactcttctgcgggcatccatcgtgctttttggctcagccagagagcggccggcgcgccgagtactccccctgtcgcgggaacacgaaaaaccgagtcggtctctcgccggccgcgcggctcatgagggggacagcgcccttgaccctgagccgcatttacgtggaaaatgattttagctgggacaagcgttactgtgaagcgtgggactcggcgtaaatgcaccttttactaaaatcgaccctgttcgcaatgacacttcgcggcttcagctccgtggtcactcaggccggagagctggtgcttggggctcctggcggctatccgaagtcgaggcaccagtgagtccggcctctcgaccacgaaccccgaggaccgccgatatatttcttaggtctcctggcccaggctccagttgcggatattttctcgagttaccgcccaataaagaatccagaggaccgggtccgaggtcaacgcctataaaagagctcaatggcgggtggcatccttttgtggcacgtgtcctcccagagcctctcctttgactccagcaacccagagccgtaggaaaacaccgtgcacaggagggtctcggagaggaaactgaggtcgttgggtctctacttcgacggctactgggggtactcggtggccgtgggcgagttcgacggggatctcaacatgaagctgccgatgacccccatgagccaccggcacccgctcaagctgcccctagagttgactacagaatatgtcgtcggtgcccccacttggagctggaccctgggagcggtggaaatttgatgtcttatacagcagccacgggggtgaacctcgacctgggaccctcgccacctttaattggattcctactaccagaggctgcatcggctgcgcggagagcagatggcgtcgtattttaacctaaggatgatggtctccgacgtagccgacgcgcctctcgtctaccgcagcataaaagggcattcagtggctgtcactgacgtcaacggggatgggaggcatgatctgctggtgggccccgtaagtcaccgacagtgactgcagttgcccctaccctccgtactagacgaccacccggctccactgtatatggagagccgggcagaccgaaaactggccgaagtggggcgtgtgtatcgaggtgacatatacctctcggcccgtctggcttttgaccggcttcaccccgcacacatattgttcctgcagccgcgaggcccccacgcgctgggtgcccccagcctcctgctgactggcaacaaggacgtcggcgctccgggggtgcgcgacccacgggggtcggaggacgactgaccgacacagctctatgggcgattcggctctgccatcgcacccctgggcgacctcgaccgggattgtgtcgagatacccgctaagccgagacggtagcgtggggacccgctggagctggccctaggctacaatgacattgcagtggctgccccctacgggggtcccagtggccggggccaagtgccgatgttactgtaacgtcaccgacgggggatgcccccagggtcaccggccccggttcacctggtgttcctgggtcagagtgaggggctgaggtcacgtccctcccaggtcctggacagcgaccacaaggacccagtctcactccccgactccagtgcagggagggtccaggacctgtcgcccttccccacaggctctgcctttggcttctcccttcgaggtgccgtagacatcgatgacgggaaggggtgtccgagacggaaaccgaagagggaagctccacggcatctgtagctactgaacggatacccagacctgatcgtgggagcttacggggccaaccaggtggctgtgtacagattgcctatgggtctggactagcaccctcgaatgccccggttggtccaccgacacatgtctgctcagccagtggtgaaggcctctgccagctactggtgcaagattcactgaatcctgctcgagtcggtcaccacttccggagacaggtcgatgaccacgttctaagtgacttaggacgagtgaagagctgtgtcctacctcagaccaagacacccgtgagctgcttcaacatccagatgcacttctcgacacaggatggagtctggttctgtgggcactcgacgaagttgtaggtctactgtgttggagccactgggcacaacattcctcagaagctatccctaaatgccgagctgcagacacaacctcggtgacccgtgttgtaaggagtcttcgatagggatttacggctcgacgtcctggaccggcagaagccccgccagggccggcgggtgctgctgctgggctctcaacaggcagacctggccgtcttcggggcggtcccggccgcccacgacgacgacccgagagttgtccgtggcaccaccctgaacctggatctgggcggaaagcacagccccatctgccacaccaccatgccgtggtgggacttggacctagacccgcctttcgtgtcggggtagacggtgtggtggtacgccttccttcgagatgaggcagacttccgggacaagctgagccccattgtgctcagcctccggaaggaagctctactccgtctgaaggccctgttcgactcggggtaacacgagtcggagaatgtgtccctaccgcccacggaggctggaatggcccctgctgtcgtgctgcatggagacttacacagggatggcgggtgcctccgaccttaccggggacgacagcacgacgtacctctgacccatgtgcaggagcagacacgaatcgtcctggactgtggggaagatgacgtatgtgtgtgggtacacgtcctcgtctgtgcttagcaggacctgacaccccttctactgcatacacacccccagcttcagctcactgccagcgtgacgggctccccgctcctagttggggcagataatggggtcgaagtcgagtgacggtcgcactgcccgaggggcgaggatcaaccccgtctattagtcctggagctgcagatggacgcagccaacgagggcgagggggcctatgaagcagagctgcaggacctcgacgtctacctgcgtcggttgctcccgctcccccggatacttcgtctcgacgccgtgcacctgccccagggcgcccactacatgcgggccctaagcaatgtcgagggctttcggcacgtggacggggtcccgcgggtgatgtacgcccgggattcgttacagctcccgaaagagagactcatctgtaatcagaagaaggagaatgagaccagggtggtgctgtgtgagctgctctctgagtagacattagtcttcttcctcttactctggtcccaccacgacacactcgacggcaaccccatgaagaagaacgcccagataggaatcgcgatgttggtgagcgtggggaatccgttggggtacttcttcttgcgggtctatccttagcgctacaaccactcgcaccccttactggaagaggctggggagtctgtgtccttccagctgcagatacggagcaagaacagccaggaccttctccgacccctcagacacaggaaggtcgacgtctatgcctcgttcttgtcggtcaatccaaacagcaagattgtgctgctggacgtgccggtccgggcagaggcccaagtggagttaggtttgtcgttctaacacgacgacctgcacggccaggcccgtctccgggttcacctcctgcgagggaactcctttccagcctccctggtggtggcagcagaagaaggtgagagggaggacgctcccttgaggaaaggtcggagggaccaccaccgtcgtcttcttccactctccctccagaacagcttggacagctggggacccaaagtggagcacacctatgagctccacaacaatgtcttgtcgaacctgtcgacccctgggtttcacctcgtgtggatactcgaggtgttgttaggccctgggactgtgaatggtcttcacctcagcatccaccttccgggacagtcccagcccccgggaccctgacacttaccagaagtggagtcgtaggtggaaggccctgtcagggtcgggtccgacctgctctacatcctggatatacagccccaggggggccttcagtgcttcccacagaggctggacgagatgtaggacctatatgtcggggtccccccggaagtcacgaagggtgtccctcctgtcaaccctctcaaggtggactgggggctgcccatccccagcccctcccccattggaggacagttgggagagttccacctgacccccgacgggtaggggtcggggagggggtaacacccggcccatcacaagcgggatcgcagacagatcttcctgccagagcccgagcagcccgtgggccgggtagtgttcgccctagcgtctgtctagaaggacggtctcgggctcgtcgggtcgaggcttcaggatccagttctcgtaagctgcgactcggcgccctgtactgtggtgcagagctccgaagtcctaggtcaagagcattcgacgctgagccgcgggacatgacaccacgtctgtgacctgcaggagatggcgcgcgggcagcgggccatggtcacggtgctggccttcctgacactggacgtcctctaccgcgcgcccgtcgcccggtaccagtgccacgaccggaaggactggctgcccagcctctaccagaggcctctggatcagtttgtgctgcagtcgcacgcatggaccgacgggtcggagatggtctccggagacctagtcaaacacgacgtcagcgtgcgtaccttcaacgtgtcctccctcccctatgcggtgcccccgctcagcctgccccgaggggaagctaagttgcacaggagggaggggatacgccacgggggcgagtcggacgggctccccttcgacaggtgtggacacagctgctccgggccttggaggagagggccattccaatctggtgggtggtccacacctgtgtcgacgaggcccggaacctcctctcccggtaaggttagaccacccacctggtgggtgtgctgggtggcctgctgctgctcaccatcctggtcctggccatgtggaaggaccacccacacgacccaccggacgacgacgagtggtaggaccaggaccggtacaccttcgtcggcttcttcaagcggaaccggccacccctggaagaagatgatgaagagggggagtgacagccgaagaagttcgccttggccggtggggaccttcttctactacttctccccctcact

SEQ ID NO: 186 - DNA, 4734 bp, Homo sapiens
atgcgagcgcgcccgcggccccggccgctctgggcgactgtgctggcgctgggggcgctgtacgctcgcgcgggcgccggggccggcgagacccgctgacacgaccgcgacccccgcgacgcgggcgttggcgtaggagggcccaacatctgtaccacgcgaggtgtgagctcctgccagcgcccgcaaccgcatcctcccgggttgtagacatggtgcgctccacactcgaggacggtccagtgcctggctgtgagccccatgtgtgcctggtgctctgatgaggccctgcctctgggcgtcacggaccgacactcggggtacacacggaccacgagactactccgggacggagacccgtcacctcgctgtgacctgaaggagaatctgctgaaggataactgtgccccagaatccatcagtggagcgacactggacttcctcttagacgacttcctattgacacggggtcttaggtaggagttcccagtgagtgaggcccgagtactagaggacaggcccctcagcgacaagggctctctcaagggtcactcactccgggctcatgatctcctgtccggggagtcgctgttcccgagaggagacagctcccaggtcactcaagtcagtccccagaggattgcactccggctccggccacctctgtcgagggtccagtgagttcagtcaggggtctcctaacgtgaggccgaggccggtgatgattcgaagaatttctccatccaagtgcggcaggtggaggattaccctgtggacatcctactaagcttcttaaagaggtaggttcacgccgtccacctcctaatgggacacctgtagtactacttgatggacctgtcttactccatgaaggatgatctgtggagcatccagaacctgatgatgaactacctggacagaatgaggtacttcctactagacacctcgtaggtcttggacggtaccaagctggccacccagatgcgaaagctcaccagtaacctgcggattggcttcgggccatggttcgaccggtgggtctacgctttcgagtggtcattggacgcctaaccgaagcccgcatttgtggacaagcctgtgtcaccatacatgtatatctccccaccagaggccctcgaacgtaaacacctgttcggacacagtggtatgtacatatagaggggtggtctccgggagcttaacccctgctatgatatgaagaccacctgcttgcccatgtttggctacaaacacgtgctgttggggacgatactatacttctggtggacgaacgggtacaaaccgatgtttgtgcacgacacgctaactgaccaggtgacccgcttcaatgaggaagtgaagaagcagagtgtgtcacggtgcgattgactggtccactgggcgaagttactccttcacttcttcgtctcacacagtgccaaccgagatgccccagagggtggctttgatgccatcatgcaggctacagtctgtgatgaattggctctacggggtctcccaccgaaactacggtagtacgtccgatgtcagacactacttaagattggctggaggaatgatgcatcccacttgctggtgtttaccactgatgccaagactttctaaccgacctccttactacgtagggtgaacgaccacaaatggtgactacggttctgacatatagcattggacggaaggctggcaggcattgtccagcctaatgacgggcagtgtcatgtatatcgtaacctgccttccgaccgtccgtaacaggtcggattactgcccgtcacagtagttggtagtgacaatcattactctgcctccactaccatggattatccctctttggggctgcaaccatcactgttagtaatgagacggaggtgatggtacctaatagggagaaaccccgacatgactgagaagctatcccagaaaaacatcaatttgatctttgcagtgactgaaaatgtatactgactcttcgatagggtctttttgtagttaaactagaaacgtcactgacttttacatgtcaatctctatcagaactatagtgagctcatcccagggaccacagttggggttctgtcccagttagagatagtcttgatatcactcgagtagggtccctggtgtcaaccccaagacaggatggattccagcaatgtcctccagctcattgttgatgcttatgggaaaatccgttctaaatacctaaggtcgttacaggaggtcgagtaacaactacgaatacccttttaggcaagatttgtagagctggaagtgcgtgacctccctgaagagttgtctctatccttcaatgccacctgccatctcgaccttcacgcactggagggacttctcaacagagataggaagttacggtggacgctcaacaatgaggtcatccctggcctcaagtcttgtatgggactcaagattggagacacggagttgttactccagtagggaccggagttcagaacataccctgagttctaacctctgtgcgtgagcttcagcattgaggccaaggtgcgaggctgtccccaggagaaggagaagtcctttcactcgaagtcgtaactccggttccacgctccgacaggggtcctcttcctcttcaggaaaaccataaagcccgtgggcttcaaggacagcctgatcgtccaggtcacctttgattgtgactggtatttcgggcacccgaagttcctgtcggactagcaggtccagtggaaactaacactgtgtgcctgccaggcccaagctgaacctaatagccatcgctgcaacaatggcaatgggaccacacggacggtccgggttcgacttggattatcggtagcgacgttgttaccgttaccctggtttgagtgtggggtatgccgttgtgggcctggctggctgggatcccagtgtgagtgctcaaaactcacaccccatacggcaacacccggaccgaccgaccctagggtcacactcacgagtgaggaggactatcgcccttcccagcaggacgaatgcagcccccgggagggtcagcccgtcctcctcctgatagcgggaagggtcgtcctgcttacgtcgggggccctcccagtcgggcagtgcagccagcggggcgagtgcctctgtggtcaatgtgtctgccacagcagtgactttggcacgtcggtcgccccgctcacggagacaccagttacacagacggtgtcgtcactgaaaccgaagatcacgggcaagtactgcgagtgtgacgacttctcctgtgtccgctacaagggggagttctagtgcccgttcatgacgctcacactgctgaagaggacacaggcgatgttccccctcatgtgctcaggccatggccagtgcagctgtggggactgcctgtgtgactccgactggacctacacgagtccggtaccggtcacgtcgacacccctgacggacacactgaggctgacctggggctactactgcaactgtaccacgcgtactgacacctgcatgtccagcaatgggctgctgccgatgatgacgttgacatggtgcgcatgactgtggacgtacaggtcgttacccgacgactgcagcggccggggcaagtgtgaatgtggcagctgtgtctgtatccagccgggctcctatacgtcgccggccccgttcacacttacaccgtcgacacagacataggtcggcccgaggataggggacacctgtgagaagtgccccacctgcccagatgcctgcacctttaagaaagaatgtcccctgtggacactcttcacggggtggacgggtctacggacgtggaaattctttcttacagtggagtgtaagaagtttgaccggggagccctacatgacgaaaatacctgcaaccgttaccacctcacattcttcaaactggcccctcgggatgtactgcttttatggacgttggcaatgtgccgtgacgagattgagtcagtgaaagagcttaaggacactggcaaggatgcagtgaatacggcactgctctaactcagtcactttctcgaattcctgtgaccgttcctacgtcacttatgtacctataagaatgaggatgactgtgtcgtcagattccagtactatgaagattctagtacatggatattcttactcctactgacacagcagtctaaggtcatgatacttctaagatcaggaaagtccatcctgtatgtggtagaagagccagagtgtcccaagggccctgacatcctgcctttcaggtaggacatacaccatcttctcggtctcacagggttcccgggactgtaggacgtggtcctgctctcagtgatgggggccattctgctcattggccttgccgccctgctcatccaccaggacgagagtcactacccccggtaagacgagtaaccggaacggcgggacgagtagtggaaactcctcatcaccatccacgaccgaaaagagttcgctaaatttgaggaagaacgcacctttgaggagtagtggtaggtgctggcttttctcaagcgatttaaactccttcttgcggccagagcaaaatgggacacagccaacaacccactgtataaagaggccacgtctaccttccggtctcgttttaccctgtgtcggttgttgggtgacatatttctccggtgcagatggaagaccaatatcacgtaccggggcacttaatggttatagtgcatggccccgtgaatt

SEQ ID NO: 187 - DNA, 2205 bp, Artificial Sequence
  DNA sequence of FVII-165
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 188 - DNA, 3147 bp, Artificial Sequence
  DNA sequence of FVII-175
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggcggtggctccggcggaggtgggtccggtggcggcggatcaggtgggggtggatcaggcggtggaggttccggtggcgggggatcccaggtgaaactgctcgagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcgccatctccagagacaattccaagaacacgctgtatctgcaaatgaacagcctgagagctgaggacacggctgtgtattactgtgcgagagcgctggggagctgggggggttgggaccactacatggacgtctggggcaaagggaccacggtcaccgtctcctcaggtggcggcggatcaggtgggggtggatcaggtggcggtggctccggtggcgggggatcagtggtgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggtcagcccggtggcggcggatcaggtgggggtggatcaggcggtggaggttccggtggcgggggatccggcggtggaggttccggtgggggtggatcaggctcgagtggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 189 - DNA, 3162 bp, Artificial Sequence
  DNA sequence of FVII-177
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaggtggcggtggctccggcggaggtgggtccggtggcggcggatcaggtgggggtggatcaggcggtggaggttccggtggcgggggatcccaggtgaaactgctcgagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcgccatctccagagacaattccaagaacacgctgtatctgcaaatgaacagcctgagagctgaggacacggctgtgtattactgtgcgagagcgctggggagctgggggggttgggaccactacatggacgtctggggcaaagggaccacggtcaccgtctcctcaggtggcggcggatcaggtgggggtggatcaggtggcggtggctccggtggcgggggatcagtggtgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggtcagcccggtggcggtggctccggcggaggtgggtccggtggcggcggatcaggtgggggtggatcaggcggtggaggttccggtggcgggggaaggaagaggaggaagaggattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgcca

SEQ ID NO: 190 - DNA, 3069 bp, Artificial Sequence
  DNA sequence of FVII-178
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccaggttcgtcttcaggtggcggtggctccggcggaggtgggtccggtggcggcggatcaggtgggggtggatcaggcggtggaggttccggtggcgggggatcccaggtgaaactgctcgagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcgccatctccagagacaattccaagaacacgctgtatctgcaaatgaacagcctgagagctgaggacacggctgtgtattactgtgcgagagcgctggggagctgggggggttgggaccactacatggacgtctggggcaaagggaccacggtcaccgtctcctcaggtggcggcggatcaggtgggggtggatcaggtggcggtggctccggtggcgggggatcagtggtgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggtcagccc

SEQ ID NO: 191 - DNA, 3264 bp, Artificial Sequence
  DNA sequence of FVII-179
gccgccaccatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggtggcggcggatcaggtgggggtggatcaggcggtggaggttccggtggcgggggatccggcggtggaggttccggtgggggtggatcaggctcgagtggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccaaccggtggtggcggtggctccggcggaggtgggtccggtggcggcggatcaggtgggggtggatcaggcggtggaggttccggtggcgggggatcaagaaagagaaggaaaagaattgtggggggcaaggtgtgccccaaaggggagtgtccatggcaggtcctgttgttggtgaatggagctcagttgtgtggggggaccctgatcaacaccatctgggtggtgtccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggcggtggctccggcggaggtgggtccggtggcggcggatcaggtgggggtggatcaggcggtggaggttccggtggcgggggatcccaggtgaaactgctcgagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcgccatctccagagacaattccaagaacacgctgtatctgcaaatgaacagcctgagagctgaggacacggctgtgtattactgtgcgagagcgctggggagctgggggggttgggaccactacatggacgtctggggcaaagggaccacggtcaccgtctcctcaggtggcggcggatcaggtgggggtggatcaggtggcggtggctccggtggcgggggatcagtggtgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggtcagccc

SEQ ID NO: 192 - DNA, 3042 bp, Artificial Sequence
  DNA sequence of FVII-200
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccaggttcgtcttcaggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctca

SEQ ID NO: 193 - DNA, 3228 bp, Artificial Sequence
  DNA sequence of FVII-211
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggtggcggcggatcaggtgggggtggatcaggcggtggaggttccggtggcgggggatccggcggtggaggttccggtgggggtggatcaggctcgagtggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccaaccggtggtggcggtggctccggcggaggtgggtccggtggcggcggatcaggtgggggtggatcaggcggtggaggttccggtggcgggggatcaagaaagagaaggaaaagaattgtggggggcaaggtgtgccccaaaggggagtgtccatggcaggtcctgttgttggtgaatggagctcagttgtgtggggggaccctgatcaacaccatctgggtggtgtccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctca

SEQ ID NO: 194 - protein, 12 aa, Artificial Sequence
  AD Family Motif
GESPGGSSGSES

SEQ ID NO: 195 - protein, 12 aa, Artificial Sequence
  AD Family Motif
GSEGSSGPGESS

SEQ ID NO: 196 - protein, 12 aa, Artificial Sequence
  AD Family Motif
GSSESGSSEGGP

SEQ ID NO: 197 - protein, 12 aa, Artificial Sequence
  AD Family Motif
GSGGEPSESGSS

SEQ ID NO: 198 - protein, 12 aa, Artificial Sequence
  AE, AM Family Motif
GSPAGSPTSTEE

SEQ ID NO: 199 - protein, 12 aa, Artificial Sequence
  AE, AM, AQ Family Motif
GSEPATSGSETP

SEQ ID NO: 200 - protein, 12 aa, Artificial Sequence
  AE, AM, AQ Family Motif
GTSESATPESGP

SEQ ID NO: 201 - protein, 12 aa, Artificial Sequence
  AE, AM, AQ Family Motif
GTSTEPSEGSAP

SEQ ID NO: 202 - protein, 12 aa, Artificial Sequence
  AF, AM Family Motif
GSTSESPSGTAP

SEQ ID NO: 203 - protein, 12 aa, Artificial Sequence
  AF, AM Family Motif
GTSTPESGSASP

SEQ ID NO: 204 - protein, 12 aa, Artificial Sequence
  AF, AM Family Motif
GTSPSGESSTAP

SEQ ID NO: 205 - protein, 12 aa, Artificial Sequence
  AF, AM Family Motif
GSTSSTAESPGP

SEQ ID NO: 206 - protein, 12 aa, Artificial Sequence
  AG, AM Family Motif
GTPGSGTASSSP

SEQ ID NO: 207 - protein, 12 aa, Artificial Sequence
  AG, AM Family Motif
GSSTPSGATGSP

SEQ ID NO: 208 - protein, 12 aa, Artificial Sequence
  AG, AM Family Motif
GSSPSASTGTGP

SEQ ID NO: 209 - protein, 12 aa, Artificial Sequence
  AG, AM Family Motif
GASPGTSSTGSP

SEQ ID NO: 210 - protein, 12 aa, Artificial Sequence
  AQ Family Motif
GEPAGSPTSTSE

SEQ ID NO: 211 - protein, 12 aa, Artificial Sequence
  AQ Family Motif
GTGEPSSTPASE

SEQ ID NO: 212 - protein, 12 aa, Artificial Sequence
  AQ Family Motif
GSGPSTESAPTE

SEQ ID NO: 213 - protein, 12 aa, Artificial Sequence
  AQ Family Motif
GSETPSGPSETA

SEQ ID NO: 214 - protein, 12 aa, Artificial Sequence
  AQ Family Motif
GPSETSTSEPGA

SEQ ID NO: 215 - protein, 12 aa, Artificial Sequence
  AQ Family Motif
GSPSEPTEGTSA

SEQ ID NO: 216 - protein, 12 aa, Artificial Sequence
  BC Family Motif
GSGASEPTSTEP

SEQ ID NO: 217 - protein, 12 aa, Artificial Sequence
  BC Family Motif
GSEPATSGTEPS

SEQ ID NO: 218 - protein, 12 aa, Artificial Sequence
  BC Family Motif
GTSEPSTSEPGA

SEQ ID NO: 219 - protein, 12 aa, Artificial Sequence
  BC Family Motif
GTSTEPSEPGSA

SEQ ID NO: 220 - protein, 12 aa, Artificial Sequence
  BD Family Motif
GSTAGSETSTEA

SEQ ID NO: 221 - protein, 12 aa, Artificial Sequence
  BD Family Motif
GSETATSGSETA

SEQ ID NO: 222 - protein, 12 aa, Artificial Sequence
  BD Family Motif
GTSESATSESGA

SEQ ID NO: 223 - protein, 12 aa, Artificial Sequence
  BD Family Motif
GTSTEASEGSAS

SEQ ID NO: 224 - protein, 42 aa, Artificial Sequence
  XTEN AE42
GAPGSPAGSPTSTEEGTSESATPESGPGSEPATSGSETPASS

SEQ ID NO: 225 - protein, 42 aa, Artificial Sequence
  XTEN AE42(2)
TGGGSPAGSPTSTEEGTSESATPESGPGSEPATSGSETPASS

SEQ ID NO: 226 - protein, 42 aa, Artificial Sequence
  XTEN AE42(3)
GTSESATPESGPGSEPATSGSETPGTSESATPESGPGSEPAT

SEQ ID NO: 227 - protein, 78 aa, Artificial Sequence
  XTEN AE72
GAPTSESATPESGPGSEPATSGSETPGTSESATPESGPGSEPATSGSETPGTSESATPESGPGTSTEPSEGSAPGASS

SEQ ID NO: 228 - protein, 72 aa, Artificial Sequence
  XTEN AE72(2)
GTSESATPESGPGSEPATSGSETPGTSESATPESGPGSEPATSGSETPGTSESATPESGPGTSTEPSEGSAP

SEQ ID NO: 229 - protein, 72 aa, Artificial Sequence
  XTEN AE72(3)
SPAGSPTSTEEGTSESATPESGPGSEPATSGSETPGTSESATPESGPGTSTEPSEGSAPGTSTEPSEGSAPG

SEQ ID NO: 230 - protein, 143 aa, Artificial Sequence
  XTEN AE144
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESAPESGPGSEPATSGSETPGTSTEPSEGSAP

SEQ ID NO: 231 - protein, 144 aa, Artificial Sequence
  XTEN AE144(2)
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEE

SEQ ID NO: 232 - protein, 144 aa, Artificial Sequence
  XTEN AE144(3)
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAP

SEQ ID NO: 233 - protein, 144 aa, Artificial Sequence
  XTEN AG144
GTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSP

SEQ ID NO: 234 - protein, 288 aa, Artificial Sequence
  XTEN AE288
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 235 - protein, 288 aa, Artificial Sequence
  XTEN AG288
PGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGS

SEQ ID NO: 236 - protein, 576 aa, Artificial Sequence
  XTEN AE576
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 237 - protein, 576 aa, Artificial Sequence
  XTEN AG576
PGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGS

SEQ ID NO: 238 - protein, 864 aa, Artificial Sequence
  XTEN AE864
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 239 - protein, 864 aa, Artificial Sequence
  XTEN AG864
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 240 - protein, 80 aa, Artificial Sequence
  Glycine Serine Peptide Linker
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 241 - protein, 160 aa, Artificial Sequence
  Glycine Serine Peptide Linker
GGSGGSGGSGGSGGSGGSGGSGGSGGSGGSGGSGGSGGSGGSGGSGGSGGSGGSGGSGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 242 - protein, 7 aa, Artificial Sequence
  Glycine Serine Peptide Linker
SGGSGGS

SEQ ID NO: 243 - protein, 15 aa, Artificial Sequence
  Glycine Serine Peptide Linker
GGSGGSGGSGGSGGG

SEQ ID NO: 244 - protein, 16 aa, Artificial Sequence
  Glycine Serine Peptide Linker
GGSGGSGGGGSGGGGS

SEQ ID NO: 245 - protein, 18 aa, Artificial Sequence
  Glycine Serine Peptide Linker
GGSGGSGGSGGSGGSGGS

SEQ ID NO: 246 - protein, 15 aa, Artificial Sequence
  Glycine Serine Peptide Linker
GGGGSGGGGSGGGGS

SEQ ID NO: 247 - protein, 400 aa, Artificial Sequence
  Glycine Serine Peptide Linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 248 - protein, 500 aa, Artificial Sequence
  Glycine Serine Peptide Linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 249 - protein, 5 aa, Artificial Sequence
  Glycine Serine Peptide Linker
GGGGS

SEQ ID NO: 250 - protein, 10 aa, Artificial Sequence
  Glycine Serine Peptide Linker
GGGGSGGGGS

SEQ ID NO: 251 - protein, 15 aa, Artificial Sequence
  Glycine Serine Peptide Linker
GGGGSGGGGSGGGGS

SEQ ID NO: 252 - protein, 20 aa, Artificial Sequence
  Glycine Serine Peptide Linker
GGGGSGGGGSGGGGSGGGGS

SEQ ID NO: 253 - protein, 25 aa, Artificial Sequence
  Glycine Serine Peptide Linker
GGGGSGGGGSGGGGSGGGGSGGGGS

SEQ ID NO: 254 - protein, 30 aa, Artificial Sequence
  Glycine Serine Peptide Linker
GGGGSGGGGSGGGGSGGGGSGGGGSGGGGS

SEQ ID NO: 255 - protein, 35 aa, Artificial Sequence
  Glycine Serine Peptide Linker
GGGGSGGGGSGGGGSGGGGSGGGGSGGGGSGGGGS

SEQ ID NO: 256 - protein, 40 aa, Artificial Sequence
  Glycine Serine Peptide Linker
GGGGSGGGGSGGGGSGGGGSGGGGSGGGGSGGGGSGGGGS

SEQ ID NO: 257 - protein, 45 aa, Artificial Sequence
  Glycine Serine Peptide Linker
GGGGSGGGGSGGGGSGGGGSGGGGSGGGGSGGGGSGGGGSGGGGS

SEQ ID NO: 258 - protein, 50 aa, Artificial Sequence
  Glycine Serine Peptide Linker
GGGGSGGGGSGGGGSGGGGSGGGGSGGGGSGGGGSGGGGSGGGGSGGGGS

SEQ ID NO: 259 - protein, 6 aa, Artificial Sequence
  Glycine Serine Peptide Linker
SGGGGS

SEQ ID NO: 260 - protein, 11 aa, Artificial Sequence
  Glycine Serine Peptide Linker
SGGGGSGGGGS

SEQ ID NO: 261 - protein, 16 aa, Artificial Sequence
  Glycine Serine Peptide Linker
SGGGGSGGGGSGGGGS

SEQ ID NO: 262 - protein, 21 aa, Artificial Sequence
  Glycine Serine Peptide Linker
SGGGGSGGGGSGGGGSGGGGS

SEQ ID NO: 263 - protein, 26 aa, Artificial Sequence
  Glycine Serine Peptide Linker
SGGGGSGGGGSGGGGSGGGGSGGGGS

SEQ ID NO: 264 - protein, 31 aa, Artificial Sequence
  Glycine Serine Peptide Linker
SGGGGSGGGGSGGGGSGGGGSGGGGSGGGGS

SEQ ID NO: 265 - protein, 36 aa, Artificial Sequence
  Glycine Serine Peptide Linker
SGGGGSGGGGSGGGGSGGGGSGGGGSGGGGSGGGGS

SEQ ID NO: 266 - protein, 41 aa, Artificial Sequence
  Glycine Serine Peptide Linker
SGGGGSGGGGSGGGGSGGGGSGGGGSGGGGSGGGGSGGGGS

SEQ ID NO: 267 - protein, 46 aa, Artificial Sequence
  Glycine Serine Peptide Linker
SGGGGSGGGGSGGGGSGGGGSGGGGSGGGGSGGGGSGGGGSGGGGS

SEQ ID NO: 268 - protein, 51 aa, Artificial Sequence
  Glycine Serine Peptide Linker
SGGGGSGGGGSGGGGSGGGGSGGGGSGGGGSGGGGSGGGGSGGGGSGGGGS

SEQ ID NO: 269 - DNA, 2616 bp, Artificial Sequence
  DNA sequence of FVII-227
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctacggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggtggcggcggatcaggtgggggtggatcaggcggtggaggttccggtggcgggggatccggcggtggaggttccggtgggggtggatcaggctcgagtagtcccgctggaagcccaactagcaccgaagaggggacctcagagtccgccacccccgagtccggccctggctctgagcctgccactagcggctccgagactcctggcacatccgaaagcgctacacccgagagtggacccggcacctctaccgagcccagtgagggctccgcccctggaacaagcaccgagcccagcgaaggcagcgccccagggaccggtggtggcggtggctccggcggaggtgggtccggtggcggcggatcaggtgggggtggatcaggcggtggaggttccggtggcgggggatcaagaaagagaaggaaaagaattgtggggggcaaggtgtgccccaaaggggagtgtccatggcaggtcctgttgttggtgaatggagctcagttgtgtggggggaccctgatcaacaccatctgggtggtgtccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctcaggtggcggaggttcccatcatcaccatcaccattga

SEQ ID NO: 270 - DNA, 2832 bp, Artificial Sequence
  DNA sequence of FVII-228
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctacggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggtggcggcggatcaggtgggggtggatcaggcggtggaggttccggtggcgggggatccggcggtggaggttccggtgggggtggatcaggctcgagtggttctccagccgggtccccaacttcgaccgaggaagggacctccgagtcagctaccccggagtccggtcctggcacctccaccgaaccatcggagggcagcgcccctgggagccctgccgggagccctacaagcaccgaagagggcaccagtacagagccaagtgaggggagcgcccctggtactagtactgaaccatccgaggggtcagctccaggcacgagtgagtccgctacccccgagagcggaccgggctcagagcccgccacgagtggcagtgaaactccaggctcagaacccgccactagtgggtcagagactccaggcagccctgccggatcccctacgtccaccgaggagggaacatctgagtccgcaacacccgaatccggtccaggcacctccacggaacctagtgaaggctcggcaccaaccggtggtggcggtggctccggcggaggtgggtccggtggcggcggatcaggtgggggtggatcaggcggtggaggttccggtggcgggggatcaagaaagagaaggaaaagaattgtggggggcaaggtgtgccccaaaggggagtgtccatggcaggtcctgttgttggtgaatggagctcagttgtgtggggggaccctgatcaacaccatctgggtggtgtccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctcaggtggcggaggttcccatcatcaccatcaccattga

SEQ ID NO: 271 - DNA, 2418 bp, Artificial Sequence
  DNA sequence of FVII-231
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccaggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctcaggtggcggaggttcccatcatcaccatcaccattga

SEQ ID NO: 272 - DNA, 2634 bp, Artificial Sequence
  DNA sequence of FVII-232
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctcaggtggcggaggttcccatcatcaccatcaccattga

SEQ ID NO: 273 - DNA, 2751 bp, Artificial Sequence
  DNA sequence of FVII-242
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctacggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggtggcggcggatcaggtgggggtggatcaggcggtggaggttccggtggcgggggatccggcggtggaggttccggtgggggtggatcaggctcgagtggtacctcagagtctgctacccccgagtcagggccaggatcagagccagccacctccgggtctgagacacccgggacttccgagagtgccacccctgagtccggacccgggtccgagcccgccactaccggtggtggcggtggctccggcggaggtgggtccggtggcggcggatcaggtgggggtggatcaggcggtggaggttccggtggcgggggatcaagaaagagaaggaaaagaattgtggggggcaaggtgtgccccaaaggggagtgtccatggcaggtcctgttgttggtgaatggagctcagttgtgtggggggaccctgatcaacaccatctgggtggtgtccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccaggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctcaggtggcggaggttcccatcatcaccatcaccattga

SEQ ID NO: 274 - DNA, 2751 bp, Artificial Sequence
  DNA sequence of FVII-243
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctacggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggtggcggcggatcaggtgggggtggatcaggcggtggaggttccggtggcgggggatccggcggtggaggttccggtgggggtggatcaggctcgagtagtcccgctggaagcccaactagcaccgaagaggggacctcagagtccgccacccccgagtccggccctggctctgagcctgccactagcggctccgagactcctggcacatccgaaagcgctacacccgagagtggacccggcacctctaccgagcccagtgagggctccgcccctggaacaagcaccgagcccagcgaaggcagcgccccagggaccggtggtggcggtggctccggcggaggtgggtccggtggcggcggatcaggtgggggtggatcaggcggtggaggttccggtggcgggggatcaagaaagagaaggaaaagaattgtggggggcaaggtgtgccccaaaggggagtgtccatggcaggtcctgttgttggtgaatggagctcagttgtgtggggggaccctgatcaacaccatctgggtggtgtccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccactggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctcaggtggcggaggttcccatcatcaccatcaccattga

SEQ ID NO: 275 - DNA, 2841 bp, Artificial Sequence
  DNA sequence of FVII-238
atggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctacggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggtggcggcggatcaggtgggggtggatcaggcggtggaggttccggtggcgggggatccggcggtggaggttccggtgggggtggatcaggctcgagtagtcccgctggaagcccaactagcaccgaagaggggacctcagagtccgccacccccgagtccggccctggctctgagcctgccactagcggctccgagactcctggcacatccgaaagcgctacacccgagagtggacccggcacctctaccgagcccagtgagggctccgcccctggaacaagcaccgagcccagcgaaggcagcgccccagggaccggtggtggcggtggctccggcggaggtgggtccggtggcggcggatcaggtgggggtggatcaggcggtggaggttccggtggcgggggatcaagaaagagaaggaaaagaattgtggggggcaaggtgtgccccaaaggggagtgtccatggcaggtcctgttgttggtgaatggagctcagttgtgtggggggaccctgatcaacaccatctgggtggtgtccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtgtcccagtacatcgagtggctgcaaaagctcatgcgctcagagccacgcccaggagtcctcctgcgagccccatttcccgggtctccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccaggtggcggtggctccggcggaggtgggtccggtggcggcggatcaggtgggggtggatcaggcggtggaggttccggtggcgggggatccgaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttagtagttccccgtggacgttcggtggaggcaccaagctggaaatcaaacgcggtggcggcggatcaggtggaggtggatcaggtggcggtggcagtggtggcggtggatcagaagtgaagctggtggagtctgggggaggcttagtgaagcctggagggtccctgaaactctcctgtgcagcctctggattcactttcagtgcctatgccatgtcttgggttcgccagactccagagaagaggctggagtgggtcgcatccattagtagtggtggtaccacctactatccagacagtgtgaaacgccgattcaccatctccagagataatgccaggaacatcctgtacctgcaaatgagcagtctgaggtctgaggacacggccatgtattactgtacaagaggcggggactacggctacgctctggactactggggtcaaggaacctcagtcaccgtctcctcaggtggcggaggttcccatcatcaccatcaccattga